SEQUENCE LISTING (421 sequences)

SEQ ID NO: 1 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCQHYGSPPSFTFGQGTKVEIK

SEQ ID NO: 2 - protein, 119 aa, Artificial Sequence
  source: /note=“Description of Artificial Sequence Synthetic polypeptide”
EVQLLESGGGLVQPGGSLRLSCAASGFTFGSYAMTWVRQAPGKGLEWVSAISGSGGNTFYADSVKGRFTISRDNSKNTLYLQMNSLRAEDTAVYYCARVSPIASGMDYWGQGTLVTVSS

SEQ ID NO: 3 - protein, 119 aa, Artificial Sequence
  source: /note=“Description of Artificial Sequence Synthetic polypeptide”
EVQLLESGGGLVQPGGSLRLSCAASGFTFGSYAMTWVRQAPGKGLEWVSAISGSGGNTFYADSVKGRFTISRDNSKNTLYLQMNSLRAEDTAVYYCARVSPIAAQMDYWGQGTLVTVSS

SEQ ID NO: 4 - protein, 109 aa, Artificial Sequence
  source: /note=“Description of Artificial Sequence Synthetic polypeptide”
EIVLTQSPGTLSLSPGERATLSCRASQSLGSFYLAWYQQKPGQAPRLLIYGASSRATGIPDRFSGSGSGTDFTLTISRLEPEDFAVYYCKHYGWPPSFTFGQGTKVEIK

SEQ ID NO: 5 - protein, 109 aa, Artificial Sequence
  source: /note=“Description of Artificial Sequence Synthetic polypeptide”
EIVLTQSPGTLSLSPGERATLSCRASQSLGSFYLAWYQQKPGQAPRLLIYGASSRATGIPDRFSGSGSGTDFTLTISRLEPEDFAVYYCQHYNYPPSFTFGQGTKVEIK

SEQ ID NO: 6 - protein, 109 aa, Artificial Sequence
  source: /note=“Description of Artificial Sequence Synthetic polypeptide”
EIVLTQSPGTLSLSPGERATLSCRASQSVGDFYLAWYQQKPGQAPRLLIYGASSRATGIPDRFSGSGSGTDFTLTISRLEPEDFAVYYCQHYNYPPSFTFGQGTKVEIK

SEQ ID NO: 7 - protein, 121 aa, Artificial Sequence
  source: /note=“Description of Artificial Sequence Synthetic polypeptide”
EVQLLESGGGLVQPGGSLRLSCAASGFTFGSYAMTWVRQAPGKGLEWVSAISGSGGNTFYADSVKGRFTISRDNSKNTLYLQMNSLRAEDTAVYYCARARVSPIAALMDYWGQGTLVTVSS

SEQ ID NO: 8 - protein, 119 aa, Artificial Sequence
  source: /note=“Description of Artificial Sequence Synthetic polypeptide”
EVQLLESGGGLVQPGGSLRLSCAASGFTFGSYAMTWVRQAPGKGLEWVSAISGSGGNTFYADSVKGRFTISRDNSKNTLYLQMNSLRAEDTAVYYCARVSPIAAPMDYWGQGTLVTVSS

SEQ ID NO: 9 - protein, 109 aa, Artificial Sequence
  source: /note=“Description of Artificial Sequence Synthetic polypeptide”
EIVLTQSPGTLSLSPGERATLSCRASQSLGSFYLAWYQQKPGQAPRLLIYGASSRATGIPDRFSGSGSGTDFTLTISRLEPEDFAVYYCQHYPYPPSFTFGQGTKVEIK

SEQ ID NO: 10 - protein, 109 aa, Artificial Sequence; identical to SEQ ID NO: 5
  source: /note=“Description of Artificial Sequence Synthetic polypeptide”

SEQ ID NO: 11 - protein, 109 aa, Artificial Sequence
  source: /note=“Description of Artificial Sequence Synthetic polypeptide”
EIVLTQSPGTLSLSPGERATLSCRASQSVGDFYLAWYQQKPGQAPRLLIYGASSRATGIPDRFSGSGSGTDFTLTISRLEPEDFAVYYCKHYGWPPSFTFGQGTKVEIK

SEQ ID NO: 12 - protein, 109 aa, Artificial Sequence
  source: /note=“Description of Artificial Sequence Synthetic polypeptide”
EIVLTQSPGTLSLSPGERATLSCRASQSVGDFYLAWYQQKPGQAPRLLIYGASSRATGIPDRFSGSGSGTDFTLTISRLEPEDFAVYYCQHYPYPPSFTFGQGTKVEIK

SEQ ID NO: 13 - protein, 109 aa, Artificial Sequence; identical to SEQ ID NO: 6
  source: /note=“Description of Artificial Sequence Synthetic polypeptide”

SEQ ID NO: 14 - protein, 109 aa, Artificial Sequence; identical to SEQ ID NO: 6
  source: /note=“Description of Artificial Sequence Synthetic polypeptide”

SEQ ID NO: 15 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCKHYGWPPSFTFGQGTKVEIK

SEQ ID NO: 16 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCQHYPYPPSFTFGQGTKVEIK

SEQ ID NO: 17 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCQHYNYPPSFTFGQGTKVEIK

SEQ ID NO: 18 - protein, 109 aa, Artificial Sequence
  source: /note=“Description of Artificial Sequence Synthetic polypeptide”
EIVLTQSPGTLSLSPGERATLSCRASQSVSSSYPSWYQQKPGQAPRLLIYGASSRATGIPDRFSGSGSGTDFTLTISRLEPEDFAVYYCQHYPYPPSFTFGQGTKVEIK

SEQ ID NO: 19 - protein, 109 aa, Artificial Sequence
  source: /note=“Description of Artificial Sequence Synthetic polypeptide”
EIVLTQSPGTLSLSPGERATLSCRASQSVSAHYLAWYQQKPGQAPRLLIYGASSRATGIPDRFSGSGSGTDFTLTISRLEPEDFAVYYCQHYPYPPSFTFGQGTKVEIK

SEQ ID NO: 20 - protein, 109 aa, Artificial Sequence
  source: /note=“Description of Artificial Sequence Synthetic polypeptide”
EIVLTQSPGTLSLSPGERATLSCRASQSVSSFFLAWYQQKPGQAPRLLIYGASSRATGIPDRFSGSGSGTDFTLTISRLEPEDFAVYYCQHYPYPPSFTFGQGTKVEIK

SEQ ID NO: 21 - protein, 109 aa, Artificial Sequence
  source: /note=“Description of Artificial Sequence Synthetic polypeptide”
EIVLTQSPGTLSLSPGERATLSCRASQSVSPHYLAWYQQKPGQAPRLLIYGASSRATGIPDRFSGSGSGTDFTLTISRLEPEDFAVYYCQHYPYPPSFTFGQGTKVEIK

SEQ ID NO: 22 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCKYYPYPPSFTFGQGTKVEIK

SEQ ID NO: 23 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRATGIPDRFSGSGSGTDFTLTISRLEPEDFAVYYCKFYPYPPSFTFGQGTKVEIK

SEQ ID NO: 24 - protein, 119 aa, Artificial Sequence
  source: /note=“Description of Artificial Sequence Synthetic polypeptide”
EVQLLESGGGLVQPGGSLRLSCAASGFTFGSYAMTWVRQAPGKGLEWVSAISGSGGNTFYADQRKGRFTISRDNSKNTLYLQMNSLRAEDTAVYYCARVSPIASGMDYWGQGTLVTVSS

SEQ ID NO: 25 - protein, 119 aa, Artificial Sequence
  source: /note=“Description of Artificial Sequence Synthetic polypeptide”
EVQLLESGGGLVQPGGSLRLSCAASGFTFGSYAMTWVRQAPGKGLEWVSAIDYSGGNTFYADSVKGRFTISRDNSKNTLYLQMNSLRAEDTAVYYCARVSPIASGMDYWGQGTLVTVSS

SEQ ID NO: 26 - protein, 119 aa, Artificial Sequence
  source: /note=“Description of Artificial Sequence Synthetic polypeptide”
EVQLLESGGGLVQPGGSLRLSCAASGFTFGSYAMTWVRQAPGKGLEWVSAISYQGGNTFYADSVKGRFTISRDNSKNTLYLQMNSLRAEDTAVYYCARVSPIASGMDYWGQGTLVTVSS

SEQ ID NO: 27 - protein, 119 aa, Artificial Sequence
  source: /note=“Description of Artificial Sequence Synthetic polypeptide”
EVQLLESGGGLVQPGGSLRLSCAASGFTFGSYAMTWVRQAPGKGLEWVSAISLTGGNTFYADSVKGRFTISRDNSKNTLYLQMNSLRAEDTAVYYCARVSPIASGMDYWGQGTLVTVSS

SEQ ID NO: 28 - protein, 119 aa, Artificial Sequence
  source: /note=“Description of Artificial Sequence Synthetic polypeptide”
EVQLLESGGGLVQPGGSLRLSCAASGFTFGSYAMTWVRQAPGKGLEWVSAISHAGGNTFYADSVKGRFTISRDNSKNTLYLQMNSLRAEDTAVYYCARVSPIASGMDYWGQGTLVTVSS

SEQ ID NO: 29 - protein, 119 aa, Artificial Sequence
  source: /note=“Description of Artificial Sequence Synthetic polypeptide”
EVQLLESGGGLVQPGGSLRLSCAASGFTFGSYAMTWVRQAPGKGLEWVSAISGSGGNTFYADQLKGRFTISRDNSKNTLYLQMNSLRAEDTAVYYCARVSPIASGMDYWGQGTLVTVSS

SEQ ID NO: 30 - protein, 119 aa, Artificial Sequence
  source: /note=“Description of Artificial Sequence Synthetic polypeptide”
EVQLLESGGGLVQPGGSLRLSCAASGFTFGSYAMTWVRQAPGKGLEWVSAISGSGGNTFYADSVKGRFTISRDNSKNTLYLQMNSLRAEDTAVYYCARVSPIYAGMDYWGQGTLVTVSS

SEQ ID NO: 31 - protein, 119 aa, Artificial Sequence
  source: /note=“Description of Artificial Sequence Synthetic polypeptide”
EVQLLESGGGLVQPGGSLRLSCAASGFTFGSYAMTWVRQAPGKGLEWVSAISGSGGNTFYADSVKGRFTISRDNSKNTLYLQMNSLRAEDTAVYYCARVSPIAAEMDYWGQGTLVTVSS

SEQ ID NO: 32 - protein, 119 aa, Artificial Sequence
  source: /note=“Description of Artificial Sequence Synthetic polypeptide”
EVQLLESGGGLVQPGGSLRLSCAASGFTFGSYAMTWVRQAPGKGLEWVSAISGSGGNTFYADSVKGRFTISRDNSKNTLYLQMNSLRAEDTAVYYCTRVSPIAAQMDYWGQGTLVTVSS

SEQ ID NO: 33 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAISDSGGSTYYADSVKGRFTISRDNSKNTLYLQMNSLRAEDTAVYYCARYWPMDIWGQGTLVTVSS

SEQ ID NO: 34 - protein, 108 aa, Artificial Sequence
  source: /note=“Description of Artificial Sequence Synthetic polypeptide”
EIVLTQSPGTLSLSPGERATLSCRASQSVSSSYLAWYQQKPGQAPRLLMYDASIRATGIPDRFSGSGSGTDFTLTISRLEPEDFAVYYCQQYGSWPLTFGQGTKVEIK

SEQ ID NO: 35 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAISDSGGSAWYADSVKGRFTISRDNSKNTLYLQMNSLRAEDTAVYYCARYWPMSLWGQGTLVTVSS

SEQ ID NO: 36 - protein, 108 aa, Artificial Sequence
  source: /note=“Description of Artificial Sequence Synthetic polypeptide”
EIVLTQSPGTLSLSPGERATLSCRASQSVSVLYLAWYQQKPGQAPRLLMYDASIRATGIPDRFSGSGSGTDFTLTISRLEPEDFAVYYCQQYQRWPLTFGQGTKVEIK

SEQ ID NO: 37 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAISDSGGSMWYADSVKGRFTISRDNSKNTLYLQMNSLRAEDTAVYYCARYWPMSLWGQGTLVTVSS

SEQ ID NO: 38 - protein, 108 aa, Artificial Sequence
  source: /note=“Description of Artificial Sequence Synthetic polypeptide”
EIVLTQSPGTLSLSPGERATLSCRASQSVSSSYLAWYQQKPGQAPRLLMYDASIRATGIPDRFSGSGSGTDFTLTISRLEPEDFAVYYCQQYQSWPLTFGQGTKVEIK

SEQ ID NO: 39 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAISDFGGSTYYADSVKGRFTISRDNSKNTLYLQMNSLRAEDTAVYYCARYWPMDIWGQGTLVTVSS

SEQ ID NO: 40 - protein, 108 aa, Artificial Sequence
  source: /note=“Description of Artificial Sequence Synthetic polypeptide”
EIVLTQSPGTLSLSPGERATLSCRASQSVSDLYLAWYQQKPGQAPRLLMYDASIRATGIPDRFSGSGSGTDFTLTISRLEPEDFAVYYCQQYQTWPLTFGQGTKVEIK

SEQ ID NO: 41 - protein, 108 aa, Artificial Sequence
  source: /note=“Description of Artificial Sequence Synthetic polypeptide”
EIVLTQSPGTLSLSPGERATLSCRASQSVSNLYLAWYQQKPGQAPRLLMYDASIRATGIPDRFSGSGSGTDFTLTISRLEPEDFAVYYCQQYQGWPLTFGQGTKVEIK

SEQ ID NO: 42 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAITASGGSTYYADSVKGRFTISRDNSKNTLYLQMNSLRAEDTAVYYCARYWPMSLWGQGTLVTVSS

SEQ ID NO: 43 - protein, 108 aa, Artificial Sequence
  source: /note=“Description of Artificial Sequence Synthetic polypeptide”
EIVLTQSPGTLSLSPGERATLSCRASQSVSAYYLAWYQQKPGQAPRLLMYDASIRATGIPDRFSGSGSGTDFTLTISRLEPEDFAVYYCQQYERWPLTFGQGTKVEIK

SEQ ID NO: 44 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAISDSGGSTYYADSVKGRFTISRDNSKNTLYLQMNSLRAEDTAVYYCARYWPMSLWGQGTLVTVSS

SEQ ID NO: 45 - protein, 108 aa, Artificial Sequence
  source: /note=“Description of Artificial Sequence Synthetic polypeptide”
EIVLTQSPGTLSLSPGERATLSCRASQSVSSLYLAWYQQKPGQAPRLLMYDASIRATGIPDRFSGSGSGTDFTLTISRLEPEDFAVYYCQQYQVWPLTFGQGTKVEIK

SEQ ID NO: 46 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAISDSGGSRWYADSVKGRFTISRDNSKNTLYLQMNSLRAEDTAVYYCARYWPMTPWGQGTLVTVSS

SEQ ID NO: 47 - protein, 108 aa, Artificial Sequence
  source: /note=“Description of Artificial Sequence Synthetic polypeptide”
EIVLTQSPGTLSLSPGERATLSCRASQSVSSSYLAWYQQKPGQAPRLLMYDASIRATGIPDRFSGSGSGTDFTLTISRLEPEDFAVYYCQQYLDWPLTFGQGTKVEIK

SEQ ID NO: 48 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAVLDSGGSTYYADSVKGRFTISRDNSKNTLYLQMNSLRAEDTAVYYCARYWPMTPWGQGTLVTVSS

SEQ ID NO: 49 - protein, 108 aa, Artificial Sequence
  source: /note=“Description of Artificial Sequence Synthetic polypeptide”
EIVLTQSPGTLSLSPGERATLSCRASQSVSSSYLAWYQQKPGQAPRLLMYDASIRATGIPDRFSGSGSGTDFTLTISRLEPEDFAVYYCQQYQVWPLTFGQGTKVEIK

SEQ ID NO: 50 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAISDSGGSRWYADSVKGRFTISRDNSKNTLYLQMNSLRAEDTAVYYCARYWPMSDWGQGTLVTVSS

SEQ ID NO: 51 - protein, 108 aa, Artificial Sequence
  source: /note=“Description of Artificial Sequence Synthetic polypeptide”
EIVLTQSPGTLSLSPGERATLSCRASQSVSVLYLAWYQQKPGQAPRLLMYDASIRATGIPDRFSGSGSGTDFTLTISRLEPEDFAVYYCQQYLAWPLTFGQGTKVEIK

SEQ ID NO: 52 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAISDSGGSKWYADSVKGRFTISRDNSKNTLYLQMNSLRAEDTAVYYCARYWPMSLWGQGTLVTVSS

SEQ ID NO: 53 - protein, 108 aa, Artificial Sequence
  source: /note=“Description of Artificial Sequence Synthetic polypeptide”
EIVLTQSPGTLSLSPGERATLSCRASQSVSSSYLAWYQQKPGQAPRLLMYDASIRATGIPDRFSGSGSGTDFTLTISRLEPEDFAVYYCQQYFTWPLTFGQGTKVEIK

SEQ ID NO: 54 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAIGGSGGSLPYADSVKGRFTISRDNSKNTLYLQMNSLRAEDTAVYYCARYWPMDSWGQGTLVTVSS

SEQ ID NO: 55 - protein, 108 aa, Artificial Sequence
  source: /note=“Description of Artificial Sequence Synthetic polypeptide”
EIVLTQSPGTLSLSPGERATLSCRASQSVSPYYLAWYQQKPGQAPRLLMYDASIRATGIPDRFSGSGSGTDFTLTISRLEPEDFAVYYCQQYERWPLTFGQGTKVEIK

SEQ ID NO: 56 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAISDSGGSGWYADSVKGRFTISRDNSKNTLYLQMNSLRAEDTAVYYCARYWPMSLWGQGTLVTVSS

SEQ ID NO: 57 - protein, 108 aa, Artificial Sequence
  source: /note=“Description of Artificial Sequence Synthetic polypeptide”
EIVLTQSPGTLSLSPGERATLSCRASQSVSVEYLAWYQQKPGQAPRLLMYDASIRATGIPDRFSGSGSGTDFTLTISRLEPEDFAVYYCQQYARWPLTFGQGTKVEIK

SEQ ID NO: 58 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAVLDSGGSTYYADSVKGRFTISRDNSKNTLYLQMNSLRAEDTAVYYCARYWPMSLWGQGTLVTVSS

SEQ ID NO: 59 - protein, 108 aa, Artificial Sequence
  source: /note=“Description of Artificial Sequence Synthetic polypeptide”
EIVLTQSPGTLSLSPGERATLSCRASQSVSELYLAWYQQKPGQAPRLLMYDASIRATGIPDRFSGSGSGTDFTLTISRLEPEDFAVYYCQQYFGWPLTFGQGTKVEIK

SEQ ID NO: 60 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAISDSGGSCWYADSVKGRFTISRDNSKNTLYLQMNSLRAEDTAVYYCARYWPMTPWGQGTLVTVSS

SEQ ID NO: 61 - protein, 108 aa, Artificial Sequence
  source: /note=“Description of Artificial Sequence Synthetic polypeptide”
EIVLTQSPGTLSLSPGERATLSCRASQSVEMSYLAWYQQKPGQAPRLLMYDASIRATGIPDRFSGSGSGTDFTLTISRLEPEDFAVYYCQQYAHWPLTFGQGTKVEIK

SEQ ID NO: 62 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAIFASGGSTYYADSVKGRFTISRDNSKNTLYLQMNSLRAEDTAVYYCARYWPMTPWGQGTLVTVSS

SEQ ID NO: 63 - protein, 108 aa, Artificial Sequence
  source: /note=“Description of Artificial Sequence Synthetic polypeptide”
EIVLTQSPGTLSLSPGERATLSCRASQSVSSSYLAWYQQKPGQAPRLLMYDASIRATGIPDRFSGSGSGTDFTLTISRLEPEDFAVYYCQQYQRWPLTFGQGTKVEIK

SEQ ID NO: 64 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAISGWGGSLPYADSVKGRFTISRDNSKNTLYLQMNSLRAEDTAVYYCARYWPMDSWGQGTLVTVSS

SEQ ID NO: 65 - protein, 108 aa, Artificial Sequence
  source: /note=“Description of Artificial Sequence Synthetic polypeptide”
EIVLTQSPGTLSLSPGERATLSCRASQSVSAQYLAWYQQKPGQAPRLLMYDASIRATGIPDRFSGSGSGTDFTLTISRLEPEDFAVYYCQQYQRWPLTFGQGTKVEIK

SEQ ID NO: 66 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAIMSSGGPLYYADSVKGRFTISRDNSKNTLYLQMNSLRAEDTAVYYCARYWPMALWGQGTLVTVSS

SEQ ID NO: 67 - protein, 108 aa, Artificial Sequence
  source: /note=“Description of Artificial Sequence Synthetic polypeptide”
EIVLTQSPGTLSLSPGERATLSCRASQSVSALYLAWYQQKPGQAPRLLMYDASIRATGIPDRFSGSGSGTDFTLTISRLEPEDFAVYYCQQYQVWPLTFGQGTKVEIK

SEQ ID NO: 68 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAILMSGGSTYYADSVKGRFTISRDNSKNTLYLQMNSLRAEDTAVYYCARYWPMSLWGQGTLVTVSS

SEQ ID NO: 69 - protein, 108 aa, Artificial Sequence
  source: /note=“Description of Artificial Sequence Synthetic polypeptide”
EIVLTQSPGTLSLSPGERATLSCGPSQSVSSSYLAWYQQKPGQAPRLLMYDASIRATGIPDRFSGSGSGTDFTLTISRLEPEDFAVYYCQQYQSWPLTFGQGTKVEIK

SEQ ID NO: 70 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAISDSGGYRYYADSVKGRFTISRDNSKNTLYLQMNSLRAEDTAVYYCARYWPMSLWGQGTLVTVSS

SEQ ID NO: 71 - protein, 108 aa, Artificial Sequence
  source: /note=“Description of Artificial Sequence Synthetic polypeptide”
EIVLTQSPGTLSLSPGERATLSCRASQSVSSSYWAWYQQKPGQAPRLLMYDASIRATGIPDRFSGSGSGTDFTLTISRLEPEDFAVYYCQQYESWPLTFGQGTKVEIK

SEQ ID NO: 72 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSS

SEQ ID NO: 73 - protein, 108 aa, Artificial Sequence
  source: /note=“Description of Artificial Sequence Synthetic polypeptide”
EIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIK

SEQ ID NO: 74 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAILDSGGSTYYADSVKGRFTISRDNSKNTLYLQMNSLRAEDTAVYYCARYWPMSPWGQGTLVTVSS

SEQ ID NO: 75 - protein, 108 aa, Artificial Sequence
  source: /note=“Description of Artificial Sequence Synthetic polypeptide”
EIVLTQSPGTLSLSPGERATLSCRASQSVSFLYLAWYQQKPGQAPRLLMYDASIRATGIPDRFSGSGSGTDFTLTISRLEPEDFAVYYCQQYGSWPLTFGQGTKVEIK

SEQ ID NO: 76 - protein, 115 aa, Artificial Sequence
  source: /note=“Description of Artificial Sequence Synthetic polypeptide”
EVQLLESGGGLVQPGGSLRLSCAASGFTFSSYPMSWVRQAPGKGLEWVSAIGGSGGSTYYADSVKGRFTISRDNSKNTLYLQMNSLRAEDTAVYYCARYWPMDSWGQGTLVTVSS

SEQ ID NO: 77 - protein, 108 aa, Artificial Sequence
  source: /note=“Description of Artificial Sequence Synthetic polypeptide”
EIVLTQSPGTLSLSPGERATLSCRASQSVSSTYLAWYQQKPGQAPRLLIYDASSRAPGIPDRFSGSGSGTDFTLTISRLEPEDFAVYYCQQYSTSPLTFGQGTKVEIK

SEQ ID NO: 78 - protein, 115 aa, Artificial Sequence
  source: /note=“Description of Artificial Sequence Synthetic polypeptide”
EVQLLESGGGLVQPGGSLRLSCAASGFTFSSYPMSWVRQAPGKGLEWVSAIGGSGGSLPYADSVKGRFTISRDNSKNTLYLQMNSLRAEDTAVYYCARYWPMDSWGQGTLVTVSS

SEQ ID NO: 79 - protein, 108 aa, Artificial Sequence
  source: /note=“Description of Artificial Sequence Synthetic polypeptide”
EIVLTQSPGTLSLSPGERATLSCRASQSVSPEYLAWYQQKPGQAPRLLIYDASSRAPGIPDRFSGSGSGTDFTLTISRLEPEDFAVYYCQQYSVWPLTFGQGTKVEIK

SEQ ID NO: 80 - protein, 108 aa, Artificial Sequence
  source: /note=“Description of Artificial Sequence Synthetic polypeptide”
EIVLTQSPGTLSLSPGERATLSCRASQLGSFYLAWYQQKPGQAPRLLIYGASSRATGIPDRFSGSGSGTDFTLTISRLEPEDFAVYYCQHYNYPPSFTFGQGTKVEIK

SEQ ID NO: 81 - protein, 108 aa, Artificial Sequence
  source: /note=“Description of Artificial Sequence Synthetic polypeptide”
EIVLTQSPGTLSLSPGERATLSCRASQSVSSTYLAWYQQKPGQAPRLLIYDASSRAPGIPDRFSGSGSGTDFTLTISRLEPEDFAVYYCQQYSRWPLTFGQGTKVEIK

SEQ ID NO: 82 - protein, 108 aa, Artificial Sequence
  source: /note=“Description of Artificial Sequence Synthetic polypeptide”
EIVLTQSPGTLSLSPGERATLSCRASQSVSPLYLAWYQQKPGQAPRLLIYDASSRAPGIPDRFSGSGSGTDFTLTISRLEPEDFAVYYCQQYSAFPLTFGQGTKVEIK

SEQ ID NO: 83 - protein, 115 aa, Artificial Sequence
  source: /note=“Description of Artificial Sequence Synthetic polypeptide”
EVQLLESGGGLVQPGGSLRLSCAASGFTFSSYPMSWVRQAPGKGLEWVSAIGGSGGWSYYADSVKGRFTISRDNSKNTLYLQMNSLRAEDTAVYYCARYWPMDSWGQGTLVTVSS

SEQ ID NO: 84 - protein, 108 aa, Artificial Sequence
  source: /note=“Description of Artificial Sequence Synthetic polypeptide”
EIVLTQSPGTLSLSPGERATLSCWLSQSVSSTYLAWYQQKPGQAPRLLIYDASSRAPGIPDRFSGSGSGTDFTLTISRLEPEDFAVYYCQQYSEWPLTFGQGTKVEIK

SEQ ID NO: 85 - protein, 108 aa, Artificial Sequence
  source: /note=“Description of Artificial Sequence Synthetic polypeptide”
EIVLTQSPGTLSLSPGERATLSCRASQSVSSTYLAWYQQKPGQAPRLLIYDASSRAPGIPDRFSGSGSGTDFTLTISRLEPEDFAVYYCQQYSSWPLTFGQGTKVEIK

SEQ ID NO: 86 - protein, 108 aa, Artificial Sequence
  source: /note=“Description of Artificial Sequence Synthetic polypeptide”
EIVLTQSPGTLSLSPGERATLSCRASQSVSSLFLAWYQQKPGQAPRLLIYDASSRAPGIPDRFSGSGSGTDFTLTISRLEPEDFAVYYCQQYSAWPLTFGQGTKVEIK

SEQ ID NO: 87 - protein, 115 aa, Artificial Sequence
  source: /note=“Description of Artificial Sequence Synthetic polypeptide”
EVQLLESGGGLVQPGGSLRLSCAASGFTFSSYPMSWVRQAPGKGLEWVSATVGSGGSIGYADSVKGRFTISRDNSKNTLYLQMNSLRAEDTAVYYCARYWPMDSWGQGTLVTVSS

SEQ ID NO: 88 - protein, 108 aa, Artificial Sequence
  source: /note=“Description of Artificial Sequence Synthetic polypeptide”
EIVLTQSPGTLSLSPGERATLSCACSQSVSSTYLAWYQQKPGQAPRLLIYDASSRAPGIPDRFSGSGSGTDFTLTISRLEPEDFAVYYCQQYSAWPLTFGQGTKVEIK

SEQ ID NO: 89 - protein, 108 aa, Artificial Sequence
  source: /note=“Description of Artificial Sequence Synthetic polypeptide”
EIVLTQSPGTLSLSPGERATLSCRASCDVSSTYLAWYQQKPGQAPRLLIYDASSRAPGIPDRFSGSGSGTDFTLTISRLEPEDFAVYYCQQYMRSPLTFGQGTKVEIK

SEQ ID NO: 90 - protein, 108 aa, Artificial Sequence
  source: /note=“Description of Artificial Sequence Synthetic polypeptide”
EIVLTQSPGTLSLSPGERATLSCRASEAVPSTYLAWYQQKPGQAPRLLIYDASSRAPGIPDRFSGSGSGTDFTLTISRLEPEDFAVYYCQQYSAFPLTFGQGTKVEIK

SEQ ID NO: 91 - protein, 108 aa, Artificial Sequence
  source: /note=“Description of Artificial Sequence Synthetic polypeptide”
EIVLTQSPGTLSLSPGERATLSCCSSQSVSSTYLAWYQQKPGQAPRLLIYDASSRAPGIPDRFSGSGSGTDFTLTISRLEPEDFAVYYCQQYSAFPLTFGQGTKVEIK

SEQ ID NO: 92 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAISDSGGSRWYADSVKGRFTISRDNSKNTLYLQMNSLRAEDTAVYYCTRYWPMDIWGQGTLVTVSS

SEQ ID NO: 93 - protein, 108 aa, Artificial Sequence
  source: /note=“Description of Artificial Sequence Synthetic polypeptide”
EIVLTQSPGTLSLSPGERATLSCRASVRVSSTYLAWYQQKPGQAPRLLMYDASIRATGIPDRFSGSGSGTDFTLTISRLEPEDFAVYYCQQYMKWPLTFGQGTKVEIK

SEQ ID NO: 94 - protein, 108 aa, Artificial Sequence
  source: /note=“Description of Artificial Sequence Synthetic polypeptide”
EIVLTQSPGTLSLSPGERATLSCRASQSVSAAYLAWYQQKPGQAPRLLMYDASIRATGIPDRFSGSGSGTDFTLTISRLEPEDFAVYYCQQYMCWPLTFGQGTKVEIK

SEQ ID NO: 95 - protein, 115 aa, Artificial Sequence
  source: /note=“Description of Artificial Sequence Synthetic polypeptide”
EVQLLESGGGLVQPGGSLRLSCAASGFTFSSYPMSWVRQAPGKGLEWVSAIGGSGGSIHYADSVKGRFTISRDNSKNTLYLQMNSLRAEDTAVYYCARYWPMDSWGQGTLVTVSS

SEQ ID NO: 96 - protein, 108 aa, Artificial Sequence
  source: /note=“Description of Artificial Sequence Synthetic polypeptide”
EIVLTQSPGTLSLSPGERATLSCRASQSVSSSYWGWYQQKPGQAPRLLMYDASIRATGIPDRFSGSGSGTDFTLTISRLEPEDFAVYYCQQYQCWPLTFGQGTKVEIK

SEQ ID NO: 97 - protein, 115 aa, Artificial Sequence
  source: /note=“Description of Artificial Sequence Synthetic polypeptide”
EVQLLESGGGLVQPGGSLRLSCAASGFTFSSYPMSWVRQAPGKGLEWVSAHIGSGGSTYYADSVKGRFTISRDNSKNTLYLQMNSLRAEDTAVYYCARYWPMDSWGQGTLVTVSS

SEQ ID NO: 98 - protein, 108 aa, Artificial Sequence
  source: /note=“Description of Artificial Sequence Synthetic polypeptide”
EIVLTQSPGTLSLSPGERATLSCRASQSVSSTYLAWYQQKPGQAPRLLMYDASIRATGIPDRFSGSGSGTDFTLTISRLEPEDFAVYYCQQYQSWPLTFGQGTKVEIK

SEQ ID NO: 99 - protein, 115 aa, Artificial Sequence
  source: /note=“Description of Artificial Sequence Synthetic polypeptide”
EVQLLESGGGLVQPGGSLRLSCAASGFTFSSYPMSWVRQAPGKGLEWVSAIGGSGGSTYYADSVKGRFTISRDNSKNTLYLQMNSLRAEDTAVYYCARYWPMDPWGQGTLVTVSS

SEQ ID NO: 100 - protein, 108 aa, Artificial Sequence
  source: /note=“Description of Artificial Sequence Synthetic polypeptide”
EIVLTQSPGTLSLSPGERATLSCRASQSVSSPYLAWYQQKPGQAPRLLMYDASIRATGIPDRFSGSGSGTDFTLTISRLEPEDFAVYYCQQYQSWPLTFGQGTKVEIK

SEQ ID NO: 101 - protein, 115 aa, Artificial Sequence
  source: /note=“Description of Artificial Sequence Synthetic polypeptide”
EVQLLESGGGLVQPGGSLRLSCAASGFTFSSYPMSWVRQAPGKGLEWVSAIGGSGGSLGYADSVKGRFTISRDNSKNTLYLQMNSLRAEDTAVYYCARYWPMDSWGQGTLVTVSS

SEQ ID NO: 102 - protein, 108 aa, Artificial Sequence
  source: /note=“Description of Artificial Sequence Synthetic polypeptide”
EIVLTQSPGTLSLSPGERATLSCRASQSVSPLYLAWYQQKPGQAPRLLMYDASIRATGIPDRFSGSGSGTDFTLTISRLEPEDFAVYYCQQYKAWPLTFGQGTKVEIK

SEQ ID NO: 103 - protein, 108 aa, Artificial Sequence
  source: /note=“Description of Artificial Sequence Synthetic polypeptide”
EIVLTQSPGTLSLSPGERATLSCRASQSVSYLYLAWYQQKPGQAPRLLMYDASIRATGIPDRFSGSGSGTDFTLTISRLEPEDFAVYYCQQYMEWPLTFGQGTKVEIK

SEQ ID NO: 104 - protein, 115 aa, Artificial Sequence
  source: /note=“Description of Artificial Sequence Synthetic polypeptide”
EVQLLESGGGLVQPGGSLRLSCAASGFTFSSYPMSWVRQAPGKGLEWVSAIFASGGSTYYADSVKGRFTISRDNSKNTLYLQMNSLRAEDTAVYYCARYWPMDSWGQGTLVTVSS

SEQ ID NO: 105 - protein, 108 aa, Artificial Sequence
  source: /note=“Description of Artificial Sequence Synthetic polypeptide”
EIVLTQSPGTLSLSPGERATLSCRASQSVSAQYLAWYQQKPGQAPRLLMYDASIRATGIPDRFSGSGSGTDFTLTISRLEPEDFAVYYCQQYQAWPLTFGQGTKVEIK

SEQ ID NO: 106 - protein, 115 aa, Artificial Sequence
  source: /note=“Description of Artificial Sequence Synthetic polypeptide”
EVQLLESGGGLVQPGGSLRLSCAASGFTFSSYPMSWVRQAPGKGLEWVSAIGGSGTWTYYADSVKGRFTISRDNSKNTLYLQMNSLRAEDTAVYYCARYWPMDSWGQGTLVTVSS

SEQ ID NO: 107 - protein, 108 aa, Artificial Sequence
  source: /note=“Description of Artificial Sequence Synthetic polypeptide”
EIVLTQSPGTLSLSPGERATLSCRASQSVSSSYLAWYQQKPGQAPRLLMYDASIRATGIPDRFSGSGSGTDFTLTISRLEPEDFAVYYCQQYQKWPLTFGQGTKVEIK

SEQ ID NO: 108 - protein, 108 aa, Artificial Sequence
  source: /note=“Description of Artificial Sequence Synthetic polypeptide”
EIVLTQSPGTLSLSPGERATLSCRASQSVSAVYLAWYQQKPGQAPRLLMYDASIRATGIPDRFSGSGSGTDFTLTISRLEPEDFAVYYCQQYRAWPLTFGQGTKVEIK

SEQ ID NO: 109 - protein, 108 aa, Artificial Sequence
  source: /note=“Description of Artificial Sequence Synthetic polypeptide”
EIVLTQSPGTLSLSPGERATLSCRASIAVSSTYLAWYQQKPGQAPRLLMYDASIRATGIPDRFSGSGSGTDFTLTISRLEPEDFAVYYCQQYMVWPLTFGQGTKVEIK

SEQ ID NO: 110 - protein, 115 aa, Artificial Sequence
  source: /note=“Description of Artificial Sequence Synthetic polypeptide”
EVQLLESGGGLVQPGGSLRLSCAASGFTFSSYPMSWVRQAPGKGLEWVSALFGSGGSTYYADSVKGRFTISRDNSKNTLYLQMNSLRAEDTAVYYCARYWPMDSWGQGTLVTVSS

SEQ ID NO: 111 - protein, 108 aa, Artificial Sequence
  source: /note=“Description of Artificial Sequence Synthetic polypeptide”
EIVLTQSPGTLSLSPGERATLSCRPRQSVSSSYLAWYQQKPGQAPRLLMYDASIRATGIPDRFSGSGSGTDFTLTISRLEPEDFAVYYCQQYQDWPLTFGQGTKVEIK

SEQ ID NO: 112 - protein, 115 aa, Artificial Sequence
  source: /note=“Description of Artificial Sequence Synthetic polypeptide”
EVQLLESGGGLVQPGGSLRLSCAASGFTFSSYPMSWVRQAPGKGLEWVSAIGGSGGSLPYADSVKGRFTISRDNSKNTLYLQMNSLRAEDTAVYYCARYWPMDIWGQGTLVTVSS

SEQ ID NO: 113 - protein, 108 aa, Artificial Sequence
  source: /note=“Description of Artificial Sequence Synthetic polypeptide”
EIVLTQSPGTLSLSPGERATLSCRASQSVSSTYLAWYQQKPGQAPRLLMYDASIRATGIPDRFSGSGSGTDFTLTISRLEPEDFAVYYCQQYQEWPLTFGQGTKVEIK

SEQ ID NO: 114 - protein, 115 aa, Artificial Sequence
  source: /note=“Description of Artificial Sequence Synthetic polypeptide”
EVQLLESGGGLVQPGGSLRLSCAASGFTFSSYPMSWVRQAPGKGLEWVSAALGSGGSTYYADSVKGRFTISRDNSKNTLYLQMNSLRAEDTAVYYCARYWPMDSWGQGTLVTVSS

SEQ ID NO: 115 - protein, 108 aa, Artificial Sequence
  source: /note=“Description of Artificial Sequence Synthetic polypeptide”
EIVLTQSPGTLSLSPGERATLSCRASQSVSASYLAWYQQKPGQAPRLLMYDASIRATGIPDRFSGSGSGTDFTLTISRLEPEDFAVYYCQQYMSWPLTFGQGTKVEIK

SEQ ID NO: 116 - protein, 108 aa, Artificial Sequence
  source: /note=“Description of Artificial Sequence Synthetic polypeptide”
EIVLTQSPGTLSLSPGERATLSCRASQSVSYMYLAWYQQKPGQAPRLLIYDASIRATGIPDRFSGSGSGTDFTLTISRLEPEDFAVYYCQQYKSWPLTFGQGTKVEIK

SEQ ID NO: 117 - protein, 108 aa, Artificial Sequence
  source: /note=“Description of Artificial Sequence Synthetic polypeptide”
EIVLTQSPGTLSLSPGERATLSCRASQSVSALYLAWYQQKPGQAPRLLMYDASIRATGIPDRFSGSGSGTDFTLTISRLEPEDFAVYYCQQYYGWPLTFGQGTKVEIK

SEQ ID NO: 118 - protein, 115 aa, Artificial Sequence
  source: /note=“Description of Artificial Sequence Synthetic polypeptide”
EVQLLESGGGLVQPGGSLRLSCAASGFTFSSYPMSWVRQAPGKGLEWVSAIGGSGGSLPYADSVKGRFTISRDNSKNTLYLQMNSLRAEDTAVYYCARYWPMADWGQGTLVTVSS

SEQ ID NO: 119 - protein, 108 aa, Artificial Sequence
  source: /note=“Description of Artificial Sequence Synthetic polypeptide”
EIVLTQSPGTLSLSPGERATLSCRASQPISSSYLAWYQQKPGQAPRLLMYDASIRATGIPDRFSGSGSGTDFTLTISRLEPEDFAVYYCQQYQGWPLTFGQGTKVEIK

SEQ ID NO: 120 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAISDSGGFVYYADSVKGRFTISRDNSKNTLYLQMNSLRAEDTAVYYCARYWPMDSWGQGTLVTVSS

SEQ ID NO: 121 - protein, 108 aa, Artificial Sequence
  source: /note=“Description of Artificial Sequence Synthetic polypeptide”
EIVLTQSPGTLSLSPGERATLSCRASQSVSSSYLAWYQQKPGQAPRLLMYDASIRATGIPDRFSGSGSGTDFTLTISRLEPEDFAVYYCQQYEFWPLTFGQGTKVEIK

SEQ ID NO: 122 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAIGGSGGSTYYADSVKGRFTISRDNSKNTLYLQMNSLRAEDTAVYYCARYWPMSLWGQGTLVTVSS

SEQ ID NO: 123 - protein, 108 aa, Artificial Sequence
  source: /note=“Description of Artificial Sequence Synthetic polypeptide”
EIVLTQSPGTLSLSPGERATLSCRASQSVSSTYLAWYQQKPGQAPRLLMYDASIRATGIPDRFSGSGSGTDFTLTISRLEPEDFAVYYCQQYMSWPLTFGQGTKVEIK

SEQ ID NO: 124 - protein, 108 aa, Artificial Sequence
  source: /note=“Description of Artificial Sequence Synthetic polypeptide”
EIVLTQSPGTLSLSPGERATLSCRASQGISSTYLAWYQQKPGQAPRLLMYDASIRATGIPDRFSGSGSGTDFTLTISRLEPEDFAVYYCQQYAYWPLTFGQGTKVEIK

SEQ ID NO: 125 - protein, 115 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSACLDSGGSTYYADSVKGRFTISRDNSKNTLYLQMNSLRAEDTAVYYCARYWPMDSWGQGTLVTVSS

SEQ ID NO: 126 - protein, 108 aa, Artificial Sequence
  source: /note=“Description of Artificial Sequence Synthetic polypeptide”
EIVLTQSPGTLSLSPGERATLSCRASQSVSSSYLAWYQQKPGQAPRLLMYDASIRATGIPDRFSGSGSGTDFTLTISRLEPEDFAVYYCQQYQGWPLTFGQGTKVEIK

SEQ ID NO: 127 - protein, 115 aa, Artificial Sequence
  source: /note=“Description of Artificial Sequence Synthetic polypeptide”
EVQLLESGGGLVQPGGSLRLSCAASGFTFSSYPMSWVRQAPGKGLEWVSAALGSGGSTYYADSVKGRFTISRDNSKNTLYLQMNSLRAEDTAVYYCARYWPMSLWGQGTLVTVSS

SEQ ID NO: 128 - protein, 108 aa, Artificial Sequence
  source: /note=“Description of Artificial Sequence Synthetic polypeptide”
EIVLTQSPGTLSLSPGERATLSCRASQSVSVRYLAWYQQKPGQAPRLLMYDASIRATGIPDRFSGSGSGTDFTLTISRLEPEDFAVYYCQQYGSWPITFGQGTKVEIK

SEQ ID NO: 129 - protein, 5 aa, Artificial Sequence
  source: /note=“Description of Artificial Sequence Synthetic peptide”
SYAMT

SEQ ID NO: 130 - protein, 7 aa, Artificial Sequence
  source: /note=“Description of Artificial Sequence Synthetic peptide”
GFTFGSY

SEQ ID NO: 131 - protein, 10 aa, Artificial Sequence
  source: /note=“Description of Artificial Sequence Synthetic peptide”
GFTFGSYAMT

SEQ ID NO: 132 - protein, 17 aa, Artificial Sequence
  source: /note=“Description of Artificial Sequence Synthetic peptide”
AISGSGGNTFYADSVKG

SEQ ID NO: 133 - protein, 6 aa, Artificial Sequence
  source: /note=“Description of Artificial Sequence Synthetic peptide”
SGSGGN

SEQ ID NO: 134 - protein, 10 aa, Artificial Sequence
  source: /note=“Description of Artificial Sequence Synthetic peptide”
VSPIASGMDY

SEQ ID NO: 135 - protein, 10 aa, Artificial Sequence
  source: /note=“Description of Artificial Sequence Synthetic peptide”
VSPIAAQMDY

SEQ ID NO: 136 - protein, 10 aa, Artificial Sequence
  source: /note=“Description of Artificial Sequence Synthetic peptide”
VSPIAALMDY

SEQ ID NO: 137 - protein, 10 aa, Artificial Sequence
  source: /note=“Description of Artificial Sequence Synthetic peptide”
VSPIAAPMDY

SEQ ID NO: 138 - protein, 17 aa, Artificial Sequence
  source: /note=“Description of Artificial Sequence Synthetic peptide”
AISGSGGNTFYADQRKG

SEQ ID NO: 139 - protein, 17 aa, Artificial Sequence
  source: /note=“Description of Artificial Sequence Synthetic peptide”
AIDYSGGNTFYADSVKG

SEQ ID NO: 140 - protein, 6 aa, Artificial Sequence
  source: /note=“Description of Artificial Sequence Synthetic peptide”
DYSSGN

SEQ ID NO: 141 - protein, 17 aa, Artificial Sequence
  source: /note=“Description of Artificial Sequence Synthetic peptide”
AISYQGGNTFYADSVKG

SEQ ID NO: 142 - protein, 6 aa, Artificial Sequence
  source: /note=“Description of Artificial Sequence Synthetic peptide”
SYQGGN

SEQ ID NO: 143 - protein, 17 aa, Artificial Sequence
  source: /note=“Description of Artificial Sequence Synthetic peptide”
AISLTGGNTFYADSVKG

SEQ ID NO: 144 - protein, 6 aa, Artificial Sequence
  source: /note=“Description of Artificial Sequence Synthetic peptide”
SLTGGN

SEQ ID NO: 145 - protein, 17 aa, Artificial Sequence
  source: /note=“Description of Artificial Sequence Synthetic peptide”
AISHAGGNTFYADSVKG

SEQ ID NO: 146 - protein, 6 aa, Artificial Sequence
  source: /note=“Description of Artificial Sequence Synthetic peptide”
SHAGGN

SEQ ID NO: 147 - protein, 17 aa, Artificial Sequence
  source: /note=“Description of Artificial Sequence Synthetic peptide”
AISGSGGNTFYADQLKG

SEQ ID NO: 148 - protein, 10 aa, Artificial Sequence
  source: /note=“Description of Artificial Sequence Synthetic peptide”
VSPIYAGMDY

SEQ ID NO: 149 - protein, 10 aa, Artificial Sequence
  source: /note=“Description of Artificial Sequence Synthetic peptide”
VSPIAAEMDY

SEQ ID NO: 150 - protein, 5 aa, Artificial Sequence
  source: /note=“Description of Artificial Sequence Synthetic peptide”
SYAMN

SEQ ID NO: 151 - protein, 7 aa, Artificial Sequence
  source: /note=“Description of Artificial Sequence Synthetic peptide”
GFTFSSY

SEQ ID NO: 152 - protein, 10 aa, Artificial Sequence
  source: /note=“Description of Artificial Sequence Synthetic peptide”
GFTFSSYAMN

SEQ ID NO: 153 - protein, 17 aa, Artificial Sequence
  source: /note=“Description of Artificial Sequence Synthetic peptide”
AISDSGGSTYYADSVKG

SEQ ID NO: 154 - protein, 6 aa, Artificial Sequence
  source: /note=“Description of Artificial Sequence Synthetic peptide”
SDSGGS

SEQ ID NO: 155 - protein, 6 aa, Artificial Sequence
  source: /note=“Description of Artificial Sequence Synthetic peptide”
YWPMDI

SEQ ID NO: 156 - protein, 5 aa, Artificial Sequence
  source: /note=“Description of Artificial Sequence Synthetic peptide”
SYPMS

SEQ ID NO: 157 - protein, 10 aa, Artificial Sequence
  source: /note=“Description of Artificial Sequence Synthetic peptide”
GFTFSSYPMS

SEQ ID NO: 158 - protein, 17 aa, Artificial Sequence
  source: /note=“Description of Artificial Sequence Synthetic peptide”
AIGGSGGSLPYADSVKG

SEQ ID NO: 159 - protein, 6 aa, Artificial Sequence
  source: /note=“Description of Artificial Sequence Synthetic peptide”
GGSGGS

SEQ ID NO: 160 - protein, 17 aa, Artificial Sequence
  source: /note=“Description of Artificial Sequence Synthetic peptide”
AILDSGGSTYYADSVKG

SEQ ID NO: 161 - protein, 6 aa, Artificial Sequence
  source: /note=“Description of Artificial Sequence Synthetic peptide”
YWPMDS

SEQ ID NO: 162 - protein, 17 aa, Artificial Sequence
  source: /note=“Description of Artificial Sequence Synthetic peptide”
AIGGSGGSTYYADSVKG

SEQ ID NO: 163 - protein, 17 aa, Artificial Sequence
  source: /note=“Description of Artificial Sequence Synthetic peptide”
AISDSGGSAWYADSVKG

SEQ ID NO: 164 - protein, 6 aa, Artificial Sequence
  source: /note=“Description of Artificial Sequence Synthetic peptide”
YWPMSL

SEQ ID NO: 165 - protein, 17 aa, Artificial Sequence
  source: /note=“Description of Artificial Sequence Synthetic peptide”
AISDFGGSTYYADSVKG

SEQ ID NO: 166 - protein, 6 aa, Artificial Sequence
  source: /note=“Description of Artificial Sequence Synthetic peptide”
SDFGGS

SEQ ID NO: 167 - protein, 17 aa, Artificial Sequence
  source: /note=“Description of Artificial Sequence Synthetic peptide”
AITASGGSTYYADSVKG

SEQ ID NO: 168 - protein, 6 aa, Artificial Sequence
  source: /note=“Description of Artificial Sequence Synthetic peptide”
TASGGS

SEQ ID NO: 169 - protein, 17 aa, Artificial Sequence
  source: /note=“Description of Artificial Sequence Synthetic peptide”
AISDSGGSRWYADSVKG

SEQ ID NO: 170 - protein, 6 aa, Artificial Sequence
  source: /note=“Description of Artificial Sequence Synthetic peptide”
YWPMTP

SEQ ID NO: 171 - protein, 17 aa, Artificial Sequence
  source: /note=“Description of Artificial Sequence Synthetic peptide”
AVLDSGGSTYYADSVKG

SEQ ID NO: 172 - protein, 6 aa, Artificial Sequence
  source: /note=“Description of Artificial Sequence Synthetic peptide”
LDSGGS

SEQ ID NO: 173 - protein, 6 aa, Artificial Sequence
  source: /note=“Description of Artificial Sequence Synthetic peptide”
YWPMSD

SEQ ID NO: 174 - protein, 17 aa, Artificial Sequence
  source: /note=“Description of Artificial Sequence Synthetic peptide”
AISDSGGSKWYADSVKG

SEQ ID NO: 175 - protein, 17 aa, Artificial Sequence
  source: /note=“Description of Artificial Sequence Synthetic peptide”
AISDSGGSGWYADSVKG

SEQ ID NO: 176 - protein, 17 aa, Artificial Sequence
  source: /note=“Description of Artificial Sequence Synthetic peptide”
AISDSGGSCWYADSVKG

SEQ ID NO: 177 - protein, 17 aa, Artificial Sequence
  source: /note=“Description of Artificial Sequence Synthetic peptide”
AIFASGGSTYYADSVKG

SEQ ID NO: 178 - protein, 6 aa, Artificial Sequence
  source: /note=“Description of Artificial Sequence Synthetic peptide”
FASGGS

SEQ ID NO: 179 - protein, 6 aa, Artificial Sequence
  source: /note=“Description of Artificial Sequence Synthetic peptide”
SGWGGS

SEQ ID NO: 180 - protein, 17 aa, Artificial Sequence
  source: /note=“Description of Artificial Sequence Synthetic peptide”
AIMSSGGPLYYADSVKG

SEQ ID NO: 181 - protein, 6 aa, Artificial Sequence
  source: /note=“Description of Artificial Sequence Synthetic peptide”
MSSGGP

SEQ ID NO: 182 - protein, 6 aa, Artificial Sequence
  source: /note=“Description of Artificial Sequence Synthetic peptide”
YWPMAL

SEQ ID NO: 183 - protein, 17 aa, Artificial Sequence
  source: /note=“Description of Artificial Sequence Synthetic peptide”
AILMSGGSTYYADSVKG

SEQ ID NO: 184 - protein, 6 aa, Artificial Sequence
  source: /note=“Description of Artificial Sequence Synthetic peptide”
LMSGGS

SEQ ID NO: 185 - protein, 17 aa, Artificial Sequence
  source: /note=“Description of Artificial Sequence Synthetic peptide”
AISDSGGYRYYADSVKG

SEQ ID NO: 186 - protein, 6 aa, Artificial Sequence
  source: /note=“Description of Artificial Sequence Synthetic peptide”
SDSGGY

SEQ ID NO: 187 - protein, 17 aa, Artificial Sequence
  source: /note=“Description of Artificial Sequence Synthetic peptide”
AILSSGGSTYYADSVKG

SEQ ID NO: 188 - protein, 6 aa, Artificial Sequence
  source: /note=“Description of Artificial Sequence Synthetic peptide”
LSSGGS

SEQ ID NO: 189 - protein, 6 aa, Artificial Sequence
  source: /note=“Description of Artificial Sequence Synthetic peptide”
YWPMSP

SEQ ID NO: 190 - protein, 17 aa, Artificial Sequence
  source: /note=“Description of Artificial Sequence Synthetic peptide”
AIGGSGGWSYYADSVKG

SEQ ID NO: 191 - protein, 6 aa, Artificial Sequence
  source: /note=“Description of Artificial Sequence Synthetic peptide”
GGSGGW

SEQ ID NO: 192 - protein, 17 aa, Artificial Sequence
  source: /note=“Description of Artificial Sequence Synthetic peptide”
ATVGSGGSIGYADSVKG

SEQ ID NO: 193 - protein, 6 aa, Artificial Sequence
  source: /note=“Description of Artificial Sequence Synthetic peptide”
VGSGGS

SEQ ID NO: 194 - protein, 17 aa, Artificial Sequence
  source: /note=“Description of Artificial Sequence Synthetic peptide”
AIGGSGGSIHYADSVKG

SEQ ID NO: 195 - protein, 17 aa, Artificial Sequence
  source: /note=“Description of Artificial Sequence Synthetic peptide”
AHIGSGGSTYYADSVKG

SEQ ID NO: 196 - protein, 6 aa, Artificial Sequence
  source: /note=“Description of Artificial Sequence Synthetic peptide”
IGSGGS

SEQ ID NO: 197 - protein, 6 aa, Artificial Sequence
  source: /note=“Description of Artificial Sequence Synthetic peptide”
YWPMDP

SEQ ID NO: 198 - protein, 17 aa, Artificial Sequence
  source: /note=“Description of Artificial Sequence Synthetic peptide”
AIGGSGGSLGYADSVKG

SEQ ID NO: 199 - protein, 17 aa, Artificial Sequence
  source: /note=“Description of Artificial Sequence Synthetic peptide”
AIGGSGTWTYYADSVKG

SEQ ID NO: 200 - protein, 6 aa, Artificial Sequence
  source: /note=“Description of Artificial Sequence Synthetic peptide”
GGSGTW

SEQ ID NO: 201 - protein, 17 aa, Artificial Sequence
  source: /note=“Description of Artificial Sequence Synthetic peptide”
ALFGSGGSTYYADSVKG

SEQ ID NO: 202 - protein, 6 aa, Artificial Sequence
  source: /note=“Description of Artificial Sequence Synthetic peptide”
FGSGGS

SEQ ID NO: 203 - protein, 17 aa, Artificial Sequence
  source: /note=“Description of Artificial Sequence Synthetic peptide”
AALGSGGSTYYADSVKG

SEQ ID NO: 204 - protein, 6 aa, Artificial Sequence
  source: /note=“Description of Artificial Sequence Synthetic peptide”
LGSGGS

SEQ ID NO: 205 - protein, 6 aa, Artificial Sequence
  source: /note=“Description of Artificial Sequence Synthetic peptide”
YWPMAD

SEQ ID NO: 206 - protein, 17 aa, Artificial Sequence
  source: /note=“Description of Artificial Sequence Synthetic peptide”
AISDSGGFVYYADSVKG

SEQ ID NO: 207 - protein, 6 aa, Artificial Sequence
  source: /note=“Description of Artificial Sequence Synthetic peptide”
SDSGGF

SEQ ID NO: 208 - protein, 17 aa, Artificial Sequence
  source: /note=“Description of Artificial Sequence Synthetic peptide”
ACLDSGGSTYYADSVKG

SEQ ID NO: 209 - protein, 12 aa, Artificial Sequence
  source: /note=“Description of Artificial Sequence Synthetic peptide”
RASQSVSSSYLA

SEQ ID NO: 210 - protein, 7 aa, Artificial Sequence
  source: /note=“Description of Artificial Sequence Synthetic peptide”
GASSRAT

SEQ ID NO: 211 - protein, 10 aa, Artificial Sequence
  source: /note=“Description of Artificial Sequence Synthetic peptide”
QHYGSPPSFT

SEQ ID NO: 212 - protein, 12 aa, Artificial Sequence
  source: /note=“Description of Artificial Sequence Synthetic peptide”
RASQSLGSFYLA

SEQ ID NO: 213 - protein, 10 aa, Artificial Sequence
  source: /note=“Description of Artificial Sequence Synthetic peptide”
KHYGWPPSFT

SEQ ID NO: 214 - protein, 10 aa, Artificial Sequence
  source: /note=“Description of Artificial Sequence Synthetic peptide”
QHYNYPPSFT

SEQ ID NO: 215 - protein, 12 aa, Artificial Sequence
  source: /note=“Description of Artificial Sequence Synthetic peptide”
RASQSVGDFYLA

SEQ ID NO: 216 - protein, 10 aa, Artificial Sequence
  source: /note=“Description of Artificial Sequence Synthetic peptide”
QHYPYPPSFT

SEQ ID NO: 217 - protein, 12 aa, Artificial Sequence
  source: /note=“Description of Artificial Sequence Synthetic peptide”
RASQSVSSSYPS

SEQ ID NO: 218 - protein, 12 aa, Artificial Sequence
  source: /note=“Description of Artificial Sequence Synthetic peptide”
RASQSVSAHYLA

SEQ ID NO: 219 - protein, 12 aa, Artificial Sequence
  source: /note=“Description of Artificial Sequence Synthetic peptide”
RASQSVSSFFLA

SEQ ID NO: 220 - protein, 10 aa, Artificial Sequence
  source: /note=“Description of Artificial Sequence Synthetic peptide”
KYYPYPPSFT

SEQ ID NO: 221 - protein, 7 aa, Artificial Sequence
  source: /note=“Description of Artificial Sequence Synthetic peptide”
DASIRAT

SEQ ID NO: 222 - protein, 9 aa, Artificial Sequence
  source: /note=“Description of Artificial Sequence Synthetic peptide”
QQYGSWPLT

SEQ ID NO: 223 - protein, 12 aa, Artificial Sequence
  source: /note=“Description of Artificial Sequence Synthetic peptide”
RASQSVSVLYLA

SEQ ID NO: 224 - protein, 9 aa, Artificial Sequence
  source: /note=“Description of Artificial Sequence Synthetic peptide”
QQYQRWPLT

SEQ ID NO: 225 - protein, 9 aa, Artificial Sequence
  source: /note=“Description of Artificial Sequence Synthetic peptide”
QQYQSWPLT

SEQ ID NO: 226 - protein, 12 aa, Artificial Sequence
  source: /note=“Description of Artificial Sequence Synthetic peptide”
RASQSVSDLYLA

SEQ ID NO: 227 - protein, 9 aa, Artificial Sequence
  source: /note=“Description of Artificial Sequence Synthetic peptide”
QQYQTWPLT

SEQ ID NO: 228 - protein, 12 aa, Artificial Sequence
  source: /note=“Description of Artificial Sequence Synthetic peptide”
RASQSVSNLYLA

SEQ ID NO: 229 - protein, 9 aa, Artificial Sequence
  source: /note=“Description of Artificial Sequence Synthetic peptide”
QQYQGWPLT

SEQ ID NO: 230 - protein, 12 aa, Artificial Sequence
  source: /note=“Description of Artificial Sequence Synthetic peptide”
RASQSVSAYYLA

SEQ ID NO: 231 - protein, 9 aa, Artificial Sequence
  source: /note=“Description of Artificial Sequence Synthetic peptide”
QQYERWPLT

SEQ ID NO: 232 - protein, 12 aa, Artificial Sequence
  source: /note=“Description of Artificial Sequence Synthetic peptide”
RASQSVSSLYLA

SEQ ID NO: 233 - protein, 9 aa, Artificial Sequence
  source: /note=“Description of Artificial Sequence Synthetic peptide”
QQYQVWPLT

SEQ ID NO: 234 - protein, 9 aa, Artificial Sequence
  source: /note=“Description of Artificial Sequence Synthetic peptide”
QQYLDWPLT

SEQ ID NO: 235 - protein, 12 aa, Artificial Sequence
  source: /note=“Description of Artificial Sequence Synthetic peptide”
RASQSVSALYLA

SEQ ID NO: 236 - protein, 9 aa, Artificial Sequence
  source: /note=“Description of Artificial Sequence Synthetic peptide”
QQYLAWPLT

SEQ ID NO: 237 - protein, 9 aa, Artificial Sequence
  source: /note=“Description of Artificial Sequence Synthetic peptide”
QQYFTWPLT

SEQ ID NO: 238 - protein, 12 aa, Artificial Sequence
  source: /note=“Description of Artificial Sequence Synthetic peptide”
RASQSVSPYYLA

SEQ ID NO: 239 - protein, 12 aa, Artificial Sequence
  source: /note=“Description of Artificial Sequence Synthetic peptide”
RASQSVSVEYLA

SEQ ID NO: 240 - protein, 9 aa, Artificial Sequence
  source: /note=“Description of Artificial Sequence Synthetic peptide”
QQYARWPLT

SEQ ID NO: 241 - protein, 12 aa, Artificial Sequence
  source: /note=“Description of Artificial Sequence Synthetic peptide”
RASQSVSELYLA

SEQ ID NO: 242 - protein, 9 aa, Artificial Sequence
  source: /note=“Description of Artificial Sequence Synthetic peptide”
QQYFGWPLT

SEQ ID NO: 243 - protein, 12 aa, Artificial Sequence
  source: /note=“Description of Artificial Sequence Synthetic peptide”
RASQSVEMSYLA

SEQ ID NO: 244 - protein, 9 aa, Artificial Sequence
  source: /note=“Description of Artificial Sequence Synthetic peptide”
QQYAHWPLT

SEQ ID NO: 245 - protein, 12 aa, Artificial Sequence
  source: /note=“Description of Artificial Sequence Synthetic peptide”
RASQSVSAQYLA

SEQ ID NO: 246 - protein, 12 aa, Artificial Sequence
  source: /note=“Description of Artificial Sequence Synthetic peptide”
GPSQSVSSSYLA

SEQ ID NO: 247 - protein, 12 aa, Artificial Sequence
  source: /note=“Description of Artificial Sequence Synthetic peptide”
RASQSVSSSYWA

SEQ ID NO: 248 - protein, 9 aa, Artificial Sequence
  source: /note=“Description of Artificial Sequence Synthetic peptide”
QQYESWPLT

SEQ ID NO: 249 - protein, 12 aa, Artificial Sequence
  source: /note=“Description of Artificial Sequence Synthetic peptide”
RGGQSVSSSYLA

SEQ ID NO: 250 - protein, 12 aa, Artificial Sequence
  source: /note=“Description of Artificial Sequence Synthetic peptide”
RASQSVSFLYLA

SEQ ID NO: 251 - protein, 12 aa, Artificial Sequence
  source: /note=“Description of Artificial Sequence Synthetic peptide”
RASQSVSSTYLA

SEQ ID NO: 252 - protein, 7 aa, Artificial Sequence
  source: /note=“Description of Artificial Sequence Synthetic peptide”
DASSRAP

SEQ ID NO: 253 - protein, 9 aa, Artificial Sequence
  source: /note=“Description of Artificial Sequence Synthetic peptide”
QQYSTSPLT

SEQ ID NO: 254 - protein, 12 aa, Artificial Sequence
  source: /note=“Description of Artificial Sequence Synthetic peptide”
RASQSVSPEYLA

SEQ ID NO: 255 - protein, 9 aa, Artificial Sequence
  source: /note=“Description of Artificial Sequence Synthetic peptide”
QQYSVWPLT

SEQ ID NO: 256 - protein, 9 aa, Artificial Sequence
  source: /note=“Description of Artificial Sequence Synthetic peptide”
QQYSAWPLT

SEQ ID NO: 257 - protein, 12 aa, Artificial Sequence
  source: /note=“Description of Artificial Sequence Synthetic peptide”
RASQSVSSVYLA

SEQ ID NO: 258 - protein, 9 aa, Artificial Sequence
  source: /note=“Description of Artificial Sequence Synthetic peptide”
QQYSTWPLT

SEQ ID NO: 259 - protein, 9 aa, Artificial Sequence
  source: /note=“Description of Artificial Sequence Synthetic peptide”
QQYSRWPLT

SEQ ID NO: 260 - protein, 12 aa, Artificial Sequence
  source: /note=“Description of Artificial Sequence Synthetic peptide”
RASQSVSPLYLA

SEQ ID NO: 261 - protein, 9 aa, Artificial Sequence
  source: /note=“Description of Artificial Sequence Synthetic peptide”
QQYSAFPLT

SEQ ID NO: 262 - protein, 12 aa, Artificial Sequence
  source: /note=“Description of Artificial Sequence Synthetic peptide”
WLSQSVSSTYLA

SEQ ID NO: 263 - protein, 9 aa, Artificial Sequence
  source: /note=“Description of Artificial Sequence Synthetic peptide”
QQYSEWPLT

SEQ ID NO: 264 - protein, 9 aa, Artificial Sequence
  source: /note=“Description of Artificial Sequence Synthetic peptide”
QQYSSWPLT

SEQ ID NO: 265 - protein, 12 aa, Artificial Sequence
  source: /note=“Description of Artificial Sequence Synthetic peptide”
RASQSVSSLFLA

SEQ ID NO: 266 - protein, 12 aa, Artificial Sequence
  source: /note=“Description of Artificial Sequence Synthetic peptide”
ACSQSVSSTYLA

SEQ ID NO: 267 - protein, 12 aa, Artificial Sequence
  source: /note=“Description of Artificial Sequence Synthetic peptide”
RASCDVSSTYLA

SEQ ID NO: 268 - protein, 9 aa, Artificial Sequence
  source: /note=“Description of Artificial Sequence Synthetic peptide”
QQYMRSPLT

SEQ ID NO: 269 - protein, 12 aa, Artificial Sequence
  source: /note=“Description of Artificial Sequence Synthetic peptide”
RASEAVPSTYLA

SEQ ID NO: 270 - protein, 12 aa, Artificial Sequence
  source: /note=“Description of Artificial Sequence Synthetic peptide”
CSSQSVSSTYLA

SEQ ID NO: 271 - protein, 12 aa, Artificial Sequence
  source: /note=“Description of Artificial Sequence Synthetic peptide”
RASVRVSSTYLA

SEQ ID NO: 272 - protein, 9 aa, Artificial Sequence
  source: /note=“Description of Artificial Sequence Synthetic peptide”
QQYMKWPLT

SEQ ID NO: 273 - protein, 12 aa, Artificial Sequence
  source: /note=“Description of Artificial Sequence Synthetic peptide”
RASQSVSAAYLA

SEQ ID NO: 274 - protein, 9 aa, Artificial Sequence
  source: /note=“Description of Artificial Sequence Synthetic peptide”
QQYMCWPLT

SEQ ID NO: 275 - protein, 12 aa, Artificial Sequence
  source: /note=“Description of Artificial Sequence Synthetic peptide”
RASQSVSSSYWG

SEQ ID NO: 276 - protein, 9 aa, Artificial Sequence
  source: /note=“Description of Artificial Sequence Synthetic peptide”
QQYQCWPLT

SEQ ID NO: 277 - protein, 12 aa, Artificial Sequence
  source: /note=“Description of Artificial Sequence Synthetic peptide”
RASQSVSSPYLA

SEQ ID NO: 278 - protein, 9 aa, Artificial Sequence
  source: /note=“Description of Artificial Sequence Synthetic peptide”
QQYKAWPLT

SEQ ID NO: 279 - protein, 12 aa, Artificial Sequence
  source: /note=“Description of Artificial Sequence Synthetic peptide”
RASQSVSYLYLA

SEQ ID NO: 280 - protein, 9 aa, Artificial Sequence
  source: /note=“Description of Artificial Sequence Synthetic peptide”
QQYMEWPLT

SEQ ID NO: 281 - protein, 9 aa, Artificial Sequence
  source: /note=“Description of Artificial Sequence Synthetic peptide”
QQYQAWPLT

SEQ ID NO: 282 - protein, 9 aa, Artificial Sequence
  source: /note=“Description of Artificial Sequence Synthetic peptide”
QQYQKWPLT

SEQ ID NO: 283 - protein, 12 aa, Artificial Sequence
  source: /note=“Description of Artificial Sequence Synthetic peptide”
RASQSVSAVYLA

SEQ ID NO: 284 - protein, 9 aa, Artificial Sequence
  source: /note=“Description of Artificial Sequence Synthetic peptide”
QQYRAWPLT

SEQ ID NO: 285 - protein, 12 aa, Artificial Sequence
  source: /note=“Description of Artificial Sequence Synthetic peptide”
RASIAVSSTYLA

SEQ ID NO: 286 - protein, 9 aa, Artificial Sequence
  source: /note=“Description of Artificial Sequence Synthetic peptide”
QQYMVWPLT

SEQ ID NO: 287 - protein, 12 aa, Artificial Sequence
  source: /note=“Description of Artificial Sequence Synthetic peptide”
RPRQSVSSSYLA

SEQ ID NO: 288 - protein, 9 aa, Artificial Sequence
  source: /note=“Description of Artificial Sequence Synthetic peptide”
QQYQDWPLT

SEQ ID NO: 289 - protein, 9 aa, Artificial Sequence
  source: /note=“Description of Artificial Sequence Synthetic peptide”
QQYQEWPLT

SEQ ID NO: 290 - protein, 12 aa, Artificial Sequence
  source: /note=“Description of Artificial Sequence Synthetic peptide”
RASQSVSASYLA

SEQ ID NO: 291 - protein, 9 aa, Artificial Sequence
  source: /note=“Description of Artificial Sequence Synthetic peptide”
QQYMSWPLT

SEQ ID NO: 292 - protein, 12 aa, Artificial Sequence
  source: /note=“Description of Artificial Sequence Synthetic peptide”
RASQSVSYMYLA

SEQ ID NO: 293 - protein, 9 aa, Artificial Sequence
  source: /note=“Description of Artificial Sequence Synthetic peptide”
QQYKSWPLT

SEQ ID NO: 294 - protein, 9 aa, Artificial Sequence
  source: /note=“Description of Artificial Sequence Synthetic peptide”
QQYYGWPLT

SEQ ID NO: 295 - protein, 12 aa, Artificial Sequence
  source: /note=“Description of Artificial Sequence Synthetic peptide”
RASQPISSSYLA

SEQ ID NO: 296 - protein, 9 aa, Artificial Sequence
  source: /note=“Description of Artificial Sequence Synthetic peptide”
QQYEFWPLT

SEQ ID NO: 297 - protein, 12 aa, Artificial Sequence
  source: /note=“Description of Artificial Sequence Synthetic peptide”
RASQGISSTYLA

SEQ ID NO: 298 - protein, 9 aa, Artificial Sequence
  source: /note=“Description of Artificial Sequence Synthetic peptide”
QQYAYWPLT

SEQ ID NO: 299 - protein, 12 aa, Artificial Sequence
  source: /note=“Description of Artificial Sequence Synthetic peptide”
RASQSVSVRYLA

SEQ ID NO: 300 - protein, 9 aa, Artificial Sequence
  source: /note=“Description of Artificial Sequence Synthetic peptide”
QQYGSWPIT

SEQ ID NO: 301 - protein, 5 aa, Artificial Sequence
  source: /note=“Description of Artificial Sequence Synthetic peptide”
  VARIANT (3)..(3): /replace=“P”
  VARIANT (5)..(5): /replace=“N” or “S”
  SITE (1)..(5): /note=“Variant residues given in the sequence have no preference with respect to those in the annotations for variant positions”
SYAMT

SEQ ID NO: 302 - protein, 7 aa, Artificial Sequence
  source: /note=“Description of Artificial Sequence Synthetic peptide”
  VARIANT (5)..(5): /replace=“S”
  SITE (1)..(7): /note=“Variant residues given in the sequence have no preference with respect to those in the annotations for variant positions”
GFTFGSY

SEQ ID NO: 303 - protein, 10 aa, Artificial Sequence
  source: /note=“Description of Artificial Sequence Synthetic peptide”
  VARIANT (5)..(5): /replace=“S”
  VARIANT (8)..(8): /replace=“P”
  VARIANT (10)..(10): /replace=“N” or “S”
  SITE (1)..(10): /note=“Variant residues given in the sequence have no preference with respect to those in the annotations for variant positions”
GFTFGSYAMT

SEQ ID NO: 304 - protein, 17 aa, Artificial Sequence
  source: /note=“Description of Artificial Sequence Synthetic peptide”
AISGWGGSLPYADSVKG

SEQ ID NO: 305 - protein, 17 aa, Artificial Sequence
  source: /note=“Description of Artificial Sequence Synthetic peptide”
  VARIANT (2)..(2): /replace=“V” or “T” or “H” or “L” or “A” or “C”
  VARIANT (3)..(3): /replace=“D” or “G” or “T” or “I” or “L” or “F” or “M” or “V”
  VARIANT (4)..(4): /replace=“Y” or “L” or “H” or “D” or “A” or “S” or “M”
  VARIANT (5)..(5): /replace=“Q” or “T” or “A” or “F” or “W”
  VARIANT (7)..(7): /replace=“T”
  VARIANT (8)..(8): /replace=“S” or “P” or “Y” or “W” or “F”
  VARIANT (9)..(9): /replace=“T” or “I” or “L” or “T” or “A” or “R” or “V” or “K” or “G” or “C”
  VARIANT (10)..(10): /replace=“Y” or “P” or “W” or “H” or “G”
  VARIANT (14)..(14): /replace=“R” or “L”
  VARIANT (15)..(15): /replace=“T”
  SITE (1)..(17): /note=“Variant residues given in the sequence have no preference with respect to those in the annotations for variant positions”
AISGSGGNSFYADVGKG

SEQ ID NO: 306 - protein, 6 aa, Artificial Sequence
  source: /note=“Description of Artificial Sequence Synthetic peptide”
  VARIANT (1)..(1): /replace=“V” or “I” or “D” or “G” or “T” or “L” or “F” or “M”
  VARIANT (2)..(2): /replace=“Y” or “L” or “H” or “D” or “A” or “S” or “M”
  VARIANT (3)..(3): /replace=“G” or “F” or “W”
  VARIANT (4)..(4): /replace=“S”
  VARIANT (5)..(5): /replace=“T”
  VARIANT (6)..(6): /replace=“S” or “P” or “Y” or “W”
  SITE (1)..(6): /note=“Variant residues given in the sequence have no preference with respect to those in the annotations for variant positions”
SGSGGN

SEQ ID NO: 307 - protein, 10 aa, Artificial Sequence
  source: /note=“Description of Artificial Sequence Synthetic peptide”
  VARIANT (5)..(5): /replace=“Y”
  VARIANT (6)..(6): /replace=“S”
  VARIANT (7)..(7): /replace=“Q” or “L” or “P” or “E”
  SITE (1)..(10): /note=“Variant residues given in the sequence have no preference with respect to those in the annotations for variant positions”
VSPIAAGMDY

SEQ ID NO: 308 - protein, 6 aa, Artificial Sequence
  source: /note=“Description of Artificial Sequence Synthetic peptide”
  VARIANT (5)..(5): /replace=“S” or “T” or “A”
  VARIANT (6)..(6): /replace=“S” or “L” or “P” or “D”
  SITE (1)..(6): /note=“Variant residues given in the sequence have no preference with respect to those in the annotations for variant positions”
YWPMDI

SEQ ID NO: 309 - protein, 12 aa, Artificial Sequence
  source: /note=“Description of Artificial Sequence Synthetic peptide”
  VARIANT (1)..(1): /replace=“G” or “W” or “A” or “C”
  VARIANT (2)..(2): /replace=“P” or “G” or “L” or “C” or “S”
  VARIANT (3)..(3): /replace=“G” or “R”
  VARIANT (4)..(4): /replace=“C” or “E” or “V” or “I”
  VARIANT (5)..(5): /replace=“P” or “G” or “A” or “R” or “D”
  VARIANT (6)..(6): /replace=“G” or “I” or “L”
  VARIANT (7)..(7): /replace=“E” or “D” or “P” or “G”
  VARIANT (8)..(8): /replace=“P” or “F” or “A” or “M” or “E” or “V” or “N” or “D” or “Y”
  VARIANT (9)..(9): /replace=“T” or “V” or “E” or “F” or “S” or “A” or “M” or “Q” or “Y” or “H” or “R”
  VARIANT (10)..(10): /replace=“F”
  VARIANT (11)..(11): /replace=“W” or “P”
  VARIANT (12)..(12): /replace=“S” or “G”
  SITE (1)..(12): /note=“Variant residues given in the sequence have no preference with respect to those in the annotations for variant positions”
RASQSVSSIYLA

SEQ ID NO: 310 - protein, 7 aa, Artificial Sequence
  source: /note=“Description of Artificial Sequence Synthetic peptide”
  VARIANT (1)..(1): /replace=“D”
  VARIANT (4)..(4): /replace=“I”
  VARIANT (7)..(7): /replace=“P”
  SITE (1)..(7): /note=“Variant residues given in the sequence have no preference with respect to those in the annotations for variant positions”
GASSRAT

SEQ ID NO: 311 - protein, 10 aa, Artificial Sequence
  source: /note=“Description of Artificial Sequence Synthetic peptide”
  VARIANT (1)..(1): /replace=“K”
  VARIANT (2)..(2): /replace=“Y”
  VARIANT (4)..(4): /replace=“N” or “P”
  VARIANT (5)..(5): /replace=“W” or “Y”
  SITE (1)..(10): /note=“Variant residues given in the sequence have no preference with respect to those in the annotations for variant positions”
QHYGSPPSFT

SEQ ID NO: 312 - protein, 9 aa, Artificial Sequence
  source: /note=“Description of Artificial Sequence Synthetic peptide”
  VARIANT (4)..(4): /replace=“Q” or “E” or “L” or “F” or “A” or “S” or “M” or “K” or “R” or “Y”
  VARIANT (5)..(5): /replace=“R” or “T” or “G” or “V” or “F” or “Y” or “D” or “A” or “H” or “V” or “E” or “K” or “C”
  VARIANT (6)..(6): /replace=“F” or “S”
  VARIANT (8)..(8): /replace=“I”
  SITE (1)..(9): /note=“Variant residues given in the sequence have no preference with respect to those in the annotations for variant positions”
QQYGSWPLT

SEQ ID NO: 313 - protein, 119 aa, Artificial Sequence
  source: /note=“Description of Artificial Sequence Synthetic polypeptide”
  VARIANT (30)..(30): /replace=“S”
  VARIANT (33)..(33): /replace=“P”
  VARIANT (35)..(35): /replace=“N” or “S”
  VARIANT (51)..(51): /replace=“V” or “T” or “H” or “L” or “A” or “C”
  VARIANT (52)..(52): /replace=“D” or “G” or “T” or “I” or “L” or “F” or “M” or “V”
  VARIANT (53)..(53): /replace=“Y” or “L” or “H” or “D” or “A” or “S” or “M”
  VARIANT (54)..(54): /replace=“Q” or “T” or “A” or “F” or “W”
  VARIANT (56)..(56): /replace=“T”
  VARIANT (57)..(57): /replace=“S” or “P” or “Y” or “W” or “F”
  VARIANT (58)..(58): /replace=“T” or “I” or “L” or “T” or “A” or “R” or “V” or “K” or “G” or “C”
  VARIANT (59)..(59): /replace=“Y” or “P” or “W” or “H” or “G”
  VARIANT (63)..(63): /replace=“R” or “L”
  VARIANT (64)..(64): /replace=“T”
  VARIANT (103)..(103): /replace=“Y”
  VARIANT (104)..(104): /replace=“S”
  VARIANT (105)..(105): /replace=“Q” or “L” or “P” or “E”
  SITE (1)..(119): /note=“Variant residues given in the sequence have no preference with respect to those in the annotations for variant positions”
EVQLLESGGGLVQPGGSLRLSCAASGFTFGSYAMTWVRQAPGKGLEWVSAISGSGGNSFYADVGKGRFTISRDNSKNTLYLQMNSLRAEDTAVYYCARVSPIAAGMDYWGQGTLVTVSS

SEQ ID NO: 314 - protein, 115 aa, Artificial Sequence
  source: /note=“Description of Artificial Sequence Synthetic polypeptide”
  VARIANT (30)..(30): /replace=“S”
  VARIANT (33)..(33): /replace=“P”
  VARIANT (35)..(35): /replace=“N” or “S”
  VARIANT (51)..(51): /replace=“V” or “T” or “H” or “L” or “A” or “C”
  VARIANT (52)..(52): /replace=“D” or “G” or “T” or “I” or “L” or “F” or “M” or “V”
  VARIANT (53)..(53): /replace=“Y” or “L” or “H” or “D” or “A” or “S” or “M”
  VARIANT (54)..(54): /replace=“Q” or “T” or “A” or “F” or “W”
  VARIANT (56)..(56): /replace=“T”
  VARIANT (57)..(57): /replace=“S” or “P” or “Y” or “W” or “F”
  VARIANT (58)..(58): /replace=“T” or “I” or “L” or “T” or “A” or “R” or “V” or “K” or “G” or “C”
  VARIANT (59)..(59): /replace=“Y” or “P” or “W” or “H” or “G”
  VARIANT (63)..(63): /replace=“R” or “L”
  VARIANT (64)..(64): /replace=“T”
  VARIANT (103)..(103): /replace=“S” or “T” or “A”
  VARIANT (104)..(104): /replace=“S” or “L” or “P” or “D”
  SITE (1)..(115): /note=“Variant residues given in the sequence have no preference with respect to those in the annotations for variant positions”
EVQLLESGGGLVQPGGSLRLSCAASGFTFGSYAMTWVRQAPGKGLEWVSAISGSGGNSFYADVGKGRFTISRDNSKNTLYLQMNSLRAEDTAVYYCARYWPMDIWGQGTLVTVSS

SEQ ID NO: 315 - protein, 109 aa, Artificial Sequence
  source: /note=“Description of Artificial Sequence Synthetic polypeptide”
  VARIANT (24)..(24): /replace=“G” or “W” or “A” or “C”
  VARIANT (25)..(25): /replace=“P” or “G” or “L” or “C” or “S”
  VARIANT (26)..(26): /replace=“G” or “R”
  VARIANT (27)..(27): /replace=“C” or “E” or “V” or “I”
  VARIANT (28)..(28): /replace=“P” or “G” or “A” or “R” or “D”
  VARIANT (29)..(29): /replace=“G” or “I” or “L”
  VARIANT (30)..(30): /replace=“E” or “D” or “P” or “G”
  VARIANT (31)..(31): /replace=“P” or “F” or “A” or “M” or “E” or “V” or “N” or “D” or “Y”
  VARIANT (32)..(32): /replace=“T” or “V” or “E” or “S” or “A” or “M” or “Q” or “Y” or “H” or “R” or “F”
  VARIANT (33)..(33): /replace=“F”
  VARIANT (34)..(34): /replace=“W” or “P”
  VARIANT (35)..(35): /replace=“S” or “G”
  VARIANT (51)..(51): /replace=“D”
  VARIANT (54)..(54): /replace=“I”
  VARIANT (57)..(57): /replace=“P”
  VARIANT (90)..(90): /replace=“K”
  VARIANT (91)..(91): /replace=“Y”
  VARIANT (93)..(93): /replace=“N” or “P”
  VARIANT (94)..(94): /replace=“W” or “Y”
  SITE (1)..(109): /note=“Variant residues given in the sequence have no preference with respect to those in the annotations for variant positions”
EIVLTQSPGTLSLSPGERATLSCRASQSVSSIYLAWYQQKPGQAPRLLMYGASSRATGIPDRFSGSGSGTDFTLTISRLEPEDFAVYYCQHYGSPPSFTFGQGTKVEIK

SEQ ID NO: 316 - protein, 107 aa, Artificial Sequence
  source: /note=“Description of Artificial Sequence Synthetic polypeptide”
  VARIANT (24)..(24): /replace=“G” or “W” or “A” or “C”
  VARIANT (25)..(25): /replace=“P” or “G” or “L” or “C” or “S”
  VARIANT (26)..(26): /replace=“G” or “R”
  VARIANT (27)..(27): /replace=“C” or “E” or “V” or “I”
  VARIANT (28)..(28): /replace=“L” or “P” or “G” or “A” or “R” or “D”
  VARIANT (29)..(29): /replace=“G” or “I”
  VARIANT (30)..(30): /replace=“E” or “D” or “P”
  VARIANT (31)..(31): /replace=“P” or “F” or “A” or “M” or “E” or “V” or “N” or “D” or “Y”
  VARIANT (32)..(32): /replace=“T” or “V” or “E” or “S” or “A” or “M” or “Q” or “Y” or “H” or “R”
  VARIANT (33)..(33): /replace=“F”
  VARIANT (34)..(34): /replace=“W” or “P”
  VARIANT (35)..(35): /replace=“S” or “G”
  VARIANT (51)..(51): /replace=“D”
  VARIANT (54)..(54): /replace=“I”
  VARIANT (57)..(57): /replace=“P”
  VARIANT (93)..(93): /replace=“Q” or “E” or “L” or “F” or “A” or “S” or “M” or “R” or “K” or “Y”
  VARIANT (94)..(94): /replace=“R” or “T” or “G” or “R” or “V” or “D” or “A” or “H” or “E” or “K” or “C” or “F” or “Y”
  VARIANT (95)..(95): /replace=“S” or “F”
  VARIANT (97)..(97): /replace=“I”
  SITE (1)..(107): /note=“Variant residues given in the sequence have no preference with respect to those in the annotations for variant positions”
EIVLTQSPGTLSLSPGERATLSCRASQSVSSIYLAWYQQKPGQAPRLLMYGASSRATGIPDRFSGSGSGTDFTLTISRLEPEDFAVYYCQQYGSWPLFGQGTKVEIK

SEQ ID NO: 317 - protein, 108 aa, Artificial Sequence
  source: /note=“Description of Artificial Sequence Synthetic polypeptide”
EIVLTQSPGTLSLSPGERATLSCRASQSVSALYLAWYQQKPGQAPRLLIYDASSRAPGIPDRFSGSGSGTDFTLTISRLEPEDFAVYYCQQYSAWPLTFGQGTKVEIK

SEQ ID NO: 318 - protein, 21 aa, Homo sapiens
MALPVTALLLPLALLLHAARP

SEQ ID NO: 319 - protein, 16 aa, Homo sapiens
GLAVSTISSFFPPGYQ

SEQ ID NO: 320 - protein, 45 aa, Homo sapiens
TTTPAPRPPTPAPTIASQPLSLRPEACRPAAGGAVHTRGLDFACD

SEQ ID NO: 321 - protein, 231 aa, Artificial Sequence
  source: /note=“Description of Artificial Sequence Synthetic polypeptide”
EPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 322 - protein, 24 aa, Homo sapiens
IYIWAPLAGTCGVLLLSLVITLYC

SEQ ID NO: 323 - protein, 42 aa, Homo sapiens
KRGRKKLLYIFKQPFMRPVQTTQEEDGCSCRFPEEEEGGCEL

SEQ ID NO: 324 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 325 - protein, 52 aa, Homo sapiens
FFIPLLVVILFAVDTGLFISTQQQVTFLLKIKRTRKGFRLLNPHPKPNPKNN

SEQ ID NO: 326 - protein, 215 aa, Homo sapiens
MDTESNRRANLALPQEPSSVPAFEVLEISPQEVSSGRLLKSASSPPLHTWLTVLKKEQEFLGVTQILTAMICLCFGTVVCSVLDISHIEGDIFSSFKAGYPFWGAIFFSISGMLSIISERRNATYLVRGSLGANTASSIAGGTGITILIINLKKSLAYIHIHSCQKFFETKCFMASFSTEIVVMMLFLTILGLGSAVSLTICGAGEELKGNKVPE

SEQ ID NO: 327 - protein, 42 aa, Homo sapiens; identical to SEQ ID NO: 323

SEQ ID NO: 328 - protein, 41 aa, Artificial Sequence
  source: /note=“Description of Artificial Sequence Synthetic polypeptide”
RSKRSRGGHSDYMNMTPRRPGPTRKHYQPYAPPRDFAAYRS

SEQ ID NO: 329 - protein, 18 aa, Homo sapiens
MIPAVVLLLLLLVEQAAA

SEQ ID NO: 330 - protein, 43 aa, Artificial Sequence
  source: /note=“Description of Artificial Sequence Synthetic polypeptide”
LGEPQLCYILDAILFLYGIVLTLLYCRLKIQVRKAAITSYEKS

SEQ ID NO: 331 - protein, 22 aa, Artificial Sequence
  source: /note=“Description of Artificial Sequence Synthetic peptide”
GSGATNFSLLKQAGDVEENPGP

SEQ ID NO: 332 - protein, 21 aa, Artificial Sequence
  source: /note=“Description of Artificial Sequence Synthetic peptide”
GSGEGRGSLLTCGDVEENPGP

SEQ ID NO: 333 - protein, 15 aa, Artificial Sequence
  source: /note=“Description of Artificial Sequence Synthetic peptide”
GGGGSGGGGSGGGGS

SEQ ID NO: 334 - protein, 530 aa, Homo sapiens
LTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGRPALE

SEQ ID NO: 335 - protein, 530 aa, Artificial Sequence
  source: /note=“Description of Artificial Sequence Synthetic polypeptide”
LTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNNGGKQALETVQRLLPVLCQAHGLTPQQVVAIASNGGGRPALE

SEQ ID NO: 336 - protein, 530 aa, Artificial Sequence
  source: /note=“Description of Artificial Sequence Synthetic polypeptide”
LTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALE

SEQ ID NO: 337 - protein, 539 aa, Artificial Sequence
  source: /note=“Description of Artificial Sequence Synthetic polypeptide”
NPQRSTVWYLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGRPALE

SEQ ID NO: 338 - protein, 530 aa, Artificial Sequence
  source: /note=“Description of Artificial Sequence Synthetic polypeptide”
LTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALE

SEQ ID NO: 339 - protein, 530 aa, Artificial Sequence
  source: /note=“Description of Artificial Sequence Synthetic polypeptide”
LTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGRPALE

SEQ ID NO: 340 - protein, 530 aa, Artificial Sequence
  source: /note=“Description of Artificial Sequence Synthetic polypeptide”
LTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALE

SEQ ID NO: 341 - protein, 530 aa, Artificial Sequence
  source: /note=“Description of Artificial Sequence Synthetic polypeptide”
LTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASNIGGKQALETVQALLPVLCQAHGLTPEQVVAIASHDGGKQALETVQRLLPVLCQAHGLTPQQVVAIASNGGGRPALE

SEQ ID NO: 342 - protein, 136 aa, Artificial Sequence
  source: /note=“Description of Artificial Sequence Synthetic polypeptide”
CPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 343 - protein, 453 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYAMNWVRQAPGKGLEWVSAISDSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRASQSVSSSYLAWYQQKPGQAPRLLMYDASIRATGIPDRFSGSGSGTDFTLTISRLEPEDFAVYYCQQYGSWPLTFGQGTKVEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 344 - protein, 482 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYAMNWVRQAPGKGLEWVSAISDSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRASQSVSSSYLAWYQQKPGQAPRLLMYDASIRATGIPDRFSGSGSGTDFTLTISRLEPEDFAVYYCQQYGSWPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 345 - protein, 668 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYAMNWVRQAPGKGLEWVSAISDSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRASQSVSSSYLAWYQQKPGQAPRLLMYDASIRATGIPDRFSGSGSGTDFTLTISRLEPEDFAVYYCQQYGSWPLTFGQGTKVEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 346 - protein, 453 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 347 - protein, 482 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 348 - protein, 668 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 349 - protein, 453 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYAMNWVRQAPGKGLEWVSAISDFGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRASQSVSDLYLAWYQQKPGQAPRLLMYDASIRATGIPDRFSGSGSGTDFTLTISRLEPEDFAVYYCQQYQTWPLTFGQGTKVEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 350 - protein, 482 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYAMNWVRQAPGKGLEWVSAISDFGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRASQSVSDLYLAWYQQKPGQAPRLLMYDASIRATGIPDRFSGSGSGTDFTLTISRLEPEDFAVYYCQQYQTWPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 351 - protein, 668 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYAMNWVRQAPGKGLEWVSAISDFGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRASQSVSDLYLAWYQQKPGQAPRLLMYDASIRATGIPDRFSGSGSGTDFTLTISRLEPEDFAVYYCQQYQTWPLTFGQGTKVEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 352 - protein, 453 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYPMSWVRQAPGKGLEWVSAIGGSGGSTYYADSVKGRFTISRDNSKNTLYLQMNSLRAEDTAVYYCARYWPMDSWGQGTLVTVSSGGGGSGGGGSGGGGSEIVLTQSPGTLSLSPGERATLSCRASQSVSSTYLAWYQQKPGQAPRLLIYDASSRAPGIPDRFSGSGSGTDFTLTISRLEPEDFAVYYCQQYSTSPLTFGQGTKVEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 353 - protein, 482 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYPMSWVRQAPGKGLEWVSAIGGSGGSTYYADSVKGRFTISRDNSKNTLYLQMNSLRAEDTAVYYCARYWPMDSWGQGTLVTVSSGGGGSGGGGSGGGGSEIVLTQSPGTLSLSPGERATLSCRASQSVSSTYLAWYQQKPGQAPRLLIYDASSRAPGIPDRFSGSGSGTDFTLTISRLEPEDFAVYYCQQYSTSPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 354 - protein, 668 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYPMSWVRQAPGKGLEWVSAIGGSGGSTYYADSVKGRFTISRDNSKNTLYLQMNSLRAEDTAVYYCARYWPMDSWGQGTLVTVSSGGGGSGGGGSGGGGSEIVLTQSPGTLSLSPGERATLSCRASQSVSSTYLAWYQQKPGQAPRLLIYDASSRAPGIPDRFSGSGSGTDFTLTISRLEPEDFAVYYCQQYSTSPLTFGQGTKVEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 355 - protein, 453 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYPMSWVRQAPGKGLEWVSAIGGSGGWSYYADSVKGRFTISRDNSKNTLYLQMNSLRAEDTAVYYCARYWPMDSWGQGTLVTVSSGGGGSGGGGSGGGGSEIVLTQSPGTLSLSPGERATLSCWLSQSVSSTYLAWYQQKPGQAPRLLIYDASSRAPGIPDRFSGSGSGTDFTLTISRLEPEDFAVYYCQQYSEWPLTFGQGTKVEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 356 - protein, 482 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYPMSWVRQAPGKGLEWVSAIGGSGGWSYYADSVKGRFTISRDNSKNTLYLQMNSLRAEDTAVYYCARYWPMDSWGQGTLVTVSSGGGGSGGGGSGGGGSEIVLTQSPGTLSLSPGERATLSCWLSQSVSSTYLAWYQQKPGQAPRLLIYDASSRAPGIPDRFSGSGSGTDFTLTISRLEPEDFAVYYCQQYSEWPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 357 - protein, 668 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYPMSWVRQAPGKGLEWVSAIGGSGGWSYYADSVKGRFTISRDNSKNTLYLQMNSLRAEDTAVYYCARYWPMDSWGQGTLVTVSSGGGGSGGGGSGGGGSEIVLTQSPGTLSLSPGERATLSCWLSQSVSSTYLAWYQQKPGQAPRLLIYDASSRAPGIPDRFSGSGSGTDFTLTISRLEPEDFAVYYCQQYSEWPLTFGQGTKVEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 358 - protein, 453 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYAMNWVRQAPGKGLEWVSAISDSGGSRWYADSVKGRFTISRDNSKNTLYLQMNSLRAEDTAVYYCTRYWPMDIWGQGTLVTVSSGGGGSGGGGSGGGGSEIVLTQSPGTLSLSPGERATLSCRASVRVSSTYLAWYQQKPGQAPRLLMYDASIRATGIPDRFSGSGSGTDFTLTISRLEPEDFAVYYCQQYMKWPLTFGQGTKVEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 359 - protein, 482 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYAMNWVRQAPGKGLEWVSAISDSGGSRWYADSVKGRFTISRDNSKNTLYLQMNSLRAEDTAVYYCTRYWPMDIWGQGTLVTVSSGGGGSGGGGSGGGGSEIVLTQSPGTLSLSPGERATLSCRASVRVSSTYLAWYQQKPGQAPRLLMYDASIRATGIPDRFSGSGSGTDFTLTISRLEPEDFAVYYCQQYMKWPLTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 360 - protein, 668 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYAMNWVRQAPGKGLEWVSAISDSGGSRWYADSVKGRFTISRDNSKNTLYLQMNSLRAEDTAVYYCTRYWPMDIWGQGTLVTVSSGGGGSGGGGSGGGGSEIVLTQSPGTLSLSPGERATLSCRASVRVSSTYLAWYQQKPGQAPRLLMYDASIRATGIPDRFSGSGSGTDFTLTISRLEPEDFAVYYCQQYMKWPLTFGQGTKVEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 361 - protein, 453 aa, Artificial Sequence
  source: /note=“Description of Artificial Sequence Synthetic polypeptide”
MALPVTALLLPLALLLHAARPEVQLLESGGGLVQPGGSLRLSCAASGFTFGSYAMTWVRQAPGKGLEWVSAIDYSGGNTFYADSVKGRFTISRDNSKNTLYLQMNSLRAEDTAVYYCARVSPIASGMDYWGQGTLVTVSSGGGGSGGGGSGGGGSEIVLTQSPGTLSLSPGERATLSCRASQSVSSSYPSWYQQKPGQAPRLLIYGASSRATGIPDRFSGSGSGTDFTLTISRLEPEDFAVYYCQHYPYPPSFTFGQGTKVEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQ

SEQ ID NO: 362 - protein, 487 aa, Artificial Sequence
  source: /note=“Description of Artificial Sequence Synthetic polypeptide”
MALPVTALLLPLALLLHAARPEVQLLESGGGLVQPGGSLRLSCAASGFTFGSYAMTWVRQAPGKGLEWVSAIDYSGGNTFYADSVKGRFTISRDNSKNTLYLQMNSLRAEDTAVYYCARVSPIASGMDYWGQGTLVTVSSGGGGSGGGGSGGGGSEIVLTQSPGTLSLSPGERATLSCRASQSVSSSYPSWYQQKPGQAPRLLIYGASSRATGIPDRFSGSGSGTDFTLTISRLEPEDFAVYYCQHYPYPPSFTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 363 - protein, 673 aa, Artificial Sequence
  source: /note=“Description of Artificial Sequence Synthetic polypeptide”
MALPVTALLLPLALLLHAARPEVQLLESGGGLVQPGGSLRLSCAASGFTFGSYAMTWVRQAPGKGLEWVSAIDYSGGNTFYADSVKGRFTISRDNSKNTLYLQMNSLRAEDTAVYYCARVSPIASGMDYWGQGTLVTVSSGGGGSGGGGSGGGGSEIVLTQSPGTLSLSPGERATLSCRASQSVSSSYPSWYQQKPGQAPRLLIYGASSRATGIPDRFSGSGSGTDFTLTISRLEPEDFAVYYCQHYPYPPSFTFGQGTKVEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 364 - protein, 453 aa, Artificial Sequence
  source: /note=“Description of Artificial Sequence Synthetic polypeptide”
MALPVTALLLPLALLLHAARPEVQLLESGGGLVQPGGSLRLSCAASGFTFGSYAMTWVRQAPGKGLEWVSAISGSGGNTFYADSVKGRFTISRDNSKNTLYLQMNSLRAEDTAVYYCARVSPIAAPMDYWGQGTLVTVSSGGGGSGGGGSGGGGSEIVLTQSPGTLSLSPGERATLSCRASQLGSFYLAWYQQKPGQAPRLLIYGASSRATGIPDRFSGSGSGTDFTLTISRLEPEDFAVYYCQHYNYPPSFTFGQGTKVEIKGLAVSTISSFFPPGYQIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQA

SEQ ID NO: 365 - protein, 486 aa, Artificial Sequence
  source: /note=“Description of Artificial Sequence Synthetic polypeptide”
MALPVTALLLPLALLLHAARPEVQLLESGGGLVQPGGSLRLSCAASGFTFGSYAMTWVRQAPGKGLEWVSAISGSGGNTFYADSVKGRFTISRDNSKNTLYLQMNSLRAEDTAVYYCARVSPIAAPMDYWGQGTLVTVSSGGGGSGGGGSGGGGSEIVLTQSPGTLSLSPGERATLSCRASQLGSFYLAWYQQKPGQAPRLLIYGASSRATGIPDRFSGSGSGTDFTLTISRLEPEDFAVYYCQHYNYPPSFTFGQGTKVEIKTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 366 - protein, 672 aa, Artificial Sequence
  source: /note=“Description of Artificial Sequence Synthetic polypeptide”
MALPVTALLLPLALLLHAARPEVQLLESGGGLVQPGGSLRLSCAASGFTFGSYAMTWVRQAPGKGLEWVSAISGSGGNTFYADSVKGRFTISRDNSKNTLYLQMNSLRAEDTAVYYCARVSPIAAPMDYWGQGTLVTVSSGGGGSGGGGSGGGGSEIVLTQSPGTLSLSPGERATLSCRASQLGSFYLAWYQQKPGQAPRLLIYGASSRATGIPDRFSGSGSGTDFTLTISRLEPEDFAVYYCQHYNYPPSFTFGQGTKVEIKEPKSPDKTHTCPPCPAPPVAGPSVFLFPPKPKDTLMIARTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 367 - DNA, 345 bp, Artificial Sequence
  source: /note=“Description of Artificial Sequence Synthetic polynucleotide”
  modified_base (293)..(293): a, c, t, g, unknown or other
gaggtgcagctgctggaatctggcggaggactggtgcagcctggcggctctctgagactgtcttgtgccgccagcggcttcaccttcagcagctacgccatgaactgggtgcgccaggcccctggcaaaggcctggaatgggtgtccgccatcagcgatagcggcggcagcacctactacgccgatagcgtgaagggccggttcaccatcagccgggacaacagcaagaacaccctgtacctgcagatgaacagcctgcgggccgaggacaccgccgtgtactactgtgccanatactggcccatggacatctggggccagggaaccttggtcaccgtctcctca

SEQ ID NO: 368 - DNA, 324 bp, Artificial Sequence
  source: /note=“Description of Artificial Sequence Synthetic polynucleotide”
gagatcgtgctgacacagagccctggcaccctgagcctgtctccaggcgaaagagccaccctgtcctgcaaagccagccagagcgtgtccagcagctacctggcctggtatcagcaaaagcccggccaggctccccggctgctgatgtacgatgccagcatcagagccaccggcatccccgacagattttccggctctggcagcggcaccgacttcaccctgaccatcagcagactggaacccgaggacttcgccgtgtactactgccagcagtacggcagctggcccctgacatttggccagggcacaaaggtggagatcaaa

SEQ ID NO: 369 - DNA, 345 bp, Artificial Sequence
  source: /note=“Description of Artificial Sequence Synthetic polynucleotide”
gaggtgcagctgctggaatctggcggaggactggtgcagcctggcggctctctgagactgtcttgtgccgccagcggcttcaccttcagcagctacgccatgaactgggtgcgccaggcccctggtaaaggtttggaatgggtttctgctattctgtcgtctggtggttctacttactatgccgattctgttaagggtagattcaccatttctagagacaactctaagaacaccttgtacttgcaaatgaactccttgagagctgaagatactgctgtttattactgtgctagatactggccaatggatatttggggtcaaggtactctggtcaccgtctcctca

SEQ ID NO: 370 - DNA, 324 bp, Artificial Sequence
  source: /note=“Description of Artificial Sequence Synthetic polynucleotide”
gagatcgtgctgacacagagccctggcaccctgagcctgtctcctggtgaaagagctactttgtcttgtagagggggtcaatccgtttcctcttcttatttggcttggtatcaacaaaaaccaggtcaagctccaagattattgatgtacgatgcttctattagagccaccggtattccagatagattttctggttctggttccggtactgatttcactttgactatctctagattggaaccagaagatttcgctgtttactactgtcaacaatatcagtcttggccattgacttttggtcaaggtacaaaggttgaaatcaaa

SEQ ID NO: 371 - DNA, 345 bp, Artificial Sequence
  source: /note=“Description of Artificial Sequence Synthetic polynucleotide”
gaggtgcagctgctggaatctggcggaggactggtgcagcctggcggctctctgagactgtcttgtgccgccagcggcttcaccttcagcagctaccctatgagctgggtgcgccaggcccctggcaaaggactggaatgggtgtccgccatcggaggctctggcggcagcacctactacgccgatagcgtgaagggccggttcaccatcagccgggacaacagcaagaacaccctgtacctgcaaatgaacagcctgcgggccgaggacaccgccgtgtactactgtgccagatactggcccatggacagctggggccagggaactttggtcaccgtctcctca

SEQ ID NO: 372 - DNA, 324 bp, Artificial Sequence
  source: /note=“Description of Artificial Sequence Synthetic polynucleotide”
gagatcgtgctgacacagagccctggcaccctgagcctgtctccaggcgaaagagccaccctgtcctgcaaagccagccagagcgtgtccagcacatacctggcctggtatcagcaaaagcccggccaggctccccggctgctgatctacgatgcctcttctagagcccctggcatccccgacagattcagcggctctggcagcggcaccgacttcaccctgaccatcagcagactggaacccgaggacttcgccgtgtactactgccagcagtacagcaccagccccctgacctttggccagggcacaaaggtggagatcaaa

SEQ ID NO: 373 - DNA, 345 bp, Artificial Sequence
  source: /note=“Description of Artificial Sequence Synthetic polynucleotide”
gaggtgcagctgctggaatctggcggaggactggtgcagcctggcggctctctgagactgtcttgtgccgccagcggcttcaccttcagcagctaccctatgagctgggtgcgccaggcccctggtaaaggtttggaatgggtttctgctattggtggttcaggtggttggagttattatgccgattctgttaagggtagattcaccatttctagagacaactctaagaacaccttgtacttgcaaatgaactccttgagagctgaagatactgctgtttattactgtgctagatactggccaatggattcttggggtcaaggtactctggtcaccgtctcctca

SEQ ID NO: 374 - DNA, 324 bp, Artificial Sequence
  source: /note=“Description of Artificial Sequence Synthetic polynucleotide”
gagatcgtgctgacacagagccctggcaccctgagcctgtctcctggtgaaagagctactttgtcttgttggttgtctcaatctgtttcctctacttacttggcttggtatcaacaaaaaccaggtcaagctccaagattattgatctacgatgcttcttctagagcaccaggtattccagatagattttctggttctggttccggtactgatttcactttgactatctctagattggaaccagaagatttcgctgtttactactgccaacaatactctgagtggccattgacttttggtcaaggtacaaaggttgaaatcaaa

SEQ ID NO: 375 - DNA, 345 bp, Artificial Sequence
  source: /note=“Description of Artificial Sequence Synthetic polynucleotide”
gaggtgcagctgctggaatctggcggaggactggtgcagcctggcggctctctgagactgtcttgtgccgccagcggcttcaccttcagcagctacgccatgaactgggtgcgccaggcccctggtaaaggtttggaatgggtttctgctatttctgattctggtggttctaggtggtatgccgattctgttaagggtagattcaccatttctagagacaactctaagaacaccttgtacttgcaaatgaactccttgagagctgaagatactgctgtttattactgtacgcggtactggccaatggatatttggggtcaaggtactctggtcaccgtctcctca

SEQ ID NO: 376 - DNA, 324 bp, Artificial Sequence; identical to SEQ ID NO: 374
  source: /note=“Description of Artificial Sequence Synthetic polynucleotide”

SEQ ID NO: 377 - protein, 11 aa, Artificial Sequence
  source: /note=“Description of Artificial Sequence Synthetic peptide”
RASQLGSFYLA

SEQ ID NO: 378 - protein, 118 aa, Artificial Sequence
  source: /note=“Description of Artificial Sequence Synthetic polypeptide”
EVQLLESGGGLVQPGGSLRLSCAASGFTFSSYAMSWVRQAPGKGLEWVSAISASGGSTYYADSVKGRFTISRDNSKNTLYLQMNSLRAEDTAVYYCARLSWSGAFDNWGQGTLVTVSS

SEQ ID NO: 379 - protein, 109 aa, Artificial Sequence
  source: /note=“Description of Artificial Sequence Synthetic polypeptide”
EIVLTQSPGTLSLSPGERATLSCRASQNVSSSYLAWYQQKPGQAPRLLIYGASYRATGIPDRFSGSGSGTDFTLTISRLEPEDFAVYYCQHYGSPPSFTFGQGTKVEIK

SEQ ID NO: 380 - protein, 118 aa, Artificial Sequence
  source: /note=“Description of Artificial Sequence Synthetic polypeptide”
EVQLLESGGGLVQPGGSLRLSCAASGFTFRSYAMSWVRQAPGKGLEWVSAISGSGGSTFYADSVKGRFTISRDNSKNTLYLQMNSLRAEDTAVYYCATVGTSGAFGIWGQGTLVTVSS

SEQ ID NO: 381 - protein, 5 aa, Artificial Sequence
  source: /note=“Description of Artificial Sequence Synthetic peptide”
SYAMS

SEQ ID NO: 382 - protein, 10 aa, Artificial Sequence
  source: /note=“Description of Artificial Sequence Synthetic peptide”
GFTFSSYAMS

SEQ ID NO: 383 - protein, 6 aa, Artificial Sequence
  source: /note=“Description of Artificial Sequence Synthetic peptide”
SASGGS

SEQ ID NO: 384 - protein, 17 aa, Artificial Sequence
  source: /note=“Description of Artificial Sequence Synthetic peptide”
AISASGGSTYYADSVKG

SEQ ID NO: 385 - protein, 9 aa, Artificial Sequence
  source: /note=“Description of Artificial Sequence Synthetic peptide”
LSWSGAFDN

SEQ ID NO: 386 - protein, 5 aa, Artificial Sequence
  source: /note=“Description of Artificial Sequence Synthetic peptide”
SYAMS

SEQ ID NO: 387 - protein, 7 aa, Artificial Sequence
  source: /note=“Description of Artificial Sequence Synthetic peptide”
GFTFRSY

SEQ ID NO: 388 - protein, 10 aa, Artificial Sequence
  source: /note=“Description of Artificial Sequence Synthetic peptide”
GFTFRSYAMS

SEQ ID NO: 389 - protein, 6 aa, Artificial Sequence
  source: /note=“Description of Artificial Sequence Synthetic peptide”
SGSGGS

SEQ ID NO: 390 - protein, 17 aa, Artificial Sequence
  source: /note=“Description of Artificial Sequence Synthetic peptide”
AISGSGGSTFYADSVKG

SEQ ID NO: 391 - protein, 9 aa, Artificial Sequence
  source: /note=“Description of Artificial Sequence Synthetic peptide”
VGTSGAFGI

SEQ ID NO: 392 - protein, 7 aa, Artificial Sequence
  source: /note=“Description of Artificial Sequence Synthetic peptide”
GASSRAY

SEQ ID NO: 393 - protein, 10 aa, Artificial Sequence
  source: /note=“Description of Artificial Sequence Synthetic peptide”
QHYGSPPLFT

SEQ ID NO: 394 - protein, 12 aa, Artificial Sequence
  source: /note=“Description of Artificial Sequence Synthetic peptide”
RASQNVSSSYLA

SEQ ID NO: 395 - protein, 7 aa, Artificial Sequence
  source: /note=“Description of Artificial Sequence Synthetic peptide”
GASYRAT

SEQ ID NO: 396 - protein, 519 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYAMNWVRQAPGKGLEWVSAILSSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRGGQSVSSSYLAWYQQKPGQAPRLLMYDASIRATGIPDRFSGSGSGTDFTLTISRLEPEDFAVYYCQQYQSWPLTFGQGTKVEIKGSGGGGSCPYSNPSLCSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 397 - protein, 9 aa, Artificial Sequence
  source: /note=“Description of Artificial Sequence Synthetic peptide”
CPYSNPSLC

SEQ ID NO: 398 - protein, 37 aa, Artificial Sequence
  source: /note=“Description of Artificial Sequence Synthetic polypeptide”
GSGGGGSCPYSNPSLCSGGGGSCPYSNPSLCSGGGGS

SEQ ID NO: 399 - DNA, 1497 bp, Artificial Sequence
  source: /note=“Description of Artificial Sequence Synthetic polynucleotide”
atggcactgcctgtgaccgccctgctgctgccactggccctgctgctgcacgccgcccggccagaggtgcagctgctggagagcggaggaggactggtgcagccaggaggaagcctgagactgtcctgcgccgcctctggcttcaccttcagctcctacgccatgaactgggtgaggcaggcaccaggcaagggactggagtgggtgtctgccatcctgtctagcggcggcagcacctactatgccgattccgtgaagggccgcttcacaatcagccgggacaactccaagaataccctgtacctgcagatgaacagcctgagagccgaggatacagccgtgtactattgcgccaggtattggcccatggacatctggggccagggcacactggtgaccgtgtcctctggaggaggaggatccggcggaggaggctctggcggcggcggcagcgagatcgtgctgacacagagcccaggcaccctgagcctgtcccctggagagagagccaccctgtcttgtaggggcggccagagcgtgagctcctcttacctggcctggtatcagcagaagccaggccaggcccccagactgctgatgtacgacgcctccatcagggcaacaggcatccccgatcggttctctggaagcggatccggaaccgactttacactgaccatctccaggctggagcctgaggatttcgccgtgtactattgccagcagtaccagtcttggccactgacatttggccagggcaccaaggtggagatcaagggatccggaggaggaggatcttgcccttattccaacccatctctgtgcaccacaacccctgcaccaaggccacctacaccagcacctaccatcgcctctcagccactgagcctgagacccgaggcctgtaggcctgcagcaggaggagcagtgcacacacggggactggactttgcctgcgatatctacatctgggcacctctggcaggaacatgtggcgtgctgctgctgagcctggtcatcaccctgtactgcaagagaggcaggaagaagctgctgtatatcttcaagcagccctttatgcgccctgtgcagacaacccaggaggaggatggctgctcctgtaggttcccagaagaggaggagggaggatgtgagctgcgcgtgaagttttctcggagcgccgacgcacctgcataccagcagggacagaatcagctgtataacgagctgaatctgggccggagagaggagtatgacgtgctggataagaggaggggaagggacccagagatgggaggcaagccacggagaaagaacccccaggagggcctgtacaatgagctgcagaaggataagatggccgaggcctatagcgagatcggcatgaagggagagaggcgccggggcaagggacacgacggactgtaccagggactgtccacagccaccaaggacacctatgatgccctgcacatgcaggccctgccaccaaggtga

SEQ ID NO: 400 - protein, 157 aa, Artificial Sequence
  source: /note=“Description of Artificial Sequence Synthetic polypeptide”
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 401 - protein, 109 aa, Artificial Sequence
  source: /note=“Description of Artificial Sequence Synthetic polypeptide”
EIVLTQSPGTLSLSPGERATLSCRASQSVSSSYLAWYQQKPGQAPRLLIYGASSRAYGIPDRFSGSGSGTDFTLTISRLEPEDFAVYYCQHYGSPPLFTFGQGTKVEIK

SEQ ID NO: 402 - protein, 5 aa, Artificial Sequence
  source: /note=“Description of Artificial Sequence Synthetic peptide”
DFYMN

SEQ ID NO: 403 - protein, 19 aa, Artificial Sequence
  source: /note=“Description of Artificial Sequence Synthetic peptide”
FIRDKAKGYTTEYNPSVKG

SEQ ID NO: 404 - protein, 10 aa, Artificial Sequence
  source: /note=“Description of Artificial Sequence Synthetic peptide”
EGHTAAPFDY

SEQ ID NO: 405 - protein, 11 aa, Artificial Sequence
  source: /note=“Description of Artificial Sequence Synthetic peptide”
KASQNIDKYLN

SEQ ID NO: 406 - protein, 7 aa, Artificial Sequence
  source: /note=“Description of Artificial Sequence Synthetic peptide”
NTNNLQT

SEQ ID NO: 407 - protein, 9 aa, Artificial Sequence
  source: /note=“Description of Artificial Sequence Synthetic peptide”
LQHISRPRT

SEQ ID NO: 408 - protein, 451 aa, Artificial Sequence
  source: /note=“Description of Artificial Sequence Synthetic polypeptide”
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 409 - DNA, 1353 bp, Artificial Sequence
  source: /note=“Description of Artificial Sequence Synthetic polynucleotide”
caagtgcagcttcaagaatccggccctggtctggtccgcccctcccaaaccctctccctgacatgcaccgtgtcgggattcacctttaccgatttctacatgaactgggtccggcagccgcccggaagaggtctggagtggatcggcttcattcgggacaaagccaaggggtacaccaccgagtacaacccgtccgtgaagggacgcgtgactatgctcgtggacacgtccaagaaccagttcagcttgaggctgagcagcgtgactgccgcggataccgcagtgtactactgtgcccgggaagggcacactgccgctccattcgactattggggccagggatcactggtcactgtgtcgtccgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggaaaa

SEQ ID NO: 410 - protein, 214 aa, Artificial Sequence
  source: /note=“Description of Artificial Sequence Synthetic polypeptide”
DIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 411 - DNA, 699 bp, Artificial Sequence
  source: /note=“Description of Artificial Sequence Synthetic polynucleotide”
atgggatggagctgtatcatcctcttcttggtagcaacagctacaggcgtgcactccgacatccaaatgacccaatccccatcctcactttccgcctccgtgggcgaccgcgtgactattacctgtaaagcgtcacagaatatcgacaagtacctgaactggtaccagcagaagcctggaaaggcccccaagctcctgatctacaacaccaacaacttgcagactggagtgccgagcagattttccggctccggctcggggactgatttcaccttcaccatctcgagcctgcagccggaggatattgctacctattactgcctgcaacacattagccggcccaggacgttcggacagggtaccaaggtcgaaatcaagcgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 412 - protein, 119 aa, Artificial Sequence
  source: /note=“Description of Artificial Sequence Synthetic polypeptide”
EVQLLESGGGLVQPGGSLRLSCAASGFTFGSYAMTWVRQAPGKGLEWVSAISGSGGNTFYADSVKGRFTISRDNSKNTLYLQMNSLRAEDTAVYYCARVSPIAALMDYWGQGTLVTVSS

SEQ ID NO: 413 - protein, 113 aa, Artificial Sequence
  source: /note=“Description of Artificial Sequence Synthetic polypeptide”
EVQLLESGGGLVQPGGSLRLSCAASGFTFSSYPMSWVRQAPGKGLEWVSAIGGSGGSLPYADSVKGTISRDNSKNTLYLQMNSLRAEDTAVYYCARYWPMDSWGQGTLVTVSS

SEQ ID NO: 414 - protein, 108 aa, Artificial Sequence
  source: /note=“Description of Artificial Sequence Synthetic polypeptide”
EIVLTQSPGTLSLSPGERATLSCRASQSVSSVYLAWYQQKPGQAPRLLIYDASSRAPGIPDRFSGSGSGTDFTLTISRLEPEDFAVYYCQQYSTWPLTFGQGTKVEIK

SEQ ID NO: 415 - protein, 12 aa, Artificial Sequence
  source: /note=“Description of Artificial Sequence Synthetic peptide”
RASQSVSPHYLA

SEQ ID NO: 416 - protein, 10 aa, Artificial Sequence
  source: /note=“Description of Artificial Sequence Synthetic peptide”
KFYPYPPSFT

SEQ ID NO: 417 - protein, 12 aa, Artificial Sequence
  source: /note=“Description of Artificial Sequence Synthetic peptide”
RASQNVSSSYLA

SEQ ID NO: 418 - protein, 519 aa, Artificial Sequence
  source: /note=“Description of Artificial Sequence Synthetic polypeptide”
MALPVTALLLPLALLLHAARPEVQLLESGGGLVQPGGSLRLSCAASGFTFSSYAMNWVRQAPGKGLEWVSAISDSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRASQSVSSSYLAWYQQKPGQAPRLLMYDASIRATGIPDRFSGSGSGTDFTLTISRLEPEDFAVYYCQQYGSWPLTFGQGTKVEIKGSGGGGSCPYSNPSLCSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 419 - protein, 498 aa, Artificial Sequence
  source: /note=“Description of Artificial Sequence Synthetic polypeptide”
EVQLLESGGGLVQPGGSLRLSCAASGFTFSSYAMNWVRQAPGKGLEWVSAISDSGGSTYYADSVKGRFTISRDNSKNTLYLQMNSLRAEDTAVYYCARYWPMDIWGQGTLVTVSSGGGGSGGGGSGGGGSEIVLTQSPGTLSLSPGERATLSCRASQSVSSSYLAWYQQKPGQAPRLLMYDASIRATGIPDRFSGSGSGTDFTLTISRLEPEDFAVYYCQQYGSWPLTFGQGTKVEIKGSGGGGSCPYSNPSLCSGGGGSCPYSNPSLCSGGGGSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 420 - protein, 121 aa, Artificial Sequence
  source: /note=“Description of Artificial Sequence Synthetic polypeptide”
QVQLQESGPGLVRPSQTLSLTCTVSGFTFTDFYMNWVRQPPGRGLEWIGFIRDKAKGYTTEYNPSVKGRVTMLVDTSKNQFSLRLSSVTAADTAVYYCAREGHTAAPFDYWGQGSLVTVSS

SEQ ID NO: 421 - protein, 108 aa, Artificial Sequence
  source: /note=“Description of Artificial Sequence Synthetic polypeptide”
DIQMTQSPSSLSASVGDRVTITCKASQNIDKYLNWYQQKPGKAPKLLIYNTNNLQTGVPSRFSGSGSGTDFTFTISSLQPEDIATYYCLQHISRPRTFGQGTKVEIKR

(19) United States
(12) Patent Application Publication
(10) Pub. No.: US 2023/0112444 A1
(43) Pub. Date: Apr. 13, 2023

(54) COMBINATION THERAPIES OF CHIMERIC ANTIGEN RECEPTORS TARGETING B-CELL MATURATION ANTIGEN AND GAMMA SECRETASE INHIBITORS

(71) Applicants: Allogene Therapeutics, Inc., South San Francisco, CA (US); SpringWorks Therapeutics, Inc., Stamford, CT (US)

(72) Inventors: Arun BALAKUMARAN, South San Francisco, CA (US); Cesar Adolfo SOMMER, South San Francisco, CA (US); Trevor Michael BENTLEY, South San Francisco, CA (US); Todd Webster SHEARER, Stamford, CT (US); Lesley Mary SMITH, Stamford, CT (US)

(73) Assignee: SpringWorks Therapeutics, Inc., Stamford, CT (US)

(21) Appl. No.: 17/758,913

(22) PCT Filed: Jan. 15, 2021
(86) PCT No.: PCT/US2021/013705

§ 371 Date: Jul. 15, 2022

Related U.S. Application Data
(60) Provisional application No. 62/962,014, filed on Jan. 16, 2020.
(60) Provisional application No. 63/117,281, filed on Nov. 23, 2020.

(51) Int. Cl. A61K 38/17; A61K 35/17; A61K 31/417; A61K 31/7076; A61K 31/675; A61K 39/395; A61K 31/55; A61K 31/5513; A61P 35/00

(52) U.S. Cl. CPC A61K 38/1774; A61K 35/17; A61K 31/417; A61K 38/1793; A61K 31/7076; A61K 31/675; A61K 39/3955; A61K 31/55; A61K 31/5513; A61P 35/00

(57) ABSTRACT

Provided herein are BCMA CARs and CAR-T cells, gamma secretase inhibitors, and the combination thereof. Also provided are the combination of BCMA CAR-T cells and gamma secretase inhibitors for use in treating cancer. In some embodiments, particular dosing regimens, redosing regimens, and combination regimens with lymphodepletion are provided, for the treatment and clinical management of multiple myeloma in subjects in need thereof.

### CROSS REFERENCE TO RELATED APPLICATIONS

The present application claims the benefit of priority to U.S. Provisional Application No. 62/962,014, filed on Jan. 16, 2020; and U.S. Provisional Application No. 63/117,281, filed on Nov. 23, 2020, the contents of all of which are hereby incorporated by reference in their entireties.

### SEQUENCE LISTING

The instant application contains a Sequence Listing which has been submitted electronically in ASCII format and is hereby incorporated by reference in its entirety. Said ASCII copy, created on Jan. 12, 2021, is named AT-033-03WO_SL.txt and is 424,127 bytes in size.

### BACKGROUND

Multiple myeloma (MM) is a malignancy characterized by an accumulation of clonal plasma cells. MM largely remains incurable, and most subjects develop resistance over time.

B-cell maturation antigen (BCMA, CD269, or TNFRSF17) is a member of the tumor necrosis factor receptor (TNFR) superfamily and is involved in pro-survival signaling. BCMA was identified in a malignant human T cell lymphoma containing a t(4;16) translocation. BCMA is expressed at high levels on normal and malignant plasma cells at all stages of MM and some other plasma cell malignancies (e.g. DLBCL). BCMA is also expressed on most or all myeloma cells, and expression absent on non-B cell lineages.

Adoptive transfer of T-cells genetically modified to recognize malignancy-associated antigens is showing promise as a new approach to treating cancer. T-cells can be genetically modified to express chimeric antigen receptors (CARs), which are fusion proteins comprised of an antigen recognition moiety and T-cell activation domains.

The ectodomain of BCMA expressed on the surface of B cells is cleaved by gamma secretase, an integral membrane protease. Shedding BCMA ectodomain from cell surface by proteolytic cleavage may be a way BCMA positive malignant plasma cells evade recognition and binding of BCMA antibody or CAR-T therapies.

There is an unmet medical need for interventions that can effectively treat MM, including relapsed/refractory MM. Provided herein are methods and compositions that address this need.

### SUMMARY

Combination cancer therapies comprising chimeric antigen receptors (CARs) that bind to BCMA and gamma secretase inhibitors are provided herein; as well as dosing paradigms for use in the treatment of cancer. In certain embodiments, the cancer is multiple myeloma (MM), including relapsed and/or refractory MM.

More specifically, in one aspect provided herein is a method of treating MM in a subject comprising administering to the subject at least one dose of allogeneic chimeric antigen receptor (CAR)-T cells comprising an anti-human BCMA CAR (BCMA CAR-T cells), in combination with a gamma secretase inhibitor (GSI). In some embodiments, the gamma secretase inhibitor is nirogacestat having the structure of:

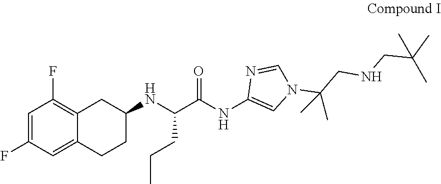 [SMILES: CCC[C@H](N[C@H]1CCc2cc(F)cc(F)c2C1)C(=O)Nc1cn(C(C)(C)CNCC(C)(C)C)cn1]

or a pharmaceutically acceptable salt thereof. In some embodiments, the pharmaceutically acceptable salt is hydrobromide. In some embodiments, the pharmaceutically acceptable salt is dihydrobromide.

In some embodiments, the subject is administered Compound I, or a pharmaceutically acceptable salt form thereof, at a dose from about 20 mg to about 220 mg once or twice daily. In some embodiments, the subject is administered Compound I, or a pharmaceutically acceptable salt form thereof, for at least one week.

In some embodiments, the at least one dose of BCMA CAR-T cells is about 7×10\hat{ }6 cells/dose to about 480×10\hat{ }6 cells/dose. In some embodiments, the at least one dose of BCMA CAR-T cells ranges from about 20×10\hat{ }6 cells/dose to about 480×10\hat{ }6 cells/dose.

In some embodiments, the at least one dose is from about 20×10\hat{ }6 cells/dose to about 40×10\hat{ }6 cells/dose, from about 40×10\hat{ }6 cells/dose to about 160×10\hat{ }6 cells/dose, from about 160×10\hat{ }6 cells/dose to about 240×10\hat{ }6 cells/dose, from about 240×10\hat{ }6 cells/dose to about 320×10\hat{ }6 cells/dose, from about 160×10\hat{ }6 cells/dose to about 320×10\hat{ }6 cells/dose, or from about 320×10\hat{ }6 cells/dose to about 480×10\hat{ }6 cells/dose. In some embodiments, the at least one dose is about 20×10\hat{ }6 cells/dose, about 40×10\hat{ }6 cells/dose, about 160×10\hat{ }6 cells/dose, about 240×10\hat{ }6 cells/dose, about 320×10\hat{ }6 cells/dose, or about 480×10\hat{ }6 cells/dose. In some embodiments, the at least one dose is about 40×10\hat{ }6 cells/dose, about 160×10\hat{ }6 cells/dose, about 320×10\hat{ }6 cells/dose, or about 480×10\hat{ }6 cells/dose. In some embodiments, the subject is administered more than one dose of the BCMA CAR-T cells and/or more than two doses of Compound I, or a pharmaceutically acceptable salt form thereof, over the course of treatment. In some embodiments, Compound I, or a pharmaceutically acceptable salt form thereof, is administered to the subject before, concomitantly, or subsequently to the administering of the at least one dose of BCMA CAR-T cells.

In some embodiments, the subject is administered Compound I, or a pharmaceutically acceptable salt form thereof, at a dose from about 20 mg to about 220 mg once or twice daily. In some embodiments, the subject is administered Compound I, or a pharmaceutically acceptable salt form thereof, at a dose at about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, or about 220 mg, once or twice daily for at least one week, at least two weeks, at least three weeks, at least four weeks, at least five weeks, at least six weeks, at least seven weeks, or at least eight weeks. In some embodiments, the subject is administered Compound I at a dose at about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, or about 220 mg once or twice daily from Day 0 to Day 10, Day 0 to Day 15, Day 0 to Day 20, Day 0 to Day 21, Day 0 to Day 22, Day 0 to Day 23, Day 0 to Day 24, Day 0 to Day 25, Day 0 to Day 26, Day 0 to Day 27, Day 0 to Day 28, Day 0 to Day 29, Day 0 to Day 30, Day 0 to Day 31, Day 0 to Day 32, Day 0 to Day 33, Day 0 to Day 34, Day 0 to Day 35, Day 0 to Day 36, Day 0 to Day 37, Day 0 to Day 38, Day 39, Day 0 to Day 40, Day 0 to Day 41, or Day 0 to Day 42. In some embodiments, the subject is administered Compound I at a dose at about 100 mg once or twice daily from Day 0 to about Day 41 and beyond.

In some embodiments, the subject is administered Compound I, or a pharmaceutically acceptable salt form thereof, at a dose at about 100 mg twice daily for at least six weeks. In some embodiments, the subject is administered Compound I, or a pharmaceutically acceptable salt form thereof, at a dose at about 100 mg twice daily for about six weeks. In some embodiments, the subject is administered Compound I, or a pharmaceutically acceptable salt form thereof, at a dose at about 100 mg twice daily from Day 0 to Day 41. In some embodiments, the subject is administered Compound I, or a pharmaceutically acceptable salt form thereof, at a dose at about 100 mg twice daily from Day 0 to about Day 41. In some embodiments, the subject is administered Compound I, or a pharmaceutically acceptable salt form thereof, at a dose at about 100 mg once or twice daily from Day 0 to Day 10, from Day 0 to Day 15, from Day 0 to Day 20, from Day 0 to Day 21, from Day 0 to Day 22, from Day 0 to Day 23, from Day 0 to Day 24, from Day 0 to Day 25, from Day 0 to Day 26, from Day 0 to Day 27, from Day 0 to Day 28, from Day 0 to Day 29, from Day 0 to Day 30, from Day 0 to Day 31, from Day 0 to Day 32, from Day 0 to Day 33, from Day 0 to Day 34, from Day 0 to Day 35, from Day 0 to Day 36, from Day 0 to Day 37, from Day 0 to Day 38, from Day 0 to Day 39, from Day 0 to Day 40, from Day 0 to Day 41, or from Day 0 to Day 42. In some embodiments, the subject is administered Compound I, or a pharmaceutically acceptable salt form thereof, at a dose at about 100 mg once or twice daily from Day 0 to Day 21. In some embodiments, the subject is administered Compound I, or a pharmaceutically acceptable salt form thereof, at a dose at about 100 mg once or twice daily from Day 0 to Day 28. In some embodiments, the subject is administered Compound I, or a pharmaceutically acceptable salt form thereof, at a dose at about 100 mg once or twice daily from Day 0 to about Day 41 and beyond.

In some embodiments, the subject is administered at least one dose of BCMA CAR-T cells on Day 0, after the first dose of Compound I, or a pharmaceutically acceptable salt form thereof. In some embodiments, the subject is administered at least one dose of BCMA CAR-T cells on Day 0, after the second dose of Compound I, or a pharmaceutically acceptable salt form thereof. In some embodiments, the at least one dose of CAR T cells is about 20×10\hat{ }6 cells/dose, about 40×10\hat{ }6 cells/dose, about 160×10\hat{ }6 cells/dose, about 240×10\hat{ }6 cells/dose, about 320×10\hat{ }6 cells/dose, or about 480×10\hat{ }6 cells/dose. In some embodiments, the at least one dose of CAR T cells is about 320×10\hat{ }6 cells/dose, or about 480×10\hat{ }6 cells/dose.

In some embodiments, the subject is administered Compound I, or a pharmaceutically acceptable salt form thereof, in tablet form. In some embodiments, the subject is administered Compound I, or a pharmaceutically acceptable salt form thereof, in suspension form or solution form.

In some embodiments, the weight of the subject is at least 50 kg, and the method comprises administering at least one dose of BCMA CAR-T cells, wherein the dose ranges from about 20×10\hat{ }6 cells/dose to about 480×10\hat{ }6 cells/dose. In some embodiments, the at least one dose is about 20×10\hat{ }6 cells/dose, about 40×10\hat{ }6 cells/dose, about 120×10\hat{ }6 cells/dose, about 360×10\hat{ }6 cells/dose, or about 480×10\hat{ }6 cells/dose. In some embodiments, the at least one dose is from about 20×10\hat{ }6 cells/dose to about 40×10\hat{ }6 cells/dose, from about 40×10\hat{ }6 cells/dose to about 120×10\hat{ }6 cells/dose, from about 120×10\hat{ }6 cells/dose to about 360×10\hat{ }6 cells/dose, or from about 360×10\hat{ }6 cells/dose to about 480×10\hat{ }6 cells/dose.

In some embodiments, the weight of the subject is greater than 50 kg, and the method comprises administering at least one dose of BCMA CAR-T cells, wherein the dose ranges from about 20×10\hat{ }6 cells/dose to about 480×10\hat{ }6 cells/dose. In some embodiments, the at least one dose is about 20×10\hat{ }6 cells/dose, about 40×10\hat{ }6 cells/dose, about 160×10\hat{ }6 cells/dose, about 240×10\hat{ }6 cells/dose, about 320×10\hat{ }6 cells/dose, or about 480×10\hat{ }6 cells/dose. In some embodiments, the at least one dose is from about 20×10\hat{ }6 cells/dose to about 40×10\hat{ }6 cells/dose, from about 40×10\hat{ }6 cells/dose to about 160×10\hat{ }6 cells/dose, from about 160×10\hat{ }6 cells/dose to about 240×10\hat{ }6 cells/dose, from about 240×10\hat{ }6 cells/dose to about 320×10\hat{ }6 cells/dose, from about 160×10\hat{ }6 cells/dose to about 320×10\hat{ }6 cells/dose, or from about 320×10\hat{ }6 cells/dose to about 480×10\hat{ }6 cells/dose.

In some embodiments, the weight of the subject is less than 50 kg, and the method comprises administering at least one dose of BCMA CAR-T cells, wherein the dose ranges from about 7×10\hat{ }6 cells/dose to about 360×10\hat{ }6 cells/dose. In some embodiments, the at least one dose is about 7×10\hat{ }6 cells/dose, about 14×10\hat{ }6 cells/dose, about 20×10\hat{ }6 cells/dose, about 80×10\hat{ }6 cells/dose, about 240×10\hat{ }6 cells/dose, or about 360×10\hat{ }6 cells/dose. In some embodiments, the at least one dose is from about 7×10\hat{ }6 or 14×10\hat{ }6 cells/dose to about 20×10\hat{ }6 cells/dose, from about 20×10\hat{ }6 cells/dose to about 80×10\hat{ }6 cells/dose, from about 80×10\hat{ }6 cells/dose to about 240×10\hat{ }6 cells/dose, or from about 240×10\hat{ }6 cells/dose to about 360×10\hat{ }6 cells/dose.

In some embodiments, the weight of the subject is no more than 50 kg, and the method comprises administering at least one dose of BCMA CAR-T cells, wherein the dose ranges from about 14×10\hat{ }6 cells/dose to about 320×10\hat{ }6 cells/dose. In some embodiments, the at least one dose is about 14×10\hat{ }6 cells/dose, about 20×10\hat{ }6 cells/dose, about 80×10\hat{ }6 cells/dose, about 160×10\hat{ }6 cells/dose about 200×10\hat{ }6 cells/dose, or about 320×10\hat{ }6 cells/dose. In some embodiments, the at least one dose is about 14×10\hat{ }6 cells/dose to about 20×10\hat{ }6 cells/dose, from about 20×10\hat{ }6 cells/dose to about 80×10\hat{ }6 cells/dose, from about 80×10\hat{ }6 cells/dose to about 200×10\hat{ }6 cells/dose, from about 80×10\hat{ }6 cells/dose to about 160×10\hat{ }6 cells/dose, from about 160×10\hat{ }6 cells/dose to about 200×10\hat{ }6 cells/dose, or from about 200×10\hat{ }6 cells/dose to about 320×10\hat{ }6 cells/dose.

In some embodiments, the subject has not received any prior therapy for multiple myeloma. In some embodiments, the subject has received at least one, two, or three prior therapies for multiple myeloma. In some embodiments, the dosing regimens are a first line therapy. In some embodiments, the dosing regimens are a second line therapy. In some embodiments, the dosing regimens are a third line therapy. In some embodiments, the dosing regimens are a fourth line therapy.

In some embodiments, the subject has received a prior chemotherapeutic regimen; a prior biologics-based regimen, and/or a prior autologous cell therapy-based regimen (e.g. stem cell therapy). In some embodiments, the subject has not received a prior chemotherapeutic regimen; a prior biologics-based regimen, and/or a prior autologous cell therapy-based regimen.

In some embodiments, the subject has relapsed MM. In some embodiments, the subject has refractory MM. In some embodiments, the subject has relapsed and refractory MM.

In some embodiments, the BCMA CAR-T cells comprise a CAR comprising an extracellular binding domain comprising a single chain Fv fragment (scFv), wherein the scFv comprises a heavy chain variable (VH) region and a light chain variable (VL) region, wherein the VH region comprises a VH complementary determining region 1 (VH CDR1), a VH complementary determining region 2 (VH CDR2), and a VH complementary determining region 3 (VH CDR3) and the VL region comprises a VL complementary determining region 1 (VL CDR1), a VL complementary determining region 2 (VL CDR2), and a VL complementary determining region 3 (VL CDR3), wherein: (a) the VH CDR1 comprises the amino acid sequence of SEQ ID NO: 150, 151, or 152; the VH CDR2 comprises the amino acid sequence of SEQ ID NO: 153 or 154; the VH CDR3 comprises the amino acid sequence of SEQ ID NO: 155; the VL CDR1 comprises the amino acid sequence of SEQ ID NO: 209; the VL CDR2 comprises the amino acid sequence of SEQ ID NO: 221; and the VL CDR3 comprises the amino acid sequence of SEQ ID NO: 222; (b) the VH CDR1 comprises the amino acid sequence of SEQ ID NO: 150, 151, or 152; the VH CDR2 comprises the amino acid sequence of SEQ ID NO: 187 or 188; the VH CDR3 comprises the amino acid sequence of SEQ ID NO: 155; the VL CDR1 comprises the amino acid sequence of SEQ ID NO: 249; the VL CDR2 comprises the amino acid sequence of SEQ ID NO: 221; and the VL CDR3 comprises the amino acid sequence of SEQ ID NO: 225; (c) the VH CDR1 comprises the amino acid sequence of SEQ ID NO: 150, 151, or 152; the VH CDR2 comprises the amino acid sequence of SEQ ID NO: 165 or 166; the VH CDR3 comprises the amino acid sequence of SEQ ID NO: 155; the VL CDR1 comprises the amino acid sequence of SEQ ID NO: 226; the VL CDR2 comprises the amino acid sequence of SEQ ID NO: 221; and the VL CDR3 comprises the amino acid sequence of SEQ ID NO: 227; (d) the VH CDR1 comprises the amino acid sequence of SEQ ID NO: 151, 156, or 157; the VH CDR2 comprises the amino acid sequence of SEQ ID NO: 159 or 162; the VH CDR3 comprises the amino acid sequence of SEQ ID NO: 161; the VL CDR1 comprises the amino acid sequence of SEQ ID NO: 251; the VL CDR2 comprises the amino acid sequence of SEQ ID NO: 252; and the VL CDR3 comprises the amino acid sequence of SEQ ID NO: 253; (e) the VH CDR1 comprises the amino acid sequence of SEQ ID NO: 151, 156, or 157; the VH CDR2 comprises the amino acid sequence of SEQ ID NO: 190 or 191; the VH CDR3 comprises the amino acid sequence of SEQ ID NO: 161; the VL CDR1 comprises the amino acid sequence of SEQ ID NO: 262; the VL CDR2 comprises the amino acid sequence of SEQ ID NO: 252; and the VL CDR3 comprises the amino acid sequence of SEQ ID NO: 263; (f) the VH CDR1 comprises the amino acid sequence of SEQ ID NO: 150, 151, or 152; the VH CDR2 comprises the amino acid sequence of SEQ ID NO: 154 or 169; the VH CDR3 comprises the amino acid sequence of SEQ ID NO: 155; the VL CDR1 comprises the amino acid sequence of SEQ ID NO: 271; the VL CDR2 comprises the amino acid sequence of SEQ ID NO: 221; and the VL CDR3 comprises the amino acid sequence of SEQ ID NO: 272; (g) the VH CDR1 comprises the amino acid sequence of SEQ ID NO: 129, 130, or 131; the VH CDR2 comprises the amino acid sequence of SEQ ID NO: 139 or 140; the VH CDR3 comprises the amino acid sequence of SEQ ID NO: 134; the VL CDR1 comprises the amino acid sequence of SEQ ID NO: 217; the VL CDR2 comprises the amino acid sequence of SEQ ID NO: 210; and the VL CDR3 comprises the amino acid sequence of SEQ ID NO: 216; (h) the VH CDR1 comprises the amino acid sequence of SEQ ID NO: 151, 156, or 157; the VH CDR2 comprises the amino acid sequence of SEQ ID NO: 158 or 159; the VH CDR3 comprises the amino acid sequence of SEQ ID NO: 155; the VL CDR1 comprises the amino acid sequence of SEQ ID NO: 209; the VL CDR2 comprises the amino acid sequence of SEQ ID NO: 221; and the VL CDR3 comprises the amino acid sequence of SEQ ID NO: 225; or (i) the VH CDR1 comprises the amino acid sequence of SEQ ID NO: 129, 130, or 131; the VH CDR2 comprises the amino acid sequence of SEQ ID NO: 132 or 133; the VH CDR3 comprises the amino acid sequence of SEQ ID NO: 137; the VL CDR1 comprises the amino acid sequence of SEQ ID NO: 377; the VL CDR2 comprises the amino acid sequence of SEQ ID NO: 210; and the VL CDR3 comprises the amino acid sequence of SEQ ID NO: 214.

In some embodiments, the VH region of the scFv of a BCMA CAR comprises a VH CDR1 comprising the amino acid sequence of SEQ ID NO: 150, 151, or 152; a VH CDR2 comprising the amino acid sequence of SEQ ID NO: 153 or 154; and a VH CDR3 comprising the amino acid sequence of SEQ ID NO: 155; and the VL region of the scFv comprises a VL CDR1 comprising the amino acid sequence of SEQ ID NO: 209; a VL CDR2 comprising the amino acid sequence of SEQ ID NO: 221; and a VL CDR3 comprising the amino acid sequence of SEQ ID NO: 222.

In some embodiments, the VH region of the scFv of a BCMA CAR comprises a VH CDR1 comprising the amino acid sequence of SEQ ID NO: 151, 156, or 157; a VH CDR2 comprising the amino acid sequence of SEQ ID NO: 158 or 159; and a VH CDR3 comprising the amino acid sequence of SEQ ID NO: 155; and the VL region of the scFv comprises a VL CDR1 comprising the amino acid sequence of SEQ ID NO: 209; a VL CDR2 comprising the amino acid sequence of SEQ ID NO: 221; and a VL CDR3 comprising the amino acid sequence of SEQ ID NO: 225.

In some embodiments, the BCMA CAR-T cells comprise a CAR comprising the amino acid sequence shown in SEQ ID NO: 344. In some of these embodiments, the CAR further comprises a CD20 epitope. In some of these embodiments, the CD20 epitope comprises the amino acid sequence shown in SEQ ID NO: 397 or SEQ ID NO: 398. In some embodiments, the BCMA CAR-T cells comprise a CAR comprising the amino acid sequence shown in SEQ ID NO: 418 or SEQ ID NO: 419.

In some embodiments, the BCMA CAR-T cells comprise a CAR comprising a CD8α signal peptide having the sequence of SEQ ID NO: 318; a VH region having the sequence of SEQ ID NO: 112; a GS linker having the sequence of SEQ ID NO: 333; a VL region having the sequence of SEQ ID NO: 38; a CD8α hinge having the sequence of SEQ ID NO: 320; a CD8α transmembrane domain having the sequence of SEQ ID NO: 322; a 4-1BB intracellular signaling domain having the sequence of SEQ ID NO: 323; and a CD3ζ intracellular signaling domain having the sequence of SEQ ID NO: 324.

In some embodiments, the BCMA CAR-T cells comprise a CAR comprising a CD8α signal peptide having the sequence of SEQ ID NO: 318; a VH region having the sequence of SEQ ID NO: 112; a GS linker having the sequence of SEQ ID NO: 333; a VL region having the sequence of SEQ ID NO: 38; a CD20 epitope having the sequence of SEQ ID NO: 398; a CD8α hinge having the sequence of SEQ ID NO: 320; a CD8α transmembrane domain having the sequence of SEQ ID NO: 322; a 4-1BB intracellular signaling domain having the sequence of SEQ ID NO: 323; and a CD3ζ intracellular signaling domain having the sequence of SEQ ID NO: 324.

In some embodiments, the BCMA CAR-T cells comprise a CAR comprising a CD8α signal peptide having the sequence of SEQ ID NO: 318; a VH region having the sequence of SEQ ID NO: 33; a GS linker having the sequence of SEQ ID NO: 333; a VL region having the sequence of SEQ ID NO: 34; a CD8α hinge having the sequence of SEQ ID NO: 320; a CD8α transmembrane domain having the sequence of SEQ ID NO: 322; a 4-1BB intracellular signaling domain having the sequence of SEQ ID NO: 323; and a CD3ζ intracellular signaling domain having the sequence of SEQ ID NO: 324

In some embodiments, the BCMA CAR-T cells comprise a CAR comprising a CD8α signal peptide having the sequence of SEQ ID NO: 318; a VH region having the sequence of SEQ ID NO: 33; a GS linker having the sequence of SEQ ID NO: 333; a VL region having the sequence of SEQ ID NO: 34; a CD20 epitope having the sequence of SEQ ID NO: 398; a CD8α hinge having the sequence of SEQ ID NO: 320; a CD8α transmembrane domain having the sequence of SEQ ID NO: 322; a 4-1BB intracellular signaling domain having the sequence of SEQ ID NO: 323; and a CD3ζ intracellular signaling domain having the sequence of SEQ ID NO: 324.

In some embodiments, the BCMA CAR-T cells comprise a CAR comprising an extracellular binding domain comprising a single chain Fv fragment (scFv), wherein the scFv comprises a VH region and a VL region, wherein the combination of VH and VL regions are chosen from the combinations presented in Table 1. In some embodiments, the BCMA CAR-T cells comprise a CAR comprising an extracellular ligand-binding domain, a first transmembrane domain, and an intracellular signaling domain, wherein the extracellular domain comprises a scFv comprising a heavy chain variable (VH) region comprising a sequence shown in SEQ ID NO: 33, 72, 39, 76, 83, 92, 25, 112, or 8 of Table 1; and a light chain variable (VL) region comprising a sequence shown in SEQ ID NO: 34, 73, 40, 77, 84, 93, 18, 38, or 80 of Table 1, wherein the first transmembrane domain comprises a CD8α chain transmembrane domain, and wherein the intracellular signaling domain comprises a CD3ζ signaling domain and/or a 4-1BB signaling domain. In some embodiments, the VH comprises SEQ ID NO: 33 and the VL comprises SEQ ID NO: 34. In some embodiments, the VH comprises SEQ ID NO: 112 and the VL comprises SEQ ID NO: 38.

In some embodiments, the CAR-T cells are deficient in CD52. In some embodiments, the CAR-T cells are deficient in TCRα and/or TCRβ. In some embodiments, the CAR-T cells do not express a safety switch. In some embodiments, the genotype of the cells is TCRαβ⁻ and CD52^{+/-}.

In some embodiments, the subject receives a first lymphodepletion regimen prior to administration of the at least one dose. In some embodiments, the first lymphodepletion regimen comprises administering fludarabine and cyclophosphamide. In some embodiments, the first lymphodepletion regimen comprises administering fludarabine, cyclophosphamide, and an anti-CD52 antibody. In some embodiments, the first lymphodepletion regimen comprises administering an anti-CD52 antibody. In some embodiments, the first lymphodepletion regimen comprises administering only an anti-CD52 antibody. In some embodiments, the fludarabine is administered at a dosage of about 30 mg/m2/day; cyclophosphamide is administered at a dosage of about 300 mg/m2/day; and CD52 antibody is administered at a dosage of about 10 to about 13 mg/day, about 13 to 20 mg/day, about 13 to 30 mg/day, or about 20 to 30 mg/day. In some embodiments, the first lymphodepletion regimen is initiated between about 1 to 15 days prior to administration of the at least one dose. In some embodiments, the first lymphodepletion regimen is administered over the course of 1, 2, 3, 4, or 5 days. In some embodiments, the first lymphodepletion regimen is administered 5 days prior to administration of the at least one dose in the course of 3 days. In some embodiments, the first lymphodepletion regimen is administered 7 days prior to administration of the at least one dose in the course of 3 days. In some embodiments, the fludarabine is administered at a total dosage of about 90 mg/m²; cyclophosphamide is administered at a dosage of about 900 mg/m²; and anti-CD52 antibody is administered at a total dosage of about 60 mg.

In some embodiments, the subject receives a subsequent dose of the CAR-T cells.

In another aspect provided herein is a formulation comprising BCMA CAR-T cells. In one embodiment the formulation comprises a solution comprising about 5% dimethyl sulfoxide (DMSO) and 14×10\hat{ }6 cells/mL. In another embodiment the cells are formulated in a 1:1 mixture of CryoStor® Basal Solution and CryoStor® CS10 resulting in a 5% final concentration of dimethyl sulfoxide, wherein the dosage strength of the formulation is 14×10\hat{ }6 cells/mL, wherein the genotype of the cells is BCMA-CAR+_TCRαβ−_CD52+/−, and wherein the BCMA CAR-T cells comprise a CAR comprising an extracellular ligand-binding domain, two rituximab-binding domains, a first transmembrane domain, and an intracellular signaling domain, wherein the extracellular domain comprises a scFv comprising a heavy chain variable (VH) region comprising a sequence shown in SEQ ID NO: 33, 72, 39, 76, 83, 92, 25, 112, or 8 of Table 1; and a light chain variable (VL) region comprising a sequence shown in SEQ ID NO: 34, 73, 40, 77, 84, 93, 18, 38, or 80 of Table 1, wherein the first transmembrane domain comprises a CD8α chain transmembrane domain, and wherein the intracellular signaling domain comprises a CD3ζ signaling domain and/or a 4-1BB signaling domain.

### BRIEF DESCRIPTION OF THE DRAWINGS

FIG. 1 shows a BCMA-containing CAR-T cell of the disclosure. The CAR has a functional off-switch activated by rituximab and an anti-BCMA scFv. The modified T-cell further has reduced expression of CD52 (to minimize rejection) and T-Cell receptor genes (TCRα, and/or TCRβ) (to avoid GvHD, graft versus host disease).

FIG. 2 shows the rituximab-mediated off switch enables detection and depletion (with a rituximab antibody) of the rituximab recognition domain-containing CAR-T cells of the disclosure.

FIG. 3 shows that an anti-BCMA scFv-containing CAR-T cell of the disclosure (BCMA-1), with its endogenous CD52 gene knocked down/knocked out, is resistant to a CD52 antibody treatment.

FIG. 4 shows expression of BCMA in target cells.

FIG. 5 shows that anti-BCMA scFv-containing CAR-T cells of the disclosure (BCMA-1), show target-dependent expansion and maintains activity after repeated stimulation.

FIG. 6 shows that anti-BCMA scFv-containing CAR-T cells of the disclosure (BCMA-1) show specific cytotoxic activity. The non-gene edited BCMA-1 refers to CAR-T cells not comprising the knockdown/knockout out of CD52 and/or TCRα and/or TCRβ.

FIG. 7 shows that anti-BCMA scFv-containing CAR-T cells of the disclosure (BCMA-1) have dose-dependent cytotoxic activity that is not inhibited by soluble BCMA.

FIGS. 8-11A and 11B Anti-BCMA scFv-containing CAR-T cells of the disclosure (BCMA-1) show anti-tumor efficacy in an orthotopic tumor model, and can be depleted with rituximab. FIG. 8 shows activity of anti-BCMA scFv-containing CAR-T cells of the disclosure (BCMA-1) in a MM.1S model. FIG. 9 shows the effect of BCMA scFv-containing CAR-T cells of the disclosure (BCMA-1) on tumor eradication after a second dose. FIG. 10 shows the long term antitumor effect of anti-BCMA scFv-containing CAR-T cells of the disclosure (BCMA-1) in mice, supplemented with IL-7/IL-15. The MOLP-8 animal model was used. NSG mice (N=10) were administered with either 5×10⁶ MM.1S cells or 2×10⁶ MOLP-8 cells. Cytokines were provided via AAV-mediated gene delivery. Results were shown as mean f SEM. FIG. 11A-11B show that rituximab depletes anti-BCMA scFv-containing CAR-T cells of the disclosure (BCMA-1) in the model (FIG. 11B), and abrogates antitumor activity (FIG. 11A).

FIGS. 12-15 depict the manufacturing processes of the BCMA CAR-T cells of the disclosure—specifically anti-BCMA scFv-containing CAR-T cells of the disclosure (BCMA-1) can be manufactured under GMP-like conditions with preservation of antitumor activity. FIG. 12 shows an exemplary allogeneic CAR-T manufacturing process for the BCMA CAR-T cells of the disclosure. FIG. 13 shows the high viability and expansion of anti-BCMA scFv-containing CAR-T cells of the disclosure (BCMA-1). FIG. 14 shows efficient enrichment of TCRαβ-negative cells. MACS: Magnetic-activated Cell Sorting system (Miltenyi Biotec). FIG. 15 shows the high antitumor effect of different doses of anti-BCMA scFv-containing CAR-T cells of the disclosure (BCMA-1), in a MM.1S orthotopic tumor model.

FIG. 16 shows that the anti-BCMA-1 scFv does not show off-target binding in tissue cross-reactivity studies, indicating the risk for off-target binding in a clinical setting to be low or non-existent.

FIG. 17 describes limitations of autologous CAR-T therapies.

FIG. 18 describes advantages of allogeneic CAR-T therapies.

FIG. 19 shows the schema for the Phase 1 (Design A or B) Study.

FIG. 20 shows the schema for the Phase 1, Design B Study.

FIG. 21 shows a schematic diagram of an exemplary vector element/construct of the disclosure.

FIGS. 22A-22B show detection of BCMA levels on BCMA-positive MM.1S and Molp-8 cells in the presence of increasing amount of the gamma secretase inhibitor PF-03084014 (nirogacestat, in the hydrobromide salt form, Sigma-Aldrich, #PZ0298). BCMA-negative REH cells were used as a negative control. FIG. 22C shows a dose-responsive curve of PF-03084014 as determined by % inhibition of gamma secretase activity measured by the surface levels of BCMA in MM.1S and Molp-8 cells that were exposed to different amounts of the inhibitor PF-03084014.

FIG. 23 shows the target-dependent fold-expansion of BCMA CAR-T cells in the presence of increasing amount of the gamma secretase inhibitor PF-03084014.

FIG. 24 shows the levels of BCMA-positive target cell killing by BCMA CAR-T cells in the presence of increasing amount of the gamma secretase inhibitor PF-03084014.

FIG. 25A shows increased levels of BCMA on multiple myeloma cells after incubating the cells with PF-03084014 for 24 hours. FIG. 25B shows results of cytolytic assay of BCMA CAR T cells after the target cells had been pre-incubated with PF-03084014.

FIG. 26 shows the schema for Phase 1 Design C, with the inclusion of nirogacestat in Part B which may occur simultaneously with dose escalation of BCMA CAR T cells. See also FIG. 20. A+=Higher dose ALLO-647 (90 mg or 60 mg total dose); FCA60=FCA ALLO-647 60 mg total dose. Nirogacestat: 100 mg BID PO.

### DETAILED DESCRIPTION

The disclosure provides chimeric antigen receptors (CARs) and immune cells (e.g. T-cells) comprising CARs (CAR-T cells) that specifically bind to BCMA, and dosing regimens for use in the treatment of MM, including refractory/relapsed MM. The disclosure also provides polynucleotides encoding these CARs, compositions comprising these CAR-T cells, and methods of making and using these CARs and CAR-T cells.

The disclosure provides CARs that bind to BCMA (e.g., human BCMA, Uniprot accession number: Q02223-2). BCMA specific CARs provided herein include single chain CARS and multichain CARs. The CARs have the ability to redirect T cell specificity and reactivity toward BCMA in a non-MHC-restricted manner, exploiting the antigen-binding properties of monoclonal antibodies. The non-MHC-restricted antigen recognition gives T cells expressing CARs the ability to recognize an antigen independent of antigen processing, thus bypassing a major mechanism of tumor escape.

I. Gamma Secretase Inhibitor

In one aspect, combination cancer therapies are provided comprising BCMA CAR-T cells and a gamma secretase inhibitor. In some embodiments, the gamma secretase inhibitor reduces proteolytic cleavage of BCMA ectodomain on the cell surface by gamma secretase and potentiates BCMA positive malignant B cell killing by BCMA CAR-T cells. In some embodiments, the treatment efficacy improves in a subject who is administered the combination therapy as compared to a subject who is administered the BCMA CAR-T cells alone. Gamma secretase inhibitors presently or previously under clinical trial studies include nirogacestat (SpringWorks), semagacestat (Eli Lilly) and BMS-986115 (Bristol-Myers Squibb). In some embodiments, the gamma secretase inhibitor is nirogacestat having the structure of:

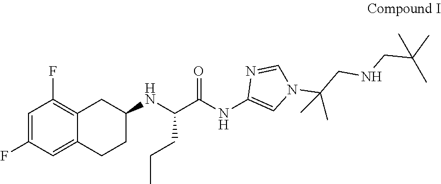 [SMILES: CCC[C@H](N[C@H]1CCc2cc(F)cc(F)c2C1)C(=O)Nc1cn(C(C)(C)CNCC(C)(C)C)cn1]

or a pharmaceutically acceptable salt thereof. In some embodiments, the gamma secretase inhibitor is nirogacestat hydrobromide. In some embodiments, the gamma secretase inhibitor is nirogacestat dihydrobromide.

II. BCMA-Specific CARS

In some embodiments, CARs provided herein comprise an extracellular ligand-binding domain (e.g., a single chain variable fragment (scFv)), a transmembrane domain, and an intracellular signaling domain. In some embodiments, the extracellular ligand-binding domain, transmembrane domain, and intracellular signaling domain are in one polypeptide, i.e., in a single chain. Multichain CARs and polypeptides are also provided herein. In some embodiments, the multichain CARs comprise: a first polypeptide comprising a transmembrane domain and at least one extracellular ligand-binding domain, and a second polypeptide comprising a transmembrane domain and at least one intracellular signaling domain, wherein the polypeptides assemble together to form a multichain CAR.

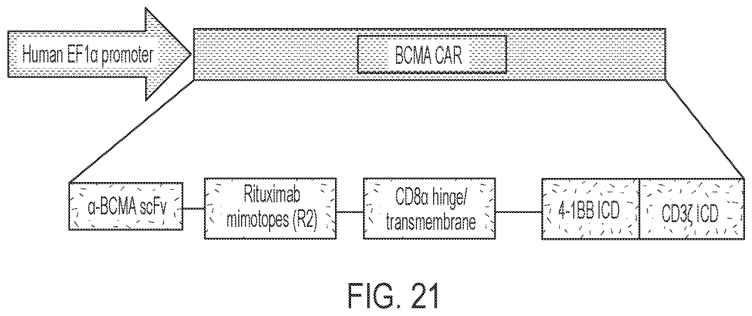

In some embodiments, a BCMA specific multichain CAR is based on the high affinity receptor for IgE (FcεRI). The FcεRI expressed on mast cells and basophiles triggers allergic reactions. FcεRI is a tetrameric complex composed of a single α subunit, a single β subunit, and two disulfide-linked 7 subunits. The α subunit contains the IgE-binding domain. The β and γ subunits contain ITAMs that mediate signal transduction. In some embodiments, the extracellular domain of the FcRα chain is deleted and replaced by a BCMA specific extracellular ligand-binding domain. In some embodiments, the multichain BCMA specific CAR comprises an scFv that binds specifically to BCMA, the CD8α hinge, and the ITAM of the FcRβ chain. In some embodiments, the CAR may or may not comprise the FcRγ chain. In some embodiments, two copies of a rituximab mimotope (e.g., CPYSNPSLC (SEQ ID NO: 397); see also WO 2016/120216, incorporated herein by reference in its entirety) are present. An exemplary construct is show in FIG. 21.

As provided herein, the extracellular ligand-binding domain of the BCMA CAR comprises an scFv comprising the light chain variable (VL) region and the heavy chain variable (VH) region of a target antigen specific monoclonal antibody joined by a flexible linker. Single chain variable region fragments are made by linking light and/or heavy chain variable regions by using a short linking peptide (Bird et al., Science 242:423-426, 1988). An example of a linking peptide is the GS linker having the amino acid sequence (GGGGS)3 (SEQ ID NO: 333), which bridges approximately 3.5 nm between the carboxy terminus of one variable region and the amino terminus of the other variable region. Linkers of other sequences have been designed and used (Bird et al., 1988, supra). In general, linkers can be short, flexible polypeptides and preferably comprised of about 20 or fewer amino acid residues. Linkers can in turn be modified for additional functions, such as attachment of drugs or attachment to solid supports. The single chain variants can be produced either recombinantly or synthetically. For synthetic production of scFv, an automated synthesizer can be used. For recombinant production of scFv, a suitable plasmid containing polynucleotide that encodes the scFv can be introduced into a suitable host cell, either eukaryotic, such as yeast, plant, insect or mammalian cells, or prokaryotic, such as E. coli. Polynucleotides encoding the scFv of interest can be made by routine manipulations such as ligation of polynucleotides. The resultant scFv can be isolated using standard protein purification techniques known in the art.

In some embodiments, provided herein is a BCMA CAR, wherein the CAR comprises an extracellular binding domain comprising a single chain Fv fragment (scFv), wherein the scFv comprises a heavy chain variable (VH) region and a light chain variable (VL) region, wherein the VH region comprises a VH complementary determining region 1 (VH CDR1), a VH complementary determining region 2 (VH CDR2), and a VH complementary determining region 3 (VH CDR3) and the VL region comprises a VL complementary determining region 1 (VL CDR1), a VL complementary determining region 2 (VL CDR2), and a VL complementary determining region 3 (VL CDR3), wherein: (a) the VH CDR1 comprises a sequence selected from the group consisting of: SEQ ID NOs.: 129, 130, 131, 150, 151, 152, 156, 157, 301, 302, 303, 381, 382, 386, 387, and 388; (b) the VH CDR2 comprises a sequence selected from the group consisting of: SEQ ID NOs.: 132, 133, 138, 139, 140, 141, 142, 143, 144, 145, 146, 147, 153, 154, 158, 159, 160, 162, 163, 165, 166, 167, 168, 169, 171, 172, 174, 175, 176, 177, 178, 179, 180, 181, 183, 184, 185, 186, 187, 188, 190, 191, 192, 193, 194, 195, 196, 198, 199, 200, 201, 202, 203, 204, 206, 207, 208, 304, 305, 306, 383, 384, 389, and 390; (c) the VH CDR3 comprises a sequence selected from the group consisting of: SEQ ID NOs.: 134, 135, 136, 137, 148, 149, 155, 161, 164, 170, 173, 182, 189, 197, 205, 307, 308, 385, and 391; (d) the VL CDR1 comprises a sequence selected from the group consisting of: SEQ ID NOs.: 209, 212, 215, 217, 218, 219, 223, 226, 228, 230, 232, 235, 238, 239, 241, 243, 245, 246, 247, 249, 250, 251, 254, 257, 260, 262, 265, 266, 267, 269, 270, 271, 273, 275, 277, 279, 283, 285, 287, 290, 292, 295, 297, 299, 309, 377, 415, and 417; (e) the VL CDR2 comprises a sequence selected from the group consisting of: SEQ ID NOs.: 210, 221, 252, 310, 392, and 395; and (f) the VL CDR3 comprises a sequence selected from the group consisting of: SEQ ID NOs.: 211, 213, 214, 216, 220, 222, 224, 225, 227, 229, 231, 233, 234, 236, 237, 240, 242, 244, 248, 253, 255, 256, 258, 259, 261, 263, 264, 268, 272, 274, 276, 278, 280, 281, 282, 284, 286, 288, 289, 291, 293, 294, 296, 298, 300, 311, 312, 393, and 416.

In some embodiments, provided herein is a BCMA CAR, wherein the CAR comprises an extracellular ligand-binding domain comprising: a VH region comprising a VH CDR1, VH CDR2, and VH CDR3 of the VH sequence shown in SEQ ID NO: 2, 3, 7, 8, 24, 25, 26, 27, 28, 29, 30, 31, 32, 33, 35, 37, 39, 42, 44, 46, 48, 50, 52, 54, 56, 58, 60, 62, 64, 66, 68, 70, 72, 74, 76, 78, 83, 87, 92, 95, 97, 99, 101, 104, 106, 110, 112, 114, 76, 118, 120, 122, 125, 127, 313, 314 or 413; and/or a VL region comprising VL CDR1, VL CDR2, and VL CDR3 of the VL sequence shown in SEQ ID NO: 1, 4, 5, 6, 9, 10, 11, 12, 13, 15, 16, 17, 18, 19, 20, 21, 22, 23, 34, 36, 38, 40, 41, 43, 45, 47, 49, 51, 53, 57, 59, 61, 63, 65, 67, 69, 71, 73, 75, 77, 79, 317, 81, 82, 84, 85, 86, 88, 89, 90, 91, 93, 94, 96, 98, 100, 102, 103, 105, 107, 108, 109, 111, 113, 115, 116, 117, 119, 121, 123, 124, 126, 128, 315, 316, or 414. In some embodiments, the VH and VL are linked together by a flexible linker. In some embodiments, a flexible linker comprises the amino acid sequence shown in SEQ ID NO: 333.

In some embodiments, a CAR of the disclosure comprises an extracellular ligand-binding domain having any one of partial light chain sequence as listed in Table 1 and/or any one of partial heavy chain sequence as listed in Table 1. In Table 1, the underlined sequences are CDR sequences according to Kabat and in bold according to Chothia, except for the following heavy chain CDR2 sequences, in which the Chothia CDR sequence is underlined and the Kabat CDR sequence is in bold: P5A2_VHVL, A02_Rd4_0.6 nM_C06, A02_Rd4_0.6 nM_C09, A02_Rd4_6 nM_C16, A02_Rd4_6 nM_C03, A02_Rd4_6 nM_C01, A02_Rd4_6 nM_C26, A02_Rd4_6 nM_C25, A02_Rd4_6 nM_C22, A02_Rd4_6 nM_C19, A02_Rd4_0.6 nM_C03, A02_Rd4_6 nM_C07, A02_Rd4_6 nM_C23, A02_Rd4_0.6 nM_C18, A02_Rd4_6 nM_C10, A02_Rd4_6 nM_C05, A02_Rd4_0.6 nM_C10, A02_Rd4_6 nM_C04, A02_Rd4_0.6 nM_C26, A02_Rd4_0.6 nM_C13, A02_Rd4_0.6 nM_C01, A02_Rd4_6 nM_C08, P5C1_VHVL, C01_Rd4_6 nM_C24, C01_Rd4_6 nM_C26, C01_Rd4_6 nM_C10, C01_Rd4_0.6 nM_C27, C01_Rd4_6 nM_C20, C01_Rd4_6 nM_C12, C01_Rd4_0.6 nM_C16, C01_Rd4_0.6 nM_C09, C01_Rd4_6 nM_C09, C01_Rd4_0.6 nM_C03, C01_Rd4_0.6 nM_C06, C01_Rd4_6 nM_C04, COMBO_Rd4_0.6 nM_C22, COMBO_Rd4_6 nM_C21, COMBO_Rd4_6 nM_C10, COMBO_Rd4_0.6 nM_C04, COMBO_Rd4_6 nM_C25, COMBO_Rd4_0.6 nM_C21, COMBO_Rd4_6 nM_C11, COMBO_Rd4_0.6 nM_C20, COMBO_Rd4_6 nM_C09, COMBO_Rd4_6 nM_C08, COMBO_Rd4_0.6 nM_C19, COMBO_Rd4_0.6 nM_C02, COMBO_Rd4_0.6 nM_C23, COMBO_Rd4_0.6 nM_C29, COMBO_Rd4_0.6 nM_C09, COMBO_Rd4_6 nM_C12, COMBO_Rd4_0.6 nM_C30, COMBO_Rd4_0.6 nM_C14, COMBO_Rd4_6 nM_C07, COMBO_Rd4_6 nM_C02, COMBO_Rd4_0.6 nM_C05, COMBO_Rd4_0.6 nM_C17, COMBO_Rd4_6 nM_C22, and COMBO_Rd4_0.6nM_C11.

TABLE 1

| Binding | | |
|---|---|---|
| Domain | Light Chain | Heavy Chain |
| P6E01/ | EIVLTQSPGTLSLSPGERATLSCRAS | EVQLLESGGGLVQPGGSLRLSCAASG |
| P6E01 | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYGSPPSFTF | TLYLQMNSLRAEDTAVYYCARVSPIA |
| | GQGTKVEIK (SEQ ID NO: 1) | SGMDYWGQGTLVTVSS (SEQ ID NO: |
| | | 2) |
| P6E01/ | EIVLTQSPGTLSLSPGERATLSCRAS | EVQLLESGGGLVQPGGSLRLSCAASG |
| H3AQ | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYGSPPSFTF | TLYLQMNSLRAEDTAVYYCARVSPIA |
| | GQGTKVEIK (SEQ ID NO: 1) | AQMDYWGQGTLVTVSS (SEQ ID NO: |
| | | 3) |
| L1.LGF/ | EIVLTQSPGTLSLSPGERATLSC | EVQLLESGGGLVQPGGSLRLSCAASG |
| L3.KW/ | RASQSLGSFYLAWYQQKPGQAPR | FTFGSYAMTWVRQAPGKGLEWVSAI |
| P6E01 | LLIYGASSRATGIPDRFSGSGSGTD | SGSGGNTFYADSVKGRFTISRDNSKN |
| | FTLTISRLEPEDFAVYYCKHYGWP | TLYLQMNSLRAEDTAVYYCARVSPIA |
| | PSFTFGQGTKVEIK (SEQ ID NO: 4) | SGMDYWGQGTLVTVSS (SEQ ID NO: |
| | | 2) |
| L1.LGF/ | EIVLTQSPGTLSLSPGERATLSCRAS | EVQLLESGGGLVQPGGSLRLSCAASG |
| L3.NY/ | QSLGSFYLAWYQQKPGQAPRLLIY | FTFGSYAMTWVRQAPGKGLEWVSAI |
| P6E01 | GASSRATGIPDRFSGSGSGTDFTLTI | SGSGGNTFYADSVKGRFTISRDNSKN |
| | SRLEPEDFAVYYCQHYNYPPSFTF | TLYLQMNSLRAEDTAVYYCARVSPIA |
| | GQGTKVEIK (SEQ ID NO: 5) | SGMDYWGQGTLVTVSS (SEQ ID NO: |
| | | 2) |
| L1.GDF/ | EIVLTQSPGTLSLSPGERATLSCRAS | EVQLLESGGGLVQPGGSLRLSCAASG |
| L3.NY/ | QSVGDFYLAWYQQKPGQAPRLLI | FTFGSYAMTWVRQAPGKGLEWVSAI |
| P6E01 | YGASSRATGIPDRFSGSGSGTDFTL | SGSGGNTFYADSVKGRFTISRDNSKN |
| | TISRLEPEDFAVYYCQHYNYPPSFT | TLYLQMNSLRAEDTAVYYCARVSPIA |
| | FGQGTKVEIK (SEQ ID NO: 6) | SGMDYWGQGTLVTVSS (SEQ ID NO: |
| | | 2) |
| L1.LGF/ | EIVLTQSPGTLSLSPGERATLSCRAS | EVQLLESGGGLVQPGGSLRLSCAASG |
| L3.KW/H3. | QSLGSFYLAWYQQKPGQAPRLLIY | FTFGSYAMTWVRQAPGKGLEWVSAI |
| AL | GASSRATGIPDRFSGSGSGTDFTLTI | SGSGGNTFYADSVKGRFTISRDNSKN |
| | SRLEPEDFAVYYCKHYGWPPSFTF | TLYLQMNSLRAEDTAVYYCARARVS |
| | GQGTKVEIK (SEQ ID NO: 4) | PIAALMDYWGQGTLVTVSS (SEQ ID |
| | | NO: 7) |
| L1.LGF/ | EIVLTQSPGTLSLSPGERATLSCRAS | EVQLLESGGGLVQPGGSLRLSCAASG |
| L3.KW/H3. | QSLGSFYLAWYQQKPGQAPRLLIY | FTFGSYAMTWVRQAPGKGLEWVSAI |
| AP | GASSRATGIPDRFSGSGSGTDFTLTI | SGSGGNTFYADSVKGRFTISRDNSKN |
| | SRLEPEDFAVYYCKHYGWPPSFTF | TLYLQMNSLRAEDTAVYYCARVSPIA |
| | GQGTKVEIK (SEQ ID NO: 4) | APMDYWGQGTLVTVSS (SEQ ID NO: |
| | | 8) |
| L1.LGF/ | EIVLTQSPGTLSLSPGERATLSCRAS | EVQLLESGGGLVQPGGSLRLSCAASG |
| L3.KW/H3. | QSLGSFYLAWYQQKPGQAPRLLIY | FTFGSYAMTWVRQAPGKGLEWVSAI |
| AQ | GASSRATGIPDRFSGSGSGTDFTLTI | SGSGGNTFYADSVKGRFTISRDNSKN |
| | SRLEPEDFAVYYCKHYGWPPSFTF | TLYLQMNSLRAEDTAVYYCARVSPIA |
| | GQGTKVEIK (SEQ ID NO: 4) | AQMDYWGQGTLVTVSS (SEQ ID NO: |
| | | 3) |
| L1.LGF/ | EIVLTQSPGTLSLSPGERATLSCRAS | EVQLLESGGGLVQPGGSLRLSCAASG |
| L3.PY/H3. | QSLGSFYLAWYQQKPGQAPRLLIY | FTFGSYAMTWVRQAPGKGLEWVSAI |
| AP | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 9) | APMDYWGQGTLVTVSS (SEQ ID NO: |
| | | 8) |
| L1.LGF/ | EIVLTQSPGTLSLSPGERATLSCRAS | EVQLLESGGGLVQPGGSLRLSCAASG |
| L3.PY/H3. | QSLGSFYLAWYQQKPGQAPRLLIY | FTFGSYAMTWVRQAPGKGLEWVSAI |
| AQ | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 9) | AQMDYWGQGTLVTVSS (SEQ ID NO: |
| | | 3) |
| L1.LGF/ | EIVLTQSPGTLSLSPGERATLSCRAS | EVQLLESGGGLVQPGGSLRLSCAASG |
| L3.NY/H3. | QSLGSFYLAWYQQKPGQAPRLLIY | FTFGSYAMTWVRQAPGKGLEWVSAI |
| AL | GASSRATGIPDRFSGSGSGTDFTLTI | SGSGGNTFYADSVKGRFTISRDNSKN |
| | SRLEPEDFAVYYCQHYNYPPSFTF | TLYLQMNSLRAEDTAVYYCARVSPIA |
| | GQGTKVEIK (SEQ ID NO: 10) | ALMDYWGQGTLVTVSS (SEQ ID NO: |
| | | 412) |
| L1.LGF/ | EIVLTQSPGTLSLSPGERATLSCRAS | EVQLLESGGGLVQPGGSLRLSCAASG |
| L3.NY/H3. | QSLGSFYLAWYQQKPGQAPRLLIY | FTFGSYAMTWVRQAPGKGLEWVSAI |
| AP | GASSRATGIPDRFSGSGSGTDFTLTI | SGSGGNTFYADSVKGRFTISRDNSKN |
| | SRLEPEDFAVYYCQHYNYPPSFTF | TLYLQMNSLRAEDTAVYYCARVSPIA |
| | GQGTKVEIK (SEQ ID NO: 10) | APMDYWGQGTLVTVSS (SEQ ID NO: |
| | | 8) |
| L1.LGF/ | EIVLTQSPGTLSLSPGERATLSCRAS | EVQLLESGGGLVQPGGSLRLSCAASG |
| L3.NY/H3. | QSLGSFYLAWYQQKPGQAPRLLIY | FTFGSYAMTWVRQAPGKGLEWVSAI |
| AQ | GASSRATGIPDRFSGSGSGTDFTLTI | SGSGGNTFYADSVKGRFTISRDNSKN |
| | SRLEPEDFAVYYCQHYNYPPSFTF | TLYLQMNSLRAEDTAVYYCARVSPIA |
| | GQGTKVEIK (SEQ ID NO: 10) | AQMDYWGQGTLVTVSS (SEQ ID NO: |
| | | 3) |
| L1.GDF/ | EIVLTQSPGTLSLSPGERATLSCRAS | EVQLLESGGGLVQPGGSLRLSCAASG |
| L3.KW/H3. | QSVGDFYLAWYQQKPGQAPRLLI | FTFGSYAMTWVRQAPGKGLEWVSAI |
| AL | YGASSRATGIPDRFSGSGSGTDFTL | SGSGGNTFYADSVKGRFTISRDNSKN |
| | TISRLEPEDFAVYYCKHYGWPPSF | TLYLQMNSLRAEDTAVYYCARVSPIA |
| | TFGQGTKVEIK (SEQ ID NO: 11) | ALMDYWGQGTLVTVSS (SEQ ID NO: |
| | | 412) |
| L1.GDF/ | EIVLTQSPGTLSLSPGERATLSCRAS | EVQLLESGGGLVQPGGSLRLSCAASG |
| L3.KW/H3. | QSVGDFYLAWYQQKPGQAPRLLI | FTFGSYAMTWVRQAPGKGLEWVSAI |
| AP | YGASSRATGIPDRFSGSGSGTDFTL | SGSGGNTFYADSVKGRFTISRDNSKN |
| | TISRLEPEDFAVYYCKHYGWPPSF | TLYLQMNSLRAEDTAVYYCARVSPIA |
| | TFGQGTKVEIK (SEQ ID NO: 11) | APMDYWGQGTLVTVSS (SEQ ID NO: |
| | | 8) |
| L1.GDF/ | EIVLTQSPGTLSLSPGERATLSCRAS | EVQLLESGGGLVQPGGSLRLSCAASG |
| L3.KW/H3. | QSVGDFYLAWYQQKPGQAPRLLI | FTFGSYAMTWVRQAPGKGLEWVSAI |
| AQ | YGASSRATGIPDRFSGSGSGTDFTL | SGSGGNTFYADSVKGRFTISRDNSKN |
| | TISRLEPEDFAVYYCKHYGWPPSF | TLYLQMNSLRAEDTAVYYCARVSPIA |
| | TFGQGTKVEIK (SEQ ID NO: 11) | AQMDYWGQGTLVTVSS (SEQ ID NO: |
| | | 3) |
| L1.GDF/ | EIVLTQSPGTLSLSPGERATLSCRAS | EVQLLESGGGLVQPGGSLRLSCAASG |
| L3.PY/H3. | QSVGDFYLAWYQQKPGQAPRLLI | FTFGSYAMTWVRQAPGKGLEWVSAI |
| AQ | YGASSRATGIPDRFSGSGSGTDFTL | SGSGGNTFYADSVKGRFTISRDNSKN |
| | TISRLEPEDFAVYYCQHYPYPPSFT | TLYLQMNSLRAEDTAVYYCARVSPIA |
| | FGQGTKVEIK (SEQ ID NO: 12) | AQMDYWGQGTLVTVSS (SEQ ID NO: |
| | | 3) |
| L1.GDF/ | EIVLTQSPGTLSLSPGERATLSCRAS | EVQLLESGGGLVQPGGSLRLSCAASG |
| L3.NY/H3. | QSVGDFYLAWYQQKPGQAPRLLI | FTFGSYAMTWVRQAPGKGLEWVSAI |
| AL | YGASSRATGIPDRFSGSGSGTDFTL | SGSGGNTFYADSVKGRFTISRDNSKN |
| | TISRLEPEDFAVYYCQHYNYPPSFT | TLYLQMNSLRAEDTAVYYCARVSPIA |
| | FGQGTKVEIK (SEQ ID NO: 13) | ALMDYWGQGTLVTVSS (SEQ ID NO: |
| | | 412) |
| L1.GDF/ | EIVLTQSPGTLSLSPGERATLSCRAS | EVQLLESGGGLVQPGGSLRLSCAASG |
| L3.NY/H3. | QSVGDFYLAWYQQKPGQAPRLLI | FTFGSYAMTWVRQAPGKGLEWVSAI |
| AP | YGASSRATGIPDRFSGSGSGTDFTL | SGSGGNTFYADSVKGRFTISRDNSKN |
| | TISRLEPEDFAVYYCQHYNYPPSFT | TLYLQMNSLRAEDTAVYYCARVSPIA |
| | FGQGTKVEIK (SEQ ID NO: 13) | APMDYWGQGTLVTVSS (SEQ ID NO: |
| | | 8) |
| L1.GDF/ | EIVLTQSPGTLSLSPGERATLSCRAS | EVQLLESGGGLVQPGGSLRLSCAASG |
| L3.NY/H3. | QSVGDFYLAWYQQKPGQAPRLLI | FTFGSYAMTWVRQAPGKGLEWVSAI |
| AQ | YGASSRATGIPDRFSGSGSGTDFTL | SGSGGNTFYADSVKGRFTISRDNSKN |
| | TISRLEPEDFAVYYCQHYNYPPSFT | TLYLQMNSLRAEDTAVYYCARVSPIA |
| | FGQGTKVEIK (SEQ ID NO: 14) | AQMDYWGQGTLVTVSS (SEQ ID NO: |
| | | 3) |
| L3.KW/ | EIVLTQSPGTLSLSPGERATLSCRAS | EVQLLESGGGLVQPGGSLRLSCAASG |
| P6E01 | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCKHYGWPPSFTF | TLYLQMNSLRAEDTAVYYCARVSPIA |
| | GQGTKVEIK (SEQ ID NO: 15) | SGMDYWGQGTLVTVSS (SEQ ID NO: |
| | | 2) |
| L3.PY/ | EIVLTQSPGTLSLSPGERATLSCRAS | EVQLLESGGGLVQPGGSLRLSCAASG |
| P6E01 | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 16) | SGMDYWGQGTLVTVSS (SEQ ID NO: |
| | | 2) |
| L3.NY/ | EIVLTQSPGTLSLSPGERATLSCRAS | EVQLLESGGGLVQPGGSLRLSCAASG |
| P6E01 | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYNYPPSFTF | TLYLQMNSLRAEDTAVYYCARVSPIA |
| | GQGTKVEIK (SEQ ID NO: 17) | SGMDYWGQGTLVTVSS (SEQ ID NO: |
| | | 2) |
| L3.PY/L1. | EIVLTQSPGTLSLSPGERATLSCRAS | EVQLLESGGGLVQPGGSLRLSCAASG |
| PS/P6E01 | QSVSSSYPSWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 18) | SGMDYWGQGTLVTVSS (SEQ ID NO: |
| | | 2) |
| L3.PY/L1. | EIVLTQSPGTLSLSPGERATLSCRAS | EVQLLESGGGLVQPGGSLRLSCAASG |
| AH/P6E01 | QSVSAHYLAWYQQKPGQAPRLLI | FTFGSYAMTWVRQAPGKGLEWVSAI |
| | YGASSRATGIPDRFSGSGSGTDFTL | SGSGGNTFYADSVKGRFTISRDNSKN |
| | TISRLEPEDFAVYYCQHYPYPPSFT | TLYLQMNSLRAEDTAVYYCARVSPIA |
| | FGQGTKVEIK (SEQ ID NO: 19) | SGMDYWGQGTLVTVSS (SEQ ID NO: |
| | | 2) |
| L3.PY/L1. | EIVLTQSPGTLSLSPGERATLSCRAS | EVQLLESGGGLVQPGGSLRLSCAASG |
| FF/P6E01 | QSVSSFFLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 20) | SGMDYWGQGTLVTVSS (SEQ ID NO: |
| | | 2) |
| L3.PY/L1. | EIVLTQSPGTLSLSPGERATLSCRAS | EVQLLESGGGLVQPGGSLRLSCAASG |
| PH/P6E01 | QSVSPHYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 21) | SGMDYWGQGTLVTVSS (SEQ ID NO: |
| | | 2) |
| L3.PY/L3. | EIVLTQSPGTLSLSPGERATLSCRAS | EVQLLESGGGLVQPGGSLRLSCAASG |
| KY/P6E01 | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCKYYPYPPSFTF | TLYLQMNSLRAEDTAVYYCARVSPIA |
| | GQGTKVEIK (SEQ ID NO: 22) | SGMDYWGQGTLVTVSS (SEQ ID NO: |
| | | 2) |
| L3.PY/L3. | EIVLTQSPGTLSLSPGERATLSCRAS | EVQLLESGGGLVQPGGSLRLSCAASG |
| KF/P6E01 | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCKFYPYPPSFTFG | TLYLQMNSLRAEDTAVYYCARVSPIA |
| | QGTKVEIK (SEQ ID NO: 23) | SGMDYWGQGTLVTVSS (SEQ ID NO: |
| | | 2) |
| L3.PY/H2. | EIVLTQSPGTLSLSPGERATLSCRAS | EVQLLESGGGLVQPGGSLRLSCAASG |
| QR | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADQRKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 16) | SGMDYWGQGTLVTVSS (SEQ ID NO: |
| | | 24) |
| L3.PY/H2. | EIVLTQSPGTLSLSPGERATLSCRAS | EVQLLESGGGLVQPGGSLRLSCAASG |
| DY | QSVSSSYLAWYQQKPGQAPRLLIY | FTFGSYAMTWVRQAPGKGLEWVSAI |
| | GASSRATGIPDRFSGSGSGTDFTLTI | DYSGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 16) | SGMDYWGQGTLVTVSS (SEQ ID NO: |
| | | 25) |
| L3.PY/H2. | EIVLTQSPGTLSLSPGERATLSCRAS | EVQLLESGGGLVQPGGSLRLSCAASG |
| YQ | QSVSSSYLAWYQQKPGQAPRLLIY | FTFGSYAMTWVRQAPGKGLEWVSAI |
| | GASSRATGIPDRFSGSGSGTDFTLTI | SYQGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 16) | SGMDYWGQGTLVTVSS (SEQ ID NO: |
| | | 26) |
| L3.PY/H2. | EIVLTQSPGTLSLSPGERATLSCRAS | EVQLLESGGGLVQPGGSLRLSCAASG |
| LT | QSVSSSYLAWYQQKPGQAPRLLIY | FTFGSYAMTWVRQAPGKGLEWVSAI |
| | GASSRATGIPDRFSGSGSGTDFTLTI | SLTGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 16) | SGMDYWGQGTLVTVSS (SEQ ID NO: |
| | | 27) |
| L3.PY/H2. | EIVLTQSPGTLSLSPGERATLSCRAS | EVQLLESGGGLVQPGGSLRLSCAASG |
| HA | QSVSSSYLAWYQQKPGQAPRLLIY | FTFGSYAMTWVRQAPGKGLEWVSAI |
| | GASSRATGIPDRFSGSGSGTDFTLTI | SHAGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 16) | SGMDYWGQGTLVTVSS (SEQ ID NO: |
| | | 28) |
| L3.PY/H2. | EIVLTQSPGTLSLSPGERATLSCRAS | EVQLLESGGGLVQPGGSLRLSCAASG |
| QL | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADQLKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 16) | SGMDYWGQGTLVTVSS (SEQ ID NO: |
| | | 29) |
| L3.PY/H3. | EIVLTQSPGTLSLSPGERATLSCRAS | EVQLLESGGGLVQPGGSLRLSCAASG |
| YA | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCARVSPIY |
| | GQGTKVEIK (SEQ ID NO: 16) | AGMDYWGQGTLVTVSS (SEQ ID NO: |
| | | 30) |
| L3.PY/H3. | EIVLTQSPGTLSLSPGERATLSCRAS | EVQLLESGGGLVQPGGSLRLSCAASG |
| AE | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 16) | AEMDYWGQGTLVTVSS (SEQ ID NO: |
| | | 31) |
| L3.PY/H3. | EIVLTQSPGTLSLSPGERATLSCRAS | EVQLLESGGGLVQPGGSLRLSCAASG |
| AQ | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 16) | AQMDYWGQGTLVTVSS (SEQ ID NO: |
| | | 3) |
| L3.PY/H3. | EIVLTQSPGTLSLSPGERATLSCRAS | EVQLLESGGGLVQPGGSLRLSCAASG |
| TAQ | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCTRVSPIA |
| | GQGTKVEIK (SEQ ID NO: 16) | AQMDYWGQGTLVTVSS (SEQ ID NO: |
| | | 32) |
| L3.PY/ | EIVLTQSPGTLSLSPGERATLSCRAS | EVQLLESGGGLVQPGGSLRLSCAASG |
| P6E01 | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 16) | SGMDYWGQGTLVTVSS (SEQ ID NO: |
| | | 2) |
| L3.PY/L1. | EIVLTQSPGTLSLSPGERATLSCRAS | EVQLLESGGGLVQPGGSLRLSCAASG |
| PS/H2.QR | QSVSSSYPSWYQQKPGQAPRLLIY | FTFGSYAMTWVRQAPGKGLEWVSAI |
| | GASSRATGIPDRFSGSGSGTDFTLTI | SGSGGNTFYADQRKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 18) | SGMDYWGQGTLVTVSS (SEQ ID NO: |
| | | 24) |
| L3.PY/L1. | EIVLTQSPGTLSLSPGERATLSCRAS | EVQLLESGGGLVQPGGSLRLSCAASG |
| PS/H2.DY | QSVSSSYPSWYQQKPGQAPRLLIY | FTFGSYAMTWVRQAPGKGLEWVSAI |
| | GASSRATGIPDRFSGSGSGTDFTLTI | DYSGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 18) | SGMDYWGQGTLVTVSS (SEQ ID NO: |
| | | 25) |
| L3.PY/L1. | EIVLTQSPGTLSLSPGERATLSCRAS | EVQLLESGGGLVQPGGSLRLSCAASG |
| PS/H2.YQ | QSVSSSYPSWYQQKPGQAPRLLIY | FTFGSYAMTWVRQAPGKGLEWVSAI |
| | GASSRATGIPDRFSGSGSGTDFTLTI | SYQGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 18) | SGMDYWGQGTLVTVSS (SEQ ID NO: |
| | | 26) |
| L3.PY/L1. | EIVLTQSPGTLSLSPGERATLSCRAS | EVQLLESGGGLVQPGGSLRLSCAASG |
| PS/H2.LT | QSVSSSYPSWYQQKPGQAPRLLIY | FTFGSYAMTWVRQAPGKGLEWVSAI |
| | GASSRATGIPDRFSGSGSGTDFTLTI | SLTGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 18) | SGMDYWGQGTLVTVSS (SEQ ID NO: |
| | | 27) |
| L3.PY/L1. | EIVLTQSPGTLSLSPGERATLSCRAS | EVQLLESGGGLVQPGGSLRLSCAASG |
| PS/H2.HA | QSVSSSYPSWYQQKPGQAPRLLIY | FTFGSYAMTWVRQAPGKGLEWVSAI |
| | GASSRATGIPDRFSGSGSGTDFTLTI | SHAGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 18) | SGMDYWGQGTLVTVSS (SEQ ID NO: |
| | | 28) |
| L3.PY/L1. | EIVLTQSPGTLSLSPGERATLSCRAS | EVQLLESGGGLVQPGGSLRLSCAASG |
| PS/H2.QL | QSVSSSYPSWYQQKPGQAPRLLIY | FTFGSYAMTWVRQAPGKGLEWVSAI |
| | GASSRATGIPDRFSGSGSGTDFTLTI | SGSGGNTFYADQLKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 18) | SGMDYWGQGTLVTVSS (SEQ ID NO: |
| | | 29) |
| L3.PY/L1. | EIVLTQSPGTLSLSPGERATLSCRAS | EVQLLESGGGLVQPGGSLRLSCAASG |
| PS/H3.YA | QSVSSSYPSWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCARVSPIY |
| | GQGTKVEIK (SEQ ID NO: 18) | AGMDYWGQGTLVTVSS (SEQ ID NO: |
| | | 30) |
| L3.PY/L1. | EIVLTQSPGTLSLSPGERATLSCRAS | EVQLLESGGGLVQPGGSLRLSCAASG |
| PS/H3.AE | QSVSSSYPSWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 18) | AEMDYWGQGTLVTVSS (SEQ ID NO: |
| | | 31) |
| L3.PY/L1. | EIVLTQSPGTLSLSPGERATLSCRAS | EVQLLESGGGLVQPGGSLRLSCAASG |
| PS/H3.AQ | QSVSSSYPSWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 18) | AQMDYWGQGTLVTVSS (SEQ ID NO: |
| | | 3) |
| L3.PY/L1. | EIVLTQSPGTLSLSPGERATLSCRAS | EVQLLESGGGLVQPGGSLRLSCAASG |
| PS/H3.TAQ | QSVSSSYPSWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCTRVSPIA |
| | GQGTKVEIK (SEQ ID NO: 18) | AQMDYWGQGTLVTVSS (SEQ ID NO: |
| | | 32) |
| L3.PY/L1. | EIVLTQSPGTLSLSPGERATLSCRAS | EVQLLESGGGLVQPGGSLRLSCAASG |
| AH/H2.QR | QSVSAHYLAWYQQKPGQAPRLLI | FTFGSYAMTWVRQAPGKGLEWVSAI |
| | YGASSRATGIPDRFSGSGSGTDFTL | SGSGGNTFYADQRKGRFTISRDNSKN |
| | TISRLEPEDFAVYYCQHYPYPPSFT | TLYLQMNSLRAEDTAVYYCARVSPIA |
| | FGQGTKVEIK (SEQ ID NO: 19) | SGMDYWGQGTLVTVSS (SEQ ID NO: |
| | | 24) |
| L3.PY/L1. | EIVLTQSPGTLSLSPGERATLSCRAS | EVQLLESGGGLVQPGGSLRLSCAASG |
| AH/H2.DY | QSVSAHYLAWYQQKPGQAPRLLI | FTFGSYAMTWVRQAPGKGLEWVSAI |
| | YGASSRATGIPDRFSGSGSGTDFTL | DYSGGNTFYADSVKGRFTISRDNSKN |
| | TISRLEPEDFAVYYCQHYPYPPSFT | TLYLQMNSLRAEDTAVYYCARVSPIA |
| | FGQGTKVEIK (SEQ ID NO: 19) | SGMDYWGQGTLVTVSS (SEQ ID NO: |
| | | 25) |
| L3.PY/L1. | EIVLTQSPGTLSLSPGERATLSCRAS | EVQLLESGGGLVQPGGSLRLSCAASG |
| AH/H2.YQ | QSVSAHYLAWYQQKPGQAPRLLI | FTFGSYAMTWVRQAPGKGLEWVSAI |
| | YGASSRATGIPDRFSGSGSGTDFTL | SYQGGNTFYADSVKGRFTISRDNSKN |
| | TISRLEPEDFAVYYCQHYPYPPSFT | TLYLQMNSLRAEDTAVYYCARVSPIA |
| | FGQGTKVEIK (SEQ ID NO: 19) | SGMDYWGQGTLVTVSS (SEQ ID NO: |
| | | 26) |
| L3.PY/L1. | EIVLTQSPGTLSLSPGERATLSCRAS | EVQLLESGGGLVQPGGSLRLSCAASG |
| AH/H2.LT | QSVSAHYLAWYQQKPGQAPRLLI | FTFGSYAMTWVRQAPGKGLEWVSAI |
| | YGASSRATGIPDRFSGSGSGTDFTL | SLTGGNTFYADSVKGRFTISRDNSKN |
| | TISRLEPEDFAVYYCQHYPYPPSFT | TLYLQMNSLRAEDTAVYYCARVSPIA |
| | FGQGTKVEIK (SEQ ID NO: 19) | SGMDYWGQGTLVTVSS (SEQ ID NO: |
| | | 27) |
| L3.PY/L1. | EIVLTQSPGTLSLSPGERATLSCRAS | EVQLLESGGGLVQPGGSLRLSCAASG |
| AH/H2.HA | QSVSAHYLAWYQQKPGQAPRLLI | FTFGSYAMTWVRQAPGKGLEWVSAI |
| | YGASSRATGIPDRFSGSGSGTDFTL | SHAGGNTFYADSVKGRFTISRDNSKN |
| | TISRLEPEDFAVYYCQHYPYPPSFT | TLYLQMNSLRAEDTAVYYCARVSPIA |
| | FGQGTKVEIK (SEQ ID NO: 19) | SGMDYWGQGTLVTVSS (SEQ ID NO: |
| | | 28) |
| L3.PY/L1. | EIVLTQSPGTLSLSPGERATLSCRAS | EVQLLESGGGLVQPGGSLRLSCAASG |
| AH/H2.QL | QSVSAHYLAWYQQKPGQAPRLLI | FTFGSYAMTWVRQAPGKGLEWVSAI |
| | YGASSRATGIPDRFSGSGSGTDFTL | SGSGGNTFYADQLKGRFTISRDNSKN |
| | TISRLEPEDFAVYYCQHYPYPPSFT | TLYLQMNSLRAEDTAVYYCARVSPIA |
| | FGQGTKVEIK (SEQ ID NO: 19) | SGMDYWGQGTLVTVSS (SEQ ID NO: |
| | | 29) |
| L3.PY/L1. | EIVLTQSPGTLSLSPGERATLSCRAS | EVQLLESGGGLVQPGGSLRLSCAASG |
| AH/H3 YA | QSVSAHYLAWYQQKPGQAPRLLI | FTFGSYAMTWVRQAPGKGLEWVSAI |
| | YGASSRATGIPDRFSGSGSGTDFTL | SGSGGNTFYADSVKGRFTISRDNSKN |
| | TISRLEPEDFAVYYCQHYPYPPSFT | TLYLQMNSLRAEDTAVYYCARVSPIY |
| | FGQGTKVEIK (SEQ ID NO: 19) | AGMDYWGQGTLVTVSS (SEQ ID NO: |
| | | 30) |
| L3.PY/L1. | EIVLTQSPGTLSLSPGERATLSCRAS | EVQLLESGGGLVQPGGSLRLSCAASG |
| AH/H3.AE | QSVSAHYLAWYQQKPGQAPRLLI | FTFGSYAMTWVRQAPGKGLEWVSAI |
| | YGASSRATGIPDRFSGSGSGTDFTL | SGSGGNTFYADSVKGRFTISRDNSKN |
| | TISRLEPEDFAVYYCQHYPYPPSFT | TLYLQMNSLRAEDTAVYYCARVSPIA |
| | FGQGTKVEIK (SEQ ID NO: 19) | AEMDYWGQGTLVTVSS (SEQ ID NO: |
| | | 31) |
| L3.PY/L1. | EIVLTQSPGTLSLSPGERATLSCRAS | EVQLLESGGGLVQPGGSLRLSCAASG |
| AH/H3.AQ | QSVSAHYLAWYQQKPGQAPRLLI | FTFGSYAMTWVRQAPGKGLEWVSAI |
| | YGASSRATGIPDRFSGSGSGTDFTL | SGSGGNTFYADSVKGRFTISRDNSKN |
| | TISRLEPEDFAVYYCQHYPYPPSFT | TLYLQMNSLRAEDTAVYYCARVSPIA |
| | FGQGTKVEIK (SEQ ID NO: 19) | AQMDYWGQGTLVTVSS (SEQ ID NO: |
| | | 3) |
| L3.PY/L1. | EIVLTQSPGTLSLSPGERATLSCRAS | EVQLLESGGGLVQPGGSLRLSCAASG |
| AH/H3.TAQ | QSVSAHYLAWYQQKPGQAPRLLI | FTFGSYAMTWVRQAPGKGLEWVSAI |
| | YGASSRATGIPDRFSGSGSGTDFTL | SGSGGNTFYADSVKGRFTISRDNSKN |
| | TISRLEPEDFAVYYCQHYPYPPSFT | TLYLQMNSLRAEDTAVYYCTRVSPIA |
| | FGQGTKVEIK (SEQ ID NO: 19) | AQMDYWGQGTLVTVSS (SEQ ID NO: |
| | | 32) |
| L3.PY/L1. | EIVLTQSPGTLSLSPGERATLSCRAS | EVQLLESGGGLVQPGGSLRLSCAASG |
| FF/H2.QR | QSVSSFFLAWYQQKPGQAPRLLIY | FTFGSYAMTWVRQAPGKGLEWVSAI |
| | GASSRATGIPDRFSGSGSGTDFTLTI | SGSGGNTFYADQRKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 20) | SGMDYWGQGTLVTVSS (SEQ ID NO: |
| | | 24) |
| L3.PY/L1. | EIVLTQSPGTLSLSPGERATLSCRAS | EVQLLESGGGLVQPGGSLRLSCAASG |
| FF/H2.DY | QSVSSFFLAWYQQKPGQAPRLLIY | FTFGSYAMTWVRQAPGKGLEWVSAI |
| | GASSRATGIPDRFSGSGSGTDFTLTI | DYSGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 20) | SGMDYWGQGTLVTVSS (SEQ ID NO: |
| | | 25) |
| L3.PY/L1. | EIVLTQSPGTLSLSPGERATLSCRAS | EVQLLESGGGLVQPGGSLRLSCAASG |
| FF/H2.YQ | QSVSSFFLAWYQQKPGQAPRLLIY | FTFGSYAMTWVRQAPGKGLEWVSAI |
| | GASSRATGIPDRFSGSGSGTDFTLTI | SYQGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 20) | SGMDYWGQGTLVTVSS (SEQ ID NO: |
| | | 26) |
| L3.PY/L1. | EIVLTQSPGTLSLSPGERATLSCRAS | EVQLLESGGGLVQPGGSLRLSCAASG |
| FF/H2.LT | QSVSSFFLAWYQQKPGQAPRLLIY | FTFGSYAMTWVRQAPGKGLEWVSAI |
| | GASSRATGIPDRFSGSGSGTDFTLTI | SLTGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 20) | SGMDYWGQGTLVTVSS (SEQ ID NO: |
| | | 27) |
| L3.PY/L1. | EIVLTQSPGTLSLSPGERATLSCRAS | EVQLLESGGGLVQPGGSLRLSCAASG |
| FF/H2.HA | QSVSSFFLAWYQQKPGQAPRLLIY | FTFGSYAMTWVRQAPGKGLEWVSAI |
| | GASSRATGIPDRFSGSGSGTDFTLTI | SHAGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 20) | SGMDYWGQGTLVTVSS (SEQ ID NO: |
| | | 28) |
| L3.PY/L1. | EIVLTQSPGTLSLSPGERATLSCRAS | EVQLLESGGGLVQPGGSLRLSCAASG |
| FF/H2.QL | QSVSSFFLAWYQQKPGQAPRLLIY | FTFGSYAMTWVRQAPGKGLEWVSAI |
| | GASSRATGIPDRFSGSGSGTDFTLTI | SGSGGNTFYADQLKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 20) | SGMDYWGQGTLVTVSS (SEQ ID NO: |
| | | 29) |
| L3.PY/L1. | EIVLTQSPGTLSLSPGERATLSCRAS | EVQLLESGGGLVQPGGSLRLSCAASG |
| FF/H3 YA | QSVSSFFLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCARVSPIY |
| | GQGTKVEIK (SEQ ID NO: 20) | AGMDYWGQGTLVTVSS (SEQ ID NO: |
| | | 30) |
| L3.PY/L1. | EIVLTQSPGTLSLSPGERATLSCRAS | EVQLLESGGGLVQPGGSLRLSCAASG |
| FF/H3.AE | QSVSSFFLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 20) | AEMDYWGQGTLVTVSS (SEQ ID NO: |
| | | 31) |
| L3.PY/L1. | EIVLTQSPGTLSLSPGERATLSCRAS | EVQLLESGGGLVQPGGSLRLSCAASG |
| FF/H3.AQ | QSVSSFFLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 20) | AQMDYWGQGTLVTVSS (SEQ ID NO: |
| | | 3) |
| L3.PY/L1. | EIVLTQSPGTLSLSPGERATLSCRAS | EVQLLESGGGLVQPGGSLRLSCAASG |
| FF/H3.TAQ | QSVSSFFLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCTRVSPIA |
| | GQGTKVEIK (SEQ ID NO: 20) | AQMDYWGQGTLVTVSS (SEQ ID NO: |
| | | 32) |
| L3.PY/L1. | EIVLTQSPGTLSLSPGERATLSCRAS | EVQLLESGGGLVQPGGSLRLSCAASG |
| PH/H2.QR | QSVSPHYLAWYQQKPGQAPRLLIY | FTFGSYAMTWVRQAPGKGLEWVSAI |
| | GASSRATGIPDRFSGSGSGTDFTLTI | SGSGGNTFYADQRKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 21) | SGMDYWGQGTLVTVSS (SEQ ID NO: |
| | | 24) |
| L3.PY/L1. | EIVLTQSPGTLSLSPGERATLSCRAS | EVQLLESGGGLVQPGGSLRLSCAASG |
| PH/H2.HA | QSVSPHYLAWYQQKPGQAPRLLIY | FTFGSYAMTWVRQAPGKGLEWVSAI |
| | GASSRATGIPDRFSGSGSGTDFTLTI | SHAGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 21) | SGMDYWGQGTLVTVSS (SEQ ID NO: |
| | | 28) |
| L3.PY/L1. | EIVLTQSPGTLSLSPGERATLSCRAS | EVQLLESGGGLVQPGGSLRLSCAASG |
| PH/H3.AE | QSVSPHYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 21) | AEMDYWGQGTLVTVSS (SEQ ID NO: |
| | | 31) |
| L3.PY/L1. | EIVLTQSPGTLSLSPGERATLSCRAS | EVQLLESGGGLVQPGGSLRLSCAASG |
| PH/H3.AQ | QSVSPHYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 21) | AQMDYWGQGTLVTVSS (SEQ ID NO: |
| | | 3) |
| L3.PY/L1. | EIVLTQSPGTLSLSPGERATLSCRAS | EVQLLESGGGLVQPGGSLRLSCAASG |
| PH/H3.TAQ | QSVSPHYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYPYPPSFTF | TLYLQMNSLRAEDTAVYYCTRVSPIA |
| | GQGTKVEIK (SEQ ID NO: 21) | AQMDYWGQGTLVTVSS (SEQ ID NO: |
| | | 32) |
| L3.PY/L3. | EIVLTQSPGTLSLSPGERATLSCRAS | EVQLLESGGGLVQPGGSLRLSCAASG |
| KY/H2.QR | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADQRKGRFTISRDNSKN |
| | SRLEPEDFAVYYCKYYPYPPSFTF | TLYLQMNSLRAEDTAVYYCARVSPIA |
| | GQGTKVEIK (SEQ ID NO: 22) | SGMDYWGQGTLVTVSS (SEQ ID NO: |
| | | 24) |
| L3.PY/L3. | EIVLTQSPGTLSLSPGERATLSCRAS | EVQLLESGGGLVQPGGSLRLSCAASG |
| KY/H2.DY | QSVSSSYLAWYQQKPGQAPRLLIY | FTFGSYAMTWVRQAPGKGLEWVSAI |
| | GASSRATGIPDRFSGSGSGTDFTLTI | DYSGGNTFYADSVKGRFTISRDNSKN |
| | SRLEPEDFAVYYCKYYPYPPSFTF | TLYLQMNSLRAEDTAVYYCARVSPIA |
| | GQGTKVEIK (SEQ ID NO: 22) | SGMDYWGQGTLVTVSS (SEQ ID NO: |
| | | 25) |
| L3.PY/L3. | EIVLTQSPGTLSLSPGERATLSCRAS | EVQLLESGGGLVQPGGSLRLSCAASG |
| KY/H2.YQ | QSVSSSYLAWYQQKPGQAPRLLIY | FTFGSYAMTWVRQAPGKGLEWVSAI |
| | GASSRATGIPDRFSGSGSGTDFTLTI | SYQGGNTFYADSVKGRFTISRDNSKN |
| | SRLEPEDFAVYYCKYYPYPPSFTF | TLYLQMNSLRAEDTAVYYCARVSPIA |
| | GQGTKVEIK (SEQ ID NO: 22) | SGMDYWGQGTLVTVSS (SEQ ID NO: |
| | | 26) |
| L3.PY/L3. | EIVLTQSPGTLSLSPGERATLSCRAS | EVQLLESGGGLVQPGGSLRLSCAASG |
| KY/H2.LT | QSVSSSYLAWYQQKPGQAPRLLIY | FTFGSYAMTWVRQAPGKGLEWVSAI |
| | GASSRATGIPDRFSGSGSGTDFTLTI | SLTGGNTFYADSVKGRFTISRDNSKN |
| | SRLEPEDFAVYYCKYYPYPPSFTF | TLYLQMNSLRAEDTAVYYCARVSPIA |
| | GQGTKVEIK (SEQ ID NO: 22) | SGMDYWGQGTLVTVSS (SEQ ID NO: |
| | | 27) |
| L3.PY/L3. | EIVLTQSPGTLSLSPGERATLSCRAS | EVQLLESGGGLVQPGGSLRLSCAASG |
| KY/H2.HA | QSVSSSYLAWYQQKPGQAPRLLIY | FTFGSYAMTWVRQAPGKGLEWVSAI |
| | GASSRATGIPDRFSGSGSGTDFTLTI | SHAGGNTFYADSVKGRFTISRDNSKN |
| | SRLEPEDFAVYYCKYYPYPPSFTF | TLYLQMNSLRAEDTAVYYCARVSPIA |
| | GQGTKVEIK (SEQ ID NO: 22) | SGMDYWGQGTLVTVSS (SEQ ID NO: |
| | | 28) |
| L3.PY/L3. | EIVLTQSPGTLSLSPGERATLSCRAS | EVQLLESGGGLVQPGGSLRLSCAASG |
| KY/H2.QL | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADQLKGRFTISRDNSKN |
| | SRLEPEDFAVYYCKYYPYPPSFTF | TLYLQMNSLRAEDTAVYYCARVSPIA |
| | GQGTKVEIK (SEQ ID NO: 22) | SGMDYWGQGTLVTVSS (SEQ ID NO: |
| | | 29) |
| L3.PY/L3. | EIVLTQSPGTLSLSPGERATLSCRAS | EVQLLESGGGLVQPGGSLRLSCAASG |
| KY/H3.YA | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCKYYPYPPSFTF | TLYLQMNSLRAEDTAVYYCARVSPIY |
| | GQGTKVEIK (SEQ ID NO: 22) | AGMDYWGQGTLVTVSS (SEQ ID NO: |
| | | 30) |
| L3.PY/L3. | EIVLTQSPGTLSLSPGERATLSCRAS | EVQLLESGGGLVQPGGSLRLSCAASG |
| KY/H3.TAQ | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCKYYPYPPSFTF | TLYLQMNSLRAEDTAVYYCTRVSPIA |
| | GQGTKVEIK (SEQ ID NO: 22) | AQMDYWGQGTLVTVSS (SEQ ID NO: |
| | | 32) |
| L3.PY/L3. | EIVLTQSPGTLSLSPGERATLSCRAS | EVQLLESGGGLVQPGGSLRLSCAASG |
| KF/H2.DY | QSVSSSYLAWYQQKPGQAPRLLIY | FTFGSYAMTWVRQAPGKGLEWVSAI |
| | GASSRATGIPDRFSGSGSGTDFTLTI | DYSGGNTFYADSVKGRFTISRDNSKN |
| | SRLEPEDFAVYYCKFYPYPPSFTFG | TLYLQMNSLRAEDTAVYYCARVSPIA |
| | QGTKVEIK (SEQ ID NO: 23) | SGMDYWGQGTLVTVSS (SEQ ID NO: |
| | | 25) |
| L3.PY/L3. | EIVLTQSPGTLSLSPGERATLSCRAS | EVQLLESGGGLVQPGGSLRLSCAASG |
| KF/H2.YQ | QSVSSSYLAWYQQKPGQAPRLLIY | FTFGSYAMTWVRQAPGKGLEWVSAI |
| | GASSRATGIPDRFSGSGSGTDFTLTI | SYQGGNTFYADSVKGRFTISRDNSKN |
| | SRLEPEDFAVYYCKFYPYPPSFTFG | TLYLQMNSLRAEDTAVYYCARVSPIA |
| | QGTKVEIK (SEQ ID NO: 23) | SGMDYWGQGTLVTVSS (SEQ ID NO: |
| | | 26) |
| L3.PY/L3 | EIVLTQSPGTLSLSPGERATLSCRAS | EVQLLESGGGLVQPGGSLRLSCAASG |
| KF/H2.LT | QSVSSSYLAWYQQKPGQAPRLLIY | FTFGSYAMTWVRQAPGKGLEWVSAI |
| | GASSRATGIPDRFSGSGSGTDFTLTI | SLTGGNTFYADSVKGRFTISRDNSKN |
| | SRLEPEDFAVYYCKFYPYPPSFTFG | TLYLQMNSLRAEDTAVYYCARVSPIA |
| | QGTKVEIK (SEQ ID NO: 23) | SGMDYWGQGTLVTVSS (SEQ ID NO: |
| | | 27) |
| L3.PY/L3. | EIVLTQSPGTLSLSPGERATLSCRAS | EVQLLESGGGLVQPGGSLRLSCAASG |
| KF/H2.QL | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADQLKGRFTISRDNSKN |
| | SRLEPEDFAVYYCKFYPYPPSFTFG | TLYLQMNSLRAEDTAVYYCARVSPIA |
| | QGTKVEIK (SEQ ID NO: 23) | SGMDYWGQGTLVTVSS (SEQ ID NO: |
| | | 29) |
| L3.PY/L3. | EIVLTQSPGTLSLSPGERATLSCRAS | EVQLLESGGGLVQPGGSLRLSCAASG |
| KF/H3.YA | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCKFYPYPPSFTFG | TLYLQMNSLRAEDTAVYYCARVSPIY |
| | QGTKVEIK (SEQ ID NO: 23) | AGMDYWGQGTLVTVSS (SEQ ID NO: |
| | | 30) |
| L3.PY/L3. | EIVLTQSPGTLSLSPGERATLSCRAS | EVQLLESGGGLVQPGGSLRLSCAASG |
| KF/H3.AE | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCKFYPYPPSFTFG | TLYLQMNSLRAEDTAVYYCARVSPIA |
| | QGTKVEIK (SEQ ID NO: 23) | AEMDYWGQGTLVTVSS (SEQ ID NO: |
| | | 31) |
| L3.PY/L3. | EIVLTQSPGTLSLSPGERATLSCRAS | EVQLLESGGGLVQPGGSLRLSCAASG |
| KF/H3.AQ | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCKFYPYPPSFTFG | TLYLQMNSLRAEDTAVYYCARVSPIA |
| | QGTKVEIK (SEQ ID NO: 23) | AQMDYWGQGTLVTVSS (SEQ ID NO: |
| | | 3) |
| L3.PY/L3. | EIVLTQSPGTLSLSPGERATLSCRAS | EVQLLESGGGLVQPGGSLRLSCAASG |
| KF/H3TAQ | QSVSSSYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCKFYPYPPSFTFG | TLYLQMNSLRAEDTAVYYCTRVSPIA |
| | QGTKVEIK (SEQ ID NO: 23) | AQMDYWGQGTLVTVSS (SEQ ID NO: |
| | | 32) |
| P5A2_VH | EIVLTQSPGTLSLSPGERATLSCRAS | EVQLLESGGGLVQPGGSLRLSCAASG |
| VL | QSVSSSYLAWYQQKPGQAPRLLM | FTFSSYAMNWVRQAPGKGLEWVSAI |
| | YDASIRATGIPDRFSGSGSGTDFTL | SDSGGSTYYADSVKGRFTISRDNSKN |
| | TISRLEPEDFAVYYCQQYGSWPLT | TLYLQMNSLRAEDTAVYYCARYWP |
| | FGQGTKVEIK (SEQ ID NO: 34) | MDIWGQGTLVTVSS (SEQ ID NO: 33) |
| A02_Rd4 | EIVLTQSPGTLSLSPGERATLSCRAS | EVQLLESGGGLVQPGGSLRLSCAASG |
| _0.6 nM_C06 | QSVSVIYLAWYQQKPGQAPRLLM | FTFSSYAMNWVRQAPGKGLEWVSAI |
| | YDASIRATGIPDRFSGSGSGTDFTL | SDSGGSAWYADSVKGRFTISRDNSKN |
| | TISRLEPEDFAVYYCQQYQRWPLT | TLYLQMNSLRAEDTAVYYCARYWP |
| | FGQGTKVEIK (SEQ ID NO: 36) | MSLWGQGTLVTVSS (SEQ ID NO: 35) |
| A02_Rd4 | EIVLTQSPGTLSLSPGERATLSCRAS | EVQLLESGGGLVQPGGSLRLSCAASG |
| _0.6 nM_C09 | QSVSSSYLAWYQQKPGQAPRLLM | FTFSSYAMNWVRQAPGKGLEWVSAI |
| | YDASIRATGIPDRFSGSGSGTDFTL | SDSGGSMWYADSVKGRFTISRDNSK |
| | TISRLEPEDFAVYYCQQYQSWPLT | NTLYLQMNSLRAEDTAVYYCARYWP |
| | FGQGTKVEIK (SEQ ID NO: 38) | MSLWGQGTLVTVSS (SEQ ID NO: 37) |
| A02_Rd4 | EIVLTQSPGTLSLSPGERATLSCRAS | EVQLLESGGGLVQPGGSLRLSCAASG |
| _6 nM_C16 | QSVSDIYLAWYQQKPGQAPRLLM | FTFSSYAMNWVRQAPGKGLEWVSAI |
| (P5AC16) | YDASIRATGIPDRFSGSGSGTDFTL | SdFGGSTYYADSVKGRFTISRDNSKN |
| | TISRLEPEDFAVYYCQQYQTWPLT | TLYLQMNSLRAEDTAVYYCARYWP |
| | FGQGTKVEIK (SEQ ID NO: 40) | MDIWGQGTLVTVSS (SEQ ID NO: 39) |
| A02_Rd4 | EIVLTQSPGTLSLSPGERATLSCRAS | EVQLLESGGGLVQPGGSLRLSCAASG |
| _6 nM_C03 | QSVSNLYLAWYQQKPGQAPRLLM | FTFSSYAMNWVRQAPGKGLEWVSAI |
| | YDASIRATGIPDRFSGSGSGTDFTL | SDSGGSTYYADSVKGRFTISRDNSKN |
| | TISRLEPEDFAVYYCQQYQGWPLT | TLYLQMNSLRAEDTAVYYCARYWP |
| | FGQGTKVEIK (SEQ ID NO: 41) | MDIWGQGTLVTVSS (SEQ ID NO: 33) |
| A02_Rd4 | EIVLTQSPGTLSLSPGERATLSCRAS | EVQLLESGGGLVQPGGSLRLSCAASG |
| _6 nM_C01 | QSVSAYYLAWYQQKPGQAPRLLM | FTFSSYAMNWVRQAPGKGLEWVSAI |
| | YDASIRATGIPDRFSGSGSGTDFTL | TASGGSTYYADSVKGRFTISRDNSKN |
| | TISRLEPEDFAVYYCQQYERWPLT | TLYLQMNSLRAEDTAVYYCARYWP |
| | FGQGTKVEIK (SEQ ID NO: 43) | MSLWGQGTLVTVSS (SEQ ID NO: 42) |
| A02_Rd4 | EIVLTQSPGTLSLSPGERATLSCRAS | EVQLLESGGGLVQPGGSLRLSCAASG |
| _6 nM_C26 | QSVSSLYLAWYQQKPGQAPRLLM | FTFSSYAMNWVRQAPGKGLEWVSAI |
| | YDASIRATGIPDRFSGSGSGTDFTL | SDSGGSTYYADSVKGRFTISRDNSKN |
| | TISRLEPEDFAVYYCQQYQVWPLT | TLYLQMNSLRAEDTAVYYCARYWP |
| | FGQGTKVEIK (SEQ ID NO: 45) | MSLWGQGTLVTVSS (SEQ ID NO: 44) |
| A02_Rd4 | EIVLTQSPGTLSLSPGERATLSCRAS | EVQLLESGGGLVQPGGSLRLSCAASG |
| _6 nM_C25 | QSVSSSYLAWYQQKPGQAPRLLM | FTFSSYAMNWVRQAPGKGLEWVSAI |
| | YDASIRATGIPDRFSGSGSGTDFTL | SdSGGSRWYADSVKGRFTISRDNSKN |
| | TISRLEPEDFAVYYCQQYLDWPLT | TLYLQMNSLRAEDTAVYYCARYWP |
| | FGQGTKVEIK (SEQ ID NO: 47) | MTPWGQGTLVTVSS (SEQ ID NO: 46) |
| A02_Rd4 | EIVLTQSPGTLSLSPGERATLSCRAS | EVQLLESGGGLVQPGGSLRLSCAASG |
| _6 nM_C22 | QSVSSSYLAWYQQKPGQAPRLLM | FTFSSYAMNWVRQAPGKGLEWVSAV |
| | YDASIRATGIPDRFSGSGSGTDFTL | LdSGGSTYYADSVKGRFTISRDNSKN |
| | TISRLEPEDFAVYYCQQYQVWPLT | TLYLQMNSLRAEDTAVYYCARYWP |
| | FGQGTKVEIK (SEQ ID NO: 49) | MTPWGQGTLVTVSS (SEQ ID NO: 48) |
| A02_Rd4 | EIVLTQSPGTLSLSPGERATLSCRAS | EVQLLESGGGLVQPGGSLRLSCAASG |
| _6 nM_C19 | QSVSVIYLAWYQQKPGQAPRLLM | FTFSSYAMNWVRQAPGKGLEWVSAI |
| | YDASIRATGIPDRFSGSGSGTDFTL | SdSGGSRWYADSVKGRFTISRDNSKN |
| | TISRLEPEDFAVYYCQQYLAWPLT | TLYLQMNSLRAEDTAVYYCARYWP |
| | FGQGTKVEIK (SEQ ID NO: 51) | MSDWGQGTLVTVSS (SEQ ID NO: 50) |
| A02_Rd4 | EIVLTQSPGTLSLSPGERATLSCRAS | EVQLLESGGGLVQPGGSLRLSCAASG |
| _0.6 nM_C03 | QSVSSSYLAWYQQKPGQAPRLLM | FTFSSYAMNWVRQAPGKGLEWVSAI |
| | YDASIRATGIPDRFSGSGSGTDFTL | SdSGGSKWYADSVKGRFTISRDNSKN |
| | TISRLEPEDFAVYYCQQYFTWPLT | TLYLQMNSLRAEDTAVYYCARYWP |
| | FGQGTKVEIK (SEQ ID NO: 53) | MSLWGQGTLVTVSS (SEQ ID NO: 52) |
| A02_Rd4 | EIVLTQSPGTLSLSPGERATLSCRAS | EVQLLESGGGLVQPGGSLRLSCAASG |
| _6 nM_C07 | QSVSPyYLAWYQQKPGQAPRLLM | FTFSSYAMNWVRQAPGKGLEWVSAI |
| | YDASIRATGIPDRFSGSGSGTDFTL | GGSGGSLPYADSVKGRFTISRDNSKN |
| | TISRLEPEDFAVYYCQQYERWPLT | TLYLQMNSLRAEDTAVYYCARYWP |
| | FGQGTKVEIK (SEQ ID NO: 55) | MDSWGQGTLVTVSS (SEQ ID NO: 54) |
| A02_Rd4 | EIVLTQSPGTLSLSPGERATLSCRAS | EVQLLESGGGLVQPGGSLRLSCAASG |
| _6 nM_C23 | QSVSVEYLAWYQQKPGQAPRLLM | FTFSSYAMNWVRQAPGKGLEWVSAI |
| | YDASIRATGIPDRFSGSGSGTDFTL | SdSGGSGWYADSVKGRFTISRDNSKN |
| | TISRLEPEDFAVYYCQQYARWPLT | TLYLQMNSLRAEDTAVYYCARYWP |
| | FGQGTKVEIK (SEQ ID NO: 57) | MSLWGQGTLVTVSS (SEQ ID NO: 56) |
| A02_Rd4 | EIVLTQSPGTLSLSPGERATLSCRAS | EVQLLESGGGLVQPGGSLRLSCAASG |
| _0.6 nM_C18 | QSVSEIYLAWYQQKPGQAPRLLM | FTFSSYAMNWVRQAPGKGLEWVSAV |
| | YDASIRATGIPDRFSGSGSGTDFTL | LdSGGSTYYADSVKGRFTISRDNSKN |
| | TISRLEPEDFAVYYCQQYFGWPLT | TLYLQMNSLRAEDTAVYYCARYWP |
| | FGQGTKVEIK (SEQ ID NO: 59) | MSLWGQGTLVTVSS (SEQ ID NO: 58) |
| A02_Rd4 | EIVLTQSPGTLSLSPGERATLSCRAS | EVQLLESGGGLVQPGGSLRLSCAASG |
| _6 nM_C10 | QSVEMSYLAWYQQKPGQAPRLLM | FTFSSYAMNWVRQAPGKGLEWVSAI |
| | YDASIRATGIPDRFSGSGSGTDFTL | SdSGGSGWYADSVKGRFTISRDNSKN |
| | TISRLEPEDFAVYYCQQYAHWPLT | TLYLQMNSLRAEDTAVYYCARYWP |
| | FGQGTKVEIK (SEQ ID NO: 61) | MTPWGQGTLVTVSS (SEQ ID NO: 60) |
| A02_Rd4 | EIVLTQSPGTLSLSPGERATLSCRAS | EVQLLESGGGLVQPGGSLRLSCAASG |
| _6 nM_C05 | QSVSSSYLAWYQQKPGQAPRLLM | FTFSSYAMNWVRQAPGKGLEWVSAI |
| | YDASIRATGIPDRFSGSGSGTDFTL | FaSGGSTYYADSVKGRFTISRDNSKN |
| | TISRLEPEDFAVYYCQQYQRWPLT | TLYLQMNSLRAEDTAVYYCARYWP |
| | FGQGTKVEIK (SEQ ID NO: 63) | MTPWGQGTLVTVSS (SEQ ID NO: 62) |
| A02_Rd4 | EIVLTQSPGTLSLSPGERATLSCRAS | EVQLLESGGGLVQPGGSLRLSCAASG |
| _0.6 nM_C10 | QSVSAQYLAWYQQKPGQAPRLLM | FTFSSYAMNWVRQAPGKGLEWVSAI |
| | YDASIRATGIPDRFSGSGSGTDFTL | SgWGGSLPYADSVKGRFTISRDNSKN |
| | TISRLEPEDFAVYYCQQYQRWPLT | TLYLQMNSLRAEDTAVYYCARYWP |
| | FGQGTKVEIK (SEQ ID NO: 65) | MDSWGQGTLVTVSS (SEQ ID NO: 64) |
| A02_Rd4 | EIVLTQSPGTLSLSPGERATLSCRAS | EVQLLESGGGLVQPGGSLRLSCAASG |
| _6 nM_C04 | QSVSAIYLAWYQQKPGQAPRLLM | FTFSSYAMNWVRQAPGKGLEWVSAI |
| | YDASIRATGIPDRFSGSGSGTDFTL | MsSGGPLYYADSVKGRFTISRDNSKN |
| | TISRLEPEDFAVYYCQQYQVWPLT | TLYLQMNSLRAEDTAVYYCARYWP |
| | FGQGTKVEIK (SEQ ID NO: 67) | MALWGQGTLVTVSS (SEQ ID NO: 66) |
| A02_Rd4 | EIVLTQSPGTLSLSPGERATLSCGPS | EVQLLESGGGLVQPGGSLRLSCAASG |
| _0.6 nM_C26 | QSVSSSYLAWYQQKPGQAPRLLM | FTFSSYAMNWVRQAPGKGLEWVSAI |
| | YDASIRATGIPDRFSGSGSGTDFTL | LmSGGSTYYADSVKGRFTISRDNSKN |
| | TISRLEPEDFAVYYCQQYQSWPLT | TLYLQMNSLRAEDTAVYYCARYWP |
| | FGQGTKVEIK (SEQ ID NO: 69) | MSLWGQGTLVTVSS (SEQ ID NO: 68) |
| A02_Rd4 | EIVLTQSPGTLSLSPGERATLSCRAS | EVQLLESGGGLVQPGGSLRLSCAASG |
| _0.6 nM_C13 | QSVSSSYWAWYQQKPGQAPRLLM | FTFSSYAMNWVRQAPGKGLEWVSAI |
| | YDASIRATGIPDRFSGSGSGTDFTL | SdSGGYRYYADSVKGRFTISRDNSKN |
| | TISRLEPEDFAVYYCQQYESWPLT | TLYLQMNSLRAEDTAVYYCARYWP |
| | FGQGTKVEIK (SEQ ID NO: 71) | MSLWGQGTLVTVSS (SEQ ID NO: 70) |
| A02_Rd4 | EIVLTQSPGTLSLSPGERATLSCRG | EVQLLESGGGLVQPGGSLRLSCAASG |
| _0.6 nM_C01 | GQSVSSSYLAWYQQKPGQAPRLL | FTFSSYAMNWVRQAPGKGLEWVSAI |
| (P5AC1) | MYDASIRATGIPDRFSGSGSGTDFT | LsSGGSTYYADSVKGRFTISRDNSKNT |
| | LTISRLEPEDFAVYYCQQYQSWPL | LYLQMNSLRAEDTAVYYCARYWPM |
| | TFGQGTKVEIK (SEQ ID NO: 73) | DIWGQGTLVTVSS (SEQ ID NO: 72) |
| A02_Rd4 | EIVLTQSPGTLSLSPGERATLSCRAS | EVQLLESGGGLVQPGGSLRLSCAASG |
| _6 nM_C08 | QSVSFIYLAWYQQKPGQAPRLLMY | FTFSSYAMNWVRQAPGKGLEWVSAI |
| | DASIRATGIPDRFSGSGSGTDFTLTI | LdSGGSTYYADSVKGRFTISRDNSKN |
| | SRLEPEDFAVYYCQQYGSWPLTF | TLYLQMNSLRAEDTAVYYCARYWP |
| | GQGTKVEIK (SEQ ID NO: 75) | MSPWGQGTLVTVSS (SEQ ID NO: 74) |
| P5C1_VH | EIVLTQSPGTLSLSPGERATLSCRAS | EVQLLESGGGLVQPGGSLRLSCAASG |
| VL (PC1) | QSVSSTYLAWYQQKPGQAPRLLIY | FTFSSYPMSWVRQAPGKGLEWVSAIG |
| | DASSRAPGIPDRFSGSGSGTDFTLTI | GSGGSTYYADSVKGRFTISRDNSKNT |
| | SRLEPEDFAVYYCQQYSTSPLTFG | LYLQMNSLRAEDTAVYYCARYWPM |
| | QGTKVEIK (SEQ ID NO: 77) | DSWGQGTLVTVSS (SEQ ID NO: 76) |
| C01_Rd4 | EIVLTQSPGTLSLSPGERATLSCRAS | EVQLLESGGGLVQPGGSLRLSCAASG |
| _6 nM_C24 | QSVSPEYLAWYQQKPGQAPRLLIY | FTFSSYPMSWVRQAPGKGLEWVSAIG |
| | DASSRAPGIPDRFSGSGSGTDFTLTI | GSGGSLPYADSVKGRFTISRDNSKNT |
| | SRLEPEDFAVYYCQQYSVWPLTFG | LYLQMNSLRAEDTAVYYCARYWPM |
| | QGTKVEIK (SEQ ID NO: 79) | DSWGQGTLVTVSS (SEQ ID NO: 78) |
| C01_Rd4 | EIVLTQSPGTLSLSPGERATLSCRAS | EVQLLESGGGLVQPGGSLRLSCAASG |
| _6 nM_C26 | QSVSAIYLAWYQQKPGQAPRLLIY | FTFSSYPMSWVRQAPGKGLEWVSAIG |
| | DASSRAPGIPDRFSGSGSGTDFTLTI | GSGGSLPYADSVKGRFTISRDNSKNT |
| | SRLEPEDFAVYYCQQYSAWPLTFG | LYLQMNSLRAEDTAVYYCARYWPM |
| | QGTKVEIK (SEQ ID NO: 317) | DSWGQGTLVTVSS |
| | | (SEQ ID NO: 78) |
| C01_Rd4 | EIVLTQSPGTLSLSPGERATLSCRAS | EVQLLESGGGLVQPGGSLRLSCAASG |
| _6 nM_C10 | QSVSSvYLAWYQQKPGQAPRLLIY | FTFSSYPMSWVRQAPGKGLEWVSAIG |
| | DASSRAPGIPDRFSGSGSGTDFTLTI | gSGGSLPYADSVKGRFTISRDNSKNTL |
| | SRLEPEDFAVYYCQQYSTWPLTFG | YLQMNSLRAEDTAVYYCARYWPMD |
| | QGTKVEIK (SEQ ID NO: 414) | SWGQGTLVTVSS (SEQ ID NO: 78) |
| C01_Rd4 | EIVLTQSPGTLSLSPGERATLSCRAS | EVQLLESGGGLVQPGGSLRLSCAASG |
| _0.6 nM_C27 | QSVSSTYLAWYQQKPGQAPRLLIY | FTFSSYPMSWVRQAPGKGLEWVSAIG |
| | DASSRAPGIPDRFSGSGSGTDFTLTI | gSGGSLPYADSVKGRFTISRDNSKNTL |
| | SRLEPEDFAVYYCQQYSRWPLTFG | YLQMNSLRAEDTAVYYCARYWPMD |
| | QGTKVEIK (SEQ ID NO: 81) | SWGQGTLVTVSS (SEQ ID NO: 78) |
| C01_Rd4 | EIVLTQSPGTLSLSPGERATLSCRAS | EVQLLESGGGLVQPGGSLRLSCAASG |
| _6 nM_C20 | QSVSPIYLAWYQQKPGQAPRLLIY | FTFSSYPMSWVRQAPGKGLEWVSAIG |
| | DASSRAPGIPDRFSGSGSGTDFTLTI | gSGGSLPYADSVKGRFTISRDNSKNTL |
| | SRLEPEDFAVYYCQQYSAFPLTFG | YLQMNSLRAEDTAVYYCARYWPMD |
| | QGTKVEIK (SEQ ID NO: 82) | SWGQGTLVTVSS (SEQ ID NO: 78) |
| C01_Rd4 | EIVLTQSPGTLSLSPGERATLSCWL | EVQLLESGGGLVQPGGSLRLSCAASG |
| _6 nM_C12 | SQSVSSTYLAWYQQKPGQAPRLLI | FTFSSYPMSWVRQAPGKGLEWVSAIG |
| (PC1C12) | YDASSRAPGIPDRFSGSGSGTDFTL | gSGGWSYYADSVKGRFTISRDNSKNT |
| | TISRLEPEDFAVYYCQQYSEWPLT | LYLQMNSLRAEDTAVYYCARYWPM |
| | FGQGTKVEIK (SEQ ID NO: 84) | DSWGQGTLVTVSS (SEQ ID NO: 83) |
| C01_Rd4 | EIVLTQSPGTLSLSPGERATLSCRAS | EVQLLESGGGLVQPGGSLRLSCAASG |
| _0.6 nM_C16 | QSVSSTYLAWYQQKPGQAPRLLIY | FTFSSYPMSWVRQAPGKGLEWVSAIG |
| | DASSRAPGIPDRFSGSGSGTDFTLTI | gSGGSLPYADSVKGRFTISRDNSKNTL |
| | SRLEPEDFAVYYCQQYSSWPLTFG | YLQMNSLRAEDTAVYYCARYWPMD |
| | QGTKVEIK (SEQ ID NO: 85) | SWGQGTLVTVSS (SEQ ID NO: 78) |
| C01_Rd4 | EIVLTQSPGTLSLSPGERATLSCRAS | EVQLLESGGGLVQPGGSLRLSCAASG |
| _0.6 nM_C09 | QSVSSIFLAWYQQKPGQAPRLLIYD | FTFSSYPMSWVRQAPGKGLEWVSAIG |
| | ASSRAPGIPDRFSGSGSGTDFTLTIS | gSGGSLPYADSVKGRFTISRDNSKNTL |
| | RLEPEDFAVYYCQQYSAWPLTFG | YLQMNSLRAEDTAVYYCARYWPMD |
| | QGTKVEIK (SEQ ID NO: 86) | SWGQGTLVTVSS (SEQ ID NO: 78) |
| C01_Rd4 | EIVLTQSPGTLSLSPGERATLSCACS | EVQLLESGGGLVQPGGSLRLSCAASG |
| _6 nM_C09 | QSVSSTYLAWYQQKPGQAPRLLIY | FTFSSYPMSWVRQAPGKGLEWVSAT |
| | DASSRAPGIPDRFSGSGSGTDFTLTI | VgSGGSIGYADSVKGRFTISRDNSKNT |
| | SRLEPEDFAVYYCQQYSAWPLTFG | LYLQMNSLRAEDTAVYYCARYWPM |
| | QGTKVEIK (SEQ ID NO: 88) | DSWGQGTLVTVSS (SEQ ID NO: 87) |
| C01_Rd4 | EIVLTQSPGTLSLSPGERATLSCRAS | EVQLLESGGGLVQPGGSLRLSCAASG |
| _0.6 nM_C03 | CDVSSTYLAWYQQKPGQAPRLLIY | FTFSSYPMSWVRQAPGKGLEWVSAIG |
| | DASSRAPGIPDRFSGSGSGTDFTLTI | gSGGSLPYADSVKGRFTISRDNSKNTL |
| | SRLEPEDFAVYYCQQYMRSPLTFG | YLQMNSLRAEDTAVYYCARYWPMD |
| | QGTKVEIK (SEQ ID NO: 89) | SWGQGTLVTVSS (SEQ ID NO: 78) |
| C01_Rd4 | EIVLTQSPGTLSLSPGERATLSCRAS | EVQLLESGGGLVQPGGSLRLSCAASG |
| _0.6 nM_C06 | EAVPSTYLAWYQQKPGQAPRLLIY | FTFSSYPMSWVRQAPGKGLEWVSAIG |
| | DASSRAPGIPDRFSGSGSGTDFTLTI | gSGGSLPYADSVKGTISRDNSKNTLY |
| | SRLEPEDFAVYYCQQYSAFPLTFG | LQMNSLRAEDTAVYYCARYWPMDS |
| | QGTKVEIK (SEQ ID NO: 90) | WGQGTLVTVSS (SEQ ID NO: 413) |
| C01_Rd4 | EIVLTQSPGTLSLSPGERATLSCCSS | EVQLLESGGGLVQPGGSLRLSCAASG |
| _6 nM_C04 | QSVSSTYLAWYQQKPGQAPRLLIY | FTFSSYPMSWVRQAPGKGLEWVSAIG |
| | DASSRAPGIPDRFSGSGSGTDFTLTI | gSGGSLPYADSVKGRFTISRDNSKNTL |
| | SRLEPEDFAVYYCQQYSAFPLTFG | YLQMNSLRAEDTAVYYCARYWPMD |
| | QGTKVEIK (SEQ ID NO: 91) | SWGQGTLVTVSS (SEQ ID NO: 78) |
| COMBO_ | EIVLTQSPGTLSLSPGERATLSCRAS | EVQLLESGGGLVQPGGSLRLSCAASG |
| Rd4_0.6 nM | VRVSSTYLAWYQQKPGQAPRLLM | FTFSSYAMNWVRQAPGKGLEWVSAI |
| _C22 | YDASIRATGIPDRFSGSGSGTDFTL | SdSGGSRWYADSVKGRFTISRDNSKN |
| (COM22) | TISRLEPEDFAVYYCQQYMKWPL | TLYLQMNSLRAEDTAVYYCTRYWPM |
| | TFGQGTKVEIK (SEQ ID NO: 93) | DIWGQGTLVTVSS (SEQ ID NO: 92) |
| COMBO_ | EIVLTQSPGTLSLSPGERATLSCRAS | EVQLLESGGGLVQPGGSLRLSCAASG |
| Rd4_6 nM | QSVSAAYLAWYQQKPGQAPRLLM | FTFSSYPMSWVRQAPGKGLEWVSAIG |
| _C21 | YDASIRATGIPDRFSGSGSGTDFTL | gSGGSLPYADSVKGRFTISRDNSKNTL |
| | TISRLEPEDFAVYYCQQYMCWPL | YLQMNSLRAEDTAVYYCARYWPMD |
| | TFGQGTKVEIK (SEQ ID NO: 94) | SWGQGTLVTVSS (SEQ ID NO: 78) |
| COMBO_ | EIVLTQSPGTLSLSPGERATLSCRAS | EVQLLESGGGLVQPGGSLRLSCAASG |
| Rd4_6 nM | QSVSSSYWGWYQQKPGQAPRLLM | FTFSSYPMSWVRQAPGKGLEWVSAIG |
| _C10 | YDASIRATGIPDRFSGSGSGTDFTL | gSGGSIHYADSVKGRFTISRDNSKNTL |
| | TISRLEPEDFAVYYCQQYQCWPLT | YLQMNSLRAEDTAVYYCARYWPMD |
| | FGQGTKVEIK (SEQ ID NO: 96) | SWGQGTLVTVSS (SEQ ID NO: 95) |
| COMBO_ | EIVLTQSPGTLSLSPGERATLSCRAS | EVQLLESGGGLVQPGGSLRLSCAASG |
| Rd4_0.6 nM | QSVSSTYLAWYQQKPGQAPRLLM | FTFSSYPMSWVRQAPGKGLEWVSAHI |
| _C04 | YDASIRATGIPDRFSGSGSGTDFTL | gSGGSTYYADSVKGRFTISRDNSKNT |
| | TISRLEPEDFAVYYCQQYQSWPLT | LYLQMNSLRAEDTAVYYCARYWPM |
| | FGQGTKVEIK (SEQ ID NO: 98) | DSWGQGTLVTVSS (SEQ ID NO: 97) |
| COMBO_ | EIVLTQSPGTLSLSPGERATLSCRAS | EVQLLESGGGLVQPGGSLRLSCAASG |
| Rd4_6 nM | QSVSSpYLAWYQQKPGQAPRLLM | FTFSSYPMSWVRQAPGKGLEWVSAIG |
| _C25 | YDASIRATGIPDRFSGSGSGTDFTL | gSGGSTYYADSVKGRFTISRDNSKNT |
| | TISRLEPEDFAVYYCQQYQSWPLT | LYLQMNSLRAEDTAVYYCARYWPM |
| | FGQGTKVEIK (SEQ ID NO: 100) | DPWGQGTLVTVSS (SEQ ID NO: 99) |
| COMBO_ | EIVLTQSPGTLSLSPGERATLSCRAS | EVQLLESGGGLVQPGGSLRLSCAASG |
| Rd4_0.6 nM | QSVSSSYLAWYQQKPGQAPRLLM | FTFSSYPMSWVRQAPGKGLEWVSAIG |
| _C21 | YDASIRATGIPDRFSGSGSGTDFTL | gSGGSLPYADSVKGRFTISRDNSKNTL |
| | TISRLEPEDFAVYYCQQYQSWPLT | YLQMNSLRAEDTAVYYCARYWPMD |
| | FGQGTKVEIK (SEQ ID NO: 38) | SWGQGTLVTVSS (SEQ ID NO: 78) |
| COMBO_ | EIVLTQSPGTLSLSPGERATLSCRAS | EVQLLESGGGLVQPGGSLRLSCAASG |
| Rd4_6 nM | QSVSPIYLAWYQQKPGQAPRLLMY | FTFSSYPMSWVRQAPGKGLEWVSAIG |
| C11 | DASIRATGIPDRFSGSGSGTDFTLTI | GSGGSLGYADSVKGRFTISRDNSKNT |
| | SRLEPEDFAVYYCQQYKAWPLTF | LYLQMNSLRAEDTAVYYCARYWPM |
| | GQGTKVEIK (SEQ ID NO: 102) | DSWGQGTLVTVSS (SEQ ID NO: 101) |
| COMBO_ | EIVLTQSPGTLSLSPGERATLSCRAS | EVQLLESGGGLVQPGGSLRLSCAASG |
| Rd4_0.6 nM | QSVSYLYLAWYQQKPGQAPRLLM | FTFSSYPMSWVRQAPGKGLEWVSAIG |
| _C20 | YDASIRATGIPDRFSGSGSGTDFTL | GSGGSLPYADSVKGRFTISRDNSKNT |
| | TISRLEPEDFAVYYCQQYMEWPLT | LYLQMNSLRAEDTAVYYCARYWPM |
| | FGQGTKVEIK (SEQ ID NO: 103) | DSWGQGTLVTVSS (SEQ ID NO: 78) |
| COMBO_ | EIVLTQSPGTLSLSPGERATLSCRAS | EVQLLESGGGLVQPGGSLRLSCAASG |
| Rd4_6 nM | QSVSAQYLAWYQQKPGQAPRLLM | FTFSSYPMSWVRQAPGKGLEWVSAIF |
| _C09 | YDASIRATGIPDRFSGSGSGTDFTL | ASGGSTYYADSVKGRFTISRDNSKNT |
| | TISRLEPEDFAVYYCQQYQAWPLT | LYLQMNSLRAEDTAVYYCARYWPM |
| | FGQGTKVEIK (SEQ ID NO: 105) | DSWGQGTLVTVSS (SEQ ID NO: 104) |
| COMBO_ | EIVLTQSPGTLSLSPGERATLSCRAS | EVQLLESGGGLVQPGGSLRLSCAASG |
| Rd4_6 nM | QSVSSSYLAWYQQKPGQAPRLLM | FTFSSYPMSWVRQAPGKGLEWVSAIG |
| _C08 | YDASIRATGIPDRFSGSGSGTDFTL | GSGTWTYYADSVKGRFTISRDNSKN |
| | TISRLEPEDFAVYYCQQYQKWPLT | TLYLQMNSLRAEDTAVYYCARYWP |
| | FGQGTKVEIK (SEQ ID NO: 107) | MDSWGQGTLVTVSS (SEQ ID NO: 106) |
| COMBO_ | EIVLTQSPGTLSLSPGERATLSCRAS | EVQLLESGGGLVQPGGSLRLSCAASG |
| Rd4_0.6 nM | QSVSAVYLAWYQQKPGQAPRLLM | FTFSSYPMSWVRQAPGKGLEWVSAIG |
| _C19 | YDASIRATGIPDRFSGSGSGTDFTL | GSGGSLPYADSVKGRFTISRDNSKNT |
| | TISRLEPEDFAVYYCQQYRAWPLT | LYLQMNSLRAEDTAVYYCARYWPM |
| | FGQGTKVEIK (SEQ ID NO: 108) | DSWGQGTLVTVSS (SEQ ID NO: 78) |
| COMBO_ | EIVLTQSPGTLSLSPGERATLSCRAS | EVQLLESGGGLVQPGGSLRLSCAASG |
| Rd4_0.6 nM | IAVSSTYLAWYQQKPGQAPRLLM | FTFSSYPMSWVRQAPGKGLEWVSAIG |
| _C02 | YDASIRATGIPDRFSGSGSGTDFTL | GSGGSLPYADSVKGRFTISRDNSKNT |
| | TISRLEPEDFAVYYCQQYMVWPL | LYLQMNSLRAEDTAVYYCARYWPM |
| | TFGQGTKVEIK (SEQ ID NO: 109) | DSWGQGTLVTVSS (SEQ ID NO: 78) |
| COMBO_ | EIVLTQSPGTLSLSPGERATLSCRPR | EVQLLESGGGLVQPGGSLRLSCAASG |
| Rd4_0.6 nM | QSVSSSYLAWYQQKPGQAPRLLM | FTFSSYPMSWVRQAPGKGLEWVSAL |
| _C23 | YDASIRATGIPDRFSGSGSGTDFTL | FGSGGSTYYADSVKGRFTISRDNSKN |
| | TISRLEPEDFAVYYCQQYQDWPLT | TLYLQMNSLRAEDTAVYYCARYWP |
| | FGQGTKVEIK (SEQ ID NO: 111) | MDSWGQGTLVTVSS (SEQ ID NO: 110) |
| COMBO_ | EIVLTQSPGTLSLSPGERATLSCRAS | EVQLLESGGGLVQPGGSLRLSCAASG |
| Rd4_0.6 nM | QSVSSSYLAWYQQKPGQAPRLLM | FTFSSYPMSWVRQAPGKGLEWVSAIG |
| _C29 | YDASIRATGIPDRFSGSGSGTDFTL | GSGGSLPYADSVKGRFTISRDNSKNT |
| | TISRLEPEDFAVYYCQQYQSWPLT | LYLQMNSLRAEDTAVYYCARYWPM |
| | FGQGTKVEIK (SEQ ID NO: 38) | DIWGQGTLVTVSS (SEQ ID NO: 112) |
| COMBO_ | EIVLTQSPGTLSLSPGERATLSCRAS | EVQLLESGGGLVQPGGSLRLSCAASG |
| Rd4_0.6 nM | QSVSSTYLAWYQQKPGQAPRLLM | FTFSSYPMSWVRQAPGKGLEWVSAIG |
| _C09 | YDASIRATGIPDRFSGSGSGTDFTL | GSGGSLPYADSVKGRFTISRDNSKNT |
| | TISRLEPEDFAVYYCQQYQEWPLT | LYLQMNSLRAEDTAVYYCARYWPM |
| | FGQGTKVEIK (SEQ ID NO: 113) | DIWGQGTLVTVSS (SEQ ID NO: 112) |
| COMBO_ | EIVLTQSPGTLSLSPGERATLSCRAS | EVQLLESGGGLVQPGGSLRLSCAASG |
| Rd4_6 nM | QSVSASYLAWYQQKPGQAPRLLM | FTFSSYPMSWVRQAPGKGLEWVSAA |
| _C12 | YDASIRATGIPDRFSGSGSGTDFTL | LGSGGSTYYADSVKGRFTISRDNSKN |
| | TISRLEPEDFAVYYCQQYMSWPLT | TLYLQMNSLRAEDTAVYYCARYWP |
| | FGQGTKVEIK (SEQ ID NO: 115) | MDSWGQGTLVTVSS (SEQ ID NO: 114) |
| COMBO_ | EIVLTQSPGTLSLSPGERATLSCRAS | EVQLLESGGGLVQPGGSLRLSCAASG |
| Rd4_0.6 nM | QSVSYMYLAWYQQKPGQAPRLLI | FTFSSYPMSWVRQAPGKGLEWVSAIG |
| _C30 | YDASIRATGIPDRFSGSGSGTDFTL | GSGGSTYYADSVKGRFTISRDNSKNT |
| | TISRLEPEDFAVYYCQQYKSWPLT | LYLQMNSLRAEDTAVYYCARYWPM |
| | FGQGTKVEIK (SEQ ID NO: 116) | DSWGQGTLVTVSS (SEQ ID NO: 76) |
| COMBO_ | EIVLTQSPGTLSLSPGERATLSCRAS | EVQLLESGGGLVQPGGSLRLSCAASG |
| Rd4_0.6 nM | QSVSALYLAWYQQKPGQAPRLLM | FTFSSYPMSWVRQAPGKGLEWVSAIG |
| _C14 | YDASIRATGIPDRFSGSGSGTDFTL | GSGGSLPYADSVKGRFTISRDNSKNT |
| | TISRLEPEDFAVYYCQQYYGWPLT | LYLQMNSLRAEDTAVYYCARYWPM |
| | FGQGTKVEIK (SEQ ID NO: 117) | DIWGQGTLVTVSS (SEQ ID NO: 112) |
| COMBO_ | EIVLTQSPGTLSLSPGERATLSCRAS | EVQLLESGGGLVQPGGSLRLSCAASG |
| Rd4_6 nM | QPISSSYLAWYQQKPGQAPRLLMY | FTFSSYPMSWVRQAPGKGLEWVSAIG |
| _C07 | DASIRATGIPDRFSGSGSGTDFTLTI | GSGGSLPYADSVKGRFTISRDNSKNT |
| | SRLEPEDFAVYYCQQYQGWPLTF | LYLQMNSLRAEDTAVYYCARYWPM |
| | GQGTKVEIK (SEQ ID NO: 119) | ADWGQGTLVTVSS (SEQ ID NO: 118) |
| COMBO_ | EIVLTQSPGTLSLSPGERATLSCRAS | EVQLLESGGGLVQPGGSLRLSCAASG |
| Rd4_6 nM | QSVSSSYLAWYQQKPGQAPRLLM | FTFSSYAMNWVRQAPGKGLEWVSAI |
| _C02 | YDASIRATGIPDRFSGSGSGTDFTL | SDSGGFVYYADSVKGRFTISRDNSKN |
| | TISRLEPEDFAVYYCQQYEFWPLT | TLYLQMNSLRAEDTAVYYCARYWP |
| | FGQGTKVEIK (SEQ ID NO: 121) | MDSWGQGTLVTVSS (SEQ ID NO: 120) |
| COMBO_ | EIVLTQSPGTLSLSPGERATLSCRAS | EVQLLESGGGLVQPGGSLRLSCAASG |
| Rd4_0.6 nM | QSVSSTYLAWYQQKPGQAPRLLM | FTFSSYAMNWVRQAPGKGLEWVSAI |
| _C05 | YDASIRATGIPDRFSGSGSGTDFTL | GGSGGSTYYADSVKGRFTISRDNSKN |
| | TISRLEPEDFAVYYCQQYMSWPLT | TLYLQMNSLRAEDTAVYYCARYWP |
| | FGQGTKVEIK (SEQ ID NO: 123) | MSLWGQGTLVTVSS (SEQ ID NO: 122) |
| COMBO_ | EIVLTQSPGTLSLSPGERATLSCRAS | EVQLLESGGGLVQPGGSLRLSCAASG |
| Rd4_0.6 nM | QGISSTYLAWYQQKPGQAPRLLM | FTFSSYPMSWVRQAPGKGLEWVSAIG |
| _C17 | YDASIRATGIPDRFSGSGSGTDFTL | GSGGSLPYADSVKGRFTISRDNSKNT |
| | TISRLEPEDFAVYYCQQYAYWPLT | LYLQMNSLRAEDTAVYYCARYWPM |
| | FGQGTKVEIK (SEQ ID NO: 124) | DIWGQGTLVTVSS (SEQ ID NO: 112) |
| COMBO_ | EIVLTQSPGTLSLSPGERATLSCRAS | EVQLLESGGGLVQPGGSLRLSCAASG |
| Rd4_6 nM | QSVSSSYLAWYQQKPGQAPRLLM | FTFSSYAMNWVRQAPGKGLEWVSAC |
| _C22 | YDASIRATGIPDRFSGSGSGTDFTL | LDSGGSTYYADSVKGRFTISRDNSKN |
| | TISRLEPEDFAVYYCQQYQGWPLT | TLYLQMNSLRAEDTAVYYCARYWP |
| | FGQGTKVEIK (SEQ ID NO: 126) | MDSWGQGTLVTVSS (SEQ ID NO: 125) |
| COMBO_ | EIVLTQSPGTLSLSPGERATLSCRAS | EVQLLESGGGLVQPGGSLRLSCAASG |
| Rd4_0.6 nM | QSVSVRYLAWYQQKPGQAPRLLM | FTFSSYPMSWVRQAPGKGLEWVSAA |
| _C11 | YDASIRATGIPDRFSGSGSGTDFTL | LGSGGSTYYADSVKGRFTISRDNSKN |
| | TISRLEPEDFAVYYCQQYGSWPITF | TLYLQMNSLRAEDTAVYYCARYWP |
| | GQGTKVEIK (SEQ ID NO: 128) | MSLWGQGTLVTVSS (SEQ ID NO: 127) |
| P6DY | EIVLTQSPGTLSLSPGERATLSCRAS | EVQLLESGGGLVQPGGSLRLSCAASG |
| | QSVSSSYPSWYQQKPGQAPRLLIY | FTFGSYAMTWVRQAPGKGLEWVSAI |
| | GASSRATGIPDRFSGSGSGTDFTLTI | DYSGGNTFYADSVKGRFTISRDNSKN |
| | SRLEPEDFAVYYCQHYPYPPSFTF | TLYLQMNSLRAEDTAVYYCARVSPIA |
| | GQGTKVEIK (SEQ ID NO: 18) | SGMDYWGQGTLVTVSS (SEQ ID NO: |
| | | 25) |
| P6AP | EIVLTQSPGTLSLSPGERATLSCRAS | EVQLLESGGGLVQPGGSLRLSCAASG |
| | QLGSFYLAWYQQKPGQAPRLLIY | FTFGSYAMTWVRQAPGKGLEWVSAI |
| | GASSRATGIPDRFSGSGSGTDFTLTI | SGSGGNTFYADSVKGRFTISRDNSKN |
| | SRLEPEDFAVYYCQHYNYPPSFTF | TLYLQMNSLRAEDTAVYYCARVSPIA |
| | GQGTKVEIK (SEQ ID NO: 80) | APMDYWGQGTLVTVSS (SEQ ID NO: |
| | | 8) |
| Consensus | EIVLTQSPGTLSLSPGERATLSC | EVQLLESGGGLVQPGGSLRLSCAASG |
| | X₁X₂X₃X₄X₅X₆X₇X₈X₉X₁₀X₁₁X₁₂WYQ | FTFX₁SYX₂MX₃WVRQAPGKGLEWVS |
| | QKPGQAPRLLMYX₁₃ASX₁₄RAX₁₅GI | AX₄X5X₆X₇GX₈X₉X₁₀X₁₁YADX₁₂X₁₃KGR |
| | PDRFSGSGSGTDFTLTISRLEPEDFA | FTISRDNSKNTLYLQMNSLRAEDTAV |
| | VYYCX₁₆X₁₇YX₁₈X₁₉PPSFTFGQGTK | YYCARVSPIX₁₄X₁₅X₁₆MDYWGQGTLV |
| | VEIK, wherein X₁ is R, G, W, A, or C; | TVSS, wherein X₁ is G or S, X₂ is A or P; |
| | X₂ is A, P, G, L, C, or S; X₃ is S, G, or | X₃ is T, N, or S; X₄ is I, V, T, H, L, A, or |
| | R; X₄ is Q, C, E, V, or I; X₅ is S, P, G, | C; X₅ is S, D, G, T, I, L, F, M, or V; X₆ is |
| | A, R, or D; X₆ is V, G, I, or L; X₇ is S, | G, Y, L, H, D, A, S, or M; X₇ is S, Q, T, A, |
| | E, D, P, or G; X₈ is S, P, F, A, M, E, V, | F, or W; X₈ is G or T; X₉ is N, S, P, Y, W, |
| | N, D, or Y; X₉ is I, T, V, E, S, A, M, Q, | or F; X₁₀ is S, T, I, L, T, A, R, V, K, G, or |
| | Y, H, R, or F; X₁₀ is Y or F; X₁₁ is L, | C; X₁₁ is F, Y, P, W, H, or G; X₁₂ is V, R, |
| | W, or P; X₁₂ is A, S, or G, X₁₃ is G or | or L; X₁₃ is G or T; X₁₄ is A or Y; X₁₅ is A |
| | D; X₁₄ is S or I; X₁₅ is T or P; X₁₆ is Q | or S; and X₁₆ is G, Q, L, P, or E (SEQ ID |
| | or K; X₁₇ is H or Y; X₁₈ is G, N, or P; | NO: 313); or |
| | and X₁₉ is S, W, or Y (SEQ ID NO: | EVQLLESGGGLVQPGGSLRLSCAASG |
| | 315); or | FTFX₁SYX₂MX₃WVRQAPGKGLEWVS |
| | EIVLTQSPGTLSLSPGERATLSC | AX₄X5X₆X7GX₈X₉X₁₀X₁₁YADX₁₂X₁₃KGR |
| | X₁X2X3X4X₅X₆X7X₈X₉X₁₀X₁₁X₁₂WYQ | FTISRDNSKNTLYLQMNSLRAEDTAV |
| | QKPGQAPRLLMYX₁₃ASX₁₄RAX₁₅GI | YYCARYWPMX₁₄X₁₅WGQGTLVTVSS, |
| | PDRFSGSGSGTDFTLTISRLEPEDFA | wherein X₁ is G or S, X₂ is A or P; X₃ is T, |
| | VYYCQQYX₁₆X₁₇X₁₈PX₁₉FGQGTKV | N, or S; X₄ is I, V, T, H, L, A, or C; X₅ is S, |
| | EIK, wherein X₁ is R, G, W, A, or C; X₂ | D, G, T, I, L, F, M, or V; X₆ is G, Y, L, H, |
| | is A, P, G, L, C, or S; X₃ is S, G, or R; | D, A, S, or M; X₇ is S, Q, T, A, F, or W; X₈ |
| | X₄ is Q, C, E, V, or I; X₅ is S, L, P, G, | is G or T; X₉ is N, S, P, Y, W, or F; X₁₀ is |
| | A, R, or D; X₆ is V, G, or I; X₇ is S, E, | S, T, I, L, T, A, R, V, K, G, or C; X₁₁ is F, |
| | D, or P; X₈ is S, P, F, A, M, E, V, N, D, | Y, P, W, H, or G; X₁₂ is V, R, or L; X₁₃ is |
| | or Y; X₉ is I, T, V, E, S, A, M, Q, Y, H, | G or T; X₁₄ is D, S, T, or A; and X₁₅ is I, S, |
| | or R; X₁₀ is Y or F; X₁₁ is L, W, or P; | L, P, or D (SEQ ID NO: 314) |
| | X₁₂ is A, S, or G, X₁₃ is G or D; X₁₄ is S | |
| | or I; X₁₅ is T or P; X₁₆ is G, Q, E, L, F, | |
| | A, S, M, R, K, or Y; X₁₇ is S, R, T, G, | |
| | R, V, D, A, H, E, K, C, F, or Y; X₁₈ is | |
| | W, S, or F; and X₁₉ is L or I (SEQ ID | |
| | NO: 316) | |
| P4G4 | EIVLTQSPGTLSLSPGERATLSCRAS | EVQLLESGGGLVQPGGSLRLSCAASG |
| | QSVSSSYLAWYQQKPGQAPRLLIY | FTFSSYAMSWVRQAPGKGLEWVSAIS |
| | GASSRAYGIPDRFSGSGSGTDFTLTI | ASGGSTYYADSVKGRFTISRDNSKNT |
| | SRLEPEDFAVYYCQHYGSPPLFTF | LYLQMNSLRAEDTAVYYCARLSWSG |
| | GQGTKVEIK (SEQ ID NO: 401) | AFDNWGQGTLVTVSS (SEQ ID NO: |
| | | 378) |
| P1A11 | EIVLTQSPGTLSLSPGERATLSCRAS | EVQLLESGGGLVQPGGSLRLSCAASG |
| | QNVSSSYLAWYQQKPGQAPRLLIY | FTFRSYAMSWVRQAPGKGLEWVSAI |
| | GASYRATGIPDRFSGSGSGTDFTLT | SGSGGSTFYADSVKGRFTISRDNSKNT |
| | ISRLEPEDFAVYYCQHYGSPPSFTF | LYLQMNSLRAEDTAVYYCATVGTSG |
| | GQGTKVEIK (SEQ ID NO: 379) | AFGIWGQGTLVTVSS (SEQ ID NO: |
| | | 380) |

Also provided herein are CDR portions of extracellular ligand-binding domains of CARs to BCMA (including Chothia, Kabat CDRs, and CDR contact regions). Determination of CDR regions is well within the skill of the art. It is understood that in some embodiments, CDRs can be a combination of the Kabat and Chothia CDR (also termed “combined CRs” or “extended CDRs”). In some embodiments, the CDRs are the Kabat CDRs. In other embodiments, the CDRs are the Chothia CDRs. In other words, in embodiments with more than one CDR, the CDRs may be any of Kabat, Chothia, combination CDRs, or combinations thereof. Table 2A and Table 2B provide examples of CDR sequences provided herein.

TABLE 2A
Heavy Chain

| Binding Domain | CDRH1 | CDRH2 | CDRH3 |
|---|---|---|---|
| P6E01 | SYAMT (SEQ ID NO: | AISGSGGNTFYA | VSPIASGMDY |
| For the following mAbs: | 129) (Kabat); | DSVKG (SEQ ID | (SEQ ID NO: |
| P6E01/P6E01;L1.LGF/L3. | GFTFGSY (SEQ ID NO: | NO: 132) (Kabat) | 134) |
| KW/P6E01; | 130) (Chothia); | SGSGGN (SEQ ID | |
| L1.LGF/L3.NY/P6E01; | GFTFGSYAMT (SEQ ID | NO: 133) (Chothia) | |
| L1 GDF/L3.NY/P6E01; | NO: 131) (extended) | | |
| L3.KW/P6E01; | | | |
| L3.PY/P6E01; | | | |
| L3.NY/P6E01; | | | |
| L3.PY/L1.PS/P6E01; | | | |
| L3.PY/L1.AH/P6E01; | | | |
| L3.PY/L1.FF/P6E01; | | | |
| L3.PY/L1.PH/P6E01; | | | |
| L3.PY/L3.KY/P6E01; | | | |
| L3.PY/L3.KF/P6E01; and | | | |
| L3.PY/P6E01. | | | |
| H3.AQ | SYAMT (SEQ ID NO: | AISGSGGNTFYA | VSPIAAQMDY |
| For the following mAbs: | 129) (Kabat); | DSVKG (SEQ ID | (SEQ ID NO: |
| P6E01/H3.AQ; | GFTFGSY (SEQ ID NO: | NO: 132) (Kabat) | 135) |
| L1.LGF/L3.KW/H3.AQ; | 130) (Chothia); | SGSGGN (SEQ ID | |
| L1.LGF/L3.PY/H3.AQ | GFTFGSYAMT (SEQ ID | NO: 133) (Chothia) | |
| | NO: 131) (extended) | | |
| H3.AL | SYAMT (SEQ ID NO: | AISGSGGNTFYA | VSPIAALMDY |
| For the following mAbs: | 129) (Kabat); | DSVKG (SEQ ID | (SEQ ID NO: |
| L1.LGF/L3.KW/H3.AL; | GFTFGSY (SEQ ID NO: | NO: 132) (Kabat) | 136) |
| L1.LGF/L3.NY/H3.AL; and | 130) (Chothia); | SGSGGN (SEQ ID | |
| L1.GDF/L3.NY/H3.AL. | GFTFGSYAMT (SEQ ID | NO: 133) (Chothia) | |
| | NO: 131) (extended) | | |
| H3.AP | SYAMT (SEQ ID NO: | AISGSGGNTFYA | VSPIAAPMDY |
| For the following mAbs: | 129) (Kabat); | DSVKG (SEQ ID | (SEQ ID NO: |
| L1.LGF/L3.KW/H3.AP; | GFTFGSY (SEQ ID NO: | NO: 132) (Kabat) | 137) |
| L1.LGF/L3.PY/H3.AP; | 130) (Chothia); | SGSGGN (SEQ ID | |
| L1.LGF/L3NY/H3.AP; | GFTFGSYAMT (SEQ ID | NO: 133) (Chothia) | |
| L1.GDF/L3.KW/H3.AP; | NO: 131) (extended) | | |
| L1.GDF/L3NY/H3.AP; | | | |
| P6AP. | | | |
| H2.QR | SYAMT (SEQ ID NO: | AISGSGGNTFYA | VSPIASGMDY |
| For the following mAbs: | 129) (Kabat); | DQRKG (SEQ ID | (SEQ ID NO: |
| L3.PY/H2.QR; | GFTFGSY (SEQ ID NO: | NO: 138) (Kabat) | 134) |
| L3.PY/L1.PS/H2.QR; | 130) (Chothia); | SGSGGN (SEQ ID | |
| L3.PY/LLAH/H2.QR; | GFTFGSYAMT (SEQ ID | NO: 133)-(Chothia) | |
| L3.PY/L1.FF/H2.QR; | NO: 131) (extended) | | |
| L3.PY/L1.PH/H2.QR; and | | | |
| L3.PY/L3.KY/H2.QR. | | | |
| H2.DY | SYAMT (SEQ ID NO: | AIDYSGGNTFYA | VSPIASGMDY |
| For the following mAbs: | 129) (Kabat); | DSVKG (SEQ ID | (SEQ ID NO: |
| L3.PY/H2.DY; P6DY; | GFTFGSY (SEQ ID NO: | NO: 139) (Kabat) | 134) |
| L3.PY/L1.PS/H2.DY; | 130) (Chothia); | DYSSGN (SEQ ID | |
| L3.PY/L1.AH/H2.DY; | GFTFGSYAMT (SEQ ID | NO: 140)-(Chothia) | |
| L3.PY/L1.FF/H2.DY; | NO: 131) (extended) | | |
| L3.PY/L3.KY/H2.DY; and | | | |
| L3.PY/L3.KF/H2.DY. | | | |
| H2.YQ | SYAMT (SEQ ID NO: | AISYQGGNTFYA | VSPIASGMDY |
| For the following mAbs: | 129) (Kabat); | DSVKG (SEQ ID | (SEQ ID NO: |
| L3.PY/H2.YQ; | GFTFGSY (SEQ ID NO: | NO: 141) (Kabat) | 134) |
| L3.PY/L1.PS/H2.YQ; | 130) (Chothia); | SYQGGN (SEQ ID | |
| L3.PY/L1.AH/H2.YQ; | GFTFGSYAMT (SEQ ID | NO: 142)-(Chothia) | |
| L3.PY/L1.FF/H2.YQ; | NO: 131) (extended) | | |
| L3.PY/L3.KY/H2.YQ; and | | | |
| L3.PY/L3.KF/H2.YQ. | | | |
| H2.LT | SYAMT (SEQ ID NO: | AISLTGGNTFYA | VSPIASGMDY |
| For the following mAbs: | 129) (Kabat); | DSVKG (SEQ ID | (SEQ ID NO: |
| L3.PY/H2.LT; | GFTFGSY (SEQ ID NO: | NO: 143) (Kabat) | 134) |
| L3.PY/L1.PS/H2.LT; | 130) (Chothia); | SLTGGN (SEQ ID | |
| L3.PY/L1.AH/H2.LT; | GFTFGSYAMT (SEQ ID | NO: 144)-(Chothia) | |
| L3.PY/L1.FF/H2.LT; | NO: 131) (extended) | | |
| L3.PY/L3.KY/H2.LT; and | | | |
| L3.PY/L3.KF/H2.LT. | | | |
| H2.HA | SYAMT (SEQ ID NO: | AISHAGGNTFYA | VSPIASGMDY |
| For the following mAbs: | 129) (Kabat); | DSVKG (SEQ ID | (SEQ ID NO: |
| L3.PY/H2.HA; | GFTFGSY (SEQ ID NO: | NO: 145) (Kabat) | 134) |
| L3.PY/L1.AH/H2.HA; | 130) (Chothia); | SHAGGN (SEQ ID | |
| L3.PY/L1.FF/H2.HA; | GFTFGSYAMT (SEQ ID | NO: 146)-(Chothia) | |
| L3.PY/L1.PH/H2.HA; and | NO: 131) (extended) | | |
| L3.PY/L3.KY/H2.HA. | | | |
| H2.QL | SYAMT (SEQ ID NO: | AISGSGGNTFYA | VSPIASGMDY |
| For the following mAbs: | 129) (Kabat); | DQLKG (SEQ ID | (SEQ ID NO: |
| L3.PY/H2.QL; | GFTFGSY (SEQ ID NO: | NO: 147) (Kabat) | 134) |
| L3.PY/L1.PS/H2.QL; | 130) (Chothia); | SGSGGN (SEQ ID | |
| L3.PY/L1.AH/H2.QL; | GFTFGSYAMT (SEQ ID | NO: 133)-(Chothia) | |
| L3.PY/L1.FF/H2.QL; | NO: 131) (extended) | | |
| L3.PY/L3.KY/H2.QL; and | | | |
| L3.PY/L3.KF/H2.QL. | | | |
| H3.YA | SYAMT (SEQ ID NO: | AISGSGGNTFYA | VSPIYAGMDY |
| For the following mAbs: | 129) (Kabat); | DSVKG (SEQ ID | (SEQ ID NO: |
| L3.PY/H3.YA; | GFTFGSY (SEQ ID NO: | NO: 132) (Kabat) | 148) |
| L3.PY/L1.PS/H3.YA; | 130) (Chothia); | SGSGGN (SEQ ID | |
| L3.PY/L1.AH/H3.YA; | GFTFGSYAMT (SEQ ID | NO: 133) (Chothia) | |
| L3.PY/L1.FF/H3.YA; | NO: 131) (extended) | | |
| L3.PY/L3.KY/H3.YA; and | | | |
| L3.PY/L3.KF/H3.YA. | | | |
| H3AE | SYAMT (SEQ ID NO: | AISGSGGNTFYA | VSPIAAEMDY |
| For the following mAbs: | 129) (Kabat); | DSVKG (SEQ ID | (SEQ ID NO: |
| L3.PY/H3.AE; | GFTFGSY (SEQ ID NO: | NO: 132) (Kabat) | 149) |
| L3.PY/L1.AH/H3.AE; | 130) (Chothia); | SGSGGN (SEQ ID | |
| L3.PY/L1.FF/H3.AE; | GFTFGSYAMT (SEQ ID | NO: 133) (Chothia) | |
| L3.PY/L1.PH/H3.AE; and | NO: 131) (extended) | | |
| L3.PY/L3.KF/H3.AE. | | | |
| H3.TAQ | SYAMT (SEQ ID NO: | AISGSGGNTFYA | VSPIAAQMDY |
| For the following mAbs: | 129) (Kabat); | DSVKG (SEQ ID | (SEQ ID NO: |
| L3.PY/H3.TAQ; | GFTFGSY (SEQ ID NO: | NO: 132) (Kabat) | 135) |
| L3.PY/L1.PS/H3.TAQ; | 130) (Chothia); | SGSGGN (SEQ ID | |
| L3.PY/L1.AH/H3.TAQ; | GFTFGSYAMT (SEQ ID | NO: 133) (Chothia) | |
| L3.PY/L1.FF/H3.TAQ; | NO: 131) (extended) | | |
| L3.PY/L1.PH/H3.TAQ; and | | | |
| L3.PY/L3.KF/H3.TAQ. | | | |
| P5A2_VHVL and | SYAMN (SEQ ID NO: | AISDSGGSTYYA | YWPMDI (SEQ |
| A02_Rd4_6nM_C03 | 150) (Kabat); | DSVKG | IDNO: 155) |
| | GFTFSSY (SEQ ID NO: | (SEQ ID NO: 153) | |
| | 151) (Chothia); | (Kabat) | |
| | GFTFSSYAMN (SEQ ID | SDSGGS (SEQ ID | |
| | NO: 152) (extended) | NO: 154) (Chothia) | |
| COMBO_Rd4_0.6 nM_C17; | SYPMS (SEQ IDNO: 156) | AIGGSGGSLPYA | YWPMDI (SEQ |
| COMBO_Rd4_0.6 nM_C14; | (Kabat); | DSVKG | IDNO: 155) |
| COMBO_Rd4_0.6 nM_C29; | GFTFSSY (SEQ ID NO: | (SEQ ID NO: 158) | |
| and | 151) (Chothia); | (Kabat) | |
| COMBO_Rd4_0.6 nM_C09 | GFTFSSYPMS (SEQ ID | GGSGGS (SEQ ID | |
| | NO: 157) (extended) | NO: 159) (Chothia) | |
| C01_Rd4_6 nM_C04; | SYPMS (SEQ ID NO: 156) | AIGGSGGSLPYA | YWPMDS |
| C01_Rd4_0.6nM_C03; | (Kabat); | DSVKG | (SEQ ID NO: |
| C01_Rd4_0.6nM_C06; | GFTFSSY (SEQ ID NO: | (SEQ ID NO: 158) | 161) |
| COMBO_Rd4_0.6 nM_C02; | 151) (Chothia); | (Kabat) | |
| COMBO_Rd4_6 nM_C21; | GFTFSSYPMS (SEQ ID | GGSGGS (SEQ ID | |
| C01_Rd4_6 nM_C26; | NO: 157) (extended) | NO: 159) (Chothia) | |
| COMBO_Rd4_0.6 nM_C 19; | | | |
| C01_Rd4_6 nM_C24; | | | |
| C01_Rd4_6 nM_C20; | | | |
| C01_Rd4_0.6 nM_C09; | | | |
| COMBO_Rd4_0.6 nM_C21; | | | |
| C01_Rd4_0.6 nM_C04_C27; | | | |
| C01_Rd4_0.6 nM_C16; | | | |
| C01_Rd4_6 nM_C10; | | | |
| COMBO_Rd40.6 nM_C20 | | | |
| P5C1_VHVL (PC1) and | SYPMS (SEQ ID NO: 156) | AIGGSGGSTYYA | YWPMDS |
| COMBO_Rd4_0.6 nM_C 0 | (Kabat); | DSVKG | (SEQ ID NO: |
| | GFTFSSY (SEQ ID NO: | (SEQ ID NO: 162) | 161) |
| | 151) (Chothia); | (Kabat) | |
| | GFTFSSYPMS (SEQ ID | GGSGGS (SEQ ID | |
| | NO: 157) (extended) | NO: 159) (Chothia) | |
| A02_Rd4_0.6 nM_C06 | SYAMN (SEQ ID NO: | AISDSGGSAWYA | YWPMSL (SEQ |
| | 150) (Kabat); | DSVKG | ID NO: 164) |
| | GFTFSSY (SEQ ID NO: | (SEQ ID NO: 163) | |
| | 151) (Chothia); | (Kabat) | |
| | GFTFSSYAMN (SEQ ID | SDSGGS (SEQ ID | |
| | NO: 152) (extended) | NO: 154) (Chothia) | |
| A02_Rd4_0.6 nM_C09 | SYAMN (SEQ ID NO: | AISDSGGSAWYA | YWPMSL (SEQ |
| | 150) (Kabat); | DSVKG | ID NO: 164) |
| | GFTFSSY (SEQ ID NO: | (SEQ ID NO: 163) | |
| | 151) (Chothia); | (Kabat) | |
| | GFTFSSYAMN (SEQ ID | SDSGGS (SEQ ID | |
| | NO: 152) (extended) | NO: 154) (Chothia) | |
| A02_Rd4_0.6 nM_C16; | SYAMN (SEQ ID NO: | AISDFGGSTYYA | YWPMDI (SEQ |
| A02_Rd4_6 nM_C16 | 150) (Kabat); | DSVKG | ID NO: 155) |
| (P5A16) | GFTFSSY (SEQ ID NO: | (SEQ ID NO: 165) | |
| | 151) (Chothia); | (Kabat) | |
| | GFTFSSYAMN (SEQ ID | SDFGGS (SEQ ID | |
| | NO: 152) (extended) | NO: 166) (Chothia) | |
| A02_Rd4_6 nM_C01 | SYAMN (SEQ ID NO: | AITASGGSTYYA | YWPMSL (SEQ |
| | 150) (Kabat); | DSVKG | ID NO: 164) |
| | GFTFSSY (SEQ ID NO: | (SEQ ID NO: 167) | |
| | 151) (Chothia); | (Kabat) | |
| | GFTFSSYAMN (SEQ ID | TASGGS (SEQ ID | |
| | NO: 152) (extended) | NO: 168) (Chothia) | |
| A02_Rd4_6 nM_C26 | SYAMN (SEQ ID NO: | AISDSGGSTYYA | YWPMSL (SEQ |
| | 150) (Kabat); | DSVKG | ID NO: 164) |
| | GFTFSSY (SEQ ID NO: | (SEQ ID NO: 153) | |
| | 151) (Chothia); | (Kabat) | |
| | GFTFSSYAMN (SEQ ID | SDSGGS (SEQ ID | |
| | NO: 152) (extended) | NO: 154) (Chothia) | |
| A02_Rd4_6 nM_C25 | SYAMN (SEQ ID NO: | AISDSGGSRWYA | YWPMTP (SEQ |
| | 150) (Kabat); | DSVKG | ID NO: 170) |
| | GFTFSSY (SEQ ID NO: | (SEQ ID NO: 169) | |
| | 151) (Chothia); | (Kabat) | |
| | GFTFSSYAMN (SEQ ID | SDSGGS (SEQ ID | |
| | NO: 152) (extended) | NO: 154) (Chothia) | |
| A02_Rd4_6 nM_C22 | SYAMN (SEQ ID NO: | AVLDSGGSTYY | YWPMTP (SEQ |
| | 150) (Kabat); | ADSVKG | ID NO: 170) |
| | GFTFSSY (SEQ ID NO: | (SEQ ID NO: 171) | |
| | 151) (Chothia); | (Kabat) | |
| | GFTFSSYAMN (SEQ ID | LDSGGS (SEQ ID | |
| | NO: 152) (extended) | NO: 172) (Chothia) | |
| A02_Rd4_6 nM_C19 | SYAMN (SEQ ID NO: | AISDSGGSRWYA | YWPMSD |
| | 150) (Kabat); | DSVKG | (SEQ ID NO: |
| | GFTFSSY (SEQ ID NO: | (SEQ ID NO: 169) | 173) |
| | 151) (Chothia); | (Kabat) | |
| | GFTFSSYAMN (SEQ ID | SDSGGS (SEQ ID | |
| | NO: 152) (extended) | NO: 154) (Chothia) | |
| A02_Rd4_0.6 nM_C03 | SYAMN (SEQ ID NO: | AISDSGGSKWYA | YWPMSL (SEQ |
| | 150) (Kabat); | DSVKG (SEQ ID | ID NO: 164) |
| | GFTFSSY (SEQ ID NO: | NO: 174) (Kabat) | |
| | 151) (Chothia); | SDSGGS (SEQ ID | |
| | GFTFSSYAMN (SEQ ID | NO: 154) (Chothia) | |
| | NO: 152) (extended) | | |
| A02_Rd4_6 nM_C07 | SYAMN (SEQ ID NO: | AIGGSGGSLPYA | YWPMDS |
| | 150) (Kabat); | DSVKG(SEQ ID | (SEQ ID NO: |
| | GFTFSSY (SEQ ID NO: | NO: 158) (Kabat) | 161) |
| | 151) (Chothia); | GGSGGS (SEQ ID | |
| | GFTFSSYAMN (SEQ ID | NO: 159) (Chothia) | |
| | NO: 152) (extended) | | |
| A02_Rd4_6 nM_C23 | SYAMN (SEQ ID NO: | AISDSGGSGWYA | YWPMSL (SEQ |
| | 150) (Kabat); | DSVKG (SEQ ID | ID NO: 164) |
| | GFTFSSY (SEQ ID NO: | NO: 175)-(Kabat) | |
| | 151) (Chothia); | SDSGGS (SEQ ID | |
| | GFTFSSYAMN (SEQ ID | NO: 154) (Chothia) | |
| | NO: 152) (extended) | | |
| A02_Rd4_0.6 nM_C18 | SYAMN (SEQ ID NO: | AVLDSGGSTYY | YWPMSL (SEQ |
| | 150) (Kabat); | ADSVKG | ID NO: 164) |
| | GFTFSSY (SEQ ID NO: | (SEQ ID NO: 171) | |
| | 151) (Chothia); | (Kabat) | |
| | GFTFSSYAMN (SEQ ID | LDSGGS (SEQ ID | |
| | NO: 152) (extended) | NO: 172) (Chothia) | |
| A02_Rd4_6 nM_C10 | SYAMN (SEQ ID NO: | AISDSGGSCWYA | YWPMTP (SEQ |
| | 150) (Kabat); | DSVKG (SEQ ID | ID NO: 170) |
| | GFTFSSY (SEQ ID NO: | NO: 176) (Kabat) | |
| | 151) (Chothia); | SDSGGS (SEQ ID | |
| | GFTFSSYAMN (SEQ ID | NO: 154) (Chothia) | |
| | NO: 152) (extended) | | |
| A02_Rd4_6 nM_C05 | SYAMN (SEQ ID NO: | AIFASGGSTYYA | YWPMTP (SEQ |
| | 150) (Kabat); | DSVKG | ID NO: 170) |
| | GFTFSSY (SEQ ID NO: | (SEQ ID NO: 177) | |
| | 151) (Chothia); | (Kabat) | |
| | GFTFSSYAMN (SEQ ID | FASGGS (SEQ ID | |
| | NO: 152) (extended) | NO: 178) (Chothia) | |
| A02_Rd4_0.6 nM_C10 | SYAMN (SEQ ID NO: | AISGWGGSLPYA | YWPMDS |
| | 150) (Kabat); | DSVKG | (SEQ ID NO: |
| | GFTFSSY (SEQ ID NO: | (SEQ ID NO: 304) | 161) |
| | 151) (Chothia); | (Kabat) | |
| | GFTFSSYAMN (SEQ ID | SGWGGS (SEQ ID | |
| | NO: 152) (extended) | NO: 179) (Chothia) | |
| A02_Rd4_6 nM_C04 | SYAMN (SEQ ID NO: | AIMSSGGPLYYA | YWPMAL |
| | 150) (Kabat); | DSVKG | (SEQ ID NO: |
| | GFTFSSY (SEQ ID NO: | (SEQ ID NO: 180) | 182) |
| | 151) (Chothia); | (Kabat) | |
| | GFTFSSYAMN (SEQ ID | MSSGGP (SEQ ID | |
| | NO: 152) (extended) | NO: 181) (Chothia) | |
| A02_Rd4_0.6 nM_C26 | SYAMN (SEQ ID NO: | AILMSGGSTYYA | YWPMSL (SEQ |
| | 150) (Kabat); | DSVKG | ID NO: 164) |
| | GFTFSSY (SEQ ID NO: | (SEQ ID NO: 183) | |
| | 151) (Chothia); | (Kabat) | |
| | GFTFSSYAMN (SEQ ID | LMSGGS (SEQ ID | |
| | NO: 152) (extended) | NO: 184) (Chothia) | |
| A02_Rd4_0.6 nM_C13 | SYAMN (SEQ ID NO: | AISDSGGYRYYA | YWPMSL (SEQ |
| | 150) (Kabat); | DSVKG | ID NO: 164) |
| | GFTFSSY (SEQ ID NO: | (SEQ ID NO: 185) | |
| | 151) (Chothia); | (Kabat) | |
| | GFTFSSYAMN (SEQ ID | SDSGGY (SEQ ID | |
| | NO: 152) (extended) | NO: 186) (Chothia) | |
| A02_Rd4_0.6 nM_C01 | SYAMN (SEQ ID NO: | AILSSGGSTYYA | YWPMDI (SEQ |
| (P5AC1) | 150) (Kabat); | DSVKG | ID NO: 155) |
| | GFTFSSY (SEQ ID NO: | (SEQ ID NO: 187) | |
| | 151) (Chothia); | (Kabat) | |
| | GFTFSSYAMN (SEQ ID | LSSGGS (SEQ ID | |
| | NO: 152) (extended) | NO: 188) (Chothia) | |
| A02_Rd4_6 nM_C08 | SYAMN (SEQ ID NO: | AILDSGGSTYYA | YWPMSP (SEQ |
| | 150) (Kabat); | DSVKG (SEQ ID | ID NO: 189) |
| | GFTFSSY (SEQ ID NO: | NO: 160) (Kabat) | |
| | 151) (Chothia); | LDSGGS (SEQ ID | |
| | GFTFSSYAMN (SEQ ID | NO: 172) (Chothia) | |
| | NO: 152) (extended) | | |
| C01_Rd4_6 nM_C12 | SYPMS (SEQ ID NO: 156) | AIGGSGGWSYY | YWPMDS |
| (PC1C12) | (Kabat); | ADSVKG | (SEQ ID NO: |
| | GFTFSSY (SEQ ID NO: | (SEQ ID NO: 190) | 161) |
| | 151) (Chothia); | (Kabat) | |
| | GFTFSSYPMS (SEQ ID | GGSGGW (SEQ ID | |
| | NO: 157) (extended) | NO: 191) (Chothia) | |
| C01_Rd4_6 nM_C09 | SYPMS (SEQ ID NO: 156) | ATVGSGGSIGYA | YWPMDS |
| | (Kabat); | DSVKG | (SEQ ID NO: |
| | GFTFSSY (SEQ ID NO: | (SEQ ID NO: 192) | 161) |
| | 151) (Chothia); | (Kabat) | |
| | GFTFSSYPMS (SEQ ID | VGSGGS (SEQ ID | |
| | NO: 157) (extended) | NO: 193) (Chothia) | |
| COMBO_Rd4_0.6 nM_C22 | SYAMN (SEQ ID NO: | AISDSGGSRWYA | YWPMDI (SEQ |
| (COM22) | 150) (Kabat); | DSVKG | IDNO: 155) |
| | GFTFSSY (SEQ ID NO: | (SEQ ID NO: 169) | |
| | 151) (Chothia); | (Kabat) | |
| | GFTFSSYAMN (SEQ ID | SDSGGS (SEQ ID | |
| | NO: 152) (extended) | NO: 154) (Chothia) | |
| COMBO_Rd4_0.6 nM_C10 | SYPMS (SEQ IDNO: 156) | AIGGSGGSIHYA | YWPMDS |
| | (Kabat); | DSVKG (SEQ ID | (SEQ ID NO: |
| | GFTFSSY (SEQ ID NO: | NO: 194) (Kabat) | 161) |
| | 151) (Chothia); | GGSGGS (SEQ ID | |
| | GFTFSSYPMS (SEQ ID | NO: 159) (Chothia) | |
| | NO: 157) (extended) | | |
| COMBO_Rd4_0.6 nM_C04 | SYPMS (SEQ ID NO: 156) | AHIGSGGSTYYA | YWPMDS |
| | (Kabat); | DSVKG (SEQ ID | (SEQ ID NO: |
| | GFTFSSY (SEQ ID NO: | NO: 195) (Kabat) | 161) |
| | 151) (Chothia); | IGSGGS (SEQ ID | |
| | GFTFSSYPMS (SEQ ID | NO: 196) (Chothia) | |
| | NO: 157) (extended) | | |
| COMBO_Rd4_0.6 nM_C25 | SYPMS (SEQ ID NO: 156) | AIGGSGGSTYYA | YWPMDP |
| | (Kabat); | DSVKG | (SEQ ID NO: |
| | GFTFSSY (SEQ ID NO: | (SEQ ID NO: 162) | 197) |
| | 151) (Chothia); | (Kabat) | |
| | GFTFSSYPMS (SEQ ID | GGSGGS (SEQ ID | |
| | NO: 157) (extended) | NO: 159) (Chothia) | |
| COMBO_Rd4_6 nM_C21 | SYPMS (SEQ ID NO: 156) | AIGGSGGSLPYA | YWPMDS |
| | (Kabat); | DSVKG | (SEQ ID NO: |
| | GFTFSSY (SEQ ID NO: | (SEQ ID NO: 158) | 161) |
| | 151) (Chothia); | (Kabat) | |
| | GFTFSSYPMS (SEQ ID | GGSGGS (SEQ ID | |
| | NO: 157) (extended) | NO: 159) (Chothia) | |
| COMBO_Rd4_6 nM_C11 | SYPMS (SEQ IDNO: 156) | AIGGSGGSLGYA | YWPMDS |
| | (Kabat); | DSVKG | (SEQ ID NO: |
| | GFTFSSY (SEQ ID NO: | (SEQ ID NO: | 161) |
| | 151) (Chothia); | 198)(Kabat) | |
| | GFTFSSYPMS (SEQ ID | GGSGGS (SEQ ID | |
| | NO: 157) (extended) | NO: 159) (Chothia) | |
| COMBO_Rd4_6 nM_C09 | SYPMS (SEQ ID NO: 156) | AIFASGGSTYYA | YWPMDS |
| | (Kabat); | DSVKG | (SEQ ID NO: |
| | GFTFSSY (SEQ ID NO: | (SEQ ID NO: 177) | 161) |
| | 151) (Chothia); | (Kabat) | |
| | GFTFSSYPMS (SEQ ID | FASGGS (SEQ ID | |
| | NO: 157) (extended) | NO: 178) (Chothia) | |
| COMBO_Rd4_6 nM_C08 | SYPMS (SEQ ID NO: 156) | AIGGSGTWTYY | YWPMDS |
| | (Kabat); | ADSVKG | (SEQ ID NO: |
| | GFTFSSY (SEQ ID NO: | (SEQ ID NO: 199) | 161) |
| | 151) (Chothia); | (Kabat) | |
| | GFTFSSYPMS (SEQ ID | GGSGTW (SEQ ID | |
| | NO: 157) (extended) | NO: 200) (Chothia) | |
| COMBO_Rd4_0.6 nM_C23 | SYPMS (SEQ ID NO: 156) | ALFGSGGSTYYA | YWPMDS |
| | (Kabat); | DSVKG | (SEQ ID NO: |
| | GFTFSSY (SEQ ID NO: | (SEQ ID NO: 201) | 161) |
| | 151) (Chothia); | (Kabat) | |
| | GFTFSSYPMS (SEQ ID | FGSGGS | |
| | NO: 157) (extended) | (SEQ ID NO: 202) | |
| | | (Chothia) | |
| COMBO_Rd4_0.6 nM_C12 | SYPMS (SEQIDNO: 156) | AALGSGGSTYY | YWPMDS |
| | (Kabat); | ADSVKG (SEQ ID | (SEQ ID NO: |
| | GFTFSSY (SEQ ID NO: | NO: 203) (Kabat) | 161) |
| | 151) (Chothia); | LGSGGS (SEQ ID | |
| | GFTFSSYPMS (SEQ ID | NO: 204) (Chothia) | |
| | NO: 157) (extended) | | |
| COMBO_Rd4_6 nM_C07 | SYPMS (SEQ ID NO: 156) | AIGGSGGSLPYA | YWPMAD |
| | (Kabat); | DSVKG | (SEQ ID NO: |
| | GFTFSSY (SEQ ID NO: | (SEQ ID NO: 158) | 205) |
| | 151) (Chothia); | (Kabat) | |
| | GFTFSSYPMS (SEQ ID | GGSGGS (SEQ ID | |
| | NO: 157) (extended) | NO: 159) (Chothia) | |
| COMBO_Rd4_6 nM_C02 | SYAMN (SEQ ID NO: | AISDSGGFVYYA | YWPMDS |
| | 150) (Kabat); | DSVKG | (SEQ ID NO: |
| | GFTFSSY (SEQ ID NO: | (SEQ ID NO: 206) | 161) |
| | 151) (Chothia); | (Kabat) | |
| | GFTFSSYAMN (SEQ ID | SDSGGF (SEQ ID | |
| | NO: 152) (extended) | NO: 207) (Chothia) | |
| COMBO_Rd4_6 nM_C05 | SYAMN (SEQ ID NO: | AIGGSGGSTYYA | YWPMSL (SEQ |
| | 150) (Kabat); | DSVKG | IDNO: 164) |
| | GFTFSSY (SEQ ID NO: | (SEQ ID NO: 162) | |
| | 151) (Chothia); | (Kabat) | |
| | GFTFSSYAMN (SEQ ID | GGSGGS (SEQ ID | |
| | NO: 152) (extended) | NO: 159) (Chothia) | |
| COMBO_Rd4_6 nM_C22 | SYAMN (SEQ ID NO: | ACLDSGGSTYYA | YWPMDS |
| | 150) (Kabat); | DSVKG (SEQ ID | (SEQ ID NO: |
| | GFTFSSY (SEQ ID NO: | NO: 208) (Kabat) | 161) |
| | 151) (Chothia); | LDSGGS (SEQ ID | |
| | GFTFSSYAMN (SEQ ID | NO: 172) (Chothia) | |
| | NO: 152) (extended) | | |
| COMBO_Rd4_6 nM_C11 | SYPMS (SEQIDNO: 156) | AALGSGGSTYY | YWPMSL (SEQ |
| | (Kabat); | ADSVKG (SEQ ID | IDNO: 164) |
| | GFTFSSY (SEQ ID NO: | NO: 203) (Chothia) | |
| | 151) (Chothia); | LGSGGS (SEQ ID | |
| | GFTFSSYPMS (SEQ ID | NO: 204) (Chothia) | |
| | NO: 157) (extended) | | |
| Heavy chain consensus | SYX₁MX₂, wherein X₁ is | AX₁X₂X₃X₄GX₅X₆ | VSPIX₁X₂X₃M |
| | A or P; and X₂ is T, N, | X₇X₈YADX₉X₁₀K | DY, wherein X₁ |
| | or S | G, wherein X₁ is 1, | is A or Y; X₂ is A |
| | (Kabat) (SEQ ID NO: 301) | V, T, H, L, A, or C; | or S; and X₃ is G, |
| | GFTFX₁SY, wherein X₁ is | X₂ is S, D, G, T, I, | Q, L, P, or E |
| | G or S (Chothia) (SEQ | L, F, M, or V; X₃ is | (SEQ ID NO: |
| | ID NO: 302) | G, Y, L, H, D, A, S, | 307) |
| | GFTFX₁SYX₂MX_{3,} | or M; X₄ is S, Q, T, | YWPMX₁X₂, |
| | wherein X₁ is G or S, | A, F, or W; X₅ is G | wherein X₁ is D, |
| | X₂ is A or P; and X₃ is | or T; X₆ is N, S, P, | S, T, or A; and |
| | T, N, or S | Y, W, or F; X₇ is S, | X₂ is I, S, L, P, |
| | (SEQ ID NO: 303) | T, I, L, T, A, R, V, | or D (SEQ ID NO: |
| | (extended) | K, G, or C; X₈ is F, | 308) |
| | | Y, P, W, H, or G; X₉ | |
| | | is V, R, or L; and | |
| | | X₁₀ is G or T | |
| | | (Kabat) | |
| | | (SEQ ID NO: 305) | |
| | | X₁X₂X₃X₄X₅X₆, | |
| | | wherein X₁ is S, V, | |
| | | I, D, G, T, L, F, or | |
| | | M; X₂ is G, Y, L, H, | |
| | | D, A, S, or M; X₃ is | |
| | | S, G, F, or W; X₄ is | |
| | | G or S; X₅ is Gor T; | |
| | | and X is N, S, P, Y, | |
| | | or W (Chothia) | |
| | | (SEQ ID NO: 306) | |
| P4G4 | SYAMS (SEQ ID NO: | SASGGS (SEQ ID | LSWSGAFDN |
| | 381) (Kabat); | NO: 383) (Chothia) | (SEQ ID NO: |
| | GFTFSSY (SEQ ID NO: | AISASGGSTYYA | 385) |
| | 151) (Chothia); | DSVKG (SEQ ID | |
| | GFTFSSYAMS (SEQ ID | NO: 384) (Kabat) | |
| | NO: 382) (extended) | | |
| P1A11 | SYAMS (SEQ ID NO: | SGSGGS (SEQ ID | VGTSGAFGI |
| | 386) (Kabat); | NO: 389) (Chothia) | (SEQ ID NO: |
| | GFTFRSY (SEQ ID NO: | AISGSGGSTFYA | 391) |
| | 387) | DSVKG (SEQ ID | |
| | GFTFRSYAMS (SEQ ID | NO: 390) (Kabat) | |
| | NO: 388) | | |

TABLE 2B
Light Chain

| Binding Domain | CDRL1 | CDRL2 | CDRL3 |
|---|---|---|---|
| P6E01 | RASQSVSSSYLA (SEQ | GASSRAT (SEQ ID | QHYGSPPSFT |
| For the following mAbs: | ID NO: 209) | NO: 210) | (SEQ ID NO: |
| P6E01/P6E01; and | | | 211) |
| P6E01/H3.AQ. | | | |
| L1.LGF/L3.KW | RASQSLGSFYLA | GASSRAT (SEQ ID | KHYGWPPSFT |
| For the following mAbs: | (SEQ ID NO: 212) | NO: 210) | (SEQ ID NO: |
| L1.LGF/L3.KW/P6E01; | | | 213) |
| L1.LGF/L3.KW/H3.AL; | | | |
| L1.LGF/L3.KW/H3.AP; and | | | |
| L1.LGF/L3.KW/H3.AQ | | | |
| L1.LGF/L3.NY | RASQSLGSFYLA | GASSRAT (SEQ ID | QHYNYPPSFT |
| For the following mAbs: | (SEQ ID NO: 212) | NO: 210) | (SEQ ID NO: |
| L1.LGF/L3.NY/P6E01; | | | 214) |
| L1.LGF/L3.NY/H3.AL; | | | |
| L1.LGF/L3.NY/H3.AP; and | | | |
| L1.LGF/L3.NY/H3AQ | | | |
| L1.GDF/L3.NY | RASQSVGDFYLA | GASSRAT (SEQ ID | QHYNYPPSFT |
| For the following mAbs: | (SEQ ID NO: 215) | NO: 210) | (SEQ ID NO: |
| L1.GDF/L3.NY/P6E01; | | | 214) |
| L1.GDF/L3.NY/H3.AL; | | | |
| L1.GDF/L3.NY/H3.AP; and | | | |
| L1.GDF/L3.NY/H3.AQ | | | |
| L1.LGF/L3.PY | RASQSLGSFYLA | GASSRAT (SEQ ID | QHYPYPPSFT |
| For the following mAbs: | (SEQ ID NO: 212) | NO: 210) | (SEQ ID NO: |
| L1.LGF/L3.PY/H3.AP; and | | | 216) |
| L1.LGF/L3.PY/H3.AQ | | | |
| L1.GDF/L3.KW | RASQSVGDFYLA | GASSRAT (SEQ ID | KHYGWPPSFT |
| For the following mAbs: | (SEQ ID NO: 215) | NO: 210) | (SEQ ID NO: |
| L1.GDF/L3.KW/H3.AL; | | | 213) |
| L1.GDF/L3.KW/H3.AP; | | | |
| and L1.GDF/L3.KW/H3.AQ | | | |
| L1.GDF/L3.PY/H3.AQ | RASQSVGDFYLA | GASSRAT (SEQ ID | QHYPYPPSFT |
| | (SEQ ID NO: 215) | NO: 210) | (SEQ ID NO: |
| | | | 216) |
| L3.KW/P6E01 | RASQSVSSSYLA (SEQ | GASSRAT (SEQ ID | KHYGWPPSFT |
| | ID NO: 209) | NO: 210) | (SEQ ID NO: |
| | | | 213) |
| L3.PY | RASQSVSSSYLA (SEQ | GASSRAT (SEQ ID | QHYPYPPSFT |
| For the following mAbs: | ID NO: 209) | NO: 210) | (SEQ ID NO: |
| L3.PY/P6E01; | | | 216) |
| L3.PY/H2.QR; | | | |
| L3.PY/H2.DY; | | | |
| L3.PY/H2.YQ; | | | |
| L3.PY/H2.LT; | | | |
| L3.PY/H2.HA; | | | |
| L3.PY/H2.QL; | | | |
| L3.PY/H3.YA; | | | |
| L3.PY/H3.AE; | | | |
| L3.PY/H3.AQ; | | | |
| L3.PY/H3.TAQ | | | |
| L3.NY/P6E01 | RASQSVSSSYLA (SEQ | GASSRAT (SEQ ID | QHYNYPPSFT |
| | ID NO: 209) | NO: 210) | (SEQ ID NO: |
| | | | 214) |
| L3.PY/L1.PS | RASQSVSSSYPS | GASSRAT (SEQ ID | QHYPYPPSFT |
| For the following mAbs: | (SEQ ID NO: 217) | NO: 210) | (SEQ ID NO: |
| L3.PY/L1.PS/P6E01; P6DY; | | | 216) |
| L3.PY/L1.PS/H2.QR; | | | |
| L3.PY/L1.PS/H2.DY; | | | |
| L3.PY/L1.PS/H2 YQ; | | | |
| L3.PY/L1.PS/H2.LT; | | | |
| L3.PY/L1.PS/H2.HA; | | | |
| L3.PY/L1.PS/H2.QL; | | | |
| L3.PY/L1.PS/H3.YA; | | | |
| L3.PY/L1.PS/H3.AE; | | | |
| L3.PY/L1.PS/H3.AQ; | | | |
| L3.PY/L1.PS/H3.TAQ; | | | |
| L3.PY/L1.AH | RASQSVSAHYLA | GASSRAT (SEQ ID | QHYPYPPSFT |
| For the following mAbs: | (SEQ ID NO: 218) | NO: 210) | (SEQ ID NO: |
| L3.PY/L1.AH/P6E01; | | | 216) |
| L3.PY/L1.AH/H2.QR; | | | |
| L3.PY/L1.AH/H2.DY; | | | |
| L3.PY/L1.AH/H2.YQ; | | | |
| L3.PY/L1.AH/H2.LT; | | | |
| L3.PY/L1.AH/H2.HA; | | | |
| L3.PY/L1.AH/H2.QL; | | | |
| L3.PY/L1.AH/H3.YA; | | | |
| L3.PY/L1.AH/H3.AE; | | | |
| L3.PY/L1.AH/H3.AQ; | | | |
| L3.PY/L1.AH/H3.TAQ | | | |
| L3.PY/L1.FF | RASQSVSSFFLA | GASSRAT (SEQ ID | QHYPYPPSFT |
| For the following mAbs: | (SEQ ID NO: 219) | NO: 210) | (SEQ ID NO: |
| L3.PY/L1.FF/P6E01; | | | 216) |
| L3.PY/L1.FF/H2.QR; | | | |
| L3.PY/L1.FF/H2.DY; | | | |
| L3.PY/L1.FF/H2.YQ; | | | |
| L3.PY/L1.FF/H2.LT; | | | |
| L3.PY/L1.FF/H2.HA; | | | |
| L3.PY/L1.FF/H2.QL; | | | |
| L3.PY/L1.FF/H3.YA; | | | |
| L3.PY/L1.FF/H3.AE; | | | |
| L3.PY/L1.FF/H3.AQ; and | | | |
| L3.PY/L1.FF/H3.TAQ | | | |
| L3.PY/L1.PH | RASQSVSPHYLA | GASSRAT (SEQ ID | QHYPYPPSFT |
| For the following mAbs: | (SEQ ID NO: 415) | NO: 210) | (SEQ ID NO: |
| L3.PY/L1.PH/P6E01; | | | 216) |
| L3.PY/L1.PH/H2.QR; | | | |
| L3.PY/L1.PH/H2.HA; | | | |
| L3.PY/L1.PH/H3.AE; | | | |
| L3.PY/L1.PH/H3.AQ; and | | | |
| L3.PY/L1.PH/H3.TAQ | | | |
| L3.PY/L3.KY | RASQSVSSSYLA | GASSRAT (SEQ ID | KYYPYPPSFT |
| For the following mAbs: | (SEQ ID NO: 209) | NO: 210) | (SEQ ID NO: |
| L3.PY/L3.KY/P6E01; | | | 220) |
| L3.PY/L3.KY/H2.QR; | | | |
| L3.PY/L3.KY/H2.DY; | | | |
| L3.PY/L3.KY/H2.YQ; | | | |
| L3.PY/L3.KY/H2.LT; | | | |
| L3.PY/L3.KY/H2.HA; | | | |
| L3.PY/L3.KY/H2.QL; | | | |
| L3.PY/L3.KY/H3.YA; and | | | |
| L3.PY/L3.KY/H3.TAQ | | | |
| L3.PY/L3.KF | RASQSVSSSYLA | GASSRAT (SEQ ID | KFYPYPPSFT |
| For the following mAbs: | (SEQ ID NO: 209) | NO: 210) | (SEQ ID NO: |
| L3.PY/L3.KF/H2.DY; | | | 416) |
| L3.PY/L3.KF/H2.YQ; | | | |
| L3.PY/L3.KF/H2.LT; | | | |
| L3.PY/L3.KF/H2.QL; | | | |
| L3.PY/L3.KF/H3.YA; | | | |
| L3.PY/L3.KF/H3.AE; | | | |
| L3.PY/L3.KF/H3.AQ; and | | | |
| L3.PY/L3.KF/H3.TAQ | | | |
| P5A2_VHVL (P5A) | RASQSVSSSYLA | DASIRAT | QQYGSWPLT |
| | (SEQ ID NO: 209) | (SEQ ID NO: 221) | (SEQ ID NO: |
| | | | 222) |
| A02_Rd4_0.6nM_C06 | RASQSVSVIYLA | DASIRAT | QQYQRWPLT |
| | (SEQ ID NO: 223) | (SEQ ID NO: 221) | (SEQ ID NO: |
| | | | 224) |
| A02_Rd4_0.6nM_C09; | RASQSVSSSYLA | DASIRAT | QQYQSWPLT |
| COMBO_Rd_0.6nM_C29; | (SEQ ID NO: 209) | (SEQ ID NO: 221) | (SEQ ID NO: |
| and | | | 225) |
| COMBO_Rd40.6nM_C21 | | | |
| A02_Rd4_6nM_C16 | RASQSVSDIYLA | DASIRAT | QQYQTWPLT |
| (P5AC16) | (SEQ ID NO: 226) | (SEQ ID NO: 221) | (SEQ ID NO: |
| | | | 227) |
| A02_Rd4_6nM_C03 | RASQSVSNIYLA (SEQ | DASIRAT | QQYQGWPLT |
| | ID NO: 228) | (SEQ ID NO: 221) | (SEQ ID NO: |
| | | | 229) |
| A02_Rd4_6nM_C01 | RASQSVSAYYLA | DASIRAT | QQYERWPLT |
| | (SEQ ID NO: 230) | (SEQ ID NO: 221) | (SEQ ID NO: |
| | | | 231) |
| A02_Rd4_6nM_C26 | RASQSVSSIYLA | DASIRAT | QQYQVWPLT |
| | (SEQ ID NO: 232) | (SEQ ID NO: 221) | (SEQ ID NO: |
| | | | 233) |
| A02_Rd4_6nM_C25 | RASQSVSSSYLA | DASIRAT | QQYLDWPLT |
| | (SEQ ID NO: 209) | (SEQ ID NO: 221) | (SEQ ID NO: |
| | | | 234) |
| A02_Rd4_6nM_C22 | RASQSVSSSYLA | DASIRAT | QQYQVWPLT |
| | (SEQ ID NO: 209) | (SEQ ID NO: 221) | (SEQ ID NO: |
| | | | 233) |
| A02_Rd4_6nM_C19 | RASQSVSVIYLA | DASIRAT | QQYLAWPLT |
| | (SEQ ID NO: 223) | (SEQ ID NO: 221) | (SEQ ID NO: |
| | | | 236) |
| A02_Rd4_0.6nM_C03 | RASQSVSSSYLA | DASIRAT | QQYFTWPLT |
| | (SEQ ID NO: 209) | (SEQ ID NO: 221) | (SEQ ID NO: |
| | | | 237) |
| A02_Rd4_6nM_C07 | RASQSVSPYYLA | DASIRAT | QQYERWPLT |
| | (SEQ ID NO: 238) | (SEQ ID NO: 221) | (SEQ ID NO: |
| | | | 231) |
| A02_Rd4_6nM_C23 | RASQSVSVEYLA | DASIRAT | QQYARWPLT |
| | (SEQ ID NO: 239) | (SEQ ID NO: 221) | (SEQ ID NO: |
| | | | 240) |
| A02_Rd4_0.6nM_C18 | RASQSVSEIYLA | DASIRAT | QQYFGWPLT |
| | (SEQ ID NO: 241) | (SEQ ID NO: 221) | (SEQ ID NO: |
| | | | 242) |
| A02_Rd4_6nM_C10 | RASQSVEMSYLA | DASIRAT | QQYAHWPLT |
| | (SEQ ID NO: 243) | (SEQ ID NO: 221) | (SEQ ID NO: |
| | | | 244) |
| A02_Rd4_6nM_C05 | RASQSVSSSYLA | DASIRAT | QQYQRWPLT |
| | (SEQ ID NO: 209) | (SEQ ID NO: 221) | (SEQ ID NO: |
| | | | 224) |
| A02_Rd4_0.6nM_C10 | RASQSVSAQYLA | DASIRAT | QQYQRWPLT |
| | (SEQ ID NO: 245) | (SEQ ID NO: 221) | (SEQ ID NO: |
| | | | 224) |
| A02_Rd4_6nM_C04 | RASQSVSAIYLA | DASIRAT | QQYQVWPLT |
| | (SEQ ID NO: 235) | (SEQ ID NO: 221) | (SEQ ID NO: |
| | | | 233) |
| A02_Rd4_0.6nM_C26 | GPSQSVSSSYLA | DASIRAT | QQYQSWPLT |
| | (SEQ ID NO: 246) | (SEQ ID NO: 221) | (SEQ ID NO: |
| | | | 225) |
| A02_Rd4_0.6nM_C13 | RASQSVSSSYWA | DASIRAT | QQYESWPLT |
| | (SEQ ID NO: 247) | (SEQ ID NO: 221) | (SEQ ID NO: |
| | | | 248) |
| A02_Rd4_0.6nM_C01 | RGGQSVSSSYLA | DASIRAT | QQYQSWPLT |
| (P5AC1) | (SEQ ID NO: 249) | (SEQ ID NO: 221) | (SEQ ID NO: |
| | | | 225) |
| A02_Rd4_6nM_C08 | RASQSVSFIYLA | DASIRAT | QQYGSWPLT |
| | (SEQ ID NO: 250) | (SEQ ID NO: 221) | (SEQ ID NO: |
| | | | 222) |
| P5C1VHVL (PCI) | RASQSVSSTYLA | DASSRAP | QQYSTSPLT |
| | (SEQ ID NO: 251) | (SEQ ID NO: 252) | (SEQ ID NO: |
| | | | 253) |
| C01_Rd4_6nM_C24 | RASQSVSPEYLA | DASSRAP | QQYSVWPLT |
| | (SEQ ID NO: 254) | (SEQ ID NO: 252) | (SEQ ID NO: |
| | | | 255) |
| C01_Rd4_6nM_C26 | RASQSVSAIYLA | DASSRAP | QQYSAWPLT |
| | (SEQ ID NO: 235) | (SEQ ID NO: 252) | (SEQ ID NO: |
| | | | 256) |
| C01_Rd4_6nM_C10 | RASQSVSSVYLA | DASSRAP | QQYSTWPLT |
| | (SEQ ID NO: 257) | (SEQ ID NO: 252) | (SEQ ID NO: |
| | | | 258) |
| C01_Rd4_0.6nM_C27 | RASQSVSSTYLA | DASSRAP | QQYSRWPLT |
| | (SEQ ID NO: 251) | (SEQ ID NO: 252) | (SEQ ID NO: |
| | | | 259) |
| C01_Rd4_6nM_C20 | RASQSVSPIYLA | DASSRAP | QQYSAFPLT |
| | (SEQ ID NO: 260) | (SEQ ID NO: 252) | (SEQ ID NO: |
| | | | 261) |
| C01_Rd4_6nM_C12 | WLSQSVSSTYLA | DASSRAP | QQYSEWPLT |
| (PC1C12) | (SEQ ID NO: 262) | (SEQ ID NO: 252) | (SEQ ID NO: |
| | | | 263) |
| C01_Rd4_0.6nM_C16 | RASQSVSSTYLA | DASSRAP | QQYSSWPLT |
| | (SEQ ID NO: 251) | (SEQ ID NO: 252) | (SEQ ID NO: |
| | | | 264) |
| C01_Rd4_0.6nM_C09 | RASQSVSS1FLA | DASSRAP | QQYSAWPLT |
| | (SEQ ID NO: 265) | (SEQ ID NO: 252) | (SEQ ID NO: |
| | | | 256) |
| C01_Rd4_6nM_C09 | ACSQSVSSTYLA | DASSRAP | QQYSAWPLT |
| | (SEQ ID NO: 266) | (SEQ ID NO: 252) | (SEQ ID NO: |
| | | | 256) |
| C01_Rd4_0.6nM_C03 | RASCDVSSTYLA | DASSRAP | QQYMRSPLT |
| | (SEQ ID NO: 267) | (SEQ ID NO: 252) | (SEQ ID NO: |
| | | | 268) |
| C01_Rd4_0.6nM_C06 | RASEAVPSTYLA | DASSRAP | QQYSAFPLT |
| | (SEQ ID NO: 269) | (SEQ ID NO: 252) | (SEQ ID NO: |
| | | | 261) |
| C01_Rd4_0.6nM_C04 | CSSQSVSSTYLA | DASSRAP | QQYSAFPLT |
| | (SEQ ID NO: 270) | (SEQ ID NO: 252) | (SEQ ID NO: |
| | | | 261) |
| COMBO_Rd4_0.6nM_C22 | RASVRVSSTYLA | DASIRAT | QQYMKWPLT |
| (COM22) | (SEQ ID NO: 271) | (SEQ ID NO: 221) | (SEQ ID NO: |
| | | | 272) |
| COMBO_Rd4_6nM_C21 | RASQSVSAAYLA | DASIRAT | QQYMCWPLT |
| | (SEQ ID NO: 273) | (SEQ ID NO: 221) | (SEQ ID NO: |
| | | | 274) |
| COMBO_Rd4_6nM_C10 | RASQSVSSSYWG | DASIRAT | QQYQCWPLT |
| | (SEQ ID NO: 275) | (SEQ ID NO: 221) | (SEQ ID NO: |
| | | | 276) |
| COMBO_Rd4_0.6nM_C04 | RASQSVSSTYLA | DASIRAT | QQYQSWPLT |
| | (SEQ ID NO: 251) | (SEQ ID NO: 221) | (SEQ ID NO: |
| | | | 225) |
| COMBO_Rd4_6nM_C25 | RASQSVSSPYLA (SEQ | DASIRAT | QQYQSWPLT |
| | ID NO: 277) | (SEQ ID NO: 221) | (SEQ ID NO: |
| | | | 225) |
| COMBO_Rd4_6nM_C11 | RASQSVSPIYLA | DASIRAT | QQYKAWPLT |
| | (SEQ ID NO: 260) | (SEQ ID NO: 221) | (SEQ ID NO: |
| | | | 278) |
| COMBO_Rd4_0.6nM_C20 | RASQSVSYLYLA | DASIRAT | QQYMEWPLT |
| | (SEQ ID NO: 279) | (SEQ ID NO: 221) | (SEQ ID NO: |
| | | | 280) |
| COMBO_Rd4_6nM_C09 | RASQSVSAQYLA | DASIRAT | QQYQAWPLT |
| | (SEQ ID NO: 245) | (SEQ ID NO: 221) | (SEQ ID NO: |
| | | | 281) |
| COMBO_Rd4_6nM_C08 | RASQSVSSSYLA | DASIRAT | QQYQKWPLT |
| | (SEQ ID NO: 209) | (SEQ ID NO: 221) | (SEQ ID NO: |
| | | | 282) |
| COMBO_Rd4_0.6nM_C19 | RASQSVSAVYLA | DASIRAT | QQYRAWPLT |
| | (SEQ ID NO: 283) | (SEQ ID NO: 221) | (SEQ ID NO: |
| | | | 284) |
| COMBO_Rd4_0.6nM_C02 | RASIAVSSTYLA | DASIRAT | QQYMVWPLT |
| | (SEQ ID NO: 285) | (SEQ ID NO: 221) | (SEQ ID NO: |
| | | | 286) |
| COMBO_Rd4_0.6nM_C23 | RPRQSVSSSYLA | DASIRAT | QQYQDWPLT |
| | (SEQ ID NO: 287) | (SEQ ID NO: 221) | (SEQ ID NO: |
| | | | 288) |
| COMBO_Rd4_0.6nM_C09 | RASQSVSSTYLA | DASIRAT | QQYQEWPLT |
| | (SEQ ID NO: 251) | (SEQ ID NO: 221) | (SEQ ID NO: |
| | | | 289) |
| COMBO_Rd4_6nM_C12 | RASQSVSASYLA | DASIRAT | QQYMSWPLT |
| | (SEQ ID NO: 290) | (SEQ ID NO: 221) | (SEQ ID NO: |
| | | | 291) |
| COMBO_Rd4_0.6nM_C30 | RASQSVSYMYLA | DASIRAT | QQYKSWPLT |
| | (SEQ ID NO: 292) | (SEQ ID NO: 221) | (SEQ ID NO: |
| | | | 293) |
| COMBO_Rd4_0.6nM_C14 | RASQSVSAIYLA | DASIRAT | QQYYGWPLT |
| | (SEQ ID NO: 235) | (SEQ ID NO: 221) | (SEQ ID NO: |
| | | | 294) |
| COMBO_Rd4_6nM_C07 | RASQPISSSYLA | DASIRAT | QQYQGWPLT |
| | (SEQ ID NO: 295) | (SEQ ID NO: 221) | (SEQ ID NO: |
| | | | 229) |
| COMBO_Rd4_6nM_C02 | RASQSVSSSYLA | DASIRAT | QQYEFWPLT |
| | (SEQ ID NO: 209) | (SEQ ID NO: 221) | (SEQ ID NO: |
| | | | 296) |
| COMBO_Rd4_0.6nM_C05 | RASQSVSSTYLA | DASIRAT | QQYMSWPLT |
| | (SEQ ID NO: 251) | (SEQ ID NO: 221) | (SEQ ID NO: |
| | | | 291) |
| COMBO_Rd4_0.6nM_C17 | RASQGISSTYLA | DASIRAT | QQYAYWPLT |
| | (SEQ ID NO: 297) | (SEQ ID NO: 221) | (SEQ ID NO: |
| | | | 298) |
| COMBO_Rd4_6nM_C22 | RASQSVSSSYLA | DASIRAT | QQYQGWPLT |
| | (SEQ ID NO: 209) | (SEQ ID NO: 221) | (SEQ ID NO: |
| | | | 229) |
| COMBO_Rd4_0.6nM_C11 | RASQSVSVRYLA | DASIRAT | QQYGSWPIT |
| | (SEQ ID NO: 299) | (SEQ ID NO: 221) | (SEQ ID NO: |
| | | | 300) |
| Light chain consensus | X₁X₂X₃X₄X₅X₆X₇X₈X₉X₁₀ | X₁ASX₂RAX₃, | X₁X₂YX₃X₄PPS |
| | X₁₁X₁₂, wherein X₁ is | wherein X₁ is G or | FT, wherein X₁ |
| | R, G, W, A, or C; X₂ | D; X₂ is S or I; | is Q or K; X₂ is |
| | is A, P, G, L, C, or | and X₃ is T or P | H or Y; X₃ is G, |
| | S; X₃ is S, G, or R; | (SEQ ID NO: 310) | N, or P; and X₄ |
| | X₄ is Q, C, E, V, or | | is S, W, or Y |
| | I; X₅ is S, P, G, A, | | (SEQ ID NO: 311) |
| | R, or D; X₆ is V, G, | | QQYX₁X₂X₃PX₄ |
| | I, or L; X₇ is S, E, | | T, wherein X₁ is |
| | D, P, or G; X₈ is S, | | G, Q, E, L, F, A, |
| | P, F, A, M, E, V, N, | | S, M, K, R, or Y; |
| | D, or Y; X₉ is I, T, | | X₂ is S, R, T, G, |
| | V, E, F S, A, M, Q, | | V, F,Y, D,A,H, |
| | Y, H, or R; X₁₀ is Y | | V, E, K, or C; X₃ |
| | or F; X₁₁ is L, W, or | | is W, F, or S; |
| | P; and X₁₂ is A, S, | | and X₄ is L or I |
| | or G (SEQ ID NO: 309) | | (SEQ ID NO: 312) |
| P4G4 | RASQSVSSSYLA (SEQ | GASSRAY (SEQ ID | QHYGSPPLFT |
| | ID NO: 209) | NO: 392) | (SEQ ID NO: |
| | | | 393) |
| P1A11 | RASQNVSSSYLA (SEQ | GASYRAT (SEQ ID | QHYGSPPSFT |
| | ID NO: 417) | NO: 395) | (SEQ ID NO: |
| | | | 211) |
| P6AP | RASQLGSFYLA (SEQ ID | GASSRAT (SEQ ID | QHYNYPPSFT |
| | NO: 377) | NO: 210) | (SEQ ID NO: |
| | | | 214) |

In some embodiments, the BCMA CAR comprises an extracellular ligand-binding domain, a first transmembrane domain, and an intracellular signaling domain, wherein the extracellular domain comprises a single chain Fv fragment (scFv) comprising a heavy chain variable (VH) region comprising three complementarity determining regions (CDRs) comprising the sequences shown in SEQ ID NO: 33, 72, 39, 76, 83, 92, 25, 112, or 8 of Table 1; and a light chain variable (VL) region comprising three CDRs comprising the sequences shown in SEQ ID NO: 34, 73, 40, 77, 84, 93, 18, 38, or 80 of Table 1, wherein the first transmembrane domain comprises a CD8α chain transmembrane domain, and wherein the intracellular signaling domain comprises a CD3ζ signaling domain and/or a 4-1BB signaling domain.

In some embodiments, the extracellular binding region of the BCMA CAR comprises a VH region that comprises the amino acid sequence shown in SEQ ID NO: 112 and the VL region comprises the amino acid sequence shown in SEQ ID NO: 38.

In some embodiments, the extracellular binding region of the BCMA CAR comprises a VH region that comprises the amino acid sequence shown in SEQ ID NO: 33 and the VL region comprises the amino acid sequence shown in SEQ ID NO: 34.

In some embodiments, the extracellular binding region of the BCMA CAR comprises a VH region that comprises a VH CDR1 comprising the amino acid sequence shown in SEQ ID NO: 150, 151, or 152; a VH CDR2 comprising the amino acid sequence shown in SEQ ID NO: 153 or 154; and a VH CDR3 comprising the amino acid sequence shown in SEQ ID NO: 155; and comprises a VL region comprising a VL CDR1 comprising the amino acid sequence shown in SEQ ID NO: 209; a VL CDR2 comprising the amino acid sequence shown in SEQ ID NO: 221; and a VL CDR3 comprising the amino acid sequence shown in SEQ ID NO: 222.

In some embodiments, the extracellular binding region of the BCMA CAR comprises a VH region that comprises a VH CDR1 comprising the amino acid sequence shown in SEQ ID NO: 151, 156, or 157; a VH CDR2 comprising the amino acid sequence shown in SEQ ID NO: 158 or 159; and a VH CDR3 comprising the amino acid sequence shown in SEQ ID NO: 155; and comprises a VL region comprising a VL CDR1 comprising the amino acid sequence shown in SEQ ID NO: 209; a VL CDR2 comprising the amino acid sequence shown in SEQ ID NO: 221; and a VL CDR3 comprising the amino acid sequence shown in SEQ ID NO: 225.

The binding affinity (K_{D}) of the BCMA specific CAR as described herein to BCMA (such as human BCMA (e.g., (SEQ ID NO: 354) can be about 0.002 to about 6500 nM. In some embodiments, the binding affinity is about any of 6500 nm, 6000 nm, 5986 nm, 5567 nm, 5500 nm, 4500 nm, 4000 nm, 3500 nm, 3000 nm, 2500 nm, 2134 nm, 2000 nm, 1500 nm, 1000 nm, 750 nm, 500 nm, 400 nm, 300 nm, 250 nm, 200 nM, 193 nM, 100 nM, 90 nM, 50 nM, 45 nM, 40 nM, 35 nM, 30 nM, 25 nM, 20 nM, 19 nm, 18 nm, 17 nm, 16 nm, 15 nM, 10 nM, 8 nM, 7.5 nM, 7 nM, 6.5 nM, 6 nM, 5.5 nM, 5 nM, 4 nM, 3 nM, 2 nM, 1 nM, 0.5 nM, 0.3 nM, 0.1 nM, 0.01 nM, or 0.002 nM. In some embodiments, the binding affinity is less than about any of 6500 nm, 6000 nm, 5500 nm, 5000 nm, 4000 nm, 3000 nm, 2000 nm, 1000 nm, 900 nm, 800 nm, 250 nM, 200 nM, 100 nM, 50 nM, 30 nM, 20 nM, 10 nM, 7.5 nM, 7 nM, 6.5 nM, 6 nM, 5 nM, 4.5 nM, 4 nM, 3.5 nM, 3 nM, 2.5 nM, 2 nM, 1.5 nM, 1 nM, or 0.5 nM.

The intracellular signaling domain of a CAR according to the disclosure is responsible for intracellular signaling following the binding of extracellular ligand-binding domain to the target resulting in the activation of the immune cell and immune response. The intracellular signaling domain has the ability to activate of at least one of the normal effector functions of the immune cell in which the CAR is expressed. For example, the effector function of a T cell can be a cytolytic activity or helper activity including the secretion of cytokines.

In some embodiments, an intracellular signaling domain for use in a CAR can be the cytoplasmic sequences of, for example without limitation, the T cell receptor and co-receptors that act in concert to initiate signal transduction following antigen receptor engagement, as well as any derivative or variant of these sequences and any synthetic sequence that has the same functional capability. Intracellular signaling domains comprise two distinct classes of cytoplasmic signaling sequences: those that initiate antigen-dependent primary activation, and those that act in an antigen-independent manner to provide a secondary or co-stimulatory signal. Primary cytoplasmic signaling sequences can comprise signaling motifs which are known as immunoreceptor tyrosine-based activation motifs of ITAMs. ITAMs are well defined signaling motifs found in the intracytoplasmic tail of a variety of receptors that serve as binding sites for syk/zap70 class tyrosine kinases. Examples of ITAM used in the disclosure can include as non limiting examples those derived from TCRζ, FcRγ, FcRβ, FcRε, CD3γ, CD3δ, CD3ε, CD5, CD22, CD79a, CD79b and CD66d. In some embodiments, the intracellular signaling domain of the CAR can comprise the CD3ζ signaling domain which has amino acid sequence with at least about 70%, preferably at least 80%, more preferably at least 90%, 95%, 97%, or 99% sequence identity with an amino acid sequence shown in SEQ. ID NO: 324. In some embodiments, the intracellular signaling domain of the CAR of the disclosure comprises a domain of a co-stimulatory molecule.

In some embodiments, the intracellular signaling domain of a CAR of the disclosure comprises a part of co-stimulatory molecule selected from the group consisting of fragment of 41BB (GenBank: AAA53133.) and CD28 (NP_006130.1). In some embodiments, the intracellular signaling domain of the CAR of the disclosure comprises amino acid sequence which comprises at least 70%, preferably at least 80%, more preferably at least 90%, 95%, 97%, or 99% sequence identity with an amino acid sequence shown in SEQ. ID NO: 323 and SEQ. ID NO: 327.

CARs are expressed on the surface membrane of the cell. Thus, the CAR can comprise a transmembrane domain. Suitable transmembrane domains for a CAR disclosed herein have the ability to (a) be expressed at the surface of a cell, preferably an immune cell such as, for example without limitation, lymphocyte cells or Natural killer (NK) cells, and (b) interact with the ligand-binding domain and intracellular signaling domain for directing cellular response of immune cell against a predefined target cell. The transmembrane domain can be derived either from a natural or from a synthetic source. The transmembrane domain can be derived from any membrane-bound or transmembrane protein. As non-limiting examples, the transmembrane polypeptide can be α subunit of the T cell receptor such as α, β, γ or δ, polypeptide constituting CD3 complex, IL-2 receptor p55 (a chain), p75 (β chain) or γ chain, subunit chain of Fc receptors, in particular Fcγ receptor III or CD proteins. Alternatively, the transmembrane domain can be synthetic and can comprise predominantly hydrophobic residues such as leucine and valine. In some embodiments, said transmembrane domain is derived from the human CD8α chain (e.g., NP_001139345.1). The transmembrane domain can further comprise a stalk domain between the extracellular ligand-binding domain and said transmembrane domain. A stalk domain may comprise up to 300 amino acids, preferably 10 to 100 amino acids and most preferably 25 to 50 amino acids. Stalk region may be derived from all or part of naturally occurring molecules, such as from all or part of the extracellular region of CD8, CD4, or CD28, or from all or part of an antibody constant region. Alternatively, the stalk domain may be a synthetic sequence that corresponds to a naturally occurring stalk sequence or may be an entirely synthetic stalk sequence. In some embodiments, said stalk domain is a part of human CD8α chain (e.g., NP_001139345.1). In another particular embodiment, said transmembrane and hinge domains comprise a part of human CD8α chain, preferably which comprises at least 70%, preferably at least 80%, more preferably at least 90%, 95% 97%, or 99% sequence identity with amino acid sequence selected from the group consisting of SEQ ID NO: 318. In some embodiments, CARs disclosed herein can comprise an extracellular ligand-binding domain that specifically binds BCMA, CD8α human hinge and transmembrane domains, the CD3ζ signaling domain, and 4-1BB signaling domain.

Table 3 provides exemplary sequences of domains which can be used in the CARs disclosed herein.

TABLE 3
Exemplary sequences of CAR Components

| | | SEQ |
|---|---|---|
| | | ID |
| Domain | Amino Acid Sequence | NO: |
| CD8α signal | MALPVTALLLPLALLLHAARP | 318 |
| peptide | | |
| FcγRIIIα hinge | GLAVSTISSFFPPGYQ | 319 |
| CD8α hinge | TTTPAPRPPTPAPTIASQPLSLRPEACRPAAGGAVHTRGL | 320 |
| | DFACD | |
| IgG1 hinge | EPKSPDKTHTCPPCPAPPVAGPSVFLFPPKPKDTLMIART | 321 |
| | PEVTCVVVDVSHEDPEVKFNWYVDGVEVHNAKTKPREEQY | |
| | NSTYRVVSVLTVLHQDWLNGKEYKCKVSNKALPAPIEKTI | |
| | SKAKGQPREPQVYTLPPSRDELTKNQVSLTCLVKGFYPSD | |
| | IAVEWESNGQPENNYKTTPPVLDSDGSFFLYSKLTVDKSR | |
| | WQQGNVFSCSVMHEALHNHYTQKSLSLSPGK | |
| CD8α | IYIWAPLAGTCGVLLLSLVITLYC | 322 |
| transmembrane | | |
| (TM) domain | | |
| 4IBB intracellular | KRGRKKLLYIFKQPFMRPVQTTQEEDGCSCRFPEEEEGGC | 323 |
| signaling domain | EL | |
| (ISD) | | |
| CD3ζ intracellular | RVKFSRSADAPAYQQGQNQLYNELNLGRREEYDVLDKRRG | 324 |
| signaling domain | RDPEMGGKPRRKNPQEGLYNELQKDKMAEAYSEIGMKGER | |
| (ISD) | RRGKGHDGLYQGLSTATKDTYDALHMQALPPR | |
| FcεRI α-TM-IC | FFIPLLVVILFAVDTGLFISTQQQVTFLLKIKRTRKGFRL | 325 |
| (FcεRI α chain | LNPHPKPNPKNN | |
| transmembrane and | | |
| intracellular | | |
| domain) | | |
| FcεRIβ-ΔITAM | MDTESNRRANLALPQEPSSVPAFEVLEISPQEVSSGRLLK | 326 |
| (FcεRI β chain | SASSPPLHTWLTVLKKEQEFLGVTQILTAMICLCFGTVVC | |
| without ITAM) | SVLDISHIEGDIFSSFKAGYPFWGAIFFSISGMLSIISER | |
| | RNATYLVRGSLGANTASSIAGGTGITILIINLKKSLAYIH | |
| | IHSCQKFFETKCFMASFSTEIVVMMLFLTILGLGSAVSLT | |
| | ICGAGEELKGNKVPE | |
| 41BB-IC (4IBB co- | KRGRKKLLYIFKQPFMRPVQTTQEEDGCSCRFPEEEEGGC | 327 |
| stimulatory domain) | EL | |
| CD28-IC (CD28 | RSKRSRGGHSDYMNMTPRRPGPTRKHYQPYAPPRDFAAYR | 328 |
| co-stimulatory | S | |
| domain) | | |
| FcεRIγ-SP (signal | MIPAVVLLLLLLVEQAAA | 329 |
| peptide) | | |
| FcεRI γ-ΔITAM | LGEPQLCYILDAILFLYGIVLTLLYCRLKIQVRKAAITSY | 330 |
| (FcεRI γ chain | EKS | |
| without ITAM) | | |
| GSG-P2A (GSG- | GSGATNFSLLKQAGDVEENPGP | 331 |
| P2A ribosomal | | |
| skip polypeptide) | | |
| GSG-T2A (GSG- | GSGEGRGSLLTCGDVEENPGP | 332 |
| T2A ribosomal | | |
| skip polypeptide) | | |

In another aspect, the disclosure provides polynucleotides encoding any of the CARs and polypeptides described herein. Polynucleotides can be made and expressed by procedures known in the art.

In another aspect, the disclosure provides compositions (such as a pharmaceutical compositions) comprising any of the cells of the disclosure.

III. Engineered Immune Cells

The disclosure provides engineered immune cells comprising any of the BCMA CAR polynucleotides described herein. In some embodiments, the BCMA CAR is introduced into an immune cell with a lentiviral vector. In some embodiments, the lentiviral vector is a self-inactivating lentiviral vector that integrates into the recipient immune cell. In some embodiments, the BCMA CAR is introduced into an immune cell as a transgene via a plasmid vector. In some embodiments, the plasmid vector can also contain, for example, a selection marker which provides for identification and/or selection of cells which received the vector. In some embodiments, the CAR can be introduced into the immune cell using non-viral methods.

An exemplary vector construct is show in FIG. 21. The amino acid sequences of exemplary BCMA CARs with two rituximab mimotopes with (SEQ ID NO: 418) or without (SEQ ID NO: 419) a signal peptide are shown below:

(SEQ ID NO: 418)
M A L P V T A L L L P L A L L L H A A R P E V Q L
L E S G G G L V Q P G G S L R L S C A A S G F T F
S S Y A M N W V R Q A P G K G L E W V S A I S D S
G G S T Y Y A D S V K G R F T I S R D N S K N T L
Y L Q M N S L R A E D T A V Y Y C A R Y W P M D I
W G Q G T L V T V S S G G G G S G G G G S G G G G
S E I V L T Q S P G T L S L S P G E R A T L S C R
A S Q S V S S S Y L A W Y Q Q K P G Q A P R L L M
Y D A S I R A T G I P D R F S G S G S G T D F T L
T I S R L E P E D F A V Y Y C Q Q Y G S W P L T F
G Q G T K V E I K G S G G G G S C P Y S N P S L C
S G G G G S C P Y S N P S L C S G G G G S T T T P
A P R P P T P A P T I A S Q P L S L R P E A C R P
A A G G A V H T R G L D F A C D I Y I W A P L A G
T C G V L L L S L V I T L Y C K R G R K K L L Y I
F K Q P F M R P V Q T T Q E E D G C S C R F P E E
E E G G C E L R V K F S R S A D A P A Y Q Q G Q N
Q L Y N E L N L G R R E E Y D V L D K R R G R D P
E M G G K P R R K N P Q E G L Y N E L Q K D K M A
E A Y S E I G M K G E R R R G K G H D G L Y Q G L
S T A T K D T Y D A L H M Q A L P P R
(SEQ ID NO: 419)
E V Q L L E S G G G L V Q P G G S L R L S C A A S
G F T F S S Y A M N W V R Q A P G K G L E W V S A
I S D S G G S T Y Y A D S V K G R F T I S R D N S
K N T L Y L Q M N S L R A E D T A V Y Y C A R Y W
P M D I W G Q G T L V T V S S G G G G S G G G G S
G G G G S E I V L T Q S P G T L S L S P G E R A T
L S C R A S Q S V S S S Y L A W Y Q Q K P G Q A P
R L L M Y D A S I R A T G I P D R F S G S G S G T
D F T L T I S R L E P E D F A V Y Y C Q Q Y G S W
P L T F G Q G T K V E I K G S G G G G S C P Y S N
P S L C S G G G G S C P Y S N P S L C S G G G G S
T T T P A P R P P T P A P T I A S Q P L S L R P E
A C R P A A G G A V H T R G L D F A C D I Y I W A
P L A G T C G V L L L S L V I T L Y C K R G R K K
L L Y I F K Q P F M R P V Q T T Q E E D G C S C R
F P E E E E G G C E L R V K F S R S A D A P A Y Q
Q G Q N Q L Y N E L N L G R R E E Y D V L D K R R
G R D P E M G G K P R R K N P Q E G L Y N E L Q K
D K M A E A Y S E I G M K G E R R R G K G H D G L
Y Q G L S T A T K D T Y D A L H M Q A L P P R

Methods of generating engineered immune cells expressing any of the BCMA CARs provided herein is described in WO/2016/166630, incorporated by reference in its entirety.

Provided herein are isolated immune cells obtained according to any one of the methods described above. Any immune cell capable of expressing heterologous DNAs can be used for the purpose of expressing the CAR of interest. In some embodiments, the immune cell is a T cell. In some embodiments, an immune cell can be derived from, for example without limitation, a stem cell. The stem cells can be adult stem cells, non-human embryonic stem cells, more particularly non-human stem cells, cord blood stem cells, progenitor cells, bone marrow stem cells, induced pluripotent stem cells, totipotent stem cells or hematopoietic stem cells. Representative human cells are CD34+ cells. The isolated cell can also be a dendritic cell, killer dendritic cell, a mast cell, a NK-cell, a B-cell or a T cell selected from the group consisting of inflammatory T-lymphocytes, cytotoxic T-lymphocytes, regulatory T-lymphocytes or helper T-lymphocytes. I n some embodiments, the cell can be derived from the group consisting of CD4+ T-lymphocytes and CD8+ T-lymphocytes.

In some embodiments, an isolated cell according to the present disclosure comprises one inactivated gene selected from the group consisting of CD52, GR, PD-1, CTLA-4, LAG3, Tim3, BTLA., BY55, TIGIT, B7H5, LAIR1, SIGLEC10, 2B4, HLA, TCRα and TCRβ and/or expresses a CAR, a multi-chain CAR and/or a pTα transgene. In some embodiments, an isolated cell comprises polynucleotides encoding polypeptides comprising a multi-chain CAR. In some embodiments, the isolated cell according to the present disclosure comprises two inactivated genes selected from the group consisting of: CD52 and GR, CD52 and TCRα, CDR52 and TCRβ, GR and TCRα, GR and TCRβ, TCRα and TCRβ, PD-1 and TCRα, PD-1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ and/or expresses a CAR, a multi-chain CAR and a pTα transgene.

Gene inactivation can be carried out by methods practiced by those with skill in the art. The methods include, but are not limited to gene inactivation by use of zinc fingers, TALEN®s, and CRISPR/Cas-based system.

In some embodiments, the BCMA CAR containing immune cell has an inactivated CD52 gene. In some embodiments, only one copy of the CD52 gene is inactivated.

In some embodiments, the BCMA CAR containing immune cell has an inactivated TCRα gene.

In some embodiments, the BCMA CAR containing immune cell has an inactivated TCRβ gene.

In some embodiments, TALEN® is used for gene inactivation. In such embodiments, the efficiency of gene inactivation with TALEN® is not 100%, and resulting TCRαβ-negative T-cells are enriched by depleting residual TCRαβ-positive T cells before cryopreservation. However, CD52-negative cells are not purified, resulting in a cell product with varying frequencies of CD52-negative cells, typically between 60-80%. Accordingly, in some embodiments, the genotype of the BCMA CAR-T cells of the disclosure is BCMA-CAR+_TCRαβ−_CD52+/− T-cells

In some embodiments, TCR is rendered not functional in the cells according to the disclosure by inactivating TCRα gene and/or TCRβ gene(s). In some embodiments, a method to obtain modified cells derived from an individual is provided, wherein the cells can proliferate independently of the major histocompatibility complex (MHC) signaling pathway. Modified cells, which can proliferate independently of the MHC signaling pathway, susceptible to be obtained by this method are encompassed in the scope of the present disclosure. Modified cells disclosed herein can be used in for treating subjects in need thereof against Host versus Graft (HvG) rejection and Graft versus Host Disease (GvHD); therefore in the scope of the present disclosure is a method of treating subjects in need thereof against Host versus Graft (HvG) rejection and Graft versus Host Disease (GvHD) comprising treating said subject by administering to said subject an effective amount of modified cells comprising inactivated TCRα and/or TCRβ genes.

In some embodiments, the immune cells are engineered to be resistant to one or more chemotherapy drugs. The chemotherapy drug can be, for example, a purine nucleotide analogue (PNA), thus making the immune cell suitable for cancer treatment combining adoptive immunotherapy and chemotherapy. Exemplary PNAs include, for example, clofarabine, fludarabine, and cytarabine, alone or in combination. PNAs are metabolized by deoxycytidine kinase (dCK) into mono-, di-, and tri-phosphate PNA. Their tri-phosphate forms compete with ATP for DNA synthesis, act as pro-apoptotic agents, and are potent inhibitors of ribonucleotide reductase (RNR), which is involved in trinucleotide production. Provided herein are BCMA specific CAR-T cells comprising an inactivated dCK gene. In some embodiments, the dCK knockout cells are made by transfection of T cells using polynucleotides encoding specific TAL-nuclease directed against dCK genes by, for example, electroporation of mRNA. The dCK knockout BCMA specific CAR-T cells are resistant to PNAs, including for example clofarabine and/or fludarabine, and maintain T cell cytotoxic activity toward BCMA-expressing cells.

In some embodiments, isolated cells or cell lines of the disclosure can comprise a pTα or a functional variant thereof. In some embodiments, an isolated cell or cell line can be further genetically modified by inactivating the TCRα gene.

In some embodiments, the CAR-T cell comprises a polynucleotide encoding a safety switch, such as for example RQR8. See, e.g., WO2013153391A, which is hereby incorporated by reference in its entirety. In CAR-T cells comprising the polynucleotide, the safety switch polypeptide is expressed at the surface of a CAR-T cell. In some embodiments, the safety switch polypeptide comprises the amino acid sequence shown in SEQ ID NO: 342.

(SEQ ID NO: 342)
CPYSNPSLCSGGGGSELPTQGTFSNVSTNVSPAKPTTTACPYSNPSLCSG
GGGSPAPRPPTPAPTIASQPLSLRPEACRPAAGGAVHTRGLDFACDIYIW
APLAGTCGVLLLSLVITLYCNHRNRRRVCKCPRPVV

The safety switch polypeptide may also comprise a signal peptide at the amino terminus. In some embodiments, the safety switch polypeptide comprises the amino acid sequence shown in SEQ ID NO: 400.

(SEQ ID NO: 400)
MGTSLLCWMALCLLGADHADACPYSNPSLCSGGGGSELPTQGTFSNVSTN
VSPAKPTTTACPYSNPSLCSGGGGSPAPRPPTPAPTIASQPLSLRPEACR
PAAGGAVHTRGLDFACDIYIWAPLAGTCGVLLLSLVITLYCNHRNRRRVC
KCPRPVV

When the safety switch polypeptide is expressed at the surface of a CAR-T cell, binding of rituximab to the R epitopes of the polypeptide causes lysis of the cell. More than one molecule of rituximab may bind per polypeptide expressed at the cell surface. Each R epitope of the polypeptide may bind a separate molecule of rituximab. Deletion of BCMA specific CAR-T cells may occur in vivo, for example by administering rituximab to a subject. The decision to delete the transferred cells may arise from undesirable effects being detected in the subject which are attributable to the transferred cells, such as for example, when unacceptable levels of toxicity are detected.

In some embodiments, the CAR-T cell comprises a selected epitope within the scFv having a specificity to be recognized by a specific antibody. See, e.g., PCT application PCT/EP2016/051467, WO2016/120216, “mAb-DRIVEN CHIMERIC ANTIGEN RECEPTOR SYSTEMS FOR SORTING/DEPLETING ENGINEERED IMMUNE CELLS,” filed on Jan. 25, 2016, which is hereby incorporated by reference in its entirety. Such an epitope facilitates sorting and/or depleting the CAR-T cells. The epitope can be selected from any number of epitopes known in the art. In some embodiments, the epitope can be a target of a monoclonal antibody approved for medical use, such as, for example without limitation, the CD20 epitope recognized by rituximab. In some embodiments, the epitope comprises the amino acid sequence shown in SEQ ID NO: 397.

| (SEQ ID NO: 397) | |
|---|---|
| | CPYSNPSLC |

In some embodiments, the epitope is located within the CAR. For example without limitation, the epitope can be located between the scFv and the hinge of a CAR. In some embodiments, two instances of the same epitope, separate by linkers, may be used in the CAR. For example, the polypeptide comprising the amino acid sequence shown in SEQ ID NO: 398 can be used within a CAR, located between the light chain variable region and the hinge.

| (SEQ ID NO: 398) | |
|---|---|
| | GSGGGGSCPYSNPSLCSGGGGSCPYSNPSLCSGGGGS |

In some embodiments, the epitope-specific antibody may be conjugated with a cytotoxic drug. It is also possible to promote CDC cytotoxicity by using engineered antibodies on which are grafted component(s) of the complement system. In some embodiments, activation of the CAR-T cells can be modulated by depleting the cells using an antibody which recognizes the epitope.

IV. Therapeutic Applications

Isolated cells obtained by the methods described above, or cell lines derived from such isolated cells, can be used as a medicament in combination with a gamma secretase inhibitor (e.g., nirogacestat or a pharmaceutically acceptable salt thereof). In some embodiments, such a combination can be used for treating MM. In some embodiments, the MM is refractory MM. In some embodiments, the MM is relapsed MM. In some embodiments, the MM is refractory/relapsed MM.

In some embodiments, the subject has not received any prior therapy for multiple myeloma. In some embodiments, the subject has received at least one, two, or three prior therapies for multiple myeloma. In some embodiments, the dosing regimens provided herein are a first line therapy. In some embodiments, the dosing regimens provided herein are a second line therapy. In some embodiments, the dosing regimens provided herein are a third line therapy. In some embodiments, the dosing regimens provided herein are a fourth line therapy. In some embodiments, the subject has relapsed MM. In some embodiments, the subject has refractory MM. In some embodiments, the subject has refractory and relapsed MM.

In some embodiments, an isolated cell according to the disclosure, or cell line derived from the isolated cells, can be used in the manufacture of a medicament in combination with a gamma secretase inhibitor (e.g., nirogacestat or a pharmaceutically acceptable salt thereof) for treatment of a cancer in a subject in need thereof.

Also provided herein are methods for treating subjects. In some embodiments, the method comprises providing an immune cell of the disclosure in combination with a gamma secretase inhibitor (e.g., nirogacestat or a pharmaceutically acceptable salt thereof) to a subject in need thereof. In some embodiments, the method comprises a step of administrating transformed immune cells of the disclosure to a subject in need thereof in combination with a gamma secretase inhibitor (e.g., nirogacestat or a pharmaceutically acceptable salt thereof). The subject can be male or female, adult, adolescent, or pediatric. In some embodiments, the subject is a human subject.

In some embodiments, T cells of the disclosure can undergo in vivo T cell expansion and can persist for an extended amount of time.

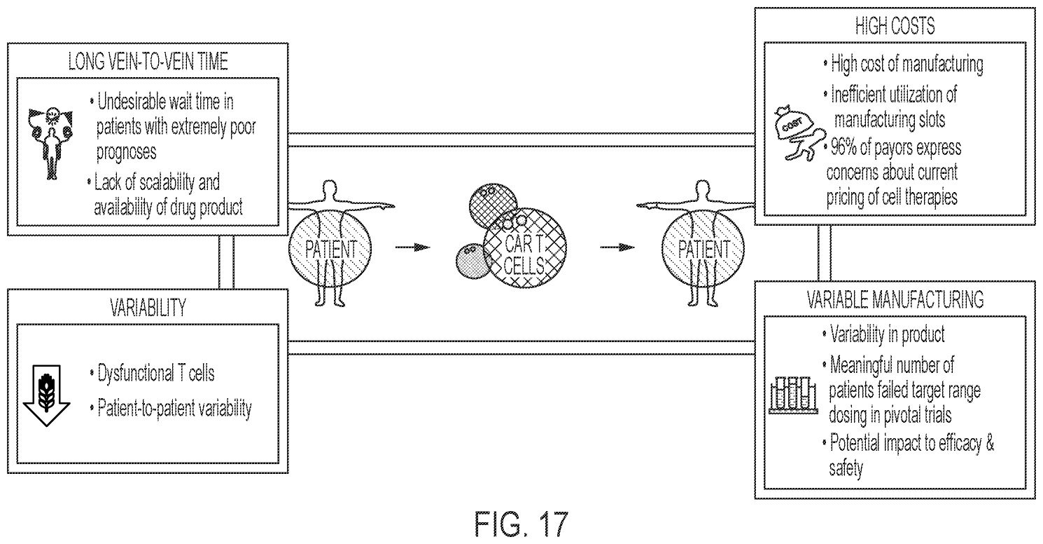

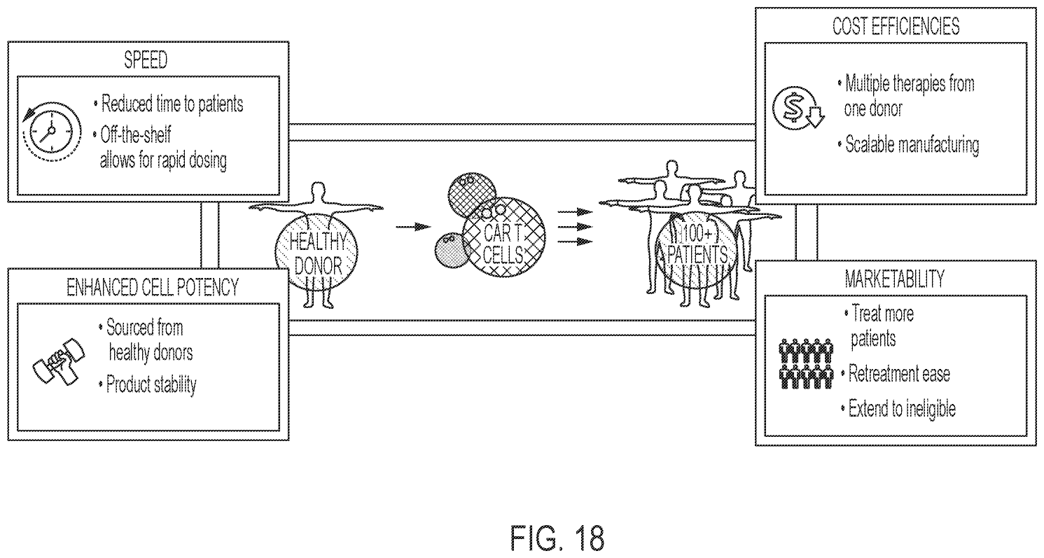

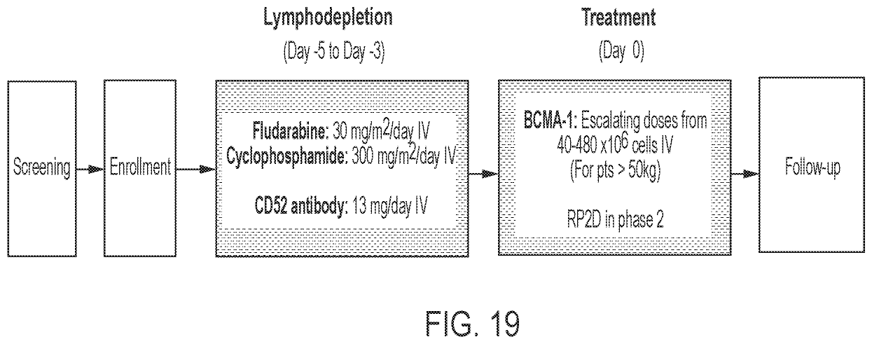

Methods of treatment of the disclosure can be ameliorating, curative or prophylactic. The method of the disclosure may be either part of an autologous immunotherapy or part of an allogenic immunotherapy treatment. The disclosure is particularly suitable for allogeneic immunotherapy. T cells from donors can be transformed into non-alloreactive cells using standard protocols and reproduced as needed, thereby producing CAR-T cells which may be administered to one or several subjects. Such CAR-T cell therapy can be made available as an “off the shelf” therapeutic product. FIGS. 17 and 18 describe the limitations of autologous CAR-T therapies, and the advantages of allogeneic therapies.

Cells that can be used with the disclosed methods are described in the previous section. Treatment can be used to treat subjects diagnosed with MM. Adult tumors/cancers and pediatric tumors/cancers are also included. In some embodiments, the treatment can be in combination with one or more therapies against MM selected from the group of antibodies therapy, chemotherapy, cytokines therapy, dendritic cell therapy, gene therapy, hormone therapy, laser light therapy and radiation therapy.

In some embodiments, treatment can be administrated into subjects undergoing an immunosuppressive treatment. Indeed, the disclosure preferably relies on cells or population of cells, which have been made resistant to at least one immunosuppressive agent due to the inactivation of a gene encoding a receptor for such immunosuppressive agent. In this aspect, the immunosuppressive treatment should help the selection and expansion of the T cells according to the disclosure within the subject.

The administration of the cells or population of cells according to the disclosure may be carried out in any convenient manner, including by aerosol inhalation, injection, ingestion, transfusion, implantation or transplantation. The compositions described herein may be administered to a subject subcutaneously, intradermally, intratumorally, intranodally, intramedullary, intramuscularly, by intravenous or intralymphatic injection, or intraperitoneally. In one embodiment, the cell compositions of the disclosure are preferably administered by intravenous injection.

In some embodiments, the engineered BCMA CAR-expressing immune cells of the disclosure are formulated for infusion. In some embodiments, the cells are formulated in a solution comprising about 5% DMSO. In one embodiment 14×10\hat{ }6 BCMA-CAR-T-cells/mL are formulated in a solution comprising about 5% DMSO. In a further embodiment the formulation comprises a 1:1 mixture of CryoStor® Basal Solution and CryoStor® CS10 resulting in a 5% final concentration of dimethyl sulfoxide. In some embodiments, the dosage strength of the formulation is 14×10\hat{ }6 BCMA-CAR-T-cells/mL. In some embodiments, this formulated drug product is supplied in a 2-mL closed-system vial with an integral stopper at a nominal volume of 1 mL.

In some embodiments, the BCMA CAR-T cells of the disclosure are BCMA-CAR+_TCRαβ−_CD52+/− T-cells and are formulated as a suspension for infusion. In some embodiments, the BCMA-CAR+_TCRαβ−_CD52+/− T-cells are formulated in a 1:1 mixture of CryoStor® Basal Solution and CryoStor® CS10 resulting in a 5% final concentration of dimethyl sulfoxide. In some embodiments, the dosage strength of the formulation is 14×10\hat{ }6 BCMA-CAR+_TCRαβ−_CD52+/− T-cells/mL.

In some embodiments, the gamma secretase inhibitor (e.g., nirogacestat or a pharmaceutically acceptable salt thereof) is formulated for oral administration (e.g., tablets, capsules, aqueous suspensions). If the gamma secretase inhibitor is to be formulated for oral administration, known carriers can be included in the pharmaceutical composition. For example, microcrystalline cellulose, sodium citrate, calcium carbonate, dicalcium phosphate and glycine may be employed along with various disintegrants such as starch (preferably corn, potato or tapioca starch), methylcellulose, alginic acid and certain complex silicates, together with granulation binders such as polyvinylpyrrolidone, sucrose, gelatin and acacia, can be included in a tablet.

Additionally, lubricating agents such as magnesium stearate, sodium lauryl sulfate and talc are often useful for tabletting purposes. Solid compositions of a similar type may also be employed as fillers in gelatin capsules. Preferred materials in this connection include lactose or milk sugar as well as high molecular weight polyethylene glycols. When aqueous suspensions and/or elixirs are desired for oral administration, the active ingredient may be combined with various sweetening or flavoring agents, coloring matter or dyes, and, if so desired, emulsifying and/or suspending agents as well, together with such diluents as water, ethanol, propylene glycol, glycerin and various like combinations thereof.

For parenteral administration, solutions containing a gamma secretase inhibitor (e.g., nirogacestat or a pharmaceutically acceptable salt thereof) can be prepared in either sesame or peanut oil, in aqueous propylene glycol, or in sterile water or saline. The aqueous solutions should be suitably buffered (preferably pH greater than 8) if necessary and the liquid diluent first rendered isotonic with sufficient saline or glucose. These aqueous solutions are suitable for intravenous injection purposes. The oily solutions are suitable for intraarticular, intramuscular and subcutaneous injection purposes. The preparation of all these solutions under sterile conditions is readily accomplished by standard pharmaceutical techniques well known to those skilled in the art.

V. Lymphodepletion

In some embodiments, a lymphodepletion (LD) regimen is administered to the subject prior to a first and/or subsequent dose of the BCMA CAR-T cells. In some embodiments, the lymphodepletion regimen is administered to the subject concurrently with a first and/or subsequent dose of CAR-T cells. In some embodiments, the lymphodepletion regimen is administered before, during, and/or after a first and/or subsequent dose of BCMA CAR-T cells.

Suitable LD regimens are described herein and/or known in the art. In some embodiments, LD starts prior to, concurrently with, or after a CAR-T infusion. Doses and timing of LD administration may be adapted with regard to the first or subsequent dosing of BCMA CAR-T. In some embodiments, the duration of LD is about 3 to 5 days. In some embodiments, a time window between the end of LD and start of CAR-T administration is between about of 2 days to about 2 weeks. In some embodiments, LD is initiated about 15 to 7 days prior to administration of a dose of CAR-T cells. In some embodiments, LD is initiated about 19 to 5 days prior to administration of a dose of CAR-T cells. In some embodiments, LD is initiated about 3, 4, 5, 6, 7, 8, 9, 10, 11, 12, 13, 14, 15, 16, 17, 18, 19, or 20 days prior to administration of a dose of CAR-T cells. In some embodiments, duration of a LD regimen is about 1, 2, 3, 4, 5, 6, 7, 8, 9, 10, 11, 12, 13, or 14 days. In some embodiments, a dose of CAR-T cells is administered about 1, 2, 3, 4, 5, 6, 7, 8, 9, 10, 11, 12, 13, or 14 days after the end of LD.

In some embodiments, a LD regimen comprises administration of one or more chemotherapeutic drugs.

In some embodiments, a LD regimen comprises administration of anti-CD52 antibody, such as an antibody that recognizes the human cluster of differentiation (CD) 52 antigen, a cell surface glycoprotein expressed on most lymphoid cells. As used herein a CD52 monoclonal antibody is one that is directed against the 21-28 kD cell surface glycoprotein CD52. CD52 is an abundant molecule (approximately 5×10⁵ antibody binding sites per cell) present on at least 95% of all human peripheral blood lymphocytes and monocytes/macrophages. Exemplary CD52 antibodies for use in the methods and compositions described herein include, for example, alemtuzumab. In some embodiments, a CD52 antibody comprises the HCDR1, HCDR2, HCDR3, LCDR1, LCDR2, and LCDR3 sequences as shown in Table 4 below.

TABLE 4
Exemplary CD52 antibody CDR sequences

| CDR | Sequence (SEQ ID NO) |
|---|---|
| HCDR1 | DFYMN (SEQ ID NO: 402) |
| HCDR2 | FIRDKAKGYTTEYNPSVKG (SEQ ID |
| | NO: 403) |
| HCDR3 | EGHTAAPFDY (SEQ ID NO: 404) |
| LCDR1 | KASQNIDKYLN (SEQ ID NO: 405) |
| LCDR2 | NTNNLQT (SEQ ID NO: 406) |
| LCDR3 | LQHISRPRT (SEQ ID NO: 407) |

In some embodiments, a CD52 antibody comprises a VH and/or a VL comprising the sequences shown in Table 5 below. In some embodiments, a CD52 antibody comprises a heavy chain (HC) and/or a light chain (LC) comprising the sequences shown in Table 5 below.

TABLE 5
Exemplary CD52 Antibody HC, LC, VH and VL sequences

| | Amino Acid Sequence | DNA Sequence |
|---|---|---|
| HC | QVQLQESGPGLVRPSQTLSLTCTVSG | caagtgcagcttcaagaatccggccctggtctggtccgcccctc |
| | FTFTDFYMNWVRQPPGRGLEWIGFIR | ccaaaccctctccctgacatgcaccgtgtcgggattcaccttta |
| | DKAKGYTTEYNPSVKGRVTMLVDTSK | ccgatttctacatgaactgggtccggcagccgcccggaagaggt |
| | NQFSLRLSSVTAADTAVYYCAREGHT | ctggagtggatcggcttcattcgggacaaagccaaggggtacac |
| | AAPFDYWGQGSLVTVSSASTKGPSVF | caccgagtacaacccgtccgtgaagggacgcgtgactatgctcg |
| | PLAPSSKSTSGGTAALGCLVKDYFPE | tggacacgtccaagaaccagttcagcttgaggctgagcagcgtg |
| | PVTVSWNSGALTSGVHTFPAVLQSSG | actgccgcggataccgcagtgtactactgtgcccgggaagggca |
| | LYSLSSVVTVPSSSLGTQTYICNVNH | cactgccgctccattcgactattggggccagggatcactggtca |
| | KPSNTKVDKKVEPKSCDKTHTCPPCP | ctgtgtcgtccgcctccaccaagggcccatcggtcttccccctg |
| | APELLGGPSVFLFPPKPKDTLMISRT | gcaccctcctccaagagcacctctgggggcacagcggccctggg |
| | PEVTCVVVDVSHEDPEVKFNWYVDGV | ctgcctggtcaaggactacttccccgaaccggtgacggtgtcgt |
| | EVHNAKTKPREEQYNSTYRVVSVLTV | ggaactcaggcgccctgaccagcggcgtgcacaccttcccggct |
| | LHQDWLNGKEYKCKVSNKALPAPIEK | gtcctacagtcctcaggactctactccctcagcagcgtagtgac |
| | TISKAKGQPREPQVYTLPPSRDELTK | cgtgccctccagcagcttgggcacccagacctacatctgcaacg |
| | NQVSLTCLVKGFYPSDIAVEWESNGQ | tgaatcacaagcccagcaacaccaaggtggacaagaaagttgag |
| | PENNYKTTPPVLDSDGSFFLYSKLTV | cccaaatcttgtgacaaaactcacacatgcccaccgtgcccagc |
| | DKSRWQQGNVFSCSVMHEALHNHYTQ | acctgaactcctggggggaccgtcagtcttcctcttccccccaa |
| | KSLSLSPGK (SEQ ID NO: 408) | aacccaaggacaccctcatgatctcccggacccctgaggtcaca |
| | | tgcgtggtggtggacgtgagccacgaagaccctgaggtcaagtt |
| | | caactggtacgtggacggcgtggaggtgcataatgccaagacaa |
| | | agccgcgggaggagcagtacaacagcacgtaccgtgtggtcagc |
| | | gtcctcaccgtcctgcaccaggactggctgaatggcaaggagta |
| | | caagtgcaaggtctccaacaaagccctcccagcccccatcgaga |
| | | aaaccatctccaaagccaaagggcagccccgagaaccacaggtg |
| | | tacaccctgcccccatcccgggacgagctgaccaagaaccaggt |
| | | cagcctgacctgcctggtcaaaggcttctatcccagcgacatcg |
| | | ccgtggagtgggagagcaatgggcagccggagaacaactacaag |
| | | accacgcctcccgtgctggactccgacggctccttcttcctcta |
| | | tagcaagctcaccgtggacaagagcaggtggcagcaggggaacg |
| | | tcttctcatgctccgtgatgcatgaggctctgcacaaccactac |
| | | acgcagaagagcctctccctgtctccgggaaaa (SEQ ID |
| | | NO: 409) |
| LC | DIQMTQSPSSLSASVGDRVTITCKAS | Atgggatggagctgtatcatcctcttcttggtagcaacagctac |
| | QNIDKYLNWYQQKPGKAPKLLIYNTN | aggcgtgcactccgacatccaaatgacccaatccccatcctcac |
| | NLQTGVPSRFSGSGSGTDFTFTISSL | tttccgcctccgtgggcgaccgcgtgactattacctgtaaagcg |
| | QPEDIATYYCLQHISRPRTFGQGTKV | tcacagaatatcgacaagtacctgaactggtaccagcagaagcc |
| | EIKRTVAAPSVFIFPPSDEQLKSGTA | tggaaaggcccccaagctcctgatctacaacaccaacaacttgc |
| | SVVCLLNNFYPREAKVQWKVDNALQS | agactggagtgccgagcagattttccggctccggctcggggact |
| | GNSQESVTEQDSKDSTYSLSSTLTLS | gatttcaccttcaccatctcgagcctgcagccggaggatattgc |
| | KADYEKHKVYACEVTHQGLSSPVTKS | tacctattactgcctgcaacacattagccggcccaggacgttcg |
| | FNRGEC (SEQ ID NO: 410) | gacagggtaccaaggtcgaaatcaagcgtacggtggctgcacca |
| | | tctgtcttcatcttcccgccatctgatgagcagttgaaatctgg |
| | | aactgcctctgttgtgtgcctgctgaataacttctatcccagag |
| | | aggccaaagtacagtggaaggtggataacgccctccaatcgggt |
| | | aactcccaggagagtgtcacagagcaggacagcaaggacagcac |
| | | ctacagcctcagcagcaccctgacgctgagcaaagcagactacg |
| | | agaaacacaaagtctacgcctgcgaagtcacccatcagggcctg |
| | | agctcgcccgtcacaaagagcttcaacaggggagagtgt |
| | | (SEQ ID NO: 411) |

| | Amino Acid Sequence |
|---|---|
| VH | QVQLQESGPGLVRPSQTLSLTCTVSGFTFTDFYMNWVRQPPGRGLEWIGFIRDKAK |
| | GYTTEYNPSVKGRVTMLVDTSKNQFSLRLSSVTAADTAVYYCAREGHTAAPFDYWG |
| | QGSLVTVSS (SEQ ID NO: 420) |
| VL | DIQMTQSPSSLSASVGDRVTITCKASQNIDKYLNWYQQKPGKAPKLLIYNTNNLQT |
| | GVPSRFSGSGSGTDFTFTISSLQPEDIATYYCLQHISRPRTFGQGTKVEIKR |
| | (SEQ ID NO: 421) |

In some embodiments, a CD52 antibody comprises a VH having the sequence of SEQ ID NO: 420, or a sequence having at least 90%, at least 91%, at least 92%, at least 93%, at least 94%, at least 95%, at least 96%, at least 97%, at least 98%, or at least 99% sequence identity to SEQ ID NO: 420. In some embodiments, a CD52 antibody comprises a VL having the sequence of SEQ ID NO: 421, or a sequence having at least 90%, at least 91%, at least 92%, at least 93%, at least 94%, at least 95%, at least 96%, at least 97%, at least 98%, or at least 99% sequence identity to SEQ ID NO: 421. In some embodiments, a CD52 antibody comprises a VH having the sequence of SEQ ID NO: 420 and a VL having the sequence of SEQ ID NO: 421.

In some embodiments, a CD52 antibody comprises a HC having the sequence of SEQ ID NO: 408, or a sequence having at least 90%, at least 91%, at least 92%, at least 93%, at least 94%, at least 95%, at least 96%, at least 97%, at least 98%, or at least 99% sequence identity to SEQ ID NO: 408. In some embodiments, a CD52 antibody comprises a LC having the sequence of SEQ ID NO: 410, or a sequence having at least 90%, at least 91%, at least 92%, at least 93%, at least 94%, at least 95%, at least 96%, at least 97%, at least 98%, or at least 99% sequence identity to SEQ ID NO: 410. In some embodiments, a CD52 antibody comprises a HC having the sequence of SEQ ID NO: 408 and a LC having the sequence of SEQ ID NO: 410. In some embodiments, a CD52 antibody comprises a HC encoded by the DNA sequence of SEQ ID NO: 409 and a LC encoded by the DNA sequence of SEQ ID NO: 411.

In some embodiments, the anti-CD52 antibody is a recombinant humanized IgG1 kappa monoclonal antibody (mAb). In some embodiments, the anti-CD52 antibody is alemtuzumab. Alemtuzumab is a recombinant DNA-derived humanized monoclonal antibody directed against the 21-28 kD cell surface glycoprotein, CD52. See, e.g., Saif et al., Pediatr Transplant 2015 March; 19(2):211-8. In some embodiments, the anti-CD52 antibody comprises one or more CDR sequences isolated or derived from the CDRs of alemtuzumab. In some embodiments, the anti-CD52 antibody comprises the sequence of SEQ ID NO: 420, or a sequence having at least 90%, at least 91%, at least 92%, at least 93%, at least 94%, at least 95%, at least 96%, at least 97%, at least 98%, or at least 99% sequence identity to SEQ ID NO: 420. In some embodiments, the anti-CD52 antibody comprises the sequence of SEQ ID NO: 421, or a sequence having at least 90%, at least 91%, at least 92%, at least 93%, at least 94%, at least 95%, at least 96%, at least 97%, at least 98%, or at least 99% sequence identity to SEQ ID NO: 421. In some embodiments, the anti-CD52 antibody comprises the sequence of SEQ ID NO: 408, or a sequence having at least 90%, at least 91%, at least 92%, at least 93%, at least 94%, at least 95%, at least 96%, at least 97%, at least 98%, or at least 99% sequence identity to SEQ ID NO: 408. In some embodiments, the anti-CD52 antibody comprises the sequence of SEQ ID NO: 410, or a sequence having at least 90%, at least 91%, at least 92%, at least 93%, at least 94%, at least 95%, at least 96%, at least 97%, at least 98%, or at least 99% sequence identity to SEQ ID NO: 410. In some embodiments, the anti-CD52 antibody comprises an HCDR1 comprising the sequence of SEQ ID NO: 402, a HCDR2 comprising the sequence of SEQ ID NO: 403, a HCDR3 comprising the sequence of SEQ ID NO: 404, a LCDR1 comprising the sequence of SEQ ID NO: 405, a LCDR1 comprising the sequence of SEQ ID NO: 406, and/or a LCDR3 comprising the sequence of SEQ ID NO: 407. In some embodiments, the anti-CD52 antibody comprises an HCDR1 comprising the sequence of SEQ ID NO: 402, a HCDR2 comprising the sequence of SEQ ID NO: 403, a HCDR3 comprising the sequence of SEQ ID NO: 404, a LCDR1 comprising the sequence of SEQ ID NO: 405, a LCDR1 comprising the sequence of SEQ ID NO: 406, and a LCDR3 comprising the sequence of SEQ ID NO: 407; wherein the anti-CD52 antibody comprises the sequence of SEQ ID NO: 408 and/or SEQ ID NO: 410, or SEQ ID NO: 420 and/or SEQ ID NO: 421, or a sequence having at least 90%, at least 91%, at least 92%, at least 93%, at least 94%, at least 95%, at least 96%, at least 97%, at least 98%, or at least 99% sequence identity to SEQ ID NO: 408 and/or SEQ ID NO: 410, or SEQ ID NO: 420 and/or SEQ ID NO: 421.

In some embodiments, LD comprises administering only a CD52 antibody.

In some embodiments, LD comprises administration of a combination of therapies. In some embodiments, the combination includes: fludarabine (range total dose about 90 to 150 mg/m²) and cyclophosphamide (range total dose about 1000 to 4000 mg/m²), with or without an anti-CD52 drug (e.g., an anti-CD52 antibody such as an antibody comprising the sequence of SEQ ID NO: 408 and/or SEQ ID NO: 410, or SEQ ID NO: 420 and/or SEQ ID NO: 421) (total dose from about 0.3 to about 1 mg/kg, or a flat dose of from about 30 mg to about 40 mg, from about 25 to about 60 mg, from about 40 mg to about 60 mg, from about 60 mg to about 90 mg, or from about 100 mg to about 120 mg). In some embodiments, the combination includes: fludarabine (about 30 mg/m²) and cyclophosphamide (range total dose about 500 to 600 mg/m²), with or without an anti-CD52 drug (e.g., CD52 antibody) (total dose from about 0.3 to about 1 mg/kg, or a flat dose of about 13 to about 30 mg/day, about 13 mg/day, about 20 mg/day, or about 30 mg/day, or total dose from about 30 mg to about 40 mg, from about 25 to about 60 mg, from about 40 mg to about 60 mg, from about 60 mg to about 90 mg, or from about 100 mg to about 120 mg). In some embodiments, the combination includes: fludarabine (about 30 mg/m²) and cyclophosphamide (about 300 mg/m²), with or without an anti-CD52 drug (e.g., CD52 antibody) (from about 0.3 to about 1 mg/kg, or a flat dose of about 13 to about 30 mg/day, about 13 mg/day, about 20 mg/day, or about 30 mg/day, or total dose from about 30 mg to about 40 mg, from about 20 mg to about 30 mg, from about 25 mg to about 60 mg, from about 40 mg to about 60 mg, from about 60 mg to about 90 mg, or from about 100 mg to about 120 mg). In some embodiments, the combination includes: fludarabine (about 90 mg/m²) and cyclophosphamide (about 900 mg/m²), with or without an anti-CD52 drug (e.g., CD52 antibody) about 0.3 to about 1 mg/kg, or a flat dose of about 13 to about 30 mg/day, about 13 mg/day, about 20 mg/day, or about 30 mg/day, or total dose from about 30 mg to about 40 mg, from about 20 mg to about 30 mg, from about 25 mg to about 60 mg, from about 40 mg to about 60 mg, from about 60 mg to about 90 mg, or from about 100 mg to about 120 mg). In some embodiments, the combination includes: fludarabine (about 90 mg/m²), cyclophosphamide (about 1500 mg/m²) and with or without an anti-CD52 drug (e.g. anti-CD52 antibody, about 1 mg/kg). In some embodiments, the combination includes: fludarabine (about 150 mg/m²) and cyclophosphamide (about 130 mg/kg), with or without an anti-CD52 drug (e.g. anti-CD52 antibody, about 0.3 to about 1 mg/kg, or a flat dose of about 13 to about 30 mg/day, about 13 mg/day, about 20 mg/day, or about 30 mg/day, or total dose from about 30 mg to about 40 mg, from about 25 to about 60 mg, from about 40 mg to about 60 mg, from about 60 mg to about 90 mg, or from about 100 mg to about 120 mg). In some embodiments, the combination includes: fludarabine (about 150 g/m²) and cyclophosphamide (about 120 mg/kg or about 130 mg/kg), with or without an anti-CD52 drug (e.g. an anti-CD52 antibody), total dose from about 0.3 to about 1 mg/kg, or a flat dose of from about 30 mg to about 40 mg, from about 25 to about 60 mg, from about 40 mg to about 60 mg, from about 60 mg to about 90 mg, or from about 100 mg to about 120 mg). In some embodiments, the combination includes: fludarabine (about 30 mg/m²/day) and cyclophosphamide (about 300 mg/m²/day), with or without an anti-CD52 drug (e.g. an anti-CD52 antibody, about 13 mg/day). In some embodiments, the combination includes: fludarabine (about 30 mg/m²/day) and cyclophosphamide (about 300 mg/m²/day), with or without an anti-CD52 drug (e.g. an anti-CD52 antibody, about 10 mg/day). In some embodiments, the combination includes: cyclophosphamide and an anti-CD52 drug (e.g. an anti-CD52 antibody). In some embodiments, these above doses are administered during the course of one day. In some embodiments, these above doses are administered over multiple days.

In some embodiments, fludarabine and cyclophosphamide are administered on a first day, and the anti-CD52 antibody (e.g., an antibody comprising the sequence of SEQ ID NO: 408 and/or SEQ ID NO: 410, or SEQ ID NO: 420 and/or SEQ ID NO: 421) is administered on a second day. In some embodiments, fludarabine and cyclophosphamide are administered on a first day before administration of the CAR-T cells, and an anti-CD52 antibody (e.g., an antibody comprising the sequence of SEQ ID NO: 408 and/or SEQ ID NO: 410, or SEQ ID NO: 420 and/or SEQ ID NO: 421) is administered on a second day; wherein the second day is the same day that CAR-T cells are administered or the second day is after the CAR-T cells are administered. In some embodiments, fludarabine and cyclophosphamide are administered on a first day, CAR-T cells are administered on a second day, and an anti-CD52 antibody (e.g., an antibody comprising the sequence of SEQ ID NO: 408 and/or SEQ ID NO: 410, or SEQ ID NO: 420 and/or SEQ ID NO: 421) is administered at least about 1, about 2, about 3, about 4, about 5, about 6, about 7, about 8, about 9, about 10, about 11, or about 12 weeks after the second day. In some embodiments, fludarabine and cyclophosphamide are administered before administration of CAR-T cells, and an anti-CD52 antibody (e.g., an antibody comprising the sequence of SEQ ID NO: 408 and/or SEQ ID NO: 410, or SEQ ID NO: 420 and/or SEQ ID NO: 421) is administered at least about 1, about 2, about 3, about 4, about 5, about 6, about 7, about 8, about 9, about 10, about 11, or about 12 weeks after administration of the CAR-T cells.

In some embodiments, a lymphodepletion regimen comprises administration of fludarabine and cyclophosphamide (FC). In some embodiments, a lymphodepletion regimen comprises administration of fludarabine and anti-CD52 antibody (e.g., an antibody comprising the sequence of SEQ ID NO: 408 and/or SEQ ID NO: 410, or SEQ ID NO: 420 and/or SEQ ID NO: 421)(FA). In some embodiments, a lymphodepletion regimen comprises administration of cyclophosphamide and an anti-CD52 antibody (e.g., an antibody comprising the sequence of SEQ ID NO: 408 and/or SEQ ID NO: 410, or SEQ ID NO: 420 and/or SEQ ID NO: 421) (CA). In some embodiments, a lymphodepletion regimen comprises administration of fludarabine, cyclophosphamide, and an anti-CD52 antibody (e.g., an antibody comprising the sequence of SEQ ID NO: 408 and/or SEQ ID NO: 410, or SEQ ID NO: 420 and/or SEQ ID NO: 421) (FCA).

The choice of specific lymphodepletion regimen drugs and dose before a first or second/subsequent dose of CAR-T cells may be determined based on hematological analysis and hematologic recovery of the patient. In the case of redosing, a second lymphodepletion regimen can be more or less intense compared to a first lymphodepletion regimen (for example, based on recovery of lymphocytes, neutrophils, and viral reactivation after a first dose). For example, at the time of redosing, if lymphocyte and neutrophil levels are high, a strong or aggressive lymphodepletion regimen may be used. Alternatively, at the time of redosing, if lymphocyte levels are low, a weaker or less aggressive lymphodepletion regimen may be used. In some embodiments, if the number of blasts at the time of redosing is high, a strong or aggressive lymphodepletion regimen is used. In some embodiments, if the number of blasts at the time of redosing is low, a weaker or less aggressive lymphodepletion regimen is used.

In some embodiments, an increased intensity of LD regimen may be applied at the time of redosing (with or without anti-CD52 drug). In some embodiments, a reduced intensity of LD regimen may be applied, for example, in case of grade 3-4 lymphopenia at time of redosing (with or without anti-CD52 drug).

In some embodiments, the components of the lymphodepletion regimen of fludarabine/cyclophosphamide (FC) or fludarabine/cyclophosphamide/anti-CD52 antibody (FCA) are administered simultaneously; in other embodiments, the components are administered serially. In some embodiments, the components of the lymphodepletion regimen of fludarabine/cyclophosphamide (FC) or fludarabine/cyclophosphamide/anti-CD52 antibody (FCA) are administered simultaneously on Day −5, Day −4 and Day −3. In some embodiments, the components of the lymphodepletion regimen of fludarabine/cyclophosphamide (FC) are administered prior to the administration of the anti-CD52 antibody. In some embodiments, the fludarabine/cyclophosphamide (FC) are administered on Day −7, Day −6 and Day −5, followed by the administration of the anti-CD52 antibody (A) on Day −4 and Day −3. In some embodiments, the fludarabine/cyclophosphamide (FC) are administered on Day −7, Day −6 and Day −5, followed by the administration of the anti-CD52 antibody (A) on Day −5, Day −4 and Day −3. In some embodiments, the subject receives a FC regimen prior to the first dose of the CAR-T cell therapy; and a FCA regimen prior to a redosing of the CAR-T cell therapy. In some embodiments, the subject receives a FCA regimen prior to the first dose of the CAR-T cell therapy; and a second FCA regimen prior to a redosing of the CAR-T cell therapy.

Exemplary LD regimens are provided in Tables 6A, 6B, 6C, 6D, 6E, 6F, 6G, and 6H. In Tables 6A-6H, the timing indicated under Schedule is relative to the timing of administration of a dose of CAR-T cells (DO), in days. Negative numbers indicate days prior to administration of CAR-T cells (at DO).

TABLE 6A

| Lymphodepletion | Dose | Total dose |
|---|---|---|
| Fludarabine | 30 mg/m²/day | 90 mg/m² |
| Cyclophosphamide | 500 mg/m²/day | 1500 mg/m² |
| Anti-CD52 antibody | 0.2 mg/kg/day | 1 mg/kg |
| (optional) | | |

TABLE 6B

| Lymphodepletion | Dose | Route |
|---|---|---|
| Fludarabine | 30 mg/m²/day | IV over 15-30 min |
| Cyclophosphamide | 500 mg/m²/day | IV over 1 hour |
| Anti-CD52 antibody | 8 mg/day | IV |

TABLE 6C

| Lymphodepletion | Dose | Route |
|---|---|---|
| Fludarabine | 30 mg/m²/day | IV over 15-30 min |
| Cyclophosphamide | 500 mg/m²/day | IV over 1 hour |
| Anti-CD52 antibody | 6 mg/day | IV |

TABLE 6D

| Lymphodepletion | Dose | Total dose |
|---|---|---|
| Fludarabine | 30 mg/m²/day | 90 mg/m² |
| Cyclophosphamide | 300 mg/m²/day | 900 mg/m² |
| Anti-CD52 antibody | 13 mg/day | 39 mg |
| (optional) | | |

TABLE 6E

| Lymphodepletion | Dose | Total dose |
|---|---|---|
| Fludarabine | 30 mg/m²/day | 90 mg/m² |
| Cyclophosphamide | 300 mg/m²/day | 900 mg/m² |
| Anti-CD52 antibody | 10 mg/day | 30 mg |
| (optional) | | |

TABLE 6F

| Lymphodepletion | Dose | Total dose |
|---|---|---|
| Fludarabine | 30 mg/m²/day | 90 mg/m² |
| Cyclophosphamide | 300 mg/m²/day | 900 mg/m² |
| Anti-CD52 antibody | 30 mg/day | 90 mg |

TABLE 6G

| Lymphodepletion | Dose | Total dose |
|---|---|---|
| Fludarabine | 30 mg/m²/day | 90 mg/m² |
| Cyclophosphamide | 300 mg/m²/day | 900 mg/m² |
| Anti-CD52 antibody | 30 mg/day | 60 mg |

TABLE 6H

| Lymphodepletion | Dose | Total dose |
|---|---|---|
| Fludarabine | 30 mg/m²/day | 90 mg/m² |
| Cyclophosphamide | 300 mg/m²/day | 900 mg/m² |
| Anti-CD52 antibody | 20 mg/day | 60 mg |

VI. Dosing Regimens

In some embodiments, allogeneic BCMA CAR-T cells and/or gamma secretase inhibitors of the disclosure are administered using a flat dose. In other embodiments, allogeneic BCMA CAR-T cells and/or gamma secretase (e.g., nirogacestat or a pharmaceutically acceptable salt thereof) are administered using dose-banding. For example, dose-banding may be used to avoid the risk of a wide range of CAR-T cell exposure. In some embodiments, a weight band may be used. For example, without limitation, subjects <66 kg may be administered X dose, and subjects ≥66 kg may be administered about 1.33× dose. In some embodiments, subjects ≥50 kg may be administered one dose, and subjects ≤50 kg may be administered a different dose.

Exemplary dose levels for a first dose of allogeneic BCMA CAR-T cells are provided in Table 7A, for use in subjects with relapsed/refractory MM. The dose level designated as “−1” is administered only as needed.

TABLE 7A

| | Dose (×10\hat{ }6 viable | Dose (×10\hat{ }6 viable |
|---|---|---|
| | CAR-T cells) | CAR-T cells) for |
| | for subject whose | subject whose |
| Dose Level | weight ≥50 kg | weight <50 kg |
| 1 (starting) | 40 | 20 |
| 2 | 120 | 80 |
| 3 | 360 | 240 |
| 4 | 480 | 360 |
| −1 | 20 | 7 or 14 |

In some embodiments, a subject whose weight is ≥50 kg is administered a dose of allogeneic BCMA CAR-T cells of the disclosure, wherein the dose ranges from about 20×10\hat{ }6 cells/dose to about 480×10\hat{ }6 cells/dose. In some embodiments, the BCMA CAR-T cells are BCMA-CAR⁺_TCRαβ⁻_CD52^{+/-} T-cells. In some embodiments, the BCMA CAR-T cells are BCMA-1 CAR-T cells (described in Example 1).

In some embodiments, a subject whose weight is ≥50 kg is administered a dose of allogeneic BCMA CAR-T cells of the disclosure, wherein the dose ranges from about 20×10\hat{ }6 cells/dose to about 40×10\hat{ }6 cells/dose, from about 40×10\hat{ }6 cells/dose to about 120×10\hat{ }6 cells/dose, from about 120×10\hat{ }6 cells/dose to about 360×10\hat{ }6 cells/dose, or from about 360×10\hat{ }6 cells/dose to about 480×10\hat{ }6 cells/dose. In some embodiments, the BCMA CAR-T cells are BCMA-CAR⁺_TCRαβ⁻_CD52^{+/-} T-cells. In some embodiments, the BCMA CAR-T cells are BCMA-1 CAR-T cells.

In some embodiments, a subject whose weight is <50 kg is administered a dose of allogeneic BCMA CAR-T cells of the disclosure, wherein the dose ranges from about 7×10\hat{ }6 cells/dose to about 360×10\hat{ }6 cells/dose. In some embodiments, the BCMA CAR-T cells are BCMA-CAR⁺_TCRαβ⁻_CD52^{+/-} T-cells. In some embodiments, the BCMA CAR-T cells are BCMA-1 CAR-T cells.

In some embodiments, a subject whose weight is <50 kg is administered a dose of allogeneic BCMA CAR-T cells of the disclosure, wherein the dose ranges from about 7×10\hat{ }6 or 14×10\hat{ }6 cells/dose to about 20×10\hat{ }6 cells/dose, from about 20×10\hat{ }6 cells/dose to about 80×10\hat{ }6 cells/dose, from about 80×10\hat{ }6 cells/dose to about 240×10\hat{ }6 cells/dose, or from about 240×10\hat{ }6 cells/dose to about 360×10\hat{ }6 cells/dose. In some embodiments, the BCMA CAR-T cells are BCMA-CAR⁺_TCRαβ⁻_CD52^{+/-} T-cells. In some embodiments, the BCMA CAR-T cells are BCMA-1 CAR-T cells.

Alternative exemplary dose levels for a first dose of allogeneic BCMA CAR-T cells are provided in Table 7B, for use in subjects with relapsed/refractory MM. The Intermediate dose level, and the dose levels designated as “4” and “−1” are administered only as needed.

TABLE 7B

| | Dose (×10\hat{ }6 viable | Dose (×10\hat{ }6 viable |
|---|---|---|
| | CAR-T cells) | CAR-T cells) |
| | for subject whose | for subject whose |
| Dose Level | weight >50 kg | weight ≤50 kg |
| 1 (starting) | 40 | 20 |
| 2 | 160 | 80 |
| 3 | 320 | 200 |
| Intermediate | 240 | 160 |
| 4 | 480 | 320 |
| −1 | 20 | 14 |

In some embodiments, a subject whose weight is >50 kg is administered a dose of allogeneic BCMA CAR-T cells of the disclosure, wherein the dose ranges from about 20×10\hat{ }6 cells/dose to about 480×10\hat{ }6 cells/dose. In some embodiments, the BCMA CAR-T cells are BCMA-CAR⁺_TCRαβ⁻_CD52^{+/-} T-cells. In some embodiments, the BCMA CAR-T cells are BCMA-1 CAR-T cells (described in Example 1).

In some embodiments, a subject whose weight is >50 kg is administered a dose of allogeneic BCMA CAR-T cells of the disclosure, wherein the dose ranges from about 20×10\hat{ }6 cells/dose to about 40×10\hat{ }6 cells/dose, from about 40×10\hat{ }6 cells/dose to about 160×10\hat{ }6 cells/dose, from about 160×10\hat{ }6 cells/dose to about 320×10\hat{ }6 cells/dose, from about 160×10\hat{ }6 cells/dose to about 240×10\hat{ }6 cells/dose, from about 240×10\hat{ }6 cells/dose to about 320×10\hat{ }6 cells/dose, from about 240×10\hat{ }6 cells/dose to about 480×10\hat{ }6 cells/dose, or from about 320×10\hat{ }6 cells/dose to about 480×10\hat{ }6 cells/dose. In some embodiments, the BCMA CAR-T cells are BCMA-1 CAR-T cells. In some embodiments, the BCMA CAR-T cells are BCMA-CAR⁺_TCRαβ⁻_CD52^{+/-} T-cells.

In some embodiments, a subject whose weight is ≤50 kg is administered a dose of allogeneic BCMA CAR-T cells of the disclosure, wherein the dose ranges from about 14×10\hat{ }6 cells/dose to about 320×10\hat{ }6 cells/dose. In some embodiments, the BCMA CAR-T cells are BCMA-CAR⁺_TCRαβ⁻_CD52^{+/-} T-cells. In some embodiments, the BCMA CAR-T cells are BCMA-1 CAR-T cells.

In some embodiments, a subject whose weight is ≤50 kg is administered a dose of allogeneic BCMA CAR-T cells of the disclosure, wherein the dose ranges from about 14×10\hat{ }6 cells/dose to about 20×10\hat{ }6 cells/dose, from about 20×10\hat{ }6 cells/dose to about 80×10\hat{ }6 cells/dose, from about 80×10\hat{ }6 cells/dose to about 160×10\hat{ }6 cells/dose, from about 80×10\hat{ }6 cells/dose to about 200×10\hat{ }6 cells/dose, from about 160×10\hat{ }6 cells/dose to about 200×10\hat{ }6 cells/dose, from about 200×10\hat{ }6 cells/dose to about 320×10\hat{ }6 cells/dose, from about 160×10\hat{ }6 cells/dose to about 320×10\hat{ }6 cells/dose or from about 200×10\hat{ }6 cells/dose to about 320×10\hat{ }6 cells/dose. In some embodiments, the BCMA CAR-T cells are BCMA-CAR⁺_TCRαβ⁻_CD52^{+/-} T-cells. In some embodiments, the BCMA CAR-T cells are BCMA-1 CAR-T cells.

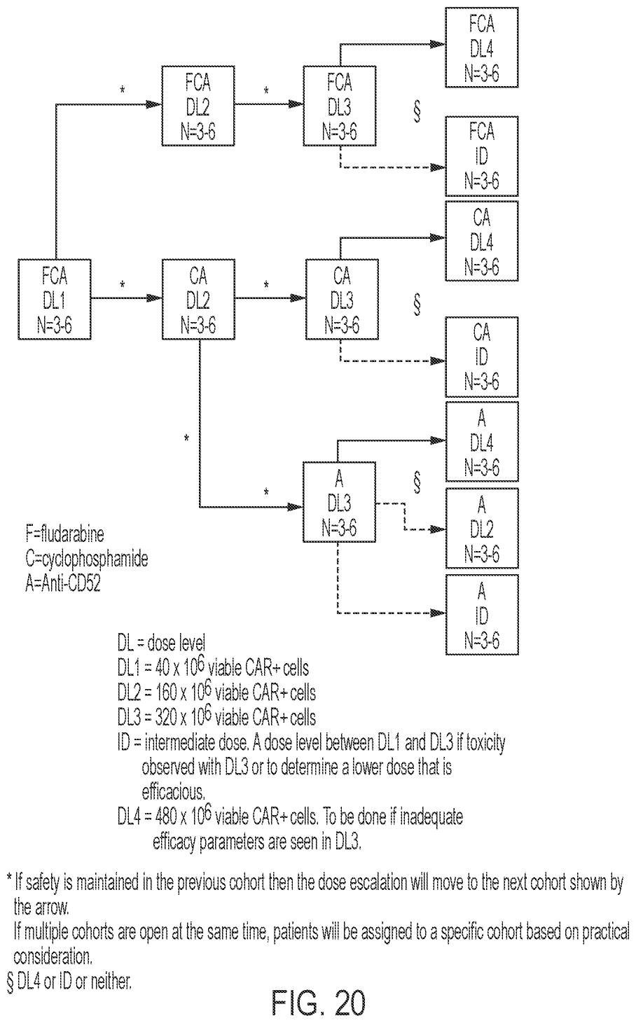

In some embodiments, a subject whose weight is >50 kg is administered a dose of allogeneic BCMA CAR-T cells of the disclosure, wherein the dose is about 40×10\hat{ }6 cells/dose, 160×10\hat{ }6 cells/dose, or 320×10\hat{ }6 cells/dose. In some embodiments, an intermediate dose of about 240×10\hat{ }6 cells/dose is administered (or another dose level between Dose Level 1 or Dose Level 3) if toxicity is observed with Dose level 3, or to determine a lower dose that is efficacious. In some embodiments, a dose level of 480×10\hat{ }6 cells/dose is administered (Dose level 4) if inadequate efficacy parameters are seen in Dose level 3. (FIG. 20). In some embodiments, the BCMA CAR-T cells are BCMA-CAR⁺_TCRαβ⁻_CD52^{+/-} T-cells. In some embodiments, the BCMA CAR-T cells are BCMA-1 CAR-T cells.

Further exemplary dose levels for a first dose of BCMA CAR-T cells of the disclosure are provided in Table 7C (phase I, Design C), for use in subjects with relapsed/refractory MM.

TABLE 7C

| | Dose (×10⁶ viable |
|---|---|
| Dose Level | CAR T cells) |
| 1 (starting) | 40 |
| 2 | 160 |
| 3 | 320 |
| Intermediate^{a} | 240 |
| 4^{a} | 480 |
| −1^{a} | 20 |

^{a}These dose levels will be administered as needed

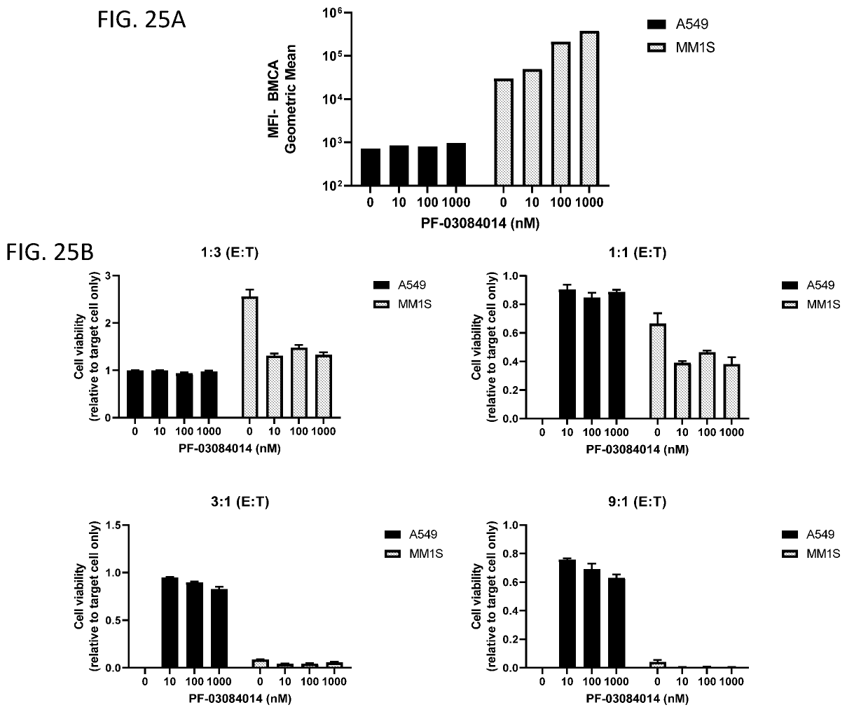

In some embodiments, a subject is administered a dose of allogeneic BCMA CAR-T cells of the disclosure, wherein the dose is about 40×10\hat{ }6 cells/dose, 160×10\hat{ }6 cells/dose, or 320×10\hat{ }6 cells/dose. In some embodiments, an intermediate dose of about 240×10\hat{ }6 cells/dose is administered (or another dose level between Dose Level 1 or Dose Level 3) if toxicity is observed with Dose level 3, or to determine a lower dose that is efficacious. In some embodiments, a dose level of 480×10\hat{ }6 cells/dose is administered (Dose level 4) if inadequate efficacy parameters are seen in Dose level 3. (FIG. 25A). In some embodiments, the BCMA CAR-T cells are BCMA-CAR⁺_TCRαβ⁻_CD52^{+/-} T-cells. In some embodiments, the BCMA CAR-T cells are BCMA-1.

The cells or population of cells can be administrated in one or more doses. In some embodiments, said effective amount of cells can be administrated as a single dose. In some embodiments, said effective amount of cells can be administrated as more than one dose over a period time. Timing of administration is within the judgment of managing physician and depends on the clinical condition of the subject. The cells or population of cells may be obtained from any source, such as a blood bank or a donor. While individual needs vary, determination of optimal ranges of effective amounts of a given cell type for a particular disease or conditions within the skill of the art. An effective amount means an amount which provides a therapeutic or prophylactic benefit. The dosage administrated will generally be dependent upon the age, health and weight of the recipient, kind of concurrent treatment, if any, frequency of treatment and the nature of the effect desired. In some embodiments, an effective amount of cells or composition comprising those cells are administrated parenterally. In some embodiments, administration can be an intravenous administration. In some embodiments, administration can be directly done by injection within a tumor.

In some embodiments, a gamma secretase inhibitor (e.g., nirogacestat or a pharmaceutically acceptable salt thereof) of the current disclosure is administered as a flat dose. In some embodiments, the gamma secretase inhibitor (e.g., nirogacestat or a pharmaceutically acceptable salt thereof) is administered at a dose of about 20 mg to about 220 mg once or twice daily. In some embodiments, the gamma secretase inhibitor (e.g., nirogacestat or a pharmaceutically acceptable salt thereof) is administered at a dose of about 20 mg to about 220 mg once or twice daily for at least one week, at least two weeks, at least three weeks, at least four weeks, at least five weeks, at least six weeks, at least seven weeks, at least eight weeks, at least nine weeks or at least ten weeks.

In some embodiments, the gamma secretase inhibitor (e.g., nirogacestat or a pharmaceutically acceptable salt thereof) is administered at a dose of about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, or about 220 mg once or twice daily from Day 0 to Day 10, Day 0 to Day 15, Day 0 to Day 20, Day 0 to Day 21, Day 0 to Day 22, Day 0 to Day 23, Day 0 to Day 24, Day 0 to Day 25, Day 0 to Day 26, Day 0 to Day 27, Day 0 to Day 28, Day 0 to Day 29, Day 0 to Day 30, Day 0 to Day 31, Day 0 to Day 32, Day 0 to Day 33, Day 0 to Day 34, Day 0 to Day 35, Day 0 to Day 36, Day 0 to Day 37, Day 0 to Day 38, Day 39, Day 0 to Day 40, Day 0 to Day 41, or Day 0 to Day 42. In some embodiments, the gamma secretase inhibitor (e.g., nirogacestat or a pharmaceutically acceptable salt thereof) is administered at a dose of about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, about 150 mg, about 160 mg, about 170 mg, about 180 mg, about 190 mg, about 200 mg, about 210 mg, or about 220 mg once or twice daily on Day 0 (before BCMA CAR T administration) through Day 41.

In some embodiments, the gamma secretase inhibitor (e.g., nirogacestat or a pharmaceutically acceptable salt thereof) is administered at a dose of about 100 mg taken by mouth once or twice daily. In some embodiments, the gamma secretase inhibitor (e.g., nirogacestat or a pharmaceutically acceptable salt thereof) is administered at a dose of about 100 mg taken by mouth twice daily. In some embodiments, the gamma secretase inhibitor (e.g., nirogacestat or a pharmaceutically acceptable salt thereof) is administered at a dose of about 100 mg once or twice daily from Day 0 to Day 10, Day 0 to Day 15, Day 0 to Day 20, Day 0 to Day 21, Day 0 to Day 22, Day 0 to Day 23, Day 0 to Day 24, Day 0 to Day 25, Day 0 to Day 26, Day 0 to Day 27, Day 0 to Day 28, Day 0 to Day 29, Day 0 to Day 30, Day 0 to Day 31, Day 0 to Day 32, Day 0 to Day 33, Day 0 to Day 34, Day 0 to Day 35, Day 0 to Day 36, Day 0 to Day 37, Day 0 to Day 38, Day 39, Day 0 to Day 40, Day 0 to Day 41, or Day 0 to Day 42. In some embodiments, the gamma secretase inhibitor (e.g., nirogacestat or a pharmaceutically acceptable salt thereof) is administered on Day 0 (before BCMA CAR T administration) through Day 41.

In some embodiments, the gamma secretase inhibitor (e.g., nirogacestat or a pharmaceutically acceptable salt thereof) is administered to the subject before, concomitantly, or subsequently to the administering of the at least one dose of BCMA CAR-T cells to the subject. The gamma secretase inhibitor (e.g., nirogacestat or a pharmaceutically acceptable salt thereof) can be administered in the same or different dosage forms.

In some embodiments, of the disclosure, a subject receiving the BCMA CAR-T cells/gamma secretase inhibitor (e.g., nirogacestat or a pharmaceutically acceptable salt thereof) combination therapies may be administered in conjunction with (e.g., before, simultaneously or following) any additional number of relevant treatment modalities, including but not limited to treatment with agents such as monoclonal antibody therapy, CCR2 antagonist (e.g., INC-8761), antiviral therapy, cidofovir and interleukin-2, Cytarabine (also known as ARA-C) or nataliziimab treatment for MS subjects or efaliztimab treatment for psoriasis subjects or other treatments for PML subjects. In some embodiments, BCMA specific CAR-T cells/gamma secretase inhibitor (e.g., nirogacestat or a pharmaceutically acceptable salt thereof) are administered to a subject in conjunction with one or more of the following: an anti-PD-1 antibody (e.g., nivolumab, pembrolizumab, or PF-06801591), an anti-PD-L1 antibody (e.g., avelumab, atezolizumab, or durvalumab), an anti-OX40 antibody (e.g., PF-04518600), an anti-4-1BB antibody (e.g., PF-05082566), an anti-MCSF antibody (e.g., PD-0360324), an anti-GITR antibody, and/or an anti-TIGIT antibody. In some embodiments, the BCMA CAR-T cells/gamma secretase inhibitor (e.g., nirogacestat or a pharmaceutically acceptable salt thereof) combination therapies of the disclosure is administered to a subject in conjunction with anti-PD-L1 antibody avelumab. In further embodiments, the BCMA CAR-T cells/gamma secretase inhibitor (e.g., nirogacestat or a pharmaceutically acceptable salt thereof) combination therapies may be used in combination with chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludaribine, cyclosporin, FK506, rapamycin, mycoplienolic acid, steroids, FR901228, cytokines, and/or irradiation. These drugs inhibit either the calcium dependent phosphatase calcineurin (cyclosporine and FK506) or inhibit the p70S6 kinase that is important for growth factor induced signaling (rapamycin) (Henderson, Naya et al. 1991; Liu, Albers et al. 1992; Bierer, Hollander et al. 1993). In a further embodiment, the BCMA CAR-T cells/gamma secretase inhibitor (e.g., nirogacestat or a pharmaceutically acceptable salt thereof) combination therapies of the disclosure are administered to a subject in conjunction with (e.g., before, simultaneously or following) bone marrow transplantation, T cell ablative therapy using either chemotherapy agents such as, fludarabine, external-beam radiation therapy (XRT), cyclophosphamide, or antibodies such as OKT3 or CAMPATH, In some embodiments, the cell compositions of the disclosure are administered following B-cell ablative therapy such as agents that react with CD20, e.g., Rituxan. For example, in one embodiment, subjects may undergo standard treatment with high dose chemotherapy followed by peripheral blood stem cell transplantation. In certain embodiments, following the transplant, subjects receive an infusion of the expanded immune cells of the disclosure. In some embodiments, expanded cells are administered before or following surgery.

VII. Methods of Retreatment with CAR-T Cells

Also provided herein are methods for retreatment (redosing) with BCMA CAR-T cells. In particular, the methods involve administering one or more subsequent doses of cells to subjects having received a first dose, and/or administering the first and one or more subsequent doses. The doses generally are administered in particular amounts and according to particular timing parameters. In some embodiments, the methods generally involve administering a first dose of cells, thereby reducing disease burden, followed by a subsequent dose of cells, administered during a particular time window with respect to the first dose, or the administration of the subsequent dose to a subject having received such a first dose. In some embodiments, additional subsequent doses then are administered, for example, within the same or a similar window of time with respect to the subsequent dose. In some embodiments, the number of cells administered and timing of the multiple doses are designed to improve one or more outcomes, such as to reduce the likelihood or degree of toxicity to the subject, improve exposure of the subject to and/or persistence of the administered cells, and/or improve therapeutic efficacy. Also provided are articles of manufacture containing the cells and designed for administration following such dosing regimens.

In some retreatment (redosing) embodiments, a subject whose weight is >50 kg is administered a re-dose of allogeneic BCMA CAR-T cells of the disclosure, wherein the dose ranges from about 20×10\hat{ }6 cells/dose to about 480×10\hat{ }6 cells/dose. In some embodiments, the BCMA CAR-T cells are BCMA-CAR⁺_TCRαβ⁻_CD52^{+/-} T-cells. In some embodiments, the BCMA CAR-T cells are BCMA-1 CAR-T cells (described in Example 1).

In some retreatment (redosing) embodiments, a subject whose weight is >50 kg is administered a re-dose of allogeneic BCMA CAR-T cells of the disclosure, wherein the dose ranges from about 20×10\hat{ }6 cells/dose to about 40×10\hat{ }6 cells/dose, from about 40×10\hat{ }6 cells/dose to about 160×10\hat{ }6 cells/dose, from about 160×10\hat{ }6 cells/dose to about 320×10\hat{ }6 cells/dose, from about 160×10\hat{ }6 cells/dose to about 240×10\hat{ }6 cells/dose, from about 240×10\hat{ }6 cells/dose to about 320×10\hat{ }6 cells/dose, from about 240×10\hat{ }6 cells/dose to about 480×10\hat{ }6 cells/dose, or from about 320×10\hat{ }6 cells/dose to about 480×10\hat{ }6 cells/dose. In some embodiments, the BCMA CAR-T cells are BCMA-1 CAR-T cells. In some embodiments, the BCMA CAR-T cells are BCMA-CAR⁺_TCRαβ⁻_CD52^{+/-} T-cells.

In some retreatment (redosing) embodiments, a subject whose weight is >50 kg is administered a re-dose of allogeneic BCMA CAR-T cells of the disclosure, wherein the dose is about 40×10\hat{ }6 cells/dose, 160×10\hat{ }6 cells/dose, or 320×10\hat{ }6 cells/dose. In some embodiments, an intermediate dose of about 240×10\hat{ }6 cells/dose is administered (or another dose level between Dose Level 1 or Dose Level 3) if toxicity is observed with Dose level 3, or to determine a lower does that is efficacious. In some embodiments, a dose level of 480×10\hat{ }6 cells/dose is administered (Dose level 4) if inadequate efficacy parameters are seen in Dose level 3. (FIG. 20). In some embodiments, the BCMA CAR-T cells are BCMA-CAR⁺_TCRαβ⁻_CD52^{+/-} T-cells. In some embodiments, the BCMA CAR-T cells are BCMA-1 CAR-T cells.

In some retreatment (redosing) embodiments, a subject whose weight is ≥50 kg is administered a re-dose of allogeneic BCMA CAR-T cells of the disclosure, wherein the dose ranges from about 20×10\hat{ }6 cells/dose to about 480×10\hat{ }6 cells/dose. In some embodiments, the BCMA CAR-T cells are BCMA-CAR⁺_TCRαβ⁻_CD52^{+/-} T-cells. In some embodiments, the BCMA CAR-T cells are BCMA-1 CAR-T cells (described in Example 1).

In some retreatment (redosing) embodiments, a subject whose weight is ≥50 kg is administered a re-dose of allogeneic BCMA CAR-T cells of the disclosure, wherein the dose ranges from about 20×10\hat{ }6 cells/dose to about 40×10\hat{ }6 cells/dose, from about 40×10\hat{ }6 cells/dose to about 120×10\hat{ }6 cells/dose, from about 120×10\hat{ }6 cells/dose to about 360×10\hat{ }6 cells/dose, or from about 360×10\hat{ }6 cells/dose to about 480×10\hat{ }6 cells/dose. In some embodiments, the BCMA CAR-T cells are BCMA-CAR⁺_TCRαβ⁻_CD52^{+/-} T-cells. In some embodiments, the BCMA CAR-T cells are BCMA-1 CAR-T cells.

In some retreatment (redosing) embodiments, a subject whose weight is ≤50 kg is administered a re-dose of allogeneic BCMA CAR-T cells of the disclosure, wherein the dose ranges from about 14×10\hat{ }6 cells/dose to about 320×10\hat{ }6 cells/dose. In some embodiments, the BCMA CAR-T cells are BCMA-CAR⁺_TCRαβ⁻_CD52^{+/-} T-cells. In some embodiments, the BCMA CAR-T cells are BCMA-1 CAR-T cells.

In some retreatment (redosing) embodiments, a subject whose weight is ≤50 kg is administered a re-dose of allogeneic BCMA CAR-T cells of the disclosure, wherein the dose ranges from about 14×10\hat{ }6 cells/dose to about 20×10\hat{ }6 cells/dose, from about 20×10\hat{ }6 cells/dose to about 80×10\hat{ }6 cells/dose, from about 80×10\hat{ }6 cells/dose to about 160×10\hat{ }6 cells/dose, from about 80×10\hat{ }6 cells/dose to about 200×10\hat{ }6 cells/dose, from about 160×10\hat{ }6 cells/dose to about 200×10\hat{ }6 cells/dose, from about 200×10\hat{ }6 cells/dose to about 320×10\hat{ }6 cells/dose, from about 160×10\hat{ }6 cells/dose to about 320×10\hat{ }6 cells/dose or from about 200×10\hat{ }6 cells/dose to about 320×10\hat{ }6 cells/dose. In some embodiments, the BCMA CAR-T cells are BCMA-CAR⁺_TCRαβ⁻_CD52^{+/-} T-cells. In some embodiments, the BCMA CAR-T cells are BCMA-1 CAR-T cells.

In some retreatment (redosing) embodiments, a subject whose weight is <50 kg is administered a re-dose of allogeneic BCMA CAR-T cells of the disclosure, wherein the dose ranges from about 7×10\hat{ }6 cells/dose to about 360×10\hat{ }6 cells/dose. In some embodiments, the BCMA CAR-T cells are BCMA-CAR⁺_TCRαβ⁻_CD52^{+/-} T-cells. In some embodiments, the BCMA CAR-T cells are BCMA-1 CAR-T cells.

In some retreatment (redosing) embodiments, a subject whose weight is <50 kg is administered a re-dose of allogeneic BCMA CAR-T cells of the disclosure, wherein the dose ranges from about 7×10\hat{ }6 or 14×10\hat{ }6 cells/dose to about 20×10\hat{ }6 cells/dose, from about 20×10\hat{ }6 cells/dose to about 80×10\hat{ }6 cells/dose, from about 80×10\hat{ }6 cells/dose to about 240×10\hat{ }6 cells/dose, or from about 240×10\hat{ }6 cells/dose to about 360×10\hat{ }6 cells/dose. In some embodiments, the BCMA CAR-T cells are BCMA-CAR⁺_TCRαβ⁻_CD52^{+/-} T-cells. In some embodiments, the BCMA CAR-T cells are BCMA-1 CAR-T cells.

VIII. Kits and Articles of Manufacture

The disclosure also provides kits and articles of manufacture for use in the disclosed methods.

Kits of the disclosure include one or more containers (e.g. glass vials) comprising a polynucleotide encoding a BCMA specific CAR, or an engineered immune cell comprising a polynucleotide encoding a BCMA specific CAR as described herein (e.g. BCMA-1 CAR-T cells, e.g. BCMA-CAR⁺_TCRαβ⁻_CD52^{+/-} T-cells), a gamma secretase inhibitor (e.g., nirogacestat or a pharmaceutically acceptable salt thereof) and instructions for use in accordance with any of the methods of the disclosure described herein. In some embodiments, the engineered immune cells are formulated in a solution comprising about 5% DMSO.

Further, the engineered immune cells can be provided in a frozen state. In some embodiments, the gamma secretase inhibitor is Compound I, or a pharmaceutically acceptable salt thereof. In some embodiments, the gamma secretase inhibitor is nirogacestat hydrobromide. In some embodiments, the gamma secretase inhibitor is nirogacestat dihydrobromide.

In some embodiments, provided herein are additional vials comprising unit doses of a CD52 antibody (which can be provided in a frozen state or as a room temperature solution comprising a buffered medium), fludarabine, and/or cyclophosphamide.

Generally, these instructions provided herein comprise a description of administration of the engineered immune cell for the above described therapeutic treatments. The instructions relating to the use of the engineered immune cells as described herein generally include information as to dosage, dosing schedule, and route of administration for the intended treatment. The containers may be unit doses, bulk packages (e.g., multi-dose packages) or sub-unit doses. Instructions supplied in the kits of the disclosure are typically written instructions on a label or package insert (e.g., a paper sheet included in the kit), but machine-readable instructions (e.g., instructions carried on a magnetic or optical storage disk) are also acceptable.

The kits of this disclosure are in suitable packaging. Suitable packaging includes, but is not limited to, vials, bottles, jars, flexible packaging (e.g., sealed Mylar or plastic bags), and the like. Also contemplated are packages for use in combination with a specific device, such as an infusion device such as a minipump. A kit may have a sterile access port (for example the container may be an intravenous solution bag or a vial having a stopper pierceable by a hypodermic injection needle). The container may also have a sterile access port (for example, the container may be an intravenous solution bag or a vial having a stopper pierceable by a hypodermic injection needle). At least one active agent in the composition is a BCMA antibody. The container may further comprise a second pharmaceutically active agent.

Kits may optionally provide additional components such as buffers and interpretive information. Normally, the kit comprises a container and a label or package insert(s) on or associated with the container.

The following examples are offered for illustrative purposes only, and are not intended to limit the scope of the disclosure in any way. Indeed, various modifications of the disclosure in addition to those shown and described herein will become apparent to those skilled in the art from the foregoing description and fall within the scope of the appended claims.

### EXAMPLES

### Example 1: Production and Testing of BCMA-1

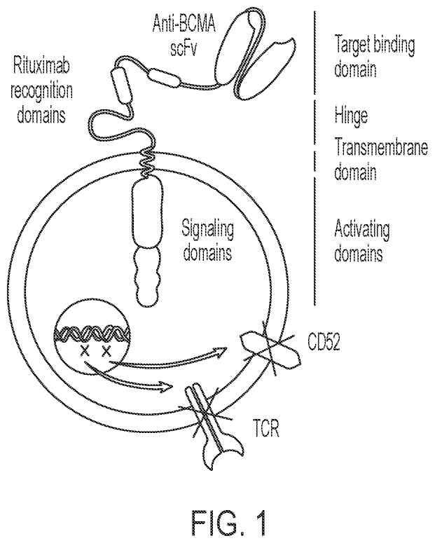

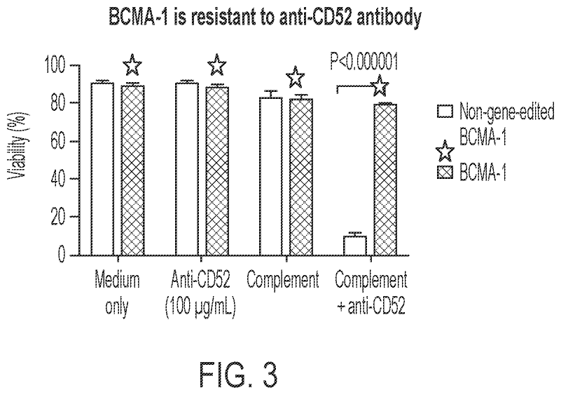

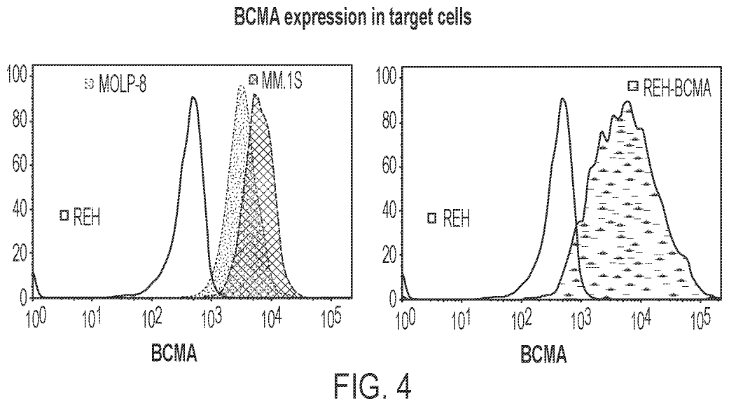

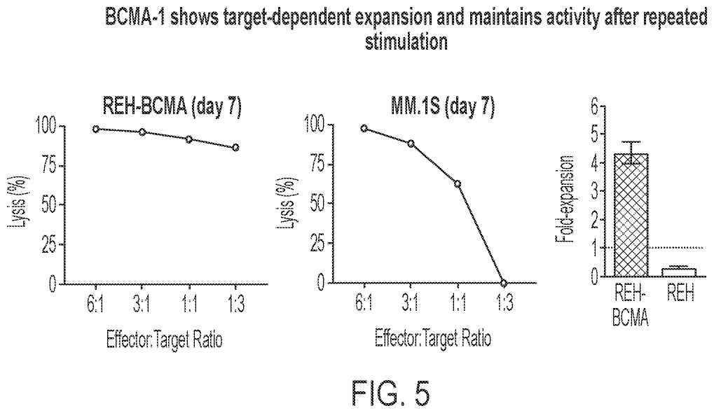

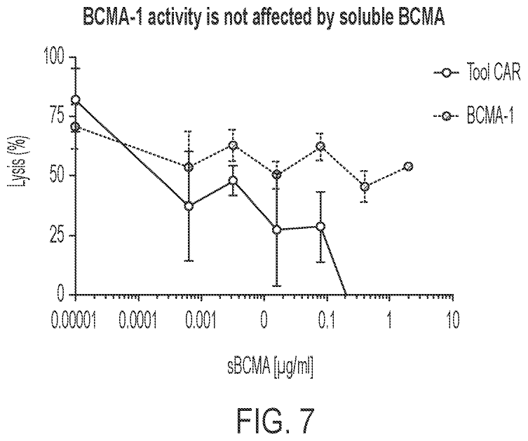

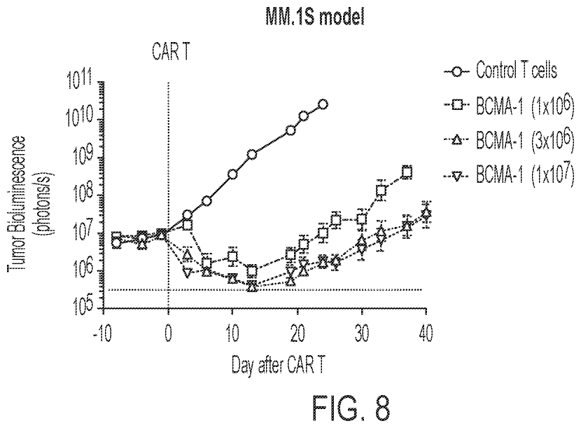

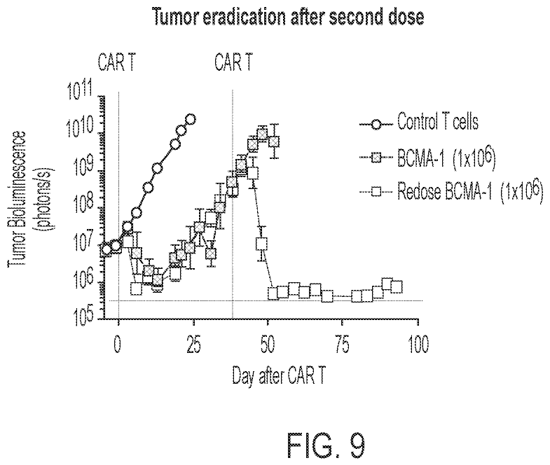

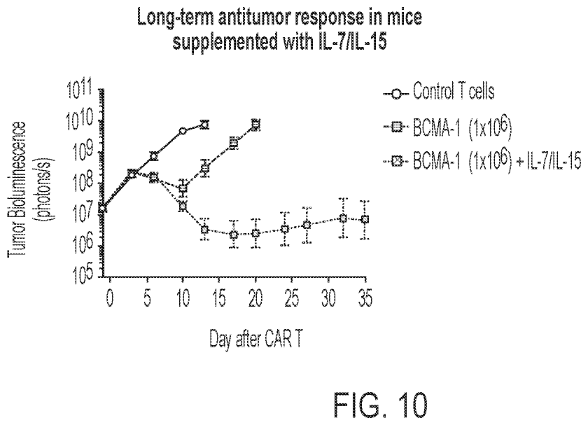

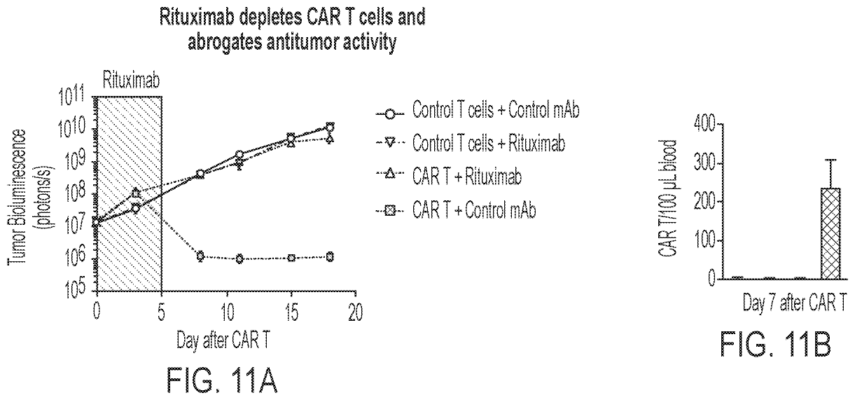

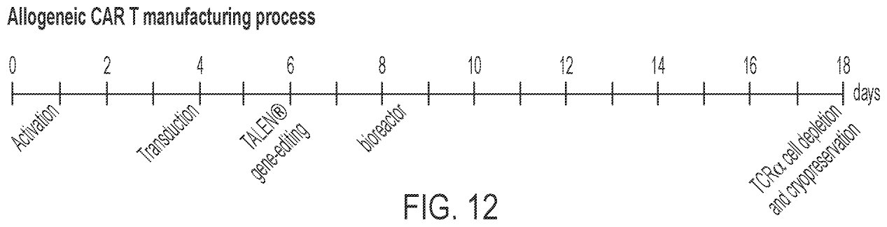

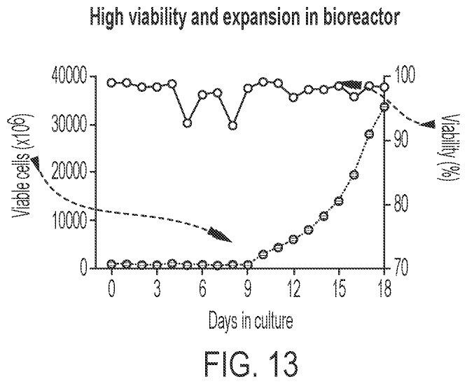

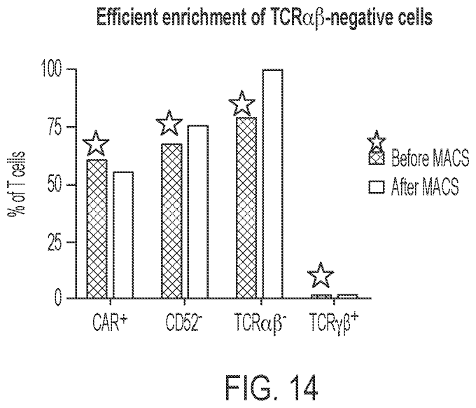

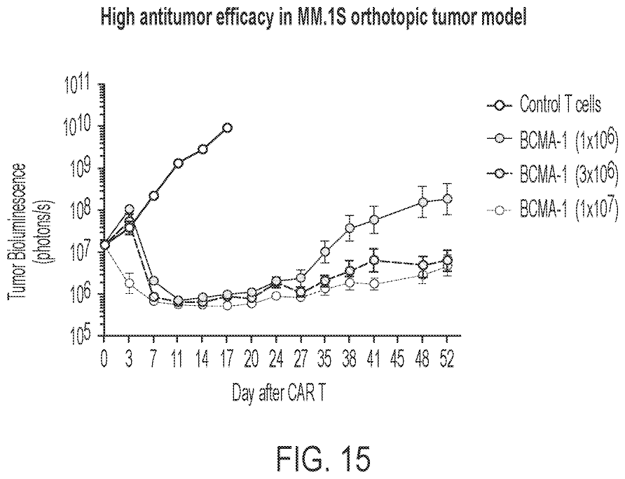

FIGS. 1-16 depict the generation and testing of BCMA-1. BCMA-1 is an allogeneic T-cell containing an integrated self-inactivating third generation, recombinant lentiviral vector that expresses a BCMA CAR. The BCMA CAR comprises a scFv, wherein the scFV of the CAR is P5A2 of Table 1. The scFV comprises a VH and a VL, wherein the VH comprises the amino acid sequence shown in SEQ ID NO: 33 and the VL comprises the amino acid sequence shown in SEQ ID NO: 34 The extracellular region of the CAR also comprises 2 mimotopes that confer recognition by rituximab.

The genotype of the BCMA CAR-T-cells is BCMA-CAR⁺_TCRαβ⁻_CD52^{+/-}. The cells can be formulated in a solution comprising 5% DMSO. In one embodiment, the cells are formulated as a suspension for infusion in a 1:1 mixture of CryoStor® Basal Solution and CryoStor®CS10 resulting in a 5% final concentration of dimethyl sulfoxide, and the resulting dosage strength of the formulation is 14×10\hat{ }6 BCMA-CAR+_TCRαβ−_CD52+/− T-cells/mL.

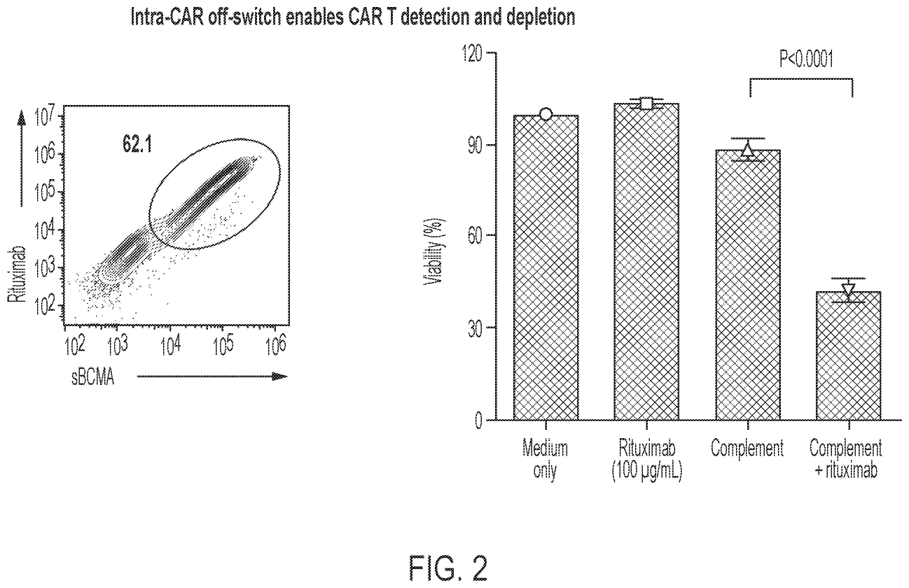

FIG. 2 shows the rituximab-mediated safety switch enables detection and depletion (with a rituximab antibody) of the BCMA-containing CAR-T cells of the disclosure. BCMA-1 cells were incubated with rabbit complement and rituximab. After 3 hours, cells were stained for CAR expression. The graph sows the percentage of live CAR+ cells (mean+/−SEM). (FIG. 2)

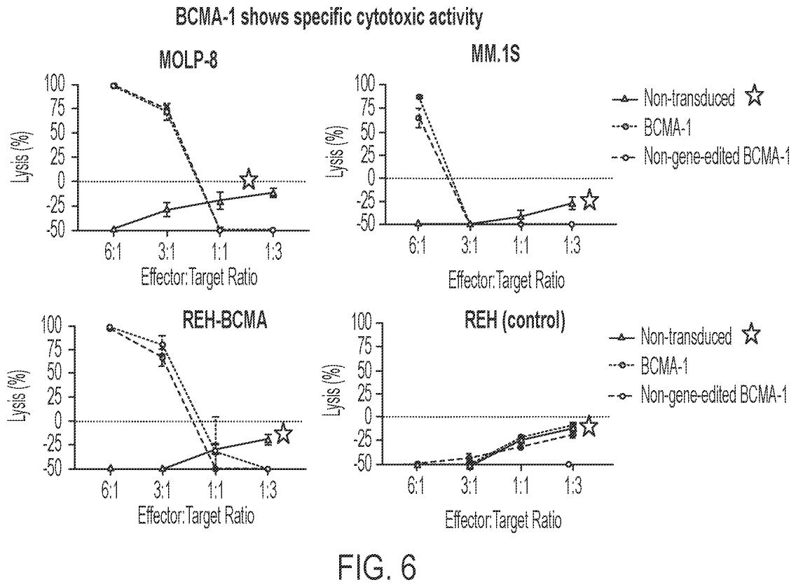

The cytotoxicity of BCMA-1 was tested against BCMA-expressing cell lines was assessed in vitro by co-culturing BCMA-1 effector cells with target cells stably expressing luciferase at increasing E:T ratios and measuring residual luciferase activity after 24 hours. BCMA-negative REH cells served as a control cell line. Compared to non-transduced control T cells (triangles), BCMA-1 (circles) exhibited dose-dependent cytotoxicity against BCMA-expressing cells but no apparent killing of control cells (REH). The killing activity of BCMA-1 and non-gene-edited BCMA-1 (open circles) was comparable. Graphs represent percentage of cell lysis relative to target cells cultured alone (FIG. 6). Results shown are mean+/−SEM of 3 donors. Negative cytotoxicity values (resulting from target cell growth or enhanced luciferase signal during the assay) were plotted as 0% lysis.

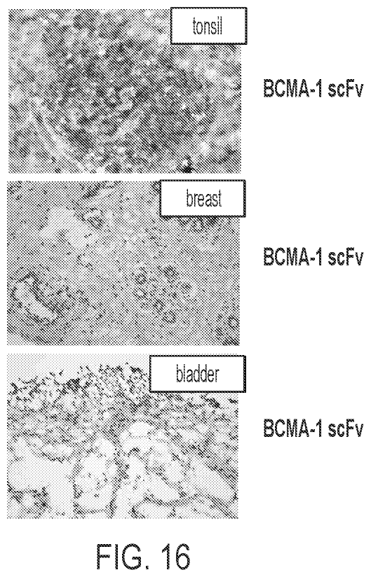

FIG. 16 shows that the scFV of BCMA-1 does not show off-target binding in tissue cross-reactivity studies, indicating the risk for off-target binding in a clinical setting to be low or non-existent. Testing was done in 13 human tissues. The extracellular domain of the CAR was fused to human IgG2dA D265A (mutation to prevent Fc binding). The method was developed for optimal staining on cell lines overexpressing BCMA. No staining observed in human tissues Result of immunohistochemistry staining in 9 human tissues—a triple signal amplification was carried out to increase signal. There was detection of expected signal in tonsil, lymph nodes, spleen tissues. There was no epithelial binding in breast, pancreas, fallopian tube, prostate, bladder tissues. Accordingly, the risk for unexpected binding is considered low or non-existent.

### Example 2: Phase 1 Study, Design A

FIG. 19 shows the outline for the Phase 1 Study (Design A) for treatment of refractory/relapsed MM. Escalating doses of 40×10\hat{ }6, 160×10\hat{ }6, and 320×10\hat{ }6 allogeneic CAR-T cells are studied (FIG. 20). The design of Design A includes a lymphodepletion phase of: fludarabine (flu) 30 mg/m2/day IV; cyclophosphamide (cy) 300 mg/m2/day IV; and CD52 antibody 13 mg/day IV, from 3 to 5 days prior to treatment; and a treatment phase (on day 0) which includes escalating doses from 20-480×10\hat{ }6 cells IV (for subjects ≥50 kg) or 7-360×10\hat{ }6 cells IV (for subjects <50 kg).

Criteria for inclusion may include one or more of the following:
Measurable MM after ≥3 prior MM regimens
   Induction+/−ASCT+/−maintenance is 1 regimen
   Received prior PI, IMiD and CD38 inhibition (unless contraindicated) with at least 2 continuous cycles of each regimen unless PD was best response
   Refractory to most recent prior regimen
ECOG PS 0-1
Adequate organ function
5 elimination half lives washout prior to LD
   4 weeks for mAb
BCMA expression may be used for patient selection.

The dose-banded levels for BCMA-1 Escalation in Phase 1 Design A is provided in Table 8.

TABLE 8

| | Dose (×10\hat{ }6 viable | Dose (×10\hat{ }6 viable |
|---|---|---|
| | CAR-T cells) | CAR-T cells) |
| | for subject whose | for subject whose |
| Dose Level | weight ≥50 kg | weight <50 kg |
| 1 (starting) | 40 | 20 |
| 2 | 120 | 80 |
| 3 | 360 | 240 |
| 4 | 480 | 360 |
| −1 | 20 | 7 or 14 |

Dose escalation will generally be governed by the 3+3 design; each dose level can receive cells from at least two different donors; up to five dose levels can be tested. The starting dose is noted as Dose Level 1 in Table 8, in some embodiments, a subject may receive a Dose level of −1 if indicated.

Redosing may be carried out, using BCMA CAR-T cells from a different donor, in a relapsed patient, using conditioning with, for example, 20 mg CD52 antibody conditioning.

### Example 3: Phase 1 Study, Design B

FIG. 19 shows the outline for the Phase 1 Study, Design B, for treatment of refractory/relapsed MM. The design of Design B includes a lymphodepletion phase of: fludarabine (flu) 30 mg/m2/day IV; cyclophosphamide (cy) 300 mg/m2/day IV; and CD52 antibody 13 mg/day IV, from 3 to 5 days prior to treatment; and a treatment phase (on day 0) which includes escalating doses from 20-480×10\hat{ }6 cells IV (for subjects ≥50 kg) or 7-360×10\hat{ }6 cells IV (for subjects <50 kg).

Criteria for inclusion may include one or more of the following:

1. Documented diagnosis of relapsed/refractory multiple myeloma (R/R MM) as defined by the IMWG consensus criteria for response and minimal residual disease assessment in multiple myeloma.

2. Subjects have measurable disease including one or more of the following criteria:
a. Serum M-protein 20.5 g/dL
b. Urine M-protein ≥200 mg/24 hours,

c. Involved serum free light chain (FLC) level ≥10 mg/dL (100 mg/L) provided serum FLC ratio is abnormal.

3. Patients have received at least ≥3 prior MM regimens:
a. Induction with or without hematopoietic stem cell transplant and with or without maintenance therapy is considered a single regimen.
b. Received prior proteasome inhibitor, immunomodulatory agent, and an anti-CD38 antibody (unless contraindicated) with at least 2 consecutive cycles of each regimen unless progressive disease was the best response to the regimen.
c. Refractory to the last treatment regimen.

4. Eastern Cooperative Oncology Group (ECOG) performance status of 0 or 1.

A cycle of treatment is considered as the combination of 1 lymphodepletion and 1 treatment period.

One goal of this study is to evaluate the MTD of BCMA-1, and/or establish its RP2D.

In some embodiments, the study includes 2 parts: dose escalation and dose expansion.

In the dose escalation part successive cohorts of patients may receive escalating doses of BCMA-1 in a 3+3 design. At each dose level, the first patient can be treated and observed for 28 days prior to treating subsequent patients with BCMA-1. All patients will generally be monitored closely for dose limiting toxicities (DLTs) during the first 28 days after BCMA-1 infusion. The target DLT rate for BCMA-1 is <33%. An intermediate dose level can be explored between DL1 and DL3 (Table 9). A dosing strategy using 2 different weight bands based on the variations in weight observed in the general population can be implemented. Patients weighing ≤50 kg can receive a dose 33% to 50% lower than that administered to patients weighing >50 kg. The provisional dose levels in BCMA-1 Escalation in Phase 1, Design B is provided in Table 9. Intermediate Dose level, Dose level 4, and Dose Level −1 can be administered as needed.

TABLE 9

| | Dose (×10\hat{ }6 viable | Dose (×10\hat{ }6 viable |
|---|---|---|
| | CAR-T cells) | CAR-T cells) |
| | for subject whose | for subject whose |
| Dose Level | weight >50 kg | weight ≤50 kg |
| 1 (starting) | 40 | 20 |
| 2 | 160 | 80 |
| 3 | 320 | 200 |
| Intermediate | 240 | 160 |
| 4 | 480 | 320 |
| −1 | 20 | 14 |

Dose escalation will generally be governed by the 3+3 design; each dose level can receive cells from at least two different donors; up to five dose levels can be tested. The starting dose is noted as Dose Level 1 in Table 9, in some embodiments, a subject may receive a Dose level of −1, a Dose level of 4, or an Intermediate Dose level (as displayed in Table 9) if indicated.

Accordingly, in Design B, BCMA-1 can be administered on Day 0 by intravenous (IV) infusion for approximately 5 minutes. Escalating doses of 40×10\hat{ }6, 160×10\hat{ }6, and 320×10\hat{ }6 allogeneic CAR-T cells can be studied for patients weighing >50 kg. The corresponding doses for patients weighing ≤50 kg are 20×10\hat{ }6, 80×10\hat{ }6, and 200×10\hat{ }6.

An anti-CD52 human IgG1 monoclonal antibody that recognizes the human CD52 antigen and can be used as a part of lymphodepletion regimen.

The anti-CD52 antibody can be administered on Day −5, Day −4, and Day −3 by IV infusion over 4 hours at a dose of 13 mg/day concomitantly with fludarabine (30 mg/m2/day) and/or cyclophosphamide (300 mg/m2/day), or the antibody alone. A lower dose at 10 mg/day is planned in case of toxicity. Fludarabine (30 mg/m2/day) can be administered for 3 days.

The overall duration of this Phase 1 study is approximately 48 months from first patient enrolled to last patient completed.

The dose expansion part can include additional cohorts added to the protocol, to characterize R2PD with the appropriate conditioning regimens of BCMA-1. Up to 3 cohorts of 12 patients in each cohort can be evaluated at the dose levels and conditioning regimens chosen based on the findings from the dose escalation.

The study can end when all patients treated with BCMA-1 have been followed for at least 24 months from the initial BCMA-1 infusion, have withdrawn consent for any further contact, been lost to follow-up, or died, unless the study is terminated by the sponsor earlier.

Redosing may be carried out, using BCMA CAR-T cells from a different donor, in a relapsed patient, using conditioning with, for example, 20 mg CD52 antibody conditioning.

Alternatively, BCMA-1 can be administered on Day 0 by intravenous (IV) infusion. Escalating doses of 40×10\hat{ }6, 160×10\hat{ }6, and 320×10\hat{ }6 allogeneic CAR-T cells without weight-banding are studied, as shown in Table 10 (Design C. See also FIG. 20.

TABLE 10

| | Dose (×10⁶ viable |
|---|---|
| Dose Level | CAR T cells) |
| 1 (starting) | 40 |
| 2 | 160 |
| 3 | 320 |
| Intermediate^{a} | 240 |
| 4^{a} | 480 |
| −1^{a} | 20 |

^{a}These dose levels will be administered as needed

### Example 4: Phase 2 Study

Phase 2 can involve testing an addition cohort of 6-12 subjects using the highest dose with acceptable toxicity from Phase 1 Design A, Design B, or Design C (either RP2D—the dose level producing around 20% of dose-limiting toxicity from Phase 1; or the dose level above the RP2D dose). Subjects may receive a CD52 antibody without flu/cy; the CD52 antibody may be administered at a dose of ˜40 mg (13 mg/day×days), ˜60 mg (e.g., 20 mg/day×3 days or 30 mg×2 days), or ˜90 mg (e.g., 30 mg/day×3 days) before the CAR-T cell treatment and repeated at 13 mg/day or 20 mg/day or 30 mg/day on Day 7, 14, and 21 after CAR-T cell treatment.

### Example 5 Assessment of BCMA Expression in Multiple Myeloma Cell Lines Exposed to the Gamma Secretase Inhibitor (GSI) PF-03084014

Nirogacestat is a selective, reversible, noncompetitive inhibitor of γ-secretase, a multiprotein protease that has been shown to activate Notch signaling. Nirogacestat was previously in development for solid tumors and hematologic malignancies and is currently in a Phase 3 trial for the treatment of desmoid tumors (NCT03785964). In this experiment, we examined the activity of PF-03084014 on the inhibition of gamma secretase cleavage of BCMA from the surface of multiple myeloma cell lines.

The BCMA-expressing multiple myeloma cell lines MM.1S and Molp-8 (ATCC), and the BCMA-negative acute lymphocytic leukemia cell line REH (ATCC) were expanded in RPMI medium containing L-glutamine and 10% FBS in a humidified CO₂ incubator set to 37 degrees. The gamma secretase inhibitor (GSI) PF-03084014 (nirogacestat in the hydrobromide salt form, Sigma-Aldrich, #PZ0298) was diluted in H₂O at mg/mL and further diluted in cell culture medium as needed. Cells were cultured in the presence of increasing concentrations of PF-03084014 or vehicle (control) in a humidified CO₂ incubator set to 37° C. for 4 hours or 24 hours. Cells were then harvested by centrifugation for 5 min at 400×g and stained with a phycoerythrin (PE)-labeled anti-human BCMA antibody (BioLegend, Cat #357504) for 30 min at 4° C. Samples were analyzed by flow cytometry using a Cytoflex flow cytometer (Beckman Coulter) and expression levels of BCMA (mean fluorescence intensity, MFI) were determined using FlowJo version 10.4.1. Fold-change in surface BCMA was calculated as the MFI value of GSI-treated cells divided by the MFI value of cells receiving vehicle control. Maximal (100%) inhibition of gamma-secretase activity was defined as the maximal fold-change in surface BCMA. To obtain half-maximal effective concentration (EC50) values, gMFI was plotted as a function of the logarithm of the concentration of PF-03084014. The data was then fitted using a non-linear regression (four-parameter logistic curve) function and EC50 was calculated using GraphPad.

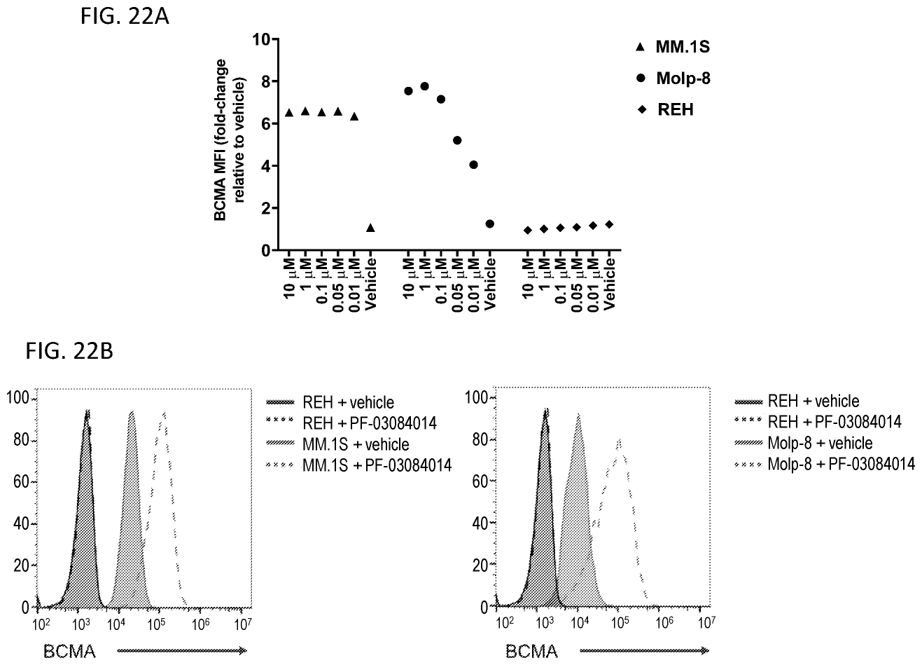

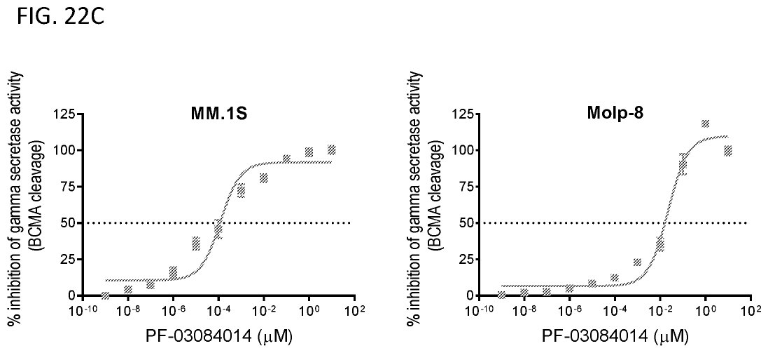

The results in FIG. 22A show increased levels of BCMA detected on the cell surface of BCMA positive MM.1S and Molp-8 cells in a dose-dependent manner following a 4-hour treatment with the GSI (PF-03084014). The dose-dependent increase of BCMA was not seen in the negative control cell line REH. FIG. 22B shows data from cells treated at the lowest dose of the GSI tested (10 nM of PF-03084014). The results in FIG. 22C show that treatment with PF-03084014 for 24 hours resulted in a dose-dependent inhibition of gamma secretase activity in both MM cell lines. The mean percent inhibition (f SEM) normalized to vehicle control is reported on the Y-axis and the concentration of PF-03084014 in μM (micromolar) is reported on the X-axis. The EC50 of PF-03084014 on gamma secretase determined using the MM.1S or Molp.8 cell line is 0.12 nM or 22.4 nM, respectively.

### Example 6 Assessment of Target-Dependent BCMA CAR-T Cell Proliferation in the Presence of the Gamma Secretase Inhibitor PF-03084014

A BCMA-negative acute lymphocytic leukemia cell line (REH) was genetically modified to overexpress BCMA with the use of a lentiviral vector encoding the human BCMA protein under regulatory control of the EF1alpha promoter (REH-BCMA cells). BCMA-overexpressing cells co-expressing luciferase and green fluorescent protein (GFP) were then created by transducing the cells with a Luc2AGFP/Blasticidin lentiviral vector following the manufacturer's recommendations. The exemplary BCMA CAR tested in the experiments comprises the VH amino acid sequence of SEQ ID NO: 33 and VL amino acid sequence of SEQ ID NO: 34). The BCMA CAR-T cells were generated and cryopreserved as previously described (Sommer et al., Molecular Therapy 2019, 27 (6): 1126-1138), thawed, and used in the assays as follows. Briefly, 5×10⁵ CAR⁺ T cells were co-cultured with 2×10⁶ REH-BCMA cells in 2.5 mL of target cell medium (RPMI with L-glutamine, 10% FBS) without IL-2 using G-Rex 24-well plates. PF-03084014 was added to cells at increasing concentrations whereas the control wells received vehicle. Every 2 days, 1×10⁶ of fresh REH-BCMA target cells were added into the same well and fresh cell culture medium containing PF-03084014 or vehicle was also added to a final volume of 3 mL and cells were returned to the incubator. On Day 5, cells were counted and the percentage of residual target cells and CAR-T cells were determined by flow cytometry analysis. Target cells can be identified as being GFP⁺ whereas CAR-T cells can be identified by staining with an anti-idiotype antibody conjugated to phycoerythrin (PE), which binds the BCMA CAR. Target-cell dependent proliferation/persistence in the presence or absence of PF-03084014 was determined by dividing the total number of CAR⁺ cells by the starting cell number (5×10⁵).

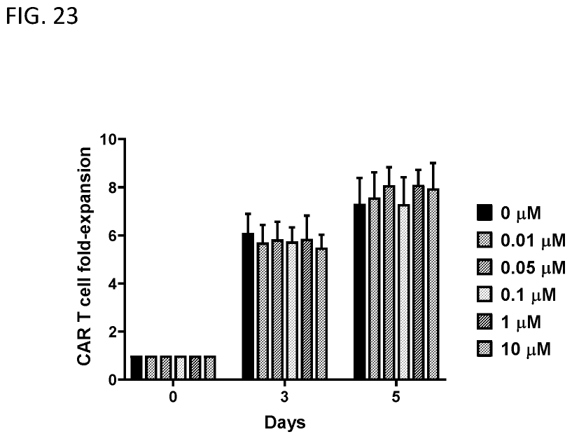

The data in FIG. 23 show that the gamma secretase inhibitor PF-03084014 did not negatively affect CAR-T cell expansion, even at the highest concentration of the inhibitor.

### Example 7 In Vitro Assessment of the Cytolytic Activity of BCMA CAR-T Cells in the Presence of the Gamma Secretase Inhibitor PF-03084014

The multiple myeloma cell lines MM.1S and Molp-8 and the BCMA-overexpressing REH cell line (REH-BCMA cells) were used in these assays. The target cell lines have been modified to constitutively express the luciferase gene which allows assessment of cell viability via luminescence. The cytotoxic activity of BCMA CAR-T cells was determined by measuring the reduction of luminescence signal from live target cells after 24 hour co-culture with BCMA CAR-T cells at increasing effector to target (E:T) ratios (1:3, 1:1, 3:1). Briefly, 2×10⁴ luciferase-expressing target cells were co-cultured with BCMA CAR-T cells at defined E:T ratios in target cell culture medium (RPMI with L-glutamine, 10% FBS) without IL-2. The GSI PF-03084014 was added to wells at increasing concentrations (0.01 μM to 10 μM) and cells were cultured in a humidified CO₂ incubator set to 37° C. for 24 hours. Following the incubation period, 100 μL of Bright-GLO reagent (Promega) were added and luminescence was read on a luminometer (Spectramax; Molecular Devices). Relative luminescence units (RLU) were converted to percentage of lysed target cells using the formula 100×[1−(RLUtest/RLUcontrol)]. Untreated target cells were used to determine RLU control.

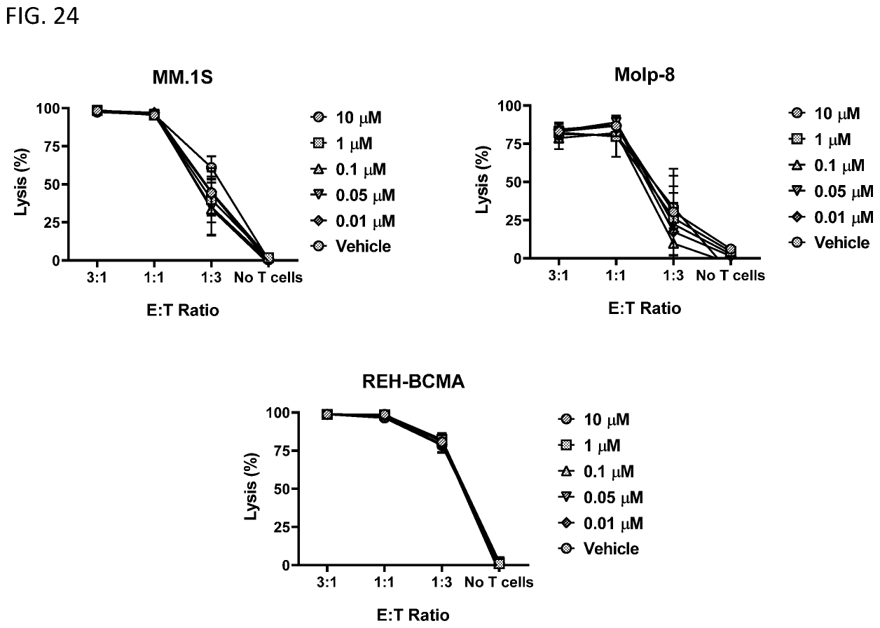

Although the gamma secretase inhibitor PF-03084014 increased cell surface levels of BCMA and did not negatively affect BCMA CAR-T cells expansion, the results in FIG. 24 indicate that, in this in vitro cytolytic assay, enhanced target cell killing was not observed. The slight variations in cell killing of MM.1S and Molp-8 cells at a low E:T ration (1:3) are not statistically significant. Without being limited to any specific mechanisms, the lack of effects of the GSI on BCMA-targeted killing shown in this experiment could be due to the high potency of the BCMA-CAR and the selection of established cell lines that are more homogenous in BCMA expression v. primary BCMA-positive cells in tumors that are less homogenous in BCMA expression.

In a separate experiment, cytolytic activities of BCMA CAR-T cells were evaluated after the target multiple myeloma cells were first pre-incubated with PF-03084014. FIG. 25A depicts the cell surface expression level of BCMA on the multiple myeloma cell line MM.1S and the BCMA negative control cell line A549 after a 24-hour culture in the presence of increasing concentrations of the gamma secretase inhibitor PF-03084014. The data is shown as mean fluorescence intensity (MFI) values of BCMA. FIG. 25B depicts the cytolytic activity of BCMA CAR T cells against MM.1S cells previously treated with PF-03084014 for 24 hours. In the cytolytic assay, BCMA CAR T cells and target cells were co-cultured at different effector to target (E:T) ratios for 24 hours and residual target cell viability was measured using bioluminescence. Results are shown as mean+/−SEM. Without wishing to be bound be particular mechanisms, the data in FIG. 25B show that enhanced cytolytic effect of BCMA CAR T cells were observed on target cells when the target cells were first pre-incubated with PF-03084014 to allow increased levels of BCMA.

The effects of the GSI on in vivo BCMA positive tumor cells (such as BCMA positive multiple myeloma) by BCMA CAR-T cells are investigated in clinical settings as exemplified below.

### Example 8: Combination Trial of BCMA CAR-T and Nirogacestat

The combination trial of BCMA CAR-T and nirogacestat is evaluated in relapsed/refractory multiple myeloma patients in cohort(s) under the protocol described herein. The proposed combination arms (or separate study) are anticipated to be studied in two cohorts of up to 12 patients each—one cohort consisting of patients who have not received any prior BCMA directed therapies, and the other cohort consisting of patients who have received a BCMA targeted therapy such a bispecific, ADC or autologous CAR-T. Objectives/endpoints include: safety; change in BCMA expression/antigen binding capacity; best overall response rate, CR/VGPR rate, MRD negative rate, change in soluble BCMA levels, and duration of response.

Eligible subjects will have relapsed/refractory multiple myeloma, with the following key eligibility criteria:

Inclusion:
Measurable disease (serum, urine, or FLC) per IMWG criteria
At least three prior lines of MM therapy, including a proteasome inhibitor, immunomodulatory agent, and anti-CD38 antibody (unless contraindicated), and refractory to the last treatment line
ECOG 0 or 1
Absence of donor (product)-specific anti-HLA antibodies
Adequate hematologic, renal, hepatic, pulmonary, and cardiac function

Exclusion:
Current or history of CNS involvement of myeloma or plasma cell leukemia
Clinically significant CNS disorder
Autologous stem cell transplant within the last 6 weeks, or any allogeneic stem cell transplant

Dose:

The dose of BCMA CAR-T cells and the conditioning regimen will be established from the phase 1 and Phase 2 studies. The dose of nirogacestat and schedule of administration pre- and post-BCMA CAR-T cells infusion will be evaluated.

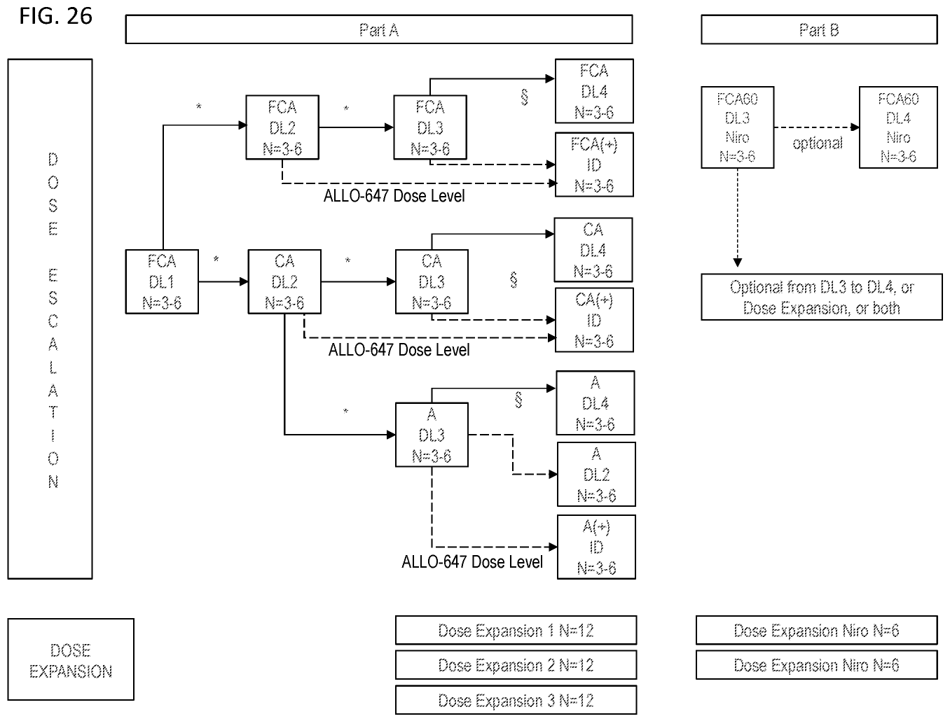

Exemplary dosing schedule is shown in FIG. 26 and the exemplary dosing escalation schema of BCMA-1 is shown in Table 10. Nirogacestat is administered after lymphodepletion regimen employing, e.g., fludarabine, cyclophosphamide and total 60 mg of alemtuzumab. Nirogacestat 100 mg BID PO is taken daily (twice per day by the mouth) starting on Day 0 (before BCMA-1 administration) through Day 41. At least the first dose of the twice daily dose of nirogacestat on Day 0 is taken before BCMA-1 administration. Nirogacestat can be taken with or without food. Nirogacestat is administered to patients receiving BCMA-1 at DL1, DL2, DL3 or DL4, preferably, DL3 or DL4.

The data presented in the instant disclosure show that nirogacestat increased the cell surface density of BCMA on multiple myeloma cell lines in a dose-dependent manner with an EC50 of 0.12 nM and 22.4 nM for the multiple myeloma cell lines MM.1S and Molp-8, respectively. See, e.g., FIG. 22C. In a Phase 1, dose-finding study (A8641014), a 100 mg BID dose was given to solid tumor patients, and the minimum concentration (Cmin) observed was 232 ng/mL or approximately 480 nM, approximately 20-fold higher than the higher EC50 value determined in the in vitro study. A dose of 100 mg BID nirogacestat is expected to maintain serum concentrations of nirogacestat at or above the levels required to prevent the cleavage of BCMA by gamma secretase, thereby leading to reduced soluble BCMA and increased membrane-bound BCMA.

At the proposed dose level of 100 mg BID in combination with BCMA CAR T cells (e.g., BCMA-1), nirogacestat is expected to have a safety profile at least as well tolerated as the 150 mg BID dose used in the solid tumor studies that have had durations of treatment and follow-up longer than 5 years.

The continuous dosing schedule and relatively long half-life of nirogacestat, is expected to provide adequate drug exposure for continued inhibition of gamma secretase, yielding sustained and rapid increases in membrane-bound BCMA and reduced levels of soluble BCMA over time.

Although the disclosed teachings have been described with reference to various applications, methods, and compositions, it will be appreciated that various changes and modifications can be made without departing from the teachings herein and the claims below. The foregoing examples are provided to better illustrate the disclosed teachings and are not intended to limit the scope of the teachings presented herein. While the present teachings have been described in terms of these exemplary embodiments, the skilled artisan will readily understand that numerous variations and modifications of these exemplary embodiments are possible without undue experimentation. All such variations and modifications are within the scope of the current teachings.

All references cited herein, including patents, patent applications, papers, text books, and the like, and the references cited therein, to the extent that they are not already, are hereby incorporated by reference in their entirety. In the event that one or more of the incorporated literature and similar materials differs from or contradicts this application, including but not limited to defined terms, term usage, described techniques, or the like, this application controls.

The foregoing description and Examples detail certain specific embodiments of the invention and describes the best mode contemplated by the inventors. It will be appreciated, however, that no matter how detailed the foregoing may appear in text, the invention may be practiced in many ways and the invention should be construed in accordance with the appended claims and any equivalents thereof.

1. A method of treating cancer in a subject comprising administering to the subject (a) at least one dose of chimeric antigen receptor (CAR)-T cells comprising CAR (BCMA CAR-T cells) and (b) a gamma secretase inhibitor.

2. The method of claim 1, wherein the gamma secretase inhibitor is Compound I having the structure: 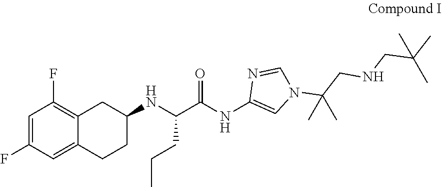 [SMILES: CCC[C@H](N[C@H]1CCc2cc(F)cc(F)c2C1)C(=O)Nc1cn(C(C)(C)CNCC(C)(C)C)cn1]
or a pharmaceutically acceptable salt thereof.

3. The method of claim 1, wherein the BCMA CAR-T cells comprise a CAR comprising an extracellular binding domain comprising a single chain Fv fragment (scFv), wherein the scFv comprises a heavy chain variable (VH) region and a light chain variable (VL) region, wherein the VH region comprises a VH complementary determining region 1 (VH CDR1), a VH complementary determining region 2 (VH CDR2), and a VH complementary determining region 3 (VH CDR3) and the VL region comprises a VL complementary determining region 1 (VL CDR1), a VL complementary determining region 2 (VL CDR2), and a VL complementary determining region 3 (VL CDR3), wherein:
(a) the VH CDR1 comprises the amino acid sequence of SEQ ID NO: 150, 151, or 152; the VH CDR2 comprises the amino acid sequence of SEQ ID NO: 153 or 154; the VH CDR3 comprises the amino acid sequence of SEQ ID NO: 155; the VL CDR1 comprises the amino acid sequence of SEQ ID NO: 209; the VL CDR2 comprises the amino acid sequence of SEQ ID NO: 221; and the VL CDR3 comprises the amino acid sequence of SEQ ID NO: 222;
(b) the VH CDR1 comprises the amino acid sequence of SEQ ID NO: 150, 151, or 152; the VH CDR2 comprises the amino acid sequence of SEQ ID NO: 187 or 188; the VH CDR3 comprises the amino acid sequence of SEQ ID NO: 155; the VL CDR1 comprises the amino acid sequence of SEQ ID NO: 249; the VL CDR2 comprises the amino acid sequence of SEQ ID NO: 221; and the VL CDR3 comprises the amino acid sequence of SEQ ID NO: 225;
(c) the VH CDR1 comprises the amino acid sequence of SEQ ID NO: 150, 151, or 152; the VH CDR2 comprises the amino acid sequence of SEQ ID NO: 165 or 166; the VH CDR3 comprises the amino acid sequence of SEQ ID NO: 155; the VL CDR1 comprises the amino acid sequence of SEQ ID NO: 226; the VL CDR2 comprises the amino acid sequence of SEQ ID NO: 221; and the VL CDR3 comprises the amino acid sequence of SEQ ID NO: 227;
(d) the VH CDR1 comprises the amino acid sequence of SEQ ID NO: 151, 156, or 157; the VH CDR2 comprises the amino acid sequence of SEQ ID NO: 159 or 162; the VH CDR3 comprises the amino acid sequence of SEQ ID NO: 161; the VL CDR1 comprises the amino acid sequence of SEQ ID NO: 251; the VL CDR2 comprises the amino acid sequence of SEQ ID NO: 252; and the VL CDR3 comprises the amino acid sequence of SEQ ID NO: 253;
(e) the VH CDR1 comprises the amino acid sequence of SEQ ID NO: 151, 156, or 157; the VH CDR2 comprises the amino acid sequence of SEQ ID NO: 190 or 191; the VH CDR3 comprises the amino acid sequence of SEQ ID NO: 161; the VL CDR1 comprises the amino acid sequence of SEQ ID NO: 262; the VL CDR2 comprises the amino acid sequence of SEQ ID NO: 252; and the VL CDR3 comprises the amino acid sequence of SEQ ID NO: 263;
(f) the VH CDR1 comprises the amino acid sequence of SEQ ID NO: 150, 151, or 152; the VH CDR2 comprises the amino acid sequence of SEQ ID NO: 154 or 169; the VH CDR3 comprises the amino acid sequence of SEQ ID NO: 155; the VL CDR1 comprises the amino acid sequence of SEQ ID NO: 271; the VL CDR2 comprises the amino acid sequence of SEQ ID NO: 221; and the VL CDR3 comprises the amino acid sequence of SEQ ID NO: 272;
(g) the VH CDR1 comprises the amino acid sequence of SEQ ID NO: 129, 130, or 131; the VH CDR2 comprises the amino acid sequence of SEQ ID NO: 139 or 140; the VH CDR3 comprises the amino acid sequence of SEQ ID NO: 134; the VL CDR1 comprises the amino acid sequence of SEQ ID NO: 217; the VL CDR2 comprises the amino acid sequence of SEQ ID NO: 210; and the VL CDR3 comprises the amino acid sequence of SEQ ID NO: 216;
(h) the VH CDR1 comprises the amino acid sequence of SEQ ID NO: 151, 156, or 157; the VH CDR2 comprises the amino acid sequence of SEQ ID NO: 158 or 159; the VH CDR3 comprises the amino acid sequence of SEQ ID NO: 155; the VL CDR1 comprises the amino acid sequence of SEQ ID NO: 209; the VL CDR2 comprises the amino acid sequence of SEQ ID NO: 221; and the VL CDR3 comprises the amino acid sequence of SEQ ID NO: 225; or
(i) the VH CDR1 comprises the amino acid sequence of SEQ ID NO: 129, 130, or 131; the VH CDR2 comprises the amino acid sequence of SEQ ID NO: 132 or 133; the VH CDR3 comprises the amino acid sequence of SEQ ID NO: 137; the VL CDR1 comprises the amino acid sequence of SEQ ID NO: 377; the VL CDR2 comprises the amino acid sequence of SEQ ID NO: 210; and the VL CDR3 comprises the amino acid sequence of SEQ ID NO: 214.

4-5. (canceled)

6. The method of claim 1, wherein the BCMA CAR-T cells comprise a CAR comprising an extracellular ligand-binding domain, a first transmembrane domain, and an intracellular signaling domain, wherein the extracellular domain comprises a scFv comprising a heavy chain variable (VH) region comprising a sequence shown in SEQ ID NO: 33, 72, 39, 76, 83, 92, 25, 112, or 8 of Table 1; and a light chain variable (VL) region comprising a sequence shown in SEQ ID NO: 34, 73, 40, 77, 84, 93, 18, 38, or 80 of Table 1, wherein the first transmembrane domain comprises a CD8α chain transmembrane domain, and wherein the intracellular signaling domain comprises a CD3ζ signaling domain and/or a 4-1BB signaling domain.

7. The method of claim 6, wherein the VH comprises SEQ ID NO: 33 and the VL comprises SEQ ID NO: 34; or
wherein the VH comprises SEQ ID NO: 112 and the VL comprises SEQ ID NO: 38.

8. (canceled)

9. The method of claim 1, wherein the BCMA CAR-T cells comprise a CAR comprising the amino acid sequence shown in SEQ ID NO: 344, SEQ ID NO: 418, or SEQ ID NO: 419.

10. The method of claim 9, wherein the CAR-T cells comprise a CAR comprising the amino acid sequence shown in SEQ ID NO: 344, and the CAR further comprises a CD20 epitope.

11.-12. (canceled)

13. The method of claim 1, wherein the BCMA CAR-T cells comprise a CAR comprising:
a CD8α signal peptide having the sequence of SEQ ID NO: 318; a VH region having the sequence of SEQ ID NO: 112; a GS linker having the sequence of SEQ ID NO: 333; a VL region having the sequence of SEQ ID NO: 38; a CD8α hinge having the sequence of SEQ ID NO: 320; a CD8α transmembrane domain having the sequence of SEQ ID NO: 322; a 4-1BB intracellular signaling domain having the sequence of SEQ ID NO: 323; and a CD3ζ intracellular signaling domain having the sequence of SEQ ID NO: 324;
a CD8α signal peptide having the sequence of SEQ ID NO: 318; a VH region having the sequence of SEQ ID NO: 112; a GS linker having the sequence of SEQ ID NO: 333; a VL region having the sequence of SEQ ID NO: 38; a CD20 epitope having the sequence of SEQ ID NO: 398; a CD8α hinge having the sequence of SEQ ID NO: 320; a CD8α transmembrane domain having the sequence of SEQ ID NO: 322; a 4-1BB intracellular signaling domain having the sequence of SEQ ID NO: 323; and a CD3ζ intracellular signaling domain having the sequence of SEQ ID NO: 324;
a CD8α signal peptide having the sequence of SEQ ID NO: 318; a VH region having the sequence of SEQ ID NO: 33; a GS linker having the sequence of SEQ ID NO: 333; a VL region having the sequence of SEQ ID NO: 34; a CD8α hinge having the sequence of SEQ ID NO: 320; a CD8α transmembrane domain having the sequence of SEQ ID NO: 322; a 4-1BB intracellular signaling domain having the sequence of SEQ ID NO: 323; and a CD3ζ intracellular signaling domain having the sequence of SEQ ID NO: 324; or
a CD8α signal peptide having the sequence of SEQ ID NO: 318; a VH region having the sequence of SEQ ID NO: 33; a GS linker having the sequence of SEQ ID NO: 333; a VL region having the sequence of SEQ ID NO: 34; a CD20 epitope having the sequence of SEQ ID NO: 398; a CD8α hinge having the sequence of SEQ ID NO: 320; a CD8α transmembrane domain having the sequence of SEQ ID NO: 322; a 4-1BB intracellular signaling domain having the sequence of SEQ ID NO: 323; and a CD3ζ intracellular signaling domain having the sequence of SEQ ID NO: 324.

14-16. (canceled)

17. The method of claim 1, wherein the gamma secretase inhibitor is administered to the subject before, concomitantly, or subsequently to the administering of the at least one dose of BCMA CAR-T cells to the subject.

18. The method of claim 1, wherein the cancer is multiple myeloma or relapsed/refractory multiple myeloma.

19. (canceled)

20. The method of claim 1, wherein the at least one dose of BCMA CAR-T cells is about 7×10\hat{ }6 cells/dose to about 480×10\hat{ }6 cells/dose.

21-23. (canceled)

24. The method of claim 1, wherein the subject receives a first lymphodepletion regimen prior to administration of the at least one dose of CAR T cells.

25. The method of claim 24, wherein the first lymphodepletion regimen comprises administering fludarabine, cyclophosphamide and/or an anti-CD52 antibody.

26.-27. (canceled)

28. The method of claim 1, wherein the subject is administered the gamma secretase inhibitor at a dose from about 20 mg to about 220 mg once or twice daily.

29. (canceled)

30. The method of claim 28, wherein the subject is administered the gamma secretase inhibitor once or twice daily for at least one week, at least two weeks, at least three weeks, at least four weeks, at least five weeks, at least six weeks, at least seven weeks, or at least eight weeks.

31-33. (canceled)

34. The method of claim 28, wherein the subject is administered the gamma secretase inhibitor at a dose of about 100 mg.

35.-36. (canceled)

37. The method of claim 1, wherein the subject is administered the gamma secretase inhibitor orally.

38. The method of claim 1, wherein the subject is administered the gamma secretase inhibitor in tablet, solution, or suspension form.

39.-40. (canceled)

41. The method of claim 1, wherein the treatment efficacy improves as compared to administering the BCMA CAR-T cells alone.

42-49. (canceled)

50. The method of claim 2, wherein Compound I is nirogacestat and the pharmaceutically acceptable salt is hydrobromide or dihydrobromide.

51. The method of claim 1, wherein the gamma secretase inhibitor is semagacestat.

52. The method of claim 1, wherein the gamma secretase inhibitor is BMS-986115.
